(19) United States
(12) Patent Application Publication
(10) Pub. No.: US 2025/0268016 A1
(43) Pub. Date: Aug. 21, 2025

(54) LIGHT-EMITTING ELEMENT, AMINE COMPOUND FOR THE LIGHT-EMITTING ELEMENT, AND DISPLAY DEVICE INCLUDING THE LIGHT-EMITTING ELEMENT

(71) Applicant: Samsung Display Co., Ltd., Yongin-si (KR)

(72) Inventors: Naoya SAKAMOTO, Yokohama (JP); Xiulan JIN, Yokohama (JP)

(73) Assignee: Samsung Display Co., Ltd., Yongin-si (KR)

(21) Appl. No.: 19/055,822

(22) Filed: Feb. 18, 2025

(30) Foreign Application Priority Data
Feb. 19, 2024 (KR) 10-2024-0023734

(51) Int. Cl. H10K 50/15; H10K 85/30; H10K 85/60; H10K 102/00

(52) U.S. Cl. CPC H10K 50/157; H10K 85/324; H10K 85/342; H10K 85/346; H10K 85/622; H10K 85/623; H10K 85/633; H10K 85/636; H10K 85/654; H10K 85/6572; H10K 85/6574; H10K 85/6576; H10K 2102/331

(57) ABSTRACT

Embodiments provide an amine compound, a light-emitting element that includes the amine compound, and a display device that includes the light-emitting element. The light-emitting element includes a first electrode, a second electrode disposed on the first electrode, an emission layer disposed between the first electrode and the second electrode, and a hole transport region disposed between the first electrode and the emission layer, wherein the hole transport region includes the amine compound. The amine compound is represented by Formula 1, which is described in the specification:

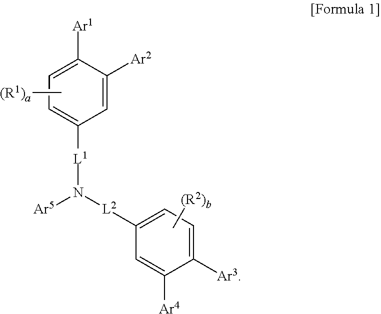

### CROSS-REFERENCE TO RELATED APPLICATION(S)

This application claims priority to and benefits of Korean Patent Application No. 10-2024-0023734 under 35 U.S.C. § 119, filed on Feb. 19, 2024, in the Korean Intellectual Property Office, the entire contents of which are incorporated herein by reference.

### BACKGROUND

### 1. Technical Field

The disclosure relates to a light-emitting element, an amine compound for the light-emitting element, and a display device including the light-emitting element.

### 2. Description of the Related Art

Ongoing development continues for an organic electroluminescence display device as an image display device. An organic electroluminescence display device includes a so-called self-luminescent light-emitting element in which holes and electrons respectively injected from a first electrode and a second electrode recombine in an emission layer, so that a luminescent material in the emission layer emits light to achieve display.

In the application of a light-emitting element to a display device, there is a persistent demand for a light-emitting element having improved efficiency and improved lifespan. Thus, continuous development is required on materials for a light-emitting element that are capable of stably achieving such characteristics.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light-emitting element having improved luminous efficiency and lifespan.

The disclosure also provides an amine compound that is a material for the light-emitting element having high efficiency and improved lifespan.

The disclosure also provides a display device that includes the light-emitting element having improved luminous efficiency and lifespan, thereby having excellent display quality.

According to embodiment, a light-emitting element may include a first electrode, a second electrode disposed on the first electrode, an emission layer disposed between the first electrode and the second electrode, and a hole transport region disposed between the first electrode and the emission layer, wherein the hole transport region includes an amine compound represented by Formula 1:

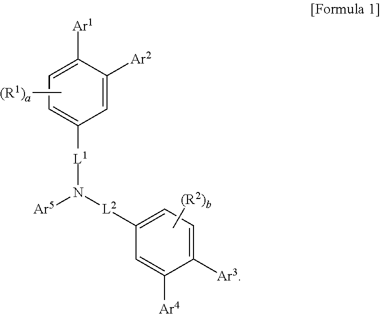

In Formula 1, L¹ and L² may each independently be a direct linkage, a substituted or unsubstituted arylene group having 6 to 30 ring-forming carbon atoms, or a substituted or unsubstituted heteroarylene group having 5 to 30 ring-forming carbon atoms; a and b may each independently be an integer from 0 to 3; R1 and R2 may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or bonded to an adjacent group to form a ring; Ar¹ to Ar⁴ may each independently be a substituted or unsubstituted aryl group having 6 to 30 ring-forming carbon atoms, or a substituted or unsubstituted heteroaryl group having 5 to 30 ring-forming carbon atoms; and Ar₅ may be a group represented by one of Formula 2-1 to Formula 2-4:

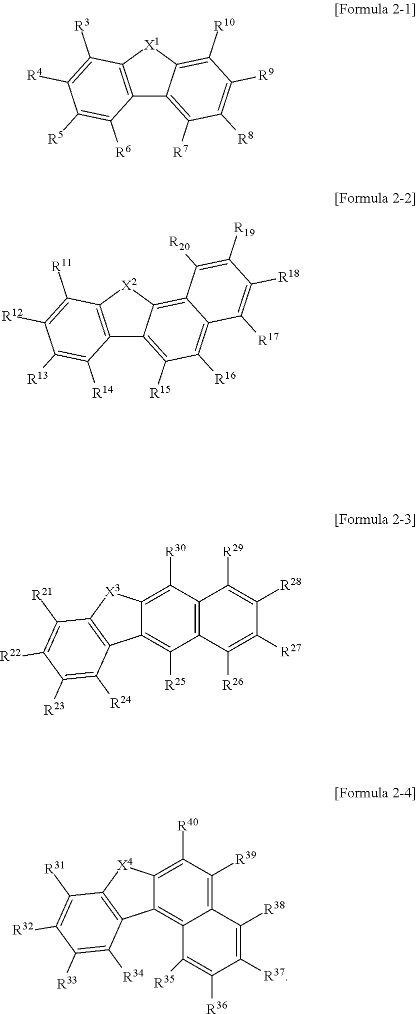

In Formula 2-1 to Formula 2-4 above, X¹ to X⁴ may each independently be O, S, or N(R⁴¹); R³ to R¹⁰ may each independently be a hydrogen atom, a deuterium atom, or a substituted or unsubstituted aryl group having 6 to 30 ring-forming carbon atoms; and R¹¹ to R⁴¹ may each independently be a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms, except that: in Formula 2-1, any one of R³ to R¹⁰ and R⁴¹ may be a position connected to Formula 1; in Formula 2-2, any one of R¹¹ to R²⁰ and R⁴¹ may be a position connected to Formula 1; in Formula 2-3, any one of R²¹ to R³⁰ and R⁴¹ may be a position connected to Formula 1; and in Formula 2-4, any one of R³¹ to R⁴¹ may be a position connected to Formula 1.

In an embodiment, the hole transport region may include at least one of a hole injection layer, a hole transport layer, an electron blocking layer, and an emission auxiliary layer; and the amine compound may be included in at least one of the hole injection layer, the hole transport layer, the electron blocking layer, and the emission auxiliary layer.

In an embodiment, the hole transport region may include a hole injection layer disposed on the first electrode, and a hole transport layer disposed on the hole injection layer; and the hole transport layer may include the amine compound.

In an embodiment, the amine compound may be represented Formula 3-1 or Formula 3-2:

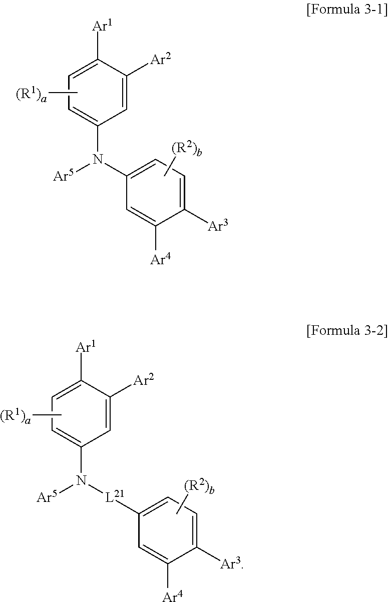

In Formula 3-2, L²¹ may be a substituted or unsubstituted phenylene group, a substituted or unsubstituted biphenylene group, a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group. In Formula 3-1 and Formula 3-2, Ar¹ to Ar⁵, R¹, R², a, and b may be the same as defined in Formula 1.

In an embodiment, the amine compound may be represented by Formula 4:

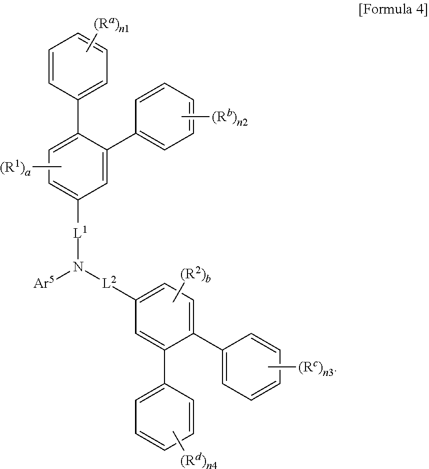

In Formula 4, n1 to n4 may each independently be an integer from 0 to 5,

In Formula 4, R^{a} to R^{d} may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring; and Ar⁵, R¹, R², L¹, L², a, and b may be the same as defined in Formula 1.

In an embodiment, in Formula 1, Ar¹ to Ar⁴ may each independently be a substituted or unsubstituted phenyl group, a substituted or unsubstituted naphthyl group, a substituted or unsubstituted phenanthryl group, a substituted or unsubstituted pyrenyl group, a substituted or unsubstituted triphenylene group, a substituted or unsubstituted carbazole group, a substituted or unsubstituted dibenzothiophene group, or a substituted or unsubstituted dibenzofuran group.

In an embodiment, at least one hydrogen atom of the amine compound may be substituted with a deuterium atom.

In an embodiment, when Ar⁵ is a group represented by Formula 2-1, Ar¹ to Art may each independently be a substituted or unsubstituted aryl group having 6 to 30 ring-forming carbon atoms.

In an embodiment, the amine compound may be selected from Compound Group 1, which is explained below.

According to an embodiment, an amine compound may be represented by Formula 1, which is explained herein.

In an embodiment, L¹ and L² may each independently be a direct linkage, a substituted or unsubstituted phenylene group, a substituted or unsubstituted biphenylene group a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group.

In an embodiment, R¹ and R² may each independently be a hydrogen atom or a deuterium atom.

In an embodiment, R³ to R⁴¹ may each independently be a hydrogen atom, a deuterium atom, a substituted or unsubstituted phenyl group, or a substituted or unsubstituted naphthyl group.

In an embodiment, when Ar⁵ is a group represented by Formula 2-1, Ar¹ to Ar⁴ may each independently be a substituted or unsubstituted phenyl group, a substituted or unsubstituted naphthyl group, a substituted or unsubstituted phenanthryl group, a substituted or unsubstituted pyrenyl group, or a substituted or unsubstituted triphenylene group.

According to an embodiment, a display device may include a circuit layer disposed on a base layer, and a display element layer disposed on the circuit layer and including a light-emitting element, wherein

the light-emitting element may include a first electrode, a second electrode disposed on the first electrode, an emission layer disposed between the first electrode and second electrode, and a hole transport region disposed between the first electrode and the emission layer; and the hole transport region may include an amine compound represented by Formula 1, which is explained herein.

In an embodiment, the display device may further include an optical control layer that includes a quantum dot.

In an embodiment, the display device may further include a color filter layer disposed on the optical control layer, wherein the color filter layer may include a first filter that transmits red light, a second filter that transmits green light, and a third filter that transmits blue light.

In an embodiment, the display device may be a television set, a monitor, a billboard, a personal computer, a laptop computer, a personal digital terminal, a display device for a vehicle, a game console, a portable electronic apparatus, or a camera

It is to be understood that the embodiments above are described in a generic and explanatory sense only and not for the purposes of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display device according to an embodiment;

FIG. 2 is a schematic cross-sectional view of a portion of a display device according to an embodiment;

FIG. 3 is a schematic cross-sectional view of a light-emitting element according to an embodiment;

FIG. 4 is a schematic cross-sectional view of a light-emitting element according to an embodiment;

FIG. 5 is a schematic cross-sectional view of a light-emitting element according to an embodiment;

FIG. 6 is a schematic cross-sectional view of a light-emitting element according to an embodiment;

FIG. 7 is a schematic cross-sectional view of a light-emitting element according to an embodiment;

FIG. 8 is a schematic cross-sectional view of a display device according to an embodiment;

FIG. 9 is a schematic cross-sectional view of a display device according to an embodiment;

FIG. 10 is a schematic cross-sectional view of a display device according to an embodiment;

FIG. 11 is a schematic cross-sectional view of a display device according to an embodiment; and

FIG. 12 is a schematic diagram of an interior of a vehicle in which a display device according to an embodiment is disposed.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for case of description and for clarity. Like reference numbers and reference characters refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for case of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within +20%, +10%, or +5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may described a group that is substituted or unsubstituted with at least one substituent selected from the group consisting of a deuterium atom, a halogen atom, a cyano group, a nitro group, an amino group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the term “bonded to an adjacent group to form a ring” may refer to a group that is bonded to an adjacent group to form a substituted or unsubstituted hydrocarbon ring or a substituted or unsubstituted heterocycle. A hydrocarbon ring may be aliphatic or aromatic. A heterocycle may be aliphatic or aromatic. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may be interpreted as a substituent that is substituted for an atom which is directly linked to an atom substituted with a corresponding substituent, as another substituent that is substituted for an atom which is substituted with a corresponding substituent, or as a substituent that is sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, and an iodine atom.

In the specification, an alkyl group may be linear or branched. The number of carbon atoms in an alkyl group may be 1 to 50, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-methylhexadecyl group, a 2-butylhexadecyl group, a 2-hexylhexadecyl group, a 2-octylhexadecyl group, an n-heptadecyl group, an n-octadecyl group, an n-nonadecyl group, an n-icosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in a cycloalkyl group may be 3 to 50, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not particularly limited, and may be 2 to 30, 2 to 20, or 2 to 10. Examples of an alkenyl group may include a vinyl group, a 1-butenyl group, a 1-pentenyl group, a 1,3-butadienyl, a styrenyl group, a styryl vinyl group, etc., but embodiments are not limited thereto.

In the specification, an alkynyl group may be a hydrocarbon group that includes at least one carbon-carbon triple bond in the middle or at a terminus of an alkyl group having 2 or more carbon atoms. An alkynyl group may be linear or branched. The number of carbon atoms in an alkynyl group is not particularly limited, and may be 2 to 30, 2 to 20, or 2 to 10. Examples of an alkynyl group may include an ethynyl group, a propynyl group, etc., but embodiments are not limited thereto.

In the specification, a hydrocarbon ring group may be any functional group or substituent derived from an aliphatic hydrocarbon ring. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of a substituted fluorenyl group may include the groups shown below. However, embodiments are not limited thereto.

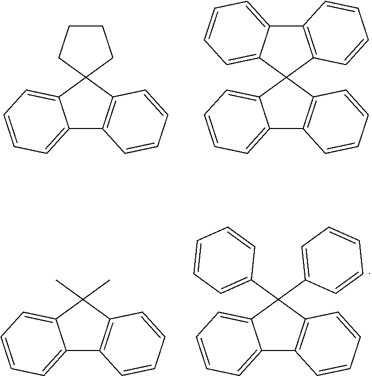

In the specification, a heterocyclic group may be any functional group or substituent derived from a ring that includes at least one of B, O, N, P, Si, S, or Se as a heteroatom. A heterocyclic group may be aliphatic or aromatic. An aromatic heterocyclic group may be a heteroaryl group. An aliphatic heterocycle and an aromatic heterocycle may each independently be monocyclic or polycyclic.

If a heterocyclic group includes two or more heteroatoms, the two or more heteroatoms may be the same as or different from each other. The number of ring-forming carbon atoms in a heterocyclic group may be 2 to 30, 2 to 20, or 2 to 10.

Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of an aryl group may be applied to an arylene group, except that an arylene group is a divalent group. In the specification, the above description of a heteroaryl group may be applied to a heteroarylene group, except that the heteroarylene group is a divalent group.

In the specification, a silyl group may be an alkylsilyl group or an arylsilyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in a carbonyl group is not particularly limited, and may be 1 to 40, 1 to 30, or 1 to 20. For example, a carbonyl group may have one of the following structures, but embodiments are not limited thereto.

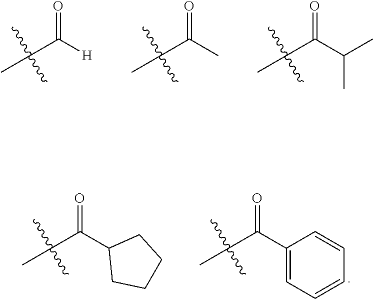

In the specification, the number of carbon atoms in a sulfinyl group or a sulfonyl group is not particularly limited, and may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkylthio group or an arylthio group. A thio group may be a sulfur atom that is bonded to an alkyl group or to an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are not limited thereto.

In the specification, an oxy group may be an oxygen atom that is bonded to an alkyl group or to an aryl group as defined above. An oxy group may be an alkoxy group or an aryl oxy group. An alkoxy group may be linear, branched, or cyclic. The number of carbon atoms in an alkoxy group is not particularly limited, and may be, for example, 1 to 20 or 1 to 10. Examples of an oxy group may include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, etc., but embodiments are not limited thereto.

In the specification, a boron group may be a boron atom that is bonded to an alkyl group or to an aryl group as defined above. A boron group may be an alkyl boron group or an aryl boron group. Examples of a boron group may include a dimethylboron group, a t-butyldimethylboron group, a diphenylboron group, a phenylboron group, etc., but embodiments are not limited thereto.

In the specification, the number of carbon atoms in an amine group is not particularly limited, and may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, a triphenylamine group, etc., but embodiments are not limited thereto.

In the specification, an alkyl group within an alkylthio group, an alkylsulfoxy group, an alkylaryl group, an alkylamino group, an alkyl boron group, an alkyl silyl group, or an alkyl amine group may be the same as an example of an alkyl group as described above.

In the specification, an aryl group within an aryloxy group, an arylthio group, an arylsulfoxy group, an arylamino group, an arylboron group, an arylsilyl group, or an arylamine group may be the same as an example of an aryl group as described above.

In the specification, a direct linkage may be a single bond.

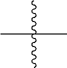

In the specification, the symbols

and —* each represent a bond to a neighboring atom in a corresponding formula or moiety.

Hereinafter, embodiments will be described with reference to the accompanying drawings.

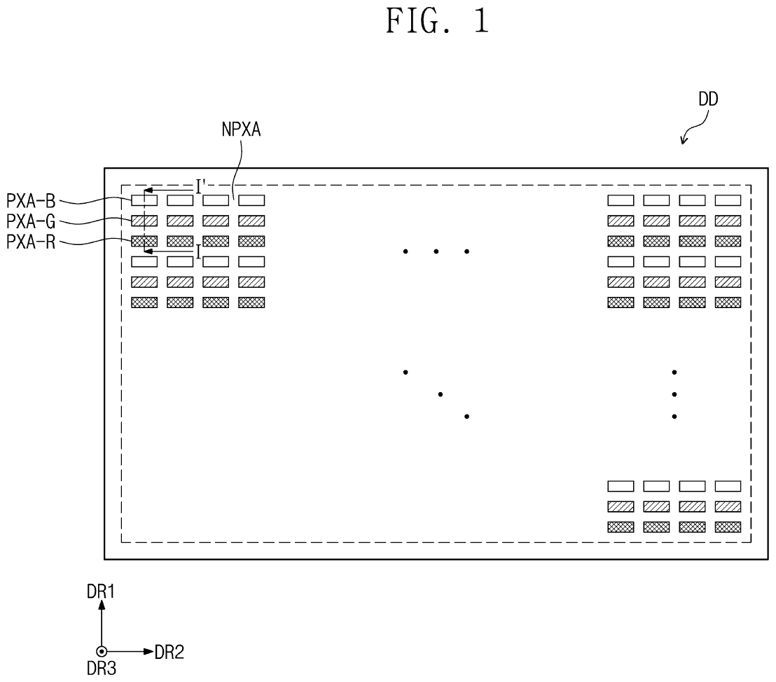

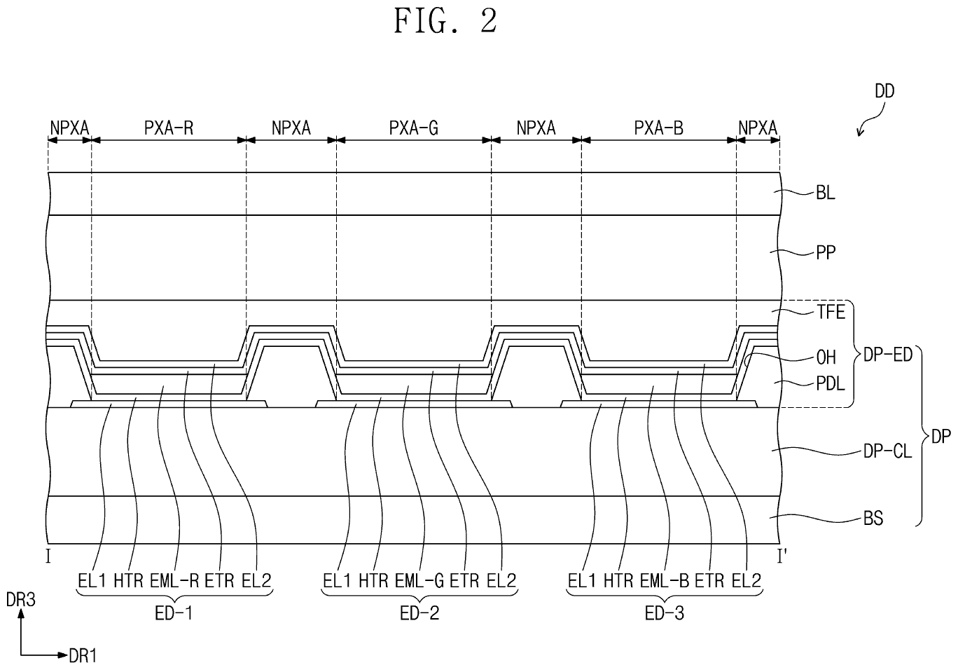

FIG. 1 is a schematic plan view of a display device DD according to an embodiment. FIG. 2 is a schematic cross-sectional view of the display device DD. FIG. 2 is a schematic cross-sectional view of a portion of the display device DD taken along virtual line I-I′ of FIG. 1.

The display device DD may include a display panel DP and an optical layer PP disposed on the display panel DP. The display panel DP includes light-emitting elements ED-1, ED-2, and ED-3. The display device DD may include multiples of each of the light-emitting elements ED-1, ED-2, and ED-3. The optical layer PP may be disposed on the display panel DP to control light that is reflected at the display panel DP from an external light. The optical layer PP may include, for example, a polarization layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between a display element layer DP-ED and the base substrate BL. The filling layer (not shown) may be an organic material layer. The filling layer (not shown) may include at least one of an acrylic-based resin, a silicone-based resin, and an epoxy-based resin.

The display panel DP may include a base layer BS, a circuit layer DP-CL provided on the base layer BS, and the display element layer DP-ED. The display element layer DP-ED may include a pixel defining film PDL, light-emitting elements ED-1, ED-2, and ED-3 disposed between portions of the pixel defining film PDL, and an encapsulation layer TFE disposed on the light-emitting elements ED-1, ED-2, and ED-3.

The base layer BS may provide a base surface on which the display element layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light-emitting elements ED-1, ED-2, and ED-3 of the display element layer DP-ED.

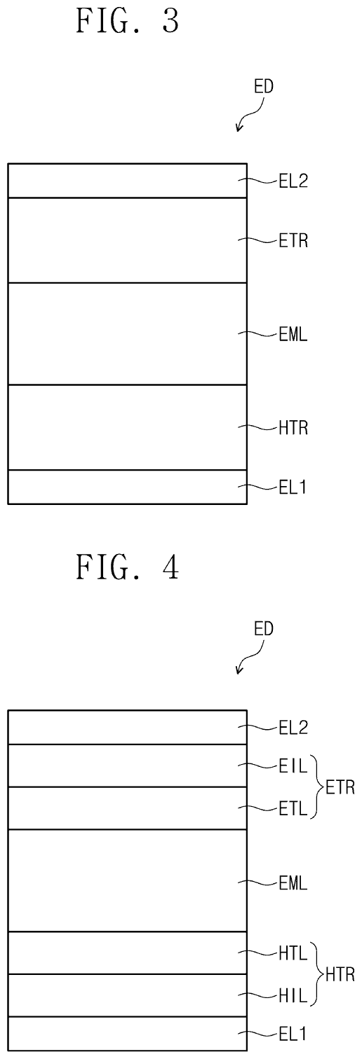

The light-emitting elements ED-1, ED-2, and ED-3 may each have a structure of a light-emitting element ED of an embodiment according to any of FIGS. 3 to 6, which will be described later. The light-emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light-emitting elements ED-1, ED-2, and ED-3 are disposed in openings OH defined in the pixel defining film PDL, and the hole transport region HTR, the electron transport region ETR, and the second electrode EL2 are each provided as a common layer for the light-emitting elements ED-1, ED-2, and ED-3. However, embodiments are not limited thereto. Although not shown in FIG. 2, the hole transport region HTR and the electron transport region ETR may each be provided by being patterned in the openings OH defined in the pixel defining film PDL. For example, in an embodiment, the hole transport region HTR, the emission layers EML-R, EML-G, and EML-B, and the electron transport region ETR of the light-emitting elements ED-1, ED-2, and ED-3 in an embodiment may each be provided by being patterned through an inkjet printing method.

The encapsulation layer TFE may cover the light-emitting elements ED-1, ED-2, and ED-3. The encapsulation layer TFE may seal the display element layer DP-ED. The encapsulation layer TFE may be a thin film encapsulation layer. The encapsulation layer TFE may be formed of a single layer or of multiple layers. The encapsulation layer TFE may include at least one insulation layer. The encapsulation layer TFE according to an embodiment may include at least one inorganic film (hereinafter, an encapsulation-inorganic film). In an embodiment, the encapsulation layer TFE may include at least one organic film (hereinafter, an encapsulation-organic film) and at least one encapsulation-inorganic film.

The encapsulation-inorganic film protects the display element layer DP-ED from moisture and/or oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region that emits light respectively generated by the light emitting elements ED-1, ED-2, and ED-3. The light emitting regions PXA-R, PXA-G, and PXA-B may be spaced apart from each other in a plan view.

The light emitting regions PXA-R, PXA-G, and PXA-B may be regions that are separated from each other by the pixel defining film PDL. The non-light emitting regions NPXA may be areas between the adjacent light emitting regions PXA-R, PXA-G, and PXA-B, and which may correspond to the pixel defining film PDL. In an embodiment, the light emitting regions PXA-R, PXA-G, and PXA-B may each correspond to a pixel. The pixel defining film PDL may separate the light emitting elements ED-1, ED-2, and ED-3. The emission layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be arranged into groups according to the color of light generated from the light emitting elements ED-1, ED-2, and ED-3. In the display device DD according to an embodiment illustrated in FIGS. 1 and 2, three light emitting regions PXA-R, PXA-G, and PXA-B, which respectively emit red light, green light, and blue light, are illustrated as an example. For example, the display device DD may include a red light emitting region PXA-R, a green light emitting region PXA-G, and a blue light emitting region PXA-B, which are distinct from each other.

In the display device DD according to an embodiment, the light-emitting elements ED-1, ED-2, and ED-3 may emit light having wavelengths that are different from each other. For example, in an embodiment, the display device DD may include a first light-emitting element ED-1 that emits red light, a second light-emitting element ED-2 that emits green light, and a third light-emitting element ED-3 that emits blue light. For example, the red light-emitting region PXA-R, the green light-emitting region PXA-G, and the blue light-emitting region PXA-B of the display device DD may respectively correspond to the first light-emitting element ED-1, the second light-emitting element ED-2, and the third light-emitting element ED-3.

However, embodiments are not limited thereto, and the first to third light-emitting elements ED-1, ED-2, and ED-3 may emit light in a same wavelength range or at least one light-emitting element may emit light in a wavelength range that is different from the remainder. For example, the first to third light-emitting elements ED-1, ED-2, and ED-3 may each emit blue light.

The light-emitting regions PXA-R, PXA-G, and PXA-B in the display device DD according to an embodiment may be arranged in a stripe configuration. Referring to FIG. 1, the red light-emitting regions PXA-R, the green light-emitting regions PXA-G, and the blue light-emitting regions PXA-B may be respectively arranged along a second directional axis DR2. In another embodiment, the red light-emitting region PXA-R, the green light-emitting region PXA-G, and the blue light-emitting region PXA-B may be arranged in this repeating order along a first directional axis DR1.

FIGS. 1 and 2 illustrate that the light-emitting regions PXA-R, PXA-G, and PXA-B all have a similar area, but embodiments are not limited thereto. In an embodiment, the light-emitting regions PXA-R, PXA-G, and PXA-B may be different in size or shape from each other, according to a wavelength range of emitted light. The areas of the light-emitting regions PXA-R, PXA-G, and PXA-B may be areas in a plan view that are defined by the first directional axis DR1 and the second directional axis DR2. A third directional axis DR3 may be perpendicular to a plane defined by the first directional axis DR1 and the second directional axis DR2.

An arrangement of the light-emitting regions PXA-R, PXA-G, and PXA-B is not limited to the configuration illustrated in FIG. 1, and the order in which the red light-emitting region PXA-R, the green light-emitting region PXA-G, and the blue light-emitting region PXA-B are arranged may be provided in various combinations, according to the display quality characteristics that are required for the display device DD. For example, the light-emitting regions PXA-R, PXA-G, and PXA-B may be arranged in a pentile configuration (such as PenTile®) or in a diamond configuration (such as Diamond Pixel®).

The areas of the light-emitting regions PXA-R, PXA-G, and PXA-B may be different in size from each other. For example, in an embodiment, an area of a green light-emitting region PXA-G may be smaller than an area of a blue light-emitting region PXA-B, but embodiments are not limited thereto.

Hereinafter, FIG. 3 to FIG. 7 are each a schematic cross-sectional view of a light-emitting element ED according to an embodiment. Embodiments provide a light-emitting element ED that may include a first electrode EL1, a hole transport region HTR, an emission layer EML, an electron transport region ETR, and a second electrode EL2, which may be stacked in that order.

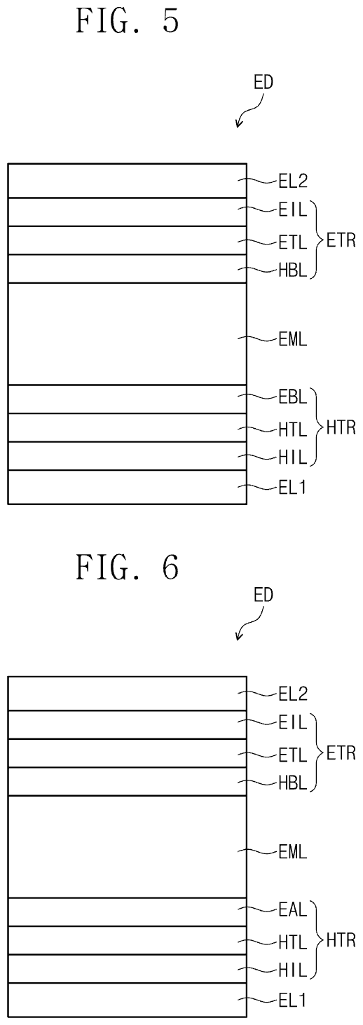

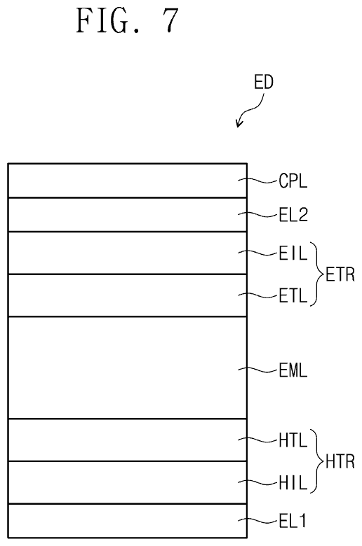

In comparison to FIG. 3, FIG. 4 is a schematic cross-sectional view of a light-emitting element ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 is a schematic cross-sectional view of a light-emitting element ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and hole blocking layer HBL. In comparison to FIG. 3, FIG. 6 is a schematic cross-sectional view of a light-emitting element ED according to an embodiment, in which a hole transport region HTR includes a hole injection layer HIL, a hole transport layer HTL, and an emission auxiliary layer EAL, and an electron transport region ETR includes an electron injection layer EIL, an electron transport layer ETL, and a hole blocking layer HBL. In comparison to FIG. 4, FIG. 7 is a schematic cross-sectional view of a light-emitting element ED according to an embodiment that includes a capping layer CPL disposed on a second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, and a mixture thereof.

If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In another embodiment, the first electrode EL1 may have a multilayered structure that includes a reflective film or a transflective film formed of the above-described materials, and a transparent conductive film formed of ITO, IZO, ZnO, ITZO, etc. For example, the first electrode EL1 may have a three-layered structure of ITO/Ag/ITO, but embodiments are not limited thereto. In an embodiment, the first electrode EL1 may include the above-described metal materials, combinations of at least two of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR may be provided on the first electrode EL1. The hole transport region HTR may be disposed between the first electrode EL1 and the emission layer EML.

The hole transport region HTR may include at least one of a hole injection layer HIL, a hole transport layer HTL, an emission auxiliary layer EAL, and an electron blocking layer EBL. The emission auxiliary layer EAL may be referred to as a buffer layer. A thickness of the hole transport region HTR may be, for example, in a range of about 50 Å to about 15,000 Å.

The hole transport region HTR may have a structure consisting of a layer consisting of a single material, a structure consisting of a layer including different materials, or a structure including multiple layers including different materials.

In embodiments, the hole transport region HTR may have a single-layered structure of a hole injection layer HIL or a hole transport layer HTL, or may have a single-layered structure that includes a hole injection material and a hole transport material. In embodiments, the hole transport region HTR may have a single-layered structure formed of different materials, or may have a structure in which a hole injection layer HIL/hole transport layer HTL, a hole injection layer HIL/hole transport layer HTL/emission auxiliary layer EAL, a hole injection layer HIL/emission auxiliary layer EAL, a hole transport layer HTL/emission auxiliary layer EAL, or a hole injection layer HIL/hole transport layer HTL/electron blocking layer EBL are stacked in its respective stated order from the first electrode EL1, but embodiments are not limited thereto. In an embodiment, the hole transport layer HTL may have a single-layered structure or a multilayered structure.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The light-emitting element ED may include an amine compound according to an embodiment in the hole transport region HTR. In an embodiment, at least one of the hole injection layer HIL, the hole transport layer HTL, the electron-blocking layer EBL, and the emission auxiliary layer EAL may each independently include an amine compound according to an embodiment. For example, in the light-emitting element ED, the hole transport layer HTL may include an amine compound according to an embodiment.

The amine compound according to an embodiment may be a monoamine compound that contains no additional amine substituent. In the amine substituent, one, two, or three hydrogen atoms in NH₃ may be substituted with a hydrocarbyl group and may include an alkyl amine group and an aryl group.

The amine compound according to an embodiment may include first, second, and third substituents. The amine compound according to an embodiment may include a terphenyl derivative connected to a nitrogen atom (N) as a first substituent and a second substituent. For example, the amine compound according to an embodiment may include two terphenyl derivatives, each connected to a nitrogen atom of an amine group. The amine compound according to an embodiment may include a substituted or unsubstituted ortho-terphenyl derivative directly bonded to a nitrogen atom (N) as a first substituent and as a second substituent, or the first substituent and the second substituent may be bonded to a nitrogen atom via a linking moiety. In the amine compound according to an embodiment, the first substituent and the second substituent may be the same as or different from each other. The amine compound according to an embodiment may further include a dibenzoheterole derivative connected to a nitrogen atom (N) as a third substituent. The amine compound according to an embodiment may include a substituted or unsubstituted dibenzoheterole group or a substituted or unsubstituted benzonaphtoheterole group, which is directly connected to a nitrogen atom (N) as the third substituent.

In the amine compound according to an embodiment, the substituted or unsubstituted ortho-terphenyl derivative may be directly bonded to a nitrogen atom at a cl position, as illustrated in Formula A below, or may be bonded to a nitrogen atom via a linking moiety. For example, the first substituent and the second substituent may each be directly bonded to a nitrogen atom at the cl position or may each be bonded to a nitrogen atom via a linking moiety. In the specification, the ortho-terphenyl derivative that is a moiety bonded to a nitrogen atom of the amine compound may be referred to as a 3,4-substituted phenyl group. In the ortho-terphenyl derivative represented by Formula A, Ara and Art may each independently be a substituted or unsubstituted aryl group, or a substituted or unsubstituted heteroaryl group. Ara may correspond to each of Ar¹ and Ar³, which will be described later, and Art may correspond to each of Ar² and Ar⁴, which will be described later.

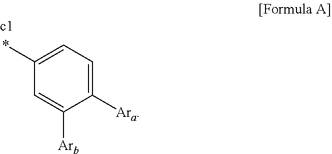

In the amine compound according to an embodiment, the third substituent may be a substituted or unsubstituted dibenzoheterole group that includes N, O, or S as a ring-forming heteroatom, or a substituted or unsubstituted benzonaphtoheterole group that includes N, O, or S as a ring-forming heteroatom. In the amine compound according to an embodiment, a position at which the third substituent is bonded to a nitrogen atom of the amine compound is not particularly limited.

Since the amine compound according to an embodiment includes the first to third substituents, charge balance may be controlled due to a three-dimensional effect, and thus excellent charge transportability may be exhibited. The amine compound according to an embodiment includes a dibenzoheterole derivative and an ortho-terphenyl derivative that are each bonded to a nitrogen atom at a specific position, and thus may have excellent charge transportability and material stability, thereby contributing to improvements in high efficiency and long lifespan of the light-emitting element.

The light-emitting element ED according to an embodiment may include an amine compound according to an embodiment that includes the first to third substituents as described above. The amine compound according to an embodiment may be represented by Formula 1:

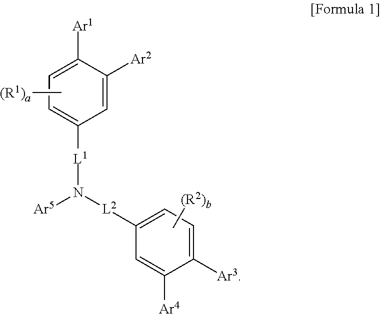

In Formula 1, L¹ and L² may each independently be a direct linkage, a substituted or unsubstituted arylene group having 6 to 30 ring-forming carbon atoms, or a substituted or unsubstituted heteroarylene group having 5 to 30 ring-forming carbon atoms. For example, in Formula 1, L¹ may be a direct linkage, a substituted or unsubstituted arylene group, or a substituted or unsubstituted divalent dibenzoheterole group.

In an embodiment, L¹ and L² may each independently be a direct linkage, a substituted or unsubstituted phenylene group, a substituted or unsubstituted biphenylene group (or a substituted or unsubstituted divalent biphenyl group), a substituted or unsubstituted naphthylene group (or a substituted or unsubstituted divalent naphthyl group), a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group. For example, L¹ and L² may each independently be a direct linkage or a group represented by one of Formula L-a to Formula L-o. However, embodiments are not limited thereto.

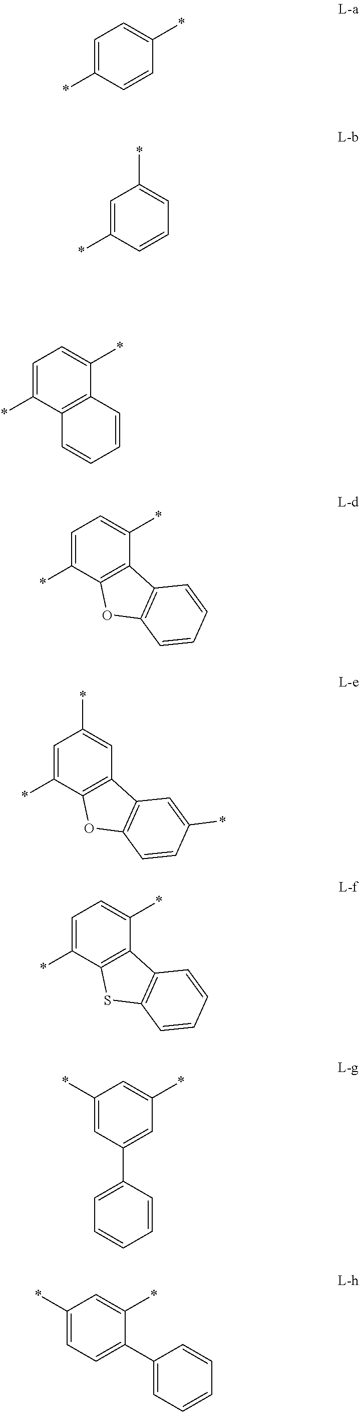

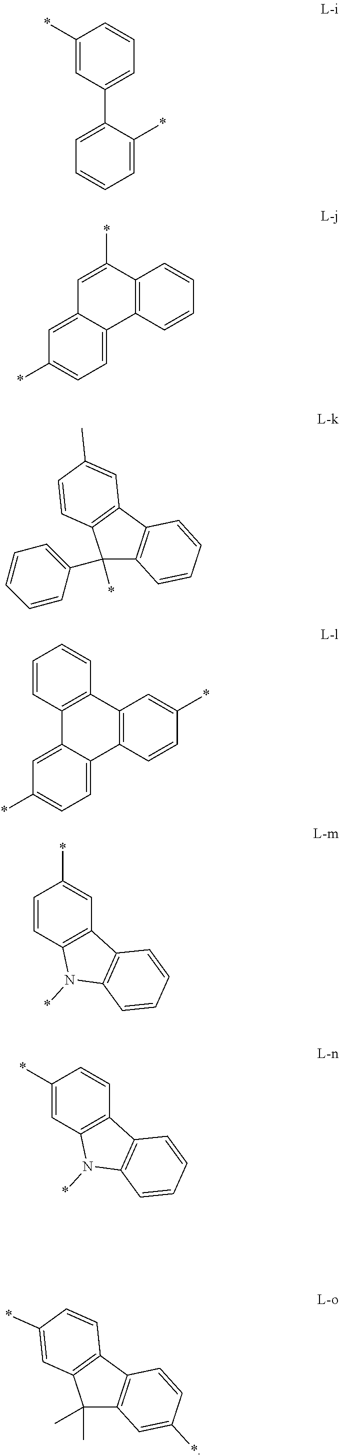

In Formula 1, a and b may each independently be an integer from 0 to 3. In Formula 1, R¹ and R² may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or bonded to an adjacent group to form a ring.

A case where a and b are each 0 may be the same as a case where a and b are each 3, and three groups of each of R¹ and R² are all hydrogen atoms. If a is at least 2, two or more of R¹ may all be the same, or at least one thereof may be different from the remainder. If b is at least 2, two or more of R² may all be the same, or at least one thereof may be different from the remainder. In an embodiment, R¹ and R² may each be a hydrogen atom. In another embodiment, at least one of R¹ and R² may be a deuterium atom. However, embodiments are not limited thereto.

In an embodiment, multiple adjacent R¹ groups may be bonded to each other to form a ring, and multiple adjacent R² groups may be bonded to each other to form a ring. In case that R¹ and R² are each bonded to an adjacent group to form a ring, R¹ and R² may be each bonded to an adjacent group to form a saturated hydrocarbon ring or an unsaturated hydrocarbon ring. For example, R¹ and R² may each be bonded to a substituted benzene ring to form a fused ring.

In Formula 1, Ar¹ to Ar⁴ may each independently be a substituted or unsubstituted aryl group having 6 to 30 ring-forming carbon atoms, or a substituted or unsubstituted heteroaryl group having 5 to 30 ring-forming carbon atoms. In an embodiment, Ar¹ to Ar⁴ may each independently be an unsubstituted phenyl group, a substituted or unsubstituted naphthyl group, a substituted or unsubstituted phenanthryl group, a substituted or unsubstituted pyrenyl group, a substituted or unsubstituted triphenylene group, a substituted or unsubstituted carbazole group, a substituted or unsubstituted dibenzothiophene group, or a substituted or unsubstituted dibenzofuran group.

In an embodiment, a phenyl group combined with Ar¹ and Ar² and a phenyl group combined with Ar³ and Art may respectively correspond to the first substituent and the second substituent as described above. For example, a phenyl group combined with Ar¹ and Ar² may correspond to the terphenyl derivative as the first substituent, and a phenyl group combined with Ar³ and Ar⁴ may correspond to the terphenyl derivative as the second substituent. In another embodiment, the phenyl group combined with Ar¹ and Ar² may correspond to the second substituent, and the phenyl group combined with Ar³ and Ar⁴ may correspond to the first substituent.

In Formula 1, Ar⁵ may correspond to the dibenzoheterole derivative as the third substituent, as described above. In Formula 1, Ar⁵ may be a group represented by one of Formula 2-1 to Formula 2-4. Formula 2-1 may correspond to a substituted or unsubstituted dibenzoheterole group directly bonded to a nitrogen atom in Formula 1. Formula 2-2 to Formula 2-4 may each correspond to a substituted or unsubstituted benzonaphtoheterole group directly bonded to a nitrogen atom in Formula 1.

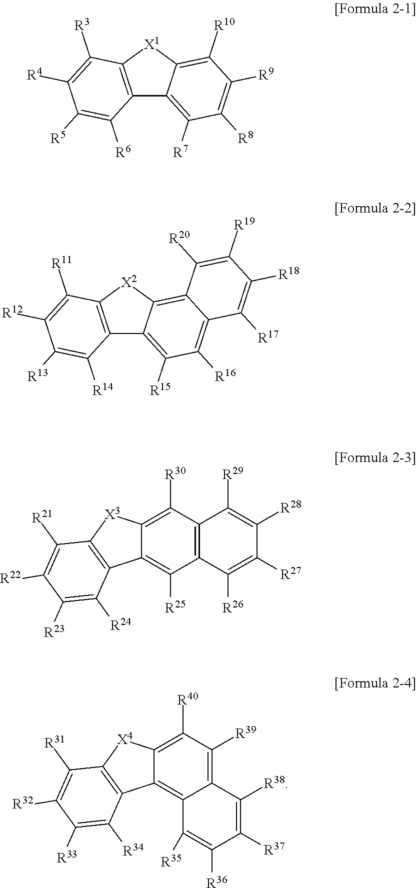

In Formula 2-1 to Formula 2-4, X¹ to X⁴ may each independently be O, S, or N(R⁴¹). If, in Formula 1, Ar⁵ is a group represented by Formula 2-1, the amine compound according to an embodiment may include, as a third substituent, a substituted or unsubstituted dibenzoheterole group that include O, S, or N as a ring-forming heteroatom. For example, if X¹ is O in Formula 2-1, the third substituent may be a substituted or unsubstituted dibenzofuran group. If X¹ is S in Formula 2-1, the third substituent may be a substituted or unsubstituted dibenzothiophene group. If X¹ is N(R⁴¹) in Formula 2-1, the third substituent may be a substituted or unsubstituted carbazole group.

In Formula 1, when Ar⁵ is a group represented by one of Formula 2-2 to Formula 2-4, the amine compound according to an embodiment may include as a third substituent, a substituted or unsubstituted benzonaphtoheterole group that includes O, S, or N as a ring-forming heteroatom. For example, when Ar⁵ is a group represented by one of Formula 2-2 to Formula 2-4, Ar⁵ may be a substituted or unsubstituted benzonaphtofuran group, a substituted or unsubstituted benzonaphthothiophene group, or a substituted or unsubstituted benzocarbazole group.

In Formula 2-1 to Formula 2-4, R³ to R¹⁰ may each independently be a hydrogen atom, a deuterium atom, or a substituted or unsubstituted aryl group having 6 to 30 ring-forming carbon atoms; and R¹¹ to R⁴¹ may each independently be a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms. In an embodiment, R³ to R⁴¹ may each independently be a hydrogen atom, a deuterium atom, a substituted or unsubstituted phenyl group, or a substituted or unsubstituted naphthyl group, but embodiments are not limited thereto.

In Formula 2-1, one of R³ to R¹⁰ and R⁴¹ may be a position connected to Formula 1; in Formula 2-2, one of R¹¹ to R²⁰ and R⁴¹ may be a position connected to Formula 1; in Formula 2-3, one of R²¹ to R³⁰ and R⁴¹ may be a position connected to Formula 1; and in Formula 2-4, one of R³¹ to R⁴¹ may be a position connected to Formula 1.

For example, in Formula 2-1, one of R³ to R¹⁰ and R⁴¹ may be a position connected to a nitrogen atom in Formula 1. In Formula 2-1, the remainder of R³ to R¹⁰ and R⁴¹, which are not connected to the nitrogen atom in Formula 1, may each independently be a hydrogen atom, a deuterium atom, or a substituted or unsubstituted aryl group having 6 to 30 ring-forming carbon atoms. In an embodiment, the remainder of R³ to R¹⁰ and R⁴¹ that are not connected to the nitrogen atom in Formula 1 may each independently be a hydrogen atom, a deuterium atom, a substituted or unsubstituted phenyl group, or a substituted or unsubstituted naphthyl group. However, embodiments are not limited thereto.

For example, in Formula 2-2, one of R¹¹ to R²⁰ and R⁴¹ may be a position connected to a nitrogen atom in Formula 1. In Formula 2-2, the remainder of R¹¹ to R²⁰ and R⁴¹, which are not connected to the nitrogen atom in Formula 1, may each independently be a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms. In an embodiment, the remainder of R¹¹ to R²⁰ and R⁴¹ that are not connected to the nitrogen atom in Formula 1 may each independently be a hydrogen atom, a deuterium atom, a substituted or unsubstituted phenyl group, or a substituted or unsubstituted naphthyl group. However, embodiments are not limited thereto.

For example, in Formula 2-3, one of R²¹ to R³⁰ and R⁴¹ may be a position connected to a nitrogen atom in Formula 1. In Formula 2-3, the remainder of R²¹ to R³⁰ and R⁴¹, which are not connected to the nitrogen atom in Formula 1, may each independently be a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms. In an embodiment, the remainder of R²¹ to R³¹ and R⁴¹ that are not connected to the nitrogen atom in Formula 1 may each independently be a hydrogen atom, a deuterium atom, a substituted or unsubstituted phenyl group, or a substituted or unsubstituted naphthyl group. However, embodiments are not limited thereto.

For example, in Formula 2-4, one of R³¹ to R⁴¹ may be a position connected to a nitrogen atom in Formula 1. In Formula 2-4, the remainder of R³¹ to R⁴¹, which are not connected to the nitrogen atom in Formula 1, may each independently be a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms. In an embodiment, the remainder of R³¹ to R⁴¹ that are not connected to the nitrogen atom in Formula 1 may each independently be a hydrogen atom, a deuterium atom, a substituted or unsubstituted phenyl group, or a substituted or unsubstituted naphthyl group. However, embodiments are not limited thereto.

In an embodiment, in Formula 1, when Ar⁵ is a group represented by Formula 2-1, R³ to R¹⁰ may each independently be a substituted or unsubstituted aryl group having 6 to 30 ring-forming carbon atoms.

In an embodiment, in Formula 1, when Ar⁵ is a group represented by Formula 2-1, Ar¹ to Art may each independently be a substituted or unsubstituted aryl group having 6 to 30 ring-forming carbon atoms. For example, in Formula 1, when Ar⁵ is a group represented by Formula 2-1, Ar¹ to Art may each independently be a substituted or unsubstituted phenyl group, a substituted or unsubstituted naphthyl group, a substituted or unsubstituted phenanthryl group, a substituted or unsubstituted pyrenyl group, or a substituted or unsubstituted triphenylene group. However, embodiments are not limited thereto.

In an embodiment, in Formula 1, at least one hydrogen atom of the amine compound may be substituted with a deuterium atom. In Formula 1, Ar¹ to Ar⁵ and R¹ to R⁴¹ may each independently be a deuterium atom or include a deuterium atom as a substituent. In an embodiment, in Formula 1, L¹ and L² may each independently include a deuterium atom as a substituent. In an embodiment, in Formula 1, Ar¹ to Ar⁵ may each independently include a deuterium atom as a substituent.

In Formula 1, Ar¹ to Ar⁵, R¹ to R⁴¹, L¹, and L² may not include a substituted or unsubstituted amine group. For example, the amine compound represented by Formula 1 may be a monoamine compound that does not include any additional amine group.

In the amine compound according to an embodiment, a 3,4-substituted phenyl group may be directly bonded to a nitrogen atom of the amine compound, or may be bonded to the nitrogen atom via a linking moiety.

In an embodiment, the amine compound represented by Formula 1 may be represented by Formula 3-1 or Formula 3-2. Formula 3-1 represents a case in which two 3,4-substituted phenyl groups are each directly bonded to a nitrogen atom. Formula 3-2 represents a case in which one of the two 3,4-substituted phenyl groups is directly bonded to a nitrogen atom, and the other 3,4-substituted phenyl group is bonded to a nitrogen atom via L²¹, which is a linking moiety.

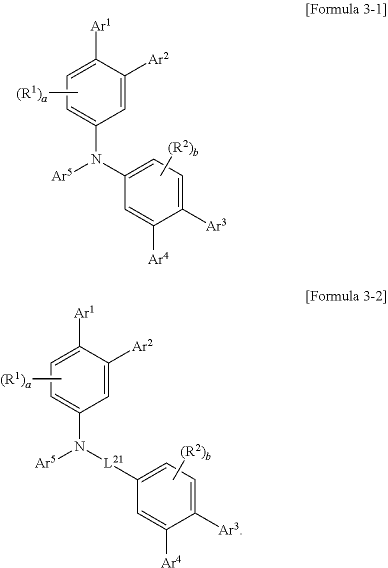

In Formula 3-2, L²¹ may be a substituted or unsubstituted phenylene group, a substituted or unsubstituted biphenylene group, a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group. For example, in an embodiment, L²¹ may be a group represented by one of Formula L-a to Formula L-o as described above. However, embodiments are not limited thereto. In Formula 3-1 and Formula 3-2, Ar¹ to Ar⁵, R¹, R², a, and b may be the same as defined in Formula 1.

In an embodiment, the amine compound represented by Formula 1 may be represented by Formula 4. Formula 4 represents a case where Ar¹ to Ar⁴ in Formula 1 are further defined.

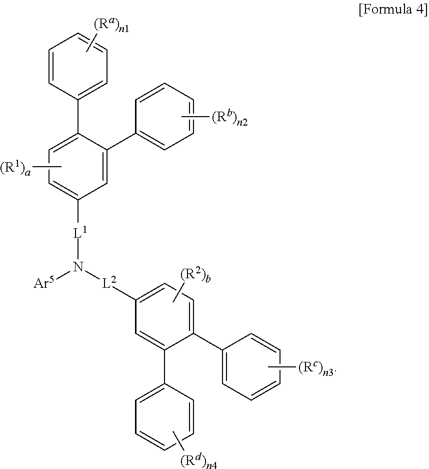

In Formula 4, n1 to n4 may each independently be an integer from 0 to 5. In Formula 4, R^{a} to R^{d} may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

A case where n1 to n4 are each 0 may be the same as a case where n1 to n4 are each 5 and five groups of each of R^{a} to R^{d} are all hydrogen atoms. If n1 is at least 2, two or more of R^{a} may all be the same, or at least one thereof may be different from the remainder. If n2 is at least 2, two or more of R^{b} may all be the same, or at least one thereof may be different from the remainder. If n3 is at least 2, two or more of Re may all be the same, or at least one thereof may be different from the remainder. If n4 is at least 2, two or more of R^{d} may all be the same, or at least one thereof may be different from the remainder. In an embodiment, multiple groups of each of R^{a} to R^{d} may all be hydrogen atoms. In another embodiment, at least one of R^{a} to R^{d} may be a deuterium atom. However, embodiments are not limited thereto.

In an embodiment, adjacent groups among R^{a}, R^{b}, R^{c}, and R^{d} may be bonded to each other to form a ring. For example, when R^{a} is bonded to an adjacent group to form a ring, R^{a} and the adjacent group may form a saturated hydrocarbon ring or an unsaturated hydrocarbon ring. For example, when R^{b} is bonded to an adjacent group to form a ring, R^{b} and the adjacent group may form a saturated hydrocarbon ring or an unsaturated hydrocarbon ring. For example, when R^{c} is bonded to an adjacent group to form a ring, Re and the adjacent group may form a saturated hydrocarbon ring or an unsaturated hydrocarbon ring. For example, when R^{d} is bonded to an adjacent group to form a ring, R^{d} and the adjacent group may form a saturated hydrocarbon ring or an unsaturated hydrocarbon ring.

In Formula 4, Ar⁵, R¹, R², L¹, L², a, and b may be the same as defined in Formula 1.

In an embodiment, the amine compound represented by Formula 1 may be any compound selected from Compound Group 1. In an embodiment, in the light-emitting element ED, the hole transport region HTR may include at least one compound selected from Compound Group 1. In Compound Group 1, D represents a deuterium atom.

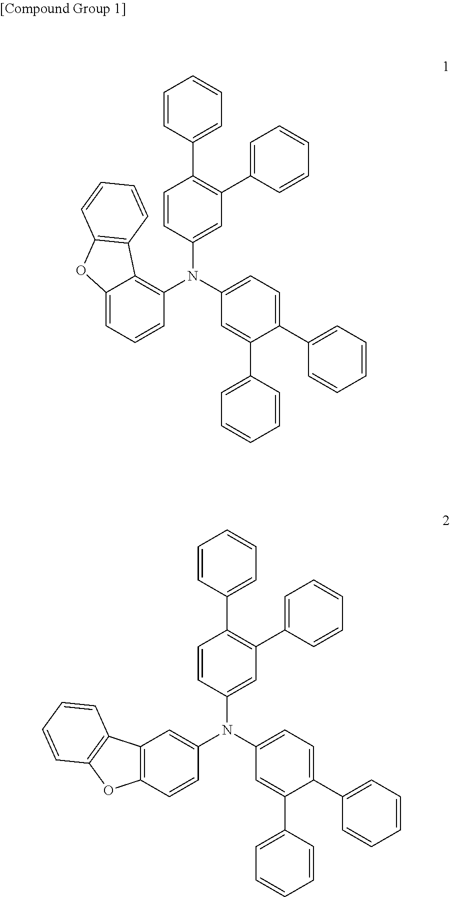 [SMILES: c1ccc(-c2ccc(N(c3ccc(-c4ccccc4)c(-c4ccccc4)c3)c3cccc4oc5ccccc5c34)cc2-c2ccccc2)cc1 ; c1ccc(-c2ccc(N(c3ccc(-c4ccccc4)c(-c4ccccc4)c3)c3ccc4oc5ccccc5c4c3)cc2-c2ccccc2)cc1]

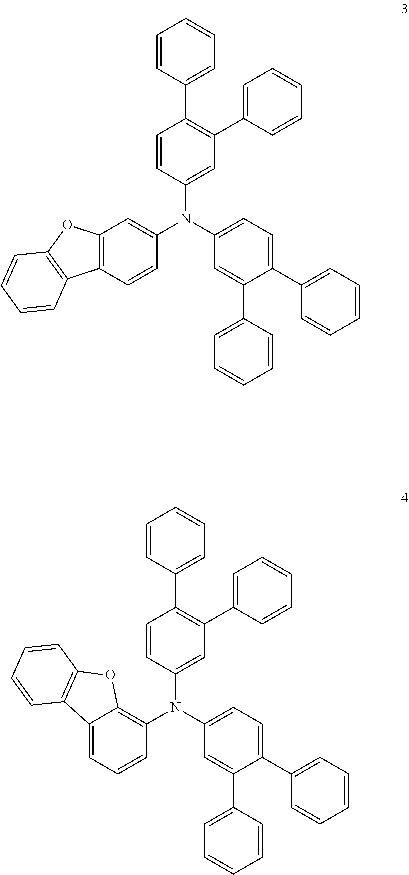 [SMILES: c1ccc(-c2ccc(N(c3ccc(-c4ccccc4)c(-c4ccccc4)c3)c3ccc4c(c3)oc3ccccc34)cc2-c2ccccc2)cc1 ; c1ccc(-c2ccc(N(c3ccc(-c4ccccc4)c(-c4ccccc4)c3)c3cccc4c3oc3ccccc34)cc2-c2ccccc2)cc1]

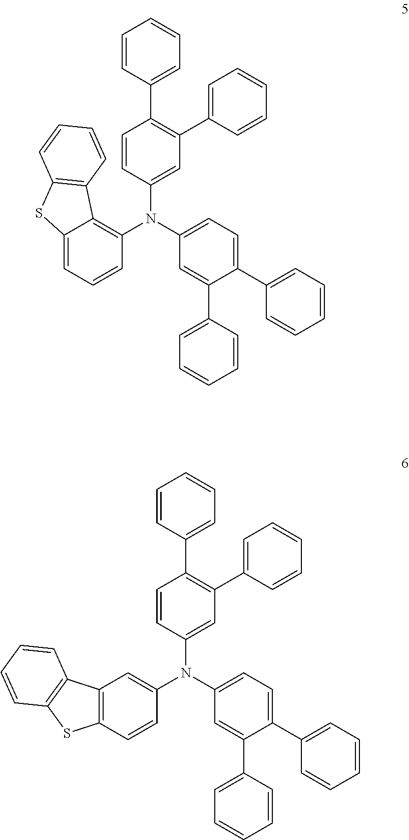 [SMILES: c1ccc(-c2ccc(N(c3ccc(-c4ccccc4)c(-c4ccccc4)c3)c3cccc4sc5ccccc5c34)cc2-c2ccccc2)cc1 ; c1ccc(-c2ccc(N(c3ccc(-c4ccccc4)c(-c4ccccc4)c3)c3ccc4sc5ccccc5c4c3)cc2-c2ccccc2)cc1]

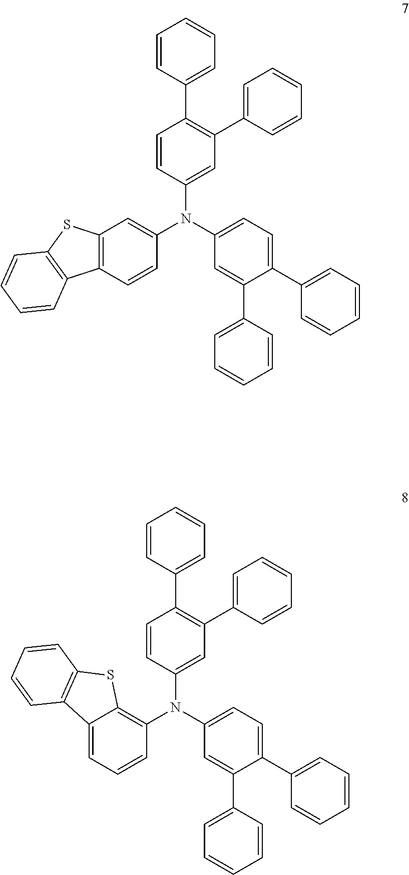 [SMILES: c1ccc(-c2ccc(N(c3ccc(-c4ccccc4)c(-c4ccccc4)c3)c3ccc4c(c3)sc3ccccc34)cc2-c2ccccc2)cc1 ; c1ccc(-c2ccc(N(c3ccc(-c4ccccc4)c(-c4ccccc4)c3)c3cccc4c3sc3ccccc34)cc2-c2ccccc2)cc1]

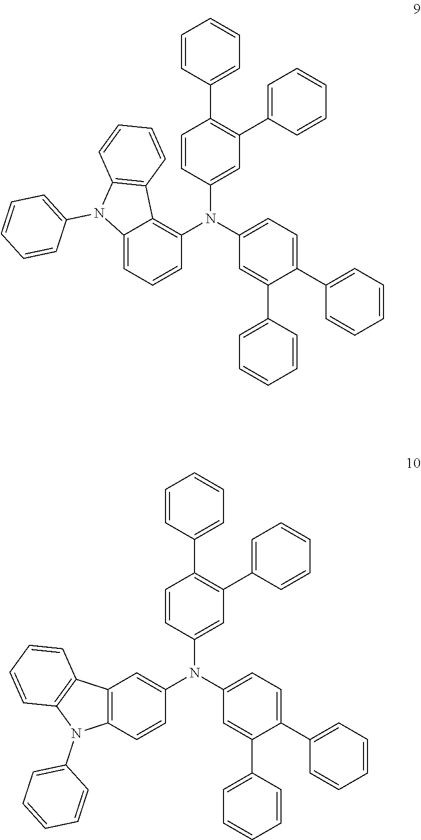 [SMILES: c1ccc(-c2ccc(N(c3ccc(-c4ccccc4)c(-c4ccccc4)c3)c3cccc4c3c3ccccc3n4-c3ccccc3)cc2-c2ccccc2)cc1 ; c1ccc(-c2ccc(N(c3ccc(-c4ccccc4)c(-c4ccccc4)c3)c3ccc4c(c3)c3ccccc3n4-c3ccccc3)cc2-c2ccccc2)cc1]

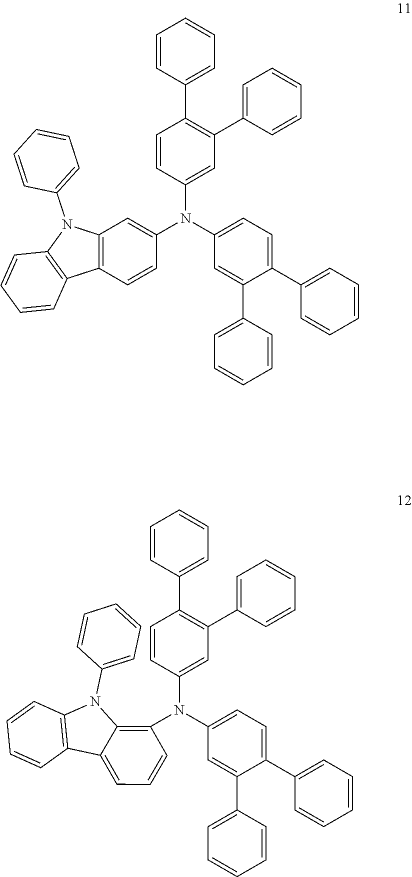 [SMILES: c1ccc(-c2ccc(N(c3ccc(-c4ccccc4)c(-c4ccccc4)c3)c3ccc4c5ccccc5n(-c5ccccc5)c4c3)cc2-c2ccccc2)cc1 ; c1ccc(-c2ccc(N(c3ccc(-c4ccccc4)c(-c4ccccc4)c3)c3cccc4c5ccccc5n(-c5ccccc5)c34)cc2-c2ccccc2)cc1]

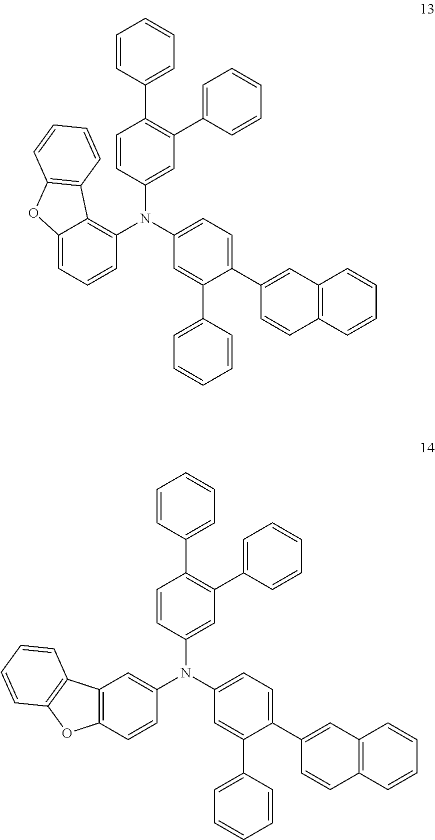 [SMILES: c1ccc(-c2ccc(N(c3ccc(-c4ccc5ccccc5c4)c(-c4ccccc4)c3)c3cccc4oc5ccccc5c34)cc2-c2ccccc2)cc1 ; c1ccc(-c2ccc(N(c3ccc(-c4ccc5ccccc5c4)c(-c4ccccc4)c3)c3ccc4oc5ccccc5c4c3)cc2-c2ccccc2)cc1]

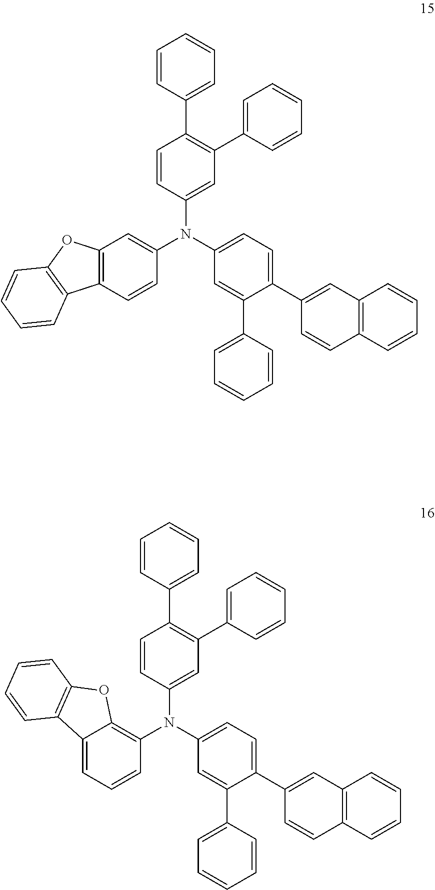 [SMILES: c1ccc(-c2ccc(N(c3ccc(-c4ccc5ccccc5c4)c(-c4ccccc4)c3)c3ccc4c(c3)oc3ccccc34)cc2-c2ccccc2)cc1 ; c1ccc(-c2ccc(N(c3ccc(-c4ccc5ccccc5c4)c(-c4ccccc4)c3)c3cccc4c3oc3ccccc34)cc2-c2ccccc2)cc1]

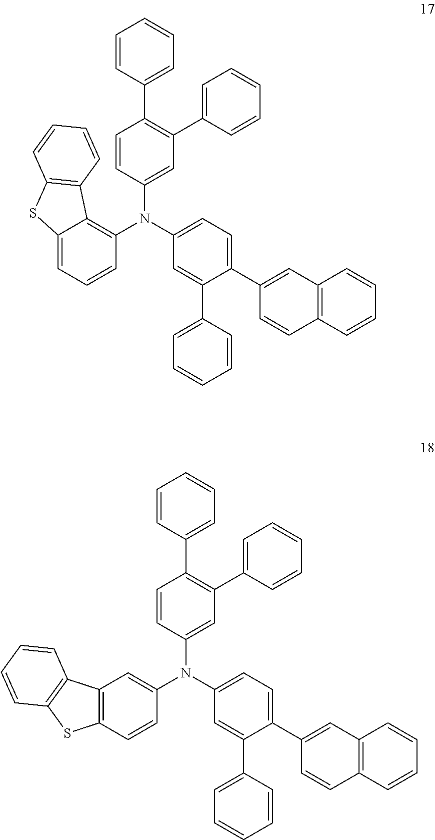 [SMILES: c1ccc(-c2ccc(N(c3ccc(-c4ccc5ccccc5c4)c(-c4ccccc4)c3)c3cccc4sc5ccccc5c34)cc2-c2ccccc2)cc1 ; c1ccc(-c2ccc(N(c3ccc(-c4ccc5ccccc5c4)c(-c4ccccc4)c3)c3ccc4sc5ccccc5c4c3)cc2-c2ccccc2)cc1]

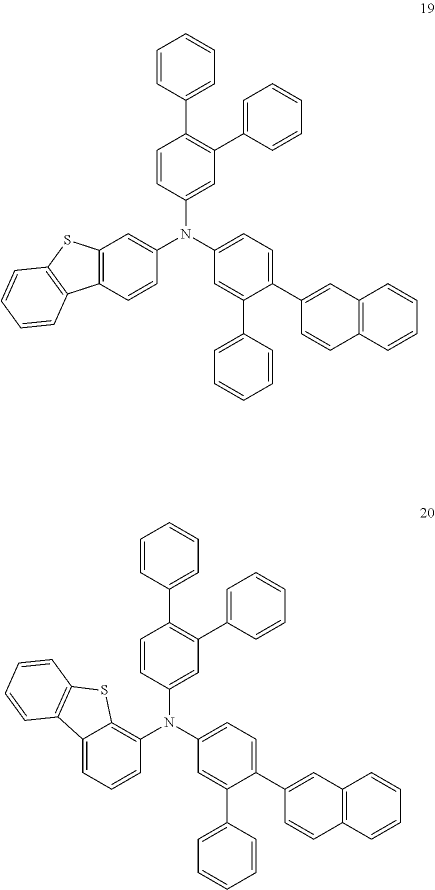 [SMILES: c1ccc(-c2ccc(N(c3ccc(-c4ccc5ccccc5c4)c(-c4ccccc4)c3)c3ccc4c(c3)sc3ccccc34)cc2-c2ccccc2)cc1 ; c1ccc(-c2ccc(N(c3ccc(-c4ccc5ccccc5c4)c(-c4ccccc4)c3)c3cccc4c3sc3ccccc34)cc2-c2ccccc2)cc1]

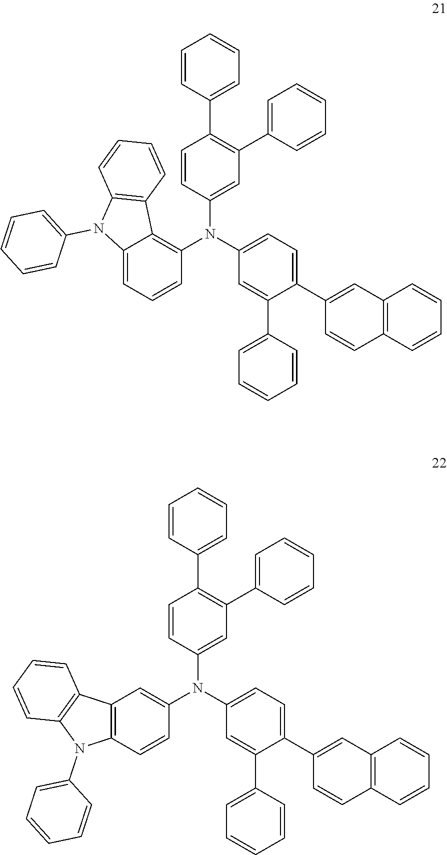 [SMILES: c1ccc(-c2ccc(N(c3ccc(-c4ccc5ccccc5c4)c(-c4ccccc4)c3)c3cccc4c3c3ccccc3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3ccccc3n4-c3ccccc3)cc2-c2ccccc2)cc1]

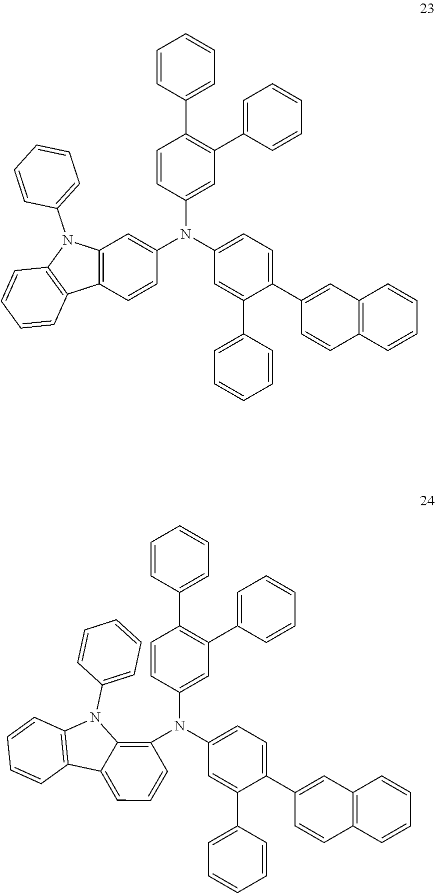 [SMILES: c1ccc(-c2ccc(N(c3ccc(-c4ccc5ccccc5c4)c(-c4ccccc4)c3)c3ccc4c5ccccc5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5ccccc5n(-c5ccccc5)c34)cc2-c2ccccc2)cc1]

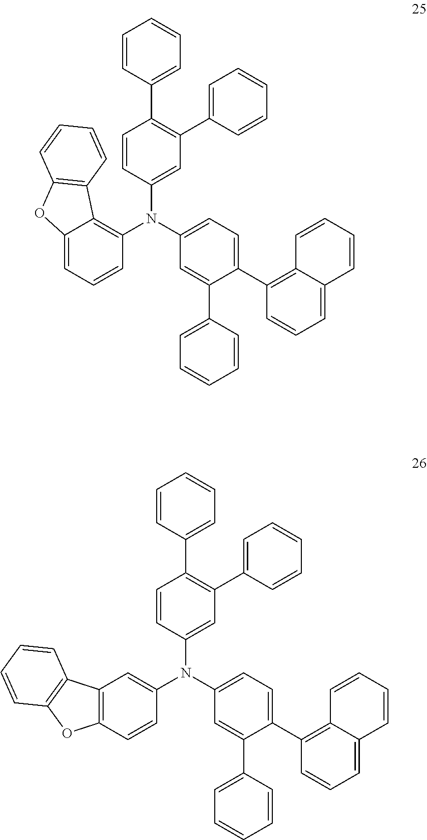 [SMILES: c1ccc(-c2ccc(N(c3ccc(-c4cccc5ccccc45)c(-c4ccccc4)c3)c3cccc4oc5ccccc5c34)cc2-c2ccccc2)cc1 ; c1ccc(-c2ccc(N(c3ccc(-c4cccc5ccccc45)c(-c4ccccc4)c3)c3ccc4oc5ccccc5c4c3)cc2-c2ccccc2)cc1]

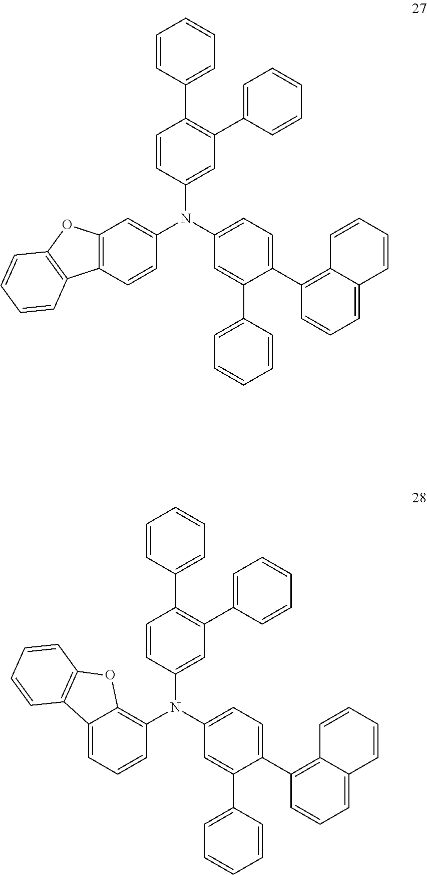 [SMILES: c1ccc(-c2ccc(N(c3ccc(-c4cccc5ccccc45)c(-c4ccccc4)c3)c3ccc4c(c3)oc3ccccc34)cc2-c2ccccc2)cc1 ; c1ccc(-c2ccc(N(c3ccc(-c4cccc5ccccc45)c(-c4ccccc4)c3)c3cccc4c3oc3ccccc34)cc2-c2ccccc2)cc1]

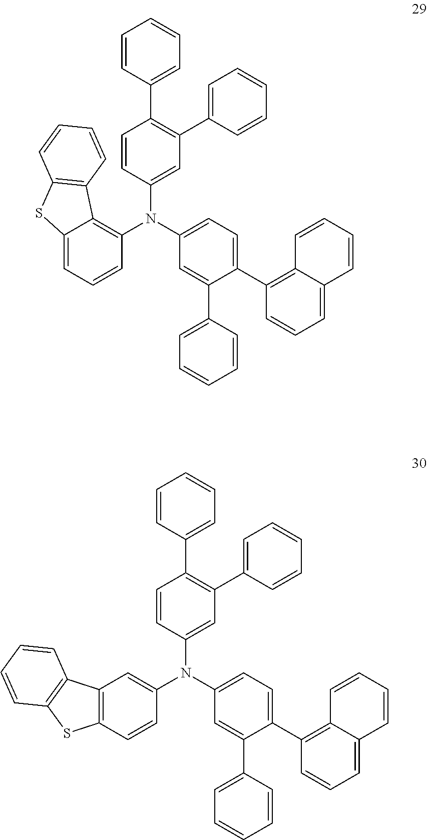 [SMILES: c1ccc(-c2ccc(N(c3ccc(-c4cccc5ccccc45)c(-c4ccccc4)c3)c3cccc4sc5ccccc5c34)cc2-c2ccccc2)cc1 ; c1ccc(-c2ccc(N(c3ccc(-c4cccc5ccccc45)c(-c4ccccc4)c3)c3ccc4sc5ccccc5c4c3)cc2-c2ccccc2)cc1]

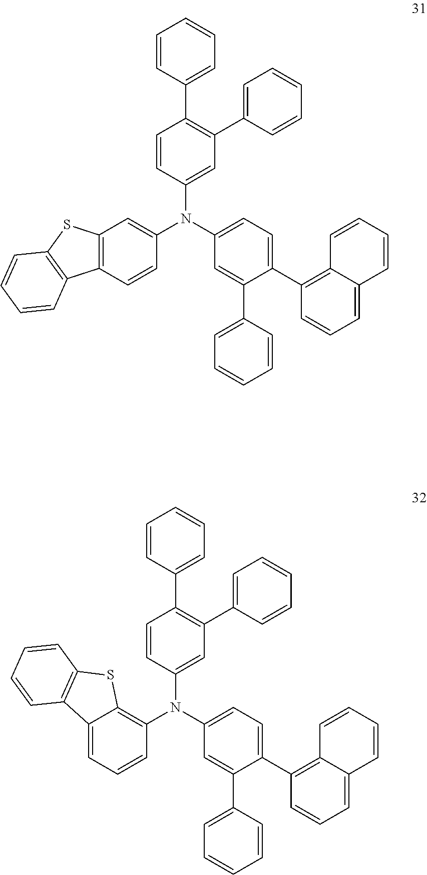 [SMILES: c1ccc(-c2ccc(N(c3ccc(-c4cccc5ccccc45)c(-c4ccccc4)c3)c3ccc4c(c3)sc3ccccc34)cc2-c2ccccc2)cc1 ; c1ccc(-c2ccc(N(c3ccc(-c4cccc5ccccc45)c(-c4ccccc4)c3)c3cccc4c3sc3ccccc34)cc2-c2ccccc2)cc1]

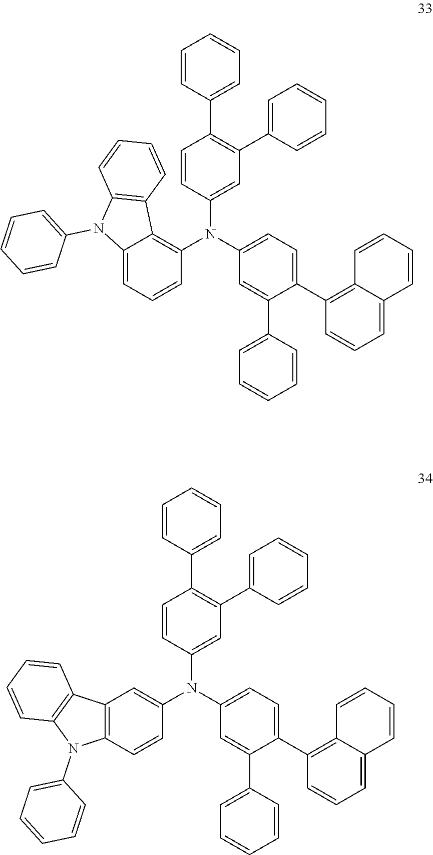 [SMILES: c1ccc(-c2ccc(N(c3ccc(-c4cccc5ccccc45)c(-c4ccccc4)c3)c3cccc4c3c3ccccc3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3ccccc3n4-c3ccccc3)cc2-c2ccccc2)cc1]

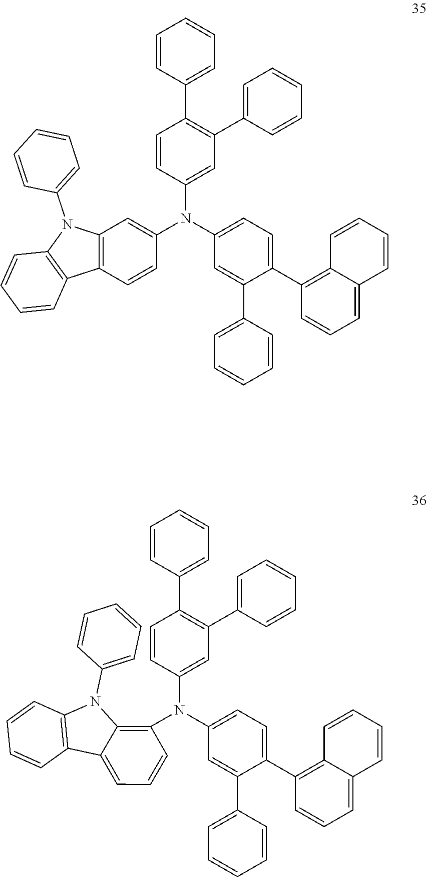 [SMILES: c1ccc(-c2ccc(N(c3ccc(-c4cccc5ccccc45)c(-c4ccccc4)c3)c3ccc4c5ccccc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5ccccc5n(-c5ccccc5)c34)cc2-c2ccccc2)cc1]

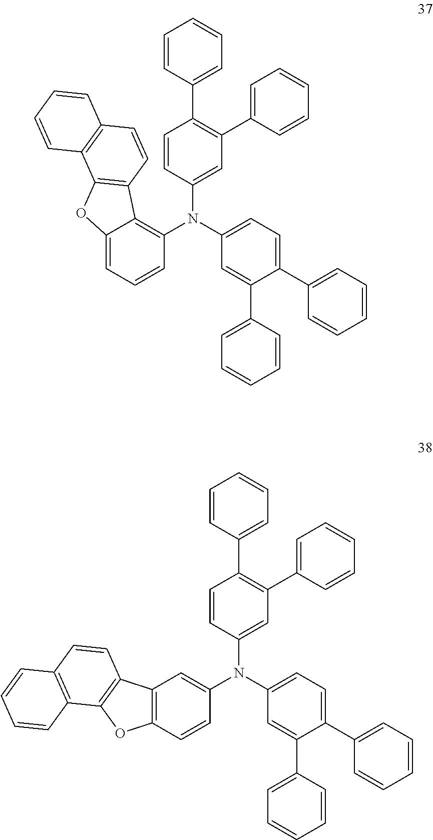 [SMILES: c1ccc(-c2ccc(N(c3ccc(-c4ccccc4)c(-c4ccccc4)c3)c3cccc4oc5c6ccccc6ccc5c34)cc2-c2ccccc2)cc1 ; c1ccc(-c2ccc(N(c3ccc(-c4ccccc4)c(-c4ccccc4)c3)c3ccc4oc5c6ccccc6ccc5c4c3)cc2-c2ccccc2)cc1]

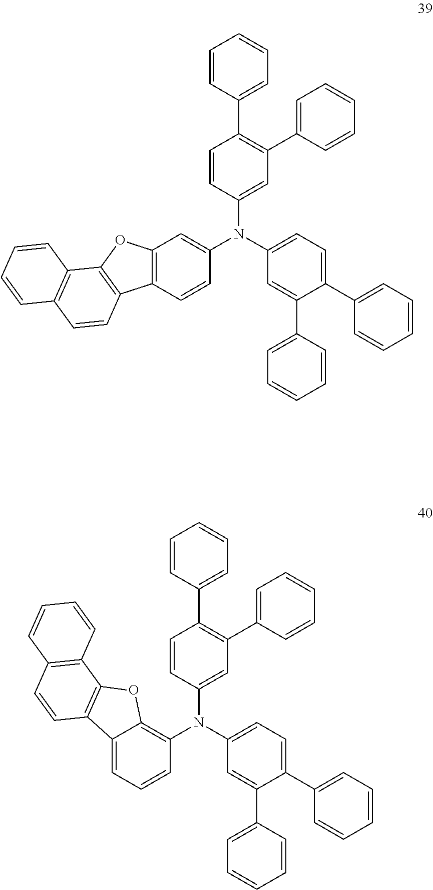 [SMILES: c1ccc(-c2ccc(N(c3ccc(-c4ccccc4)c(-c4ccccc4)c3)c3ccc4c(c3)oc3c5ccccc5ccc43)cc2-c2ccccc2)cc1 ; c1ccc(-c2ccc(N(c3ccc(-c4ccccc4)c(-c4ccccc4)c3)c3cccc4c3oc3c5ccccc5ccc43)cc2-c2ccccc2)cc1]

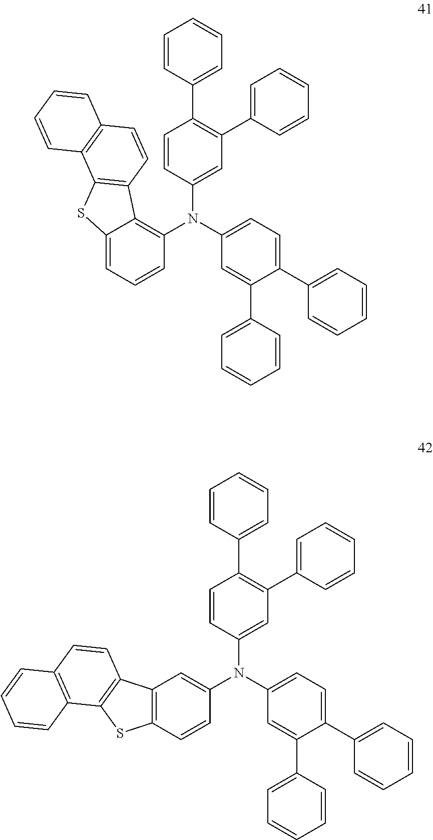 [SMILES: c1ccc(-c2ccc(N(c3ccc(-c4ccccc4)c(-c4ccccc4)c3)c3cccc4sc5c6ccccc6ccc5c34)cc2-c2ccccc2)cc1 ; c1ccc(-c2ccc(N(c3ccc(-c4ccccc4)c(-c4ccccc4)c3)c3ccc4sc5c6ccccc6ccc5c4c3)cc2-c2ccccc2)cc1]

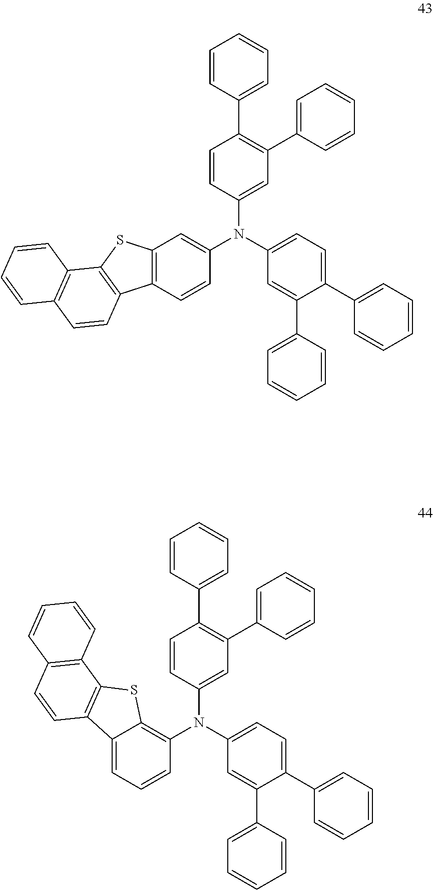 [SMILES: c1ccc(-c2ccc(N(c3ccc(-c4ccccc4)c(-c4ccccc4)c3)c3ccc4c(c3)sc3c5ccccc5ccc43)cc2-c2ccccc2)cc1 ; c1ccc(-c2ccc(N(c3ccc(-c4ccccc4)c(-c4ccccc4)c3)c3cccc4c3sc3c5ccccc5ccc43)cc2-c2ccccc2)cc1]

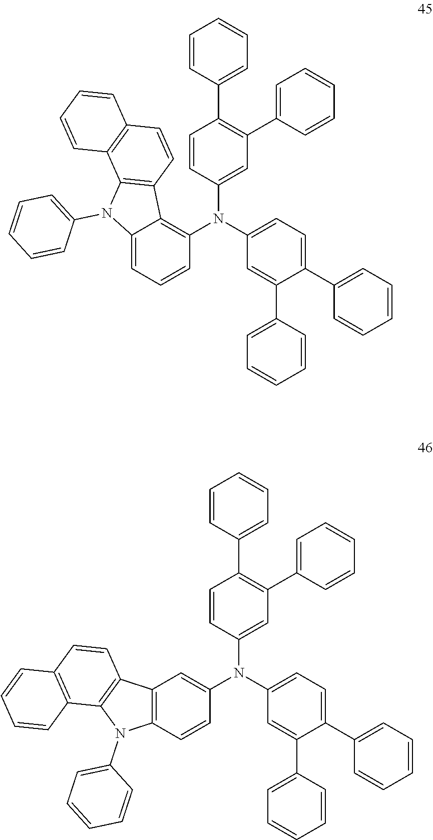 [SMILES: c1ccc(-c2ccc(N(c3ccc(-c4ccccc4)c(-c4ccccc4)c3)c3cccc4c3c3ccc5ccccc5c3n4-c3ccccc3)cc2-c2ccccc2)cc1 ; c1ccc(-c2ccc(N(c3ccc(-c4ccccc4)c(-c4ccccc4)c3)c3ccc4c(c3)c3ccc5ccccc5c3n4-c3ccccc3)cc2-c2ccccc2)cc1]

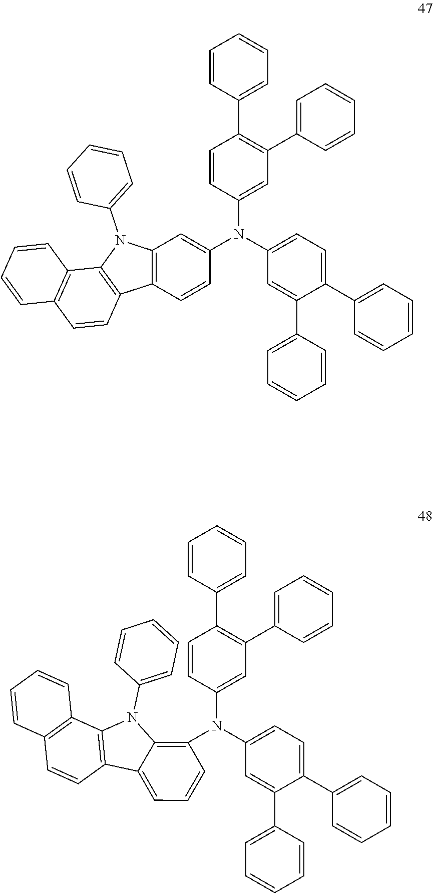 [SMILES: c1ccc(-c2ccc(N(c3ccc(-c4ccccc4)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4ccccc4)c(-c4ccccc4)c3)c3cccc4c5ccc6ccccc6c5n(-c5ccccc5)c34)cc2-c2ccccc2)cc1]

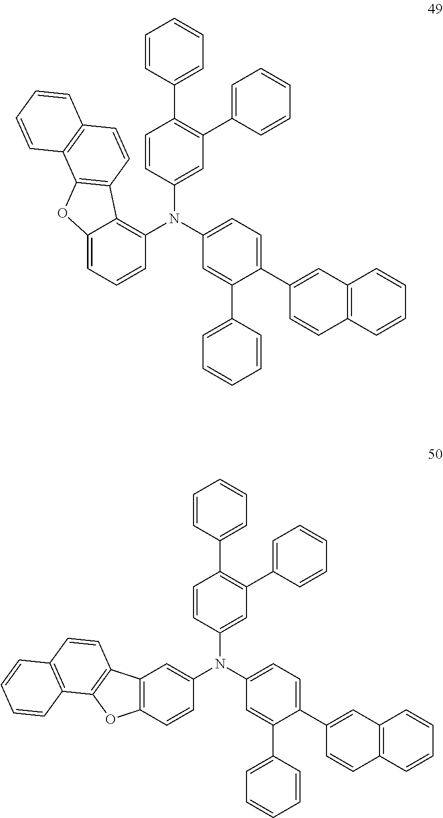 [SMILES: c1ccc(-c2ccc(N(c3ccc(-c4ccc5ccccc5c4)c(-c4ccccc4)c3)c3cccc4oc5c6ccccc6ccc5c34)cc2-c2ccccc2)cc1 ; c1ccc(-c2ccc(N(c3ccc(-c4ccc5ccccc5c4)c(-c4ccccc4)c3)c3ccc4oc5c6ccccc6ccc5c4c3)cc2-c2ccccc2)cc1]

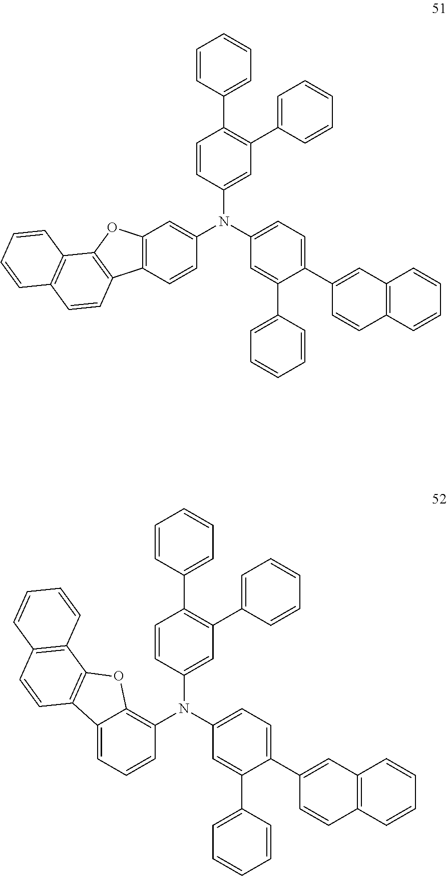 [SMILES: c1ccc(-c2ccc(N(c3ccc(-c4ccc5ccccc5c4)c(-c4ccccc4)c3)c3ccc4c(c3)oc3c5ccccc5ccc43)cc2-c2ccccc2)cc1 ; c1ccc(-c2ccc(N(c3ccc(-c4ccc5ccccc5c4)c(-c4ccccc4)c3)c3cccc4c3oc3c5ccccc5ccc43)cc2-c2ccccc2)cc1]

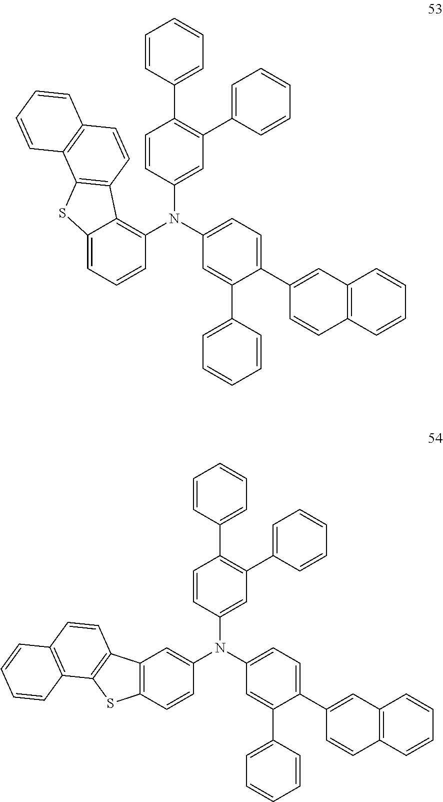 [SMILES: c1ccc(-c2ccc(N(c3ccc(-c4ccc5ccccc5c4)c(-c4ccccc4)c3)c3cccc4sc5c6ccccc6ccc5c34)cc2-c2ccccc2)cc1 ; c1ccc(-c2ccc(N(c3ccc(-c4ccc5ccccc5c4)c(-c4ccccc4)c3)c3ccc4sc5c6ccccc6ccc5c4c3)cc2-c2ccccc2)cc1]

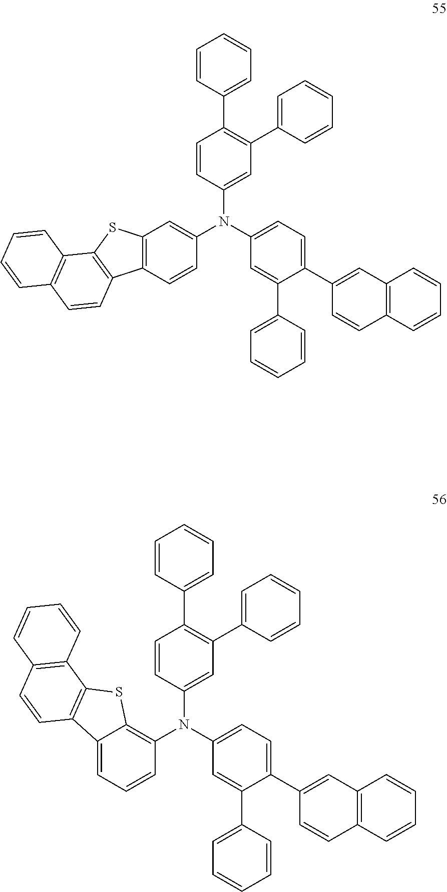 [SMILES: c1ccc(-c2ccc(N(c3ccc(-c4ccc5ccccc5c4)c(-c4ccccc4)c3)c3ccc4c(c3)sc3c5ccccc5ccc43)cc2-c2ccccc2)cc1 ; c1ccc(-c2ccc(N(c3ccc(-c4ccc5ccccc5c4)c(-c4ccccc4)c3)c3cccc4c3sc3c5ccccc5ccc43)cc2-c2ccccc2)cc1]

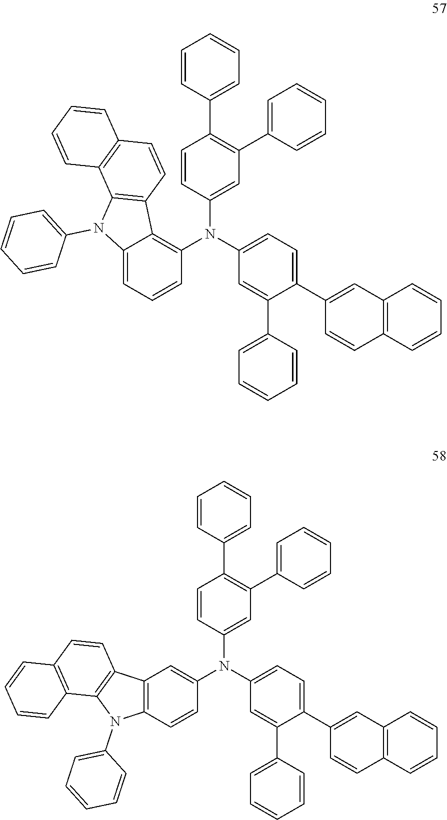 [SMILES: c1ccc(-c2ccc(N(c3ccc(-c4ccc5ccccc5c4)c(-c4ccccc4)c3)c3cccc4c3c3ccc5ccccc5c3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3ccc5ccccc5c3n4-c3ccccc3)cc2-c2ccccc2)cc1]

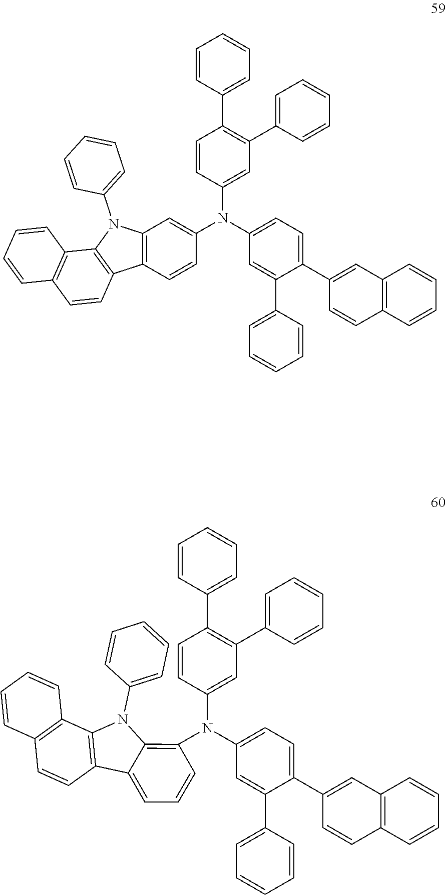 [SMILES: c1ccc(-c2ccc(N(c3ccc(-c4ccc5ccccc5c4)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5ccc6ccccc6c5n(-c5ccccc5)c34)cc2-c2ccccc2)cc1]

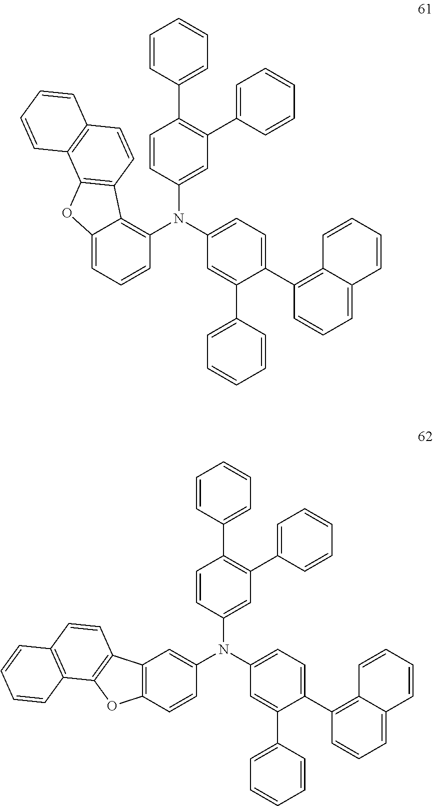 [SMILES: c1ccc(-c2ccc(N(c3ccc(-c4cccc5ccccc45)c(-c4ccccc4)c3)c3cccc4oc5c6ccccc6ccc5c34)cc2-c2ccccc2)cc1 ; c1ccc(-c2ccc(N(c3ccc(-c4cccc5ccccc45)c(-c4ccccc4)c3)c3ccc4oc5c6ccccc6ccc5c4c3)cc2-c2ccccc2)cc1]

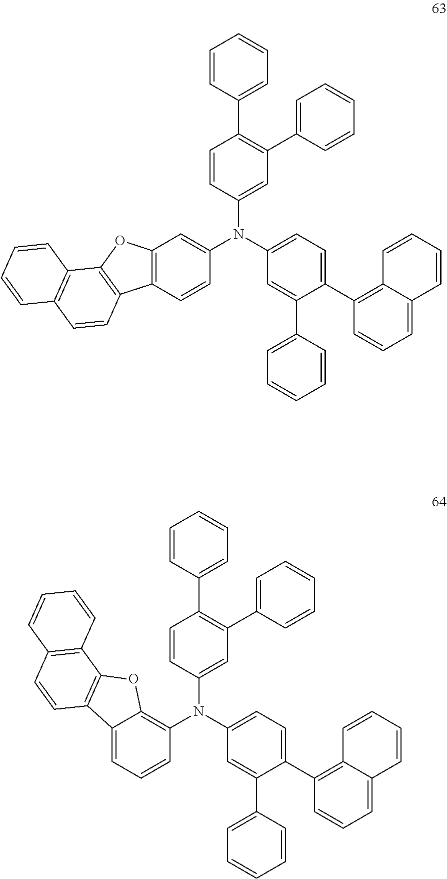 [SMILES: c1ccc(-c2ccc(N(c3ccc(-c4cccc5ccccc45)c(-c4ccccc4)c3)c3ccc4c(c3)oc3c5ccccc5ccc43)cc2-c2ccccc2)cc1 ; c1ccc(-c2ccc(N(c3ccc(-c4cccc5ccccc45)c(-c4ccccc4)c3)c3cccc4c3oc3c5ccccc5ccc43)cc2-c2ccccc2)cc1]

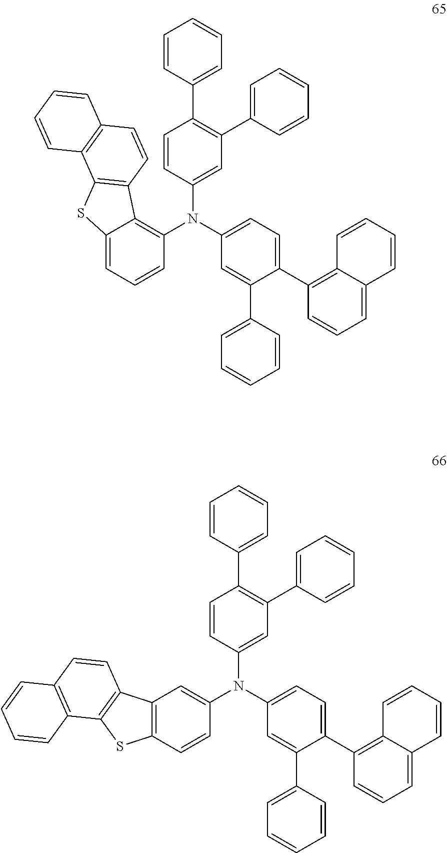 [SMILES: c1ccc(-c2ccc(N(c3ccc(-c4cccc5ccccc45)c(-c4ccccc4)c3)c3cccc4sc5c6ccccc6ccc5c34)cc2-c2ccccc2)cc1 ; c1ccc(-c2ccc(N(c3ccc(-c4cccc5ccccc45)c(-c4ccccc4)c3)c3ccc4sc5c6ccccc6ccc5c4c3)cc2-c2ccccc2)cc1]

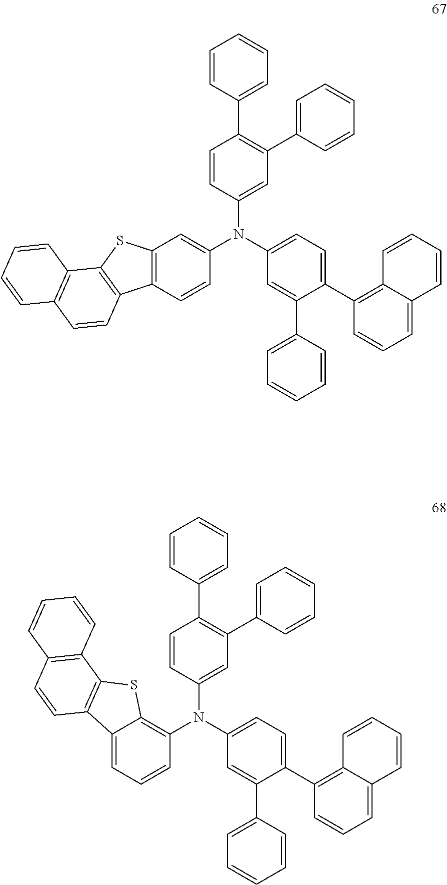 [SMILES: c1ccc(-c2ccc(N(c3ccc(-c4cccc5ccccc45)c(-c4ccccc4)c3)c3ccc4c(c3)sc3c5ccccc5ccc43)cc2-c2ccccc2)cc1 ; c1ccc(-c2ccc(N(c3ccc(-c4cccc5ccccc45)c(-c4ccccc4)c3)c3cccc4c3sc3c5ccccc5ccc43)cc2-c2ccccc2)cc1]

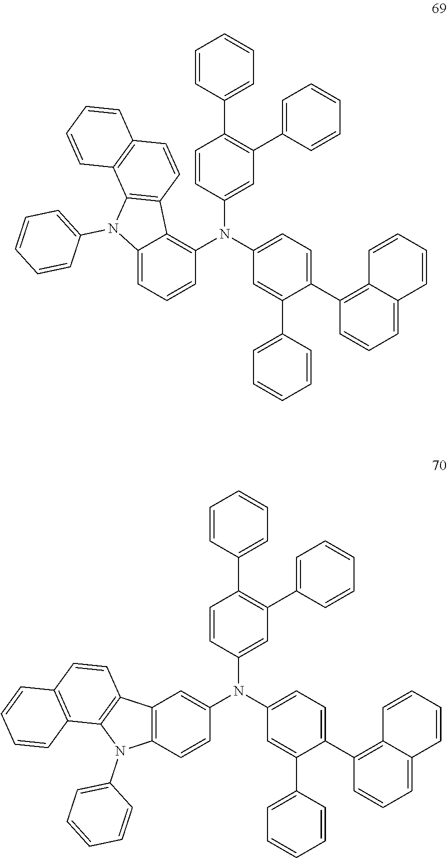 [SMILES: c1ccc(-c2ccc(N(c3ccc(-c4cccc5ccccc45)c(-c4ccccc4)c3)c3cccc4c3c3ccc5ccccc5c3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3ccc5ccccc5c3n4-c3ccccc3)cc2-c2ccccc2)cc1]

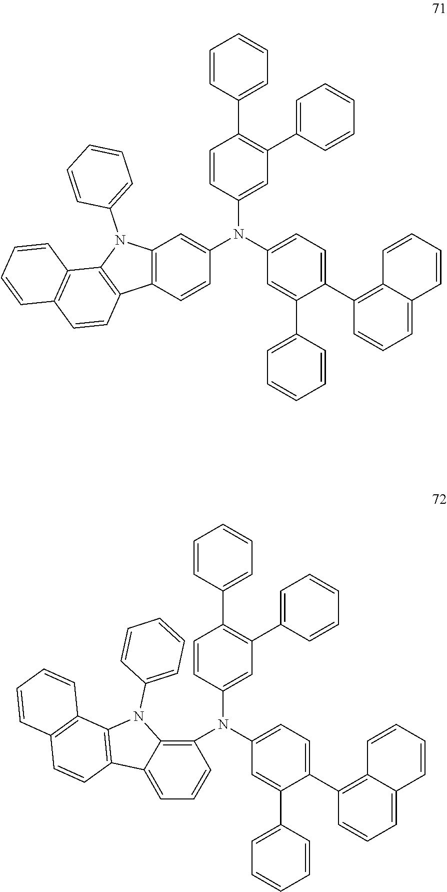 [SMILES: c1ccc(-c2ccc(N(c3ccc(-c4cccc5ccccc45)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5ccc6ccccc6c5n(-c5ccccc5)c34)cc2-c2ccccc2)cc1]

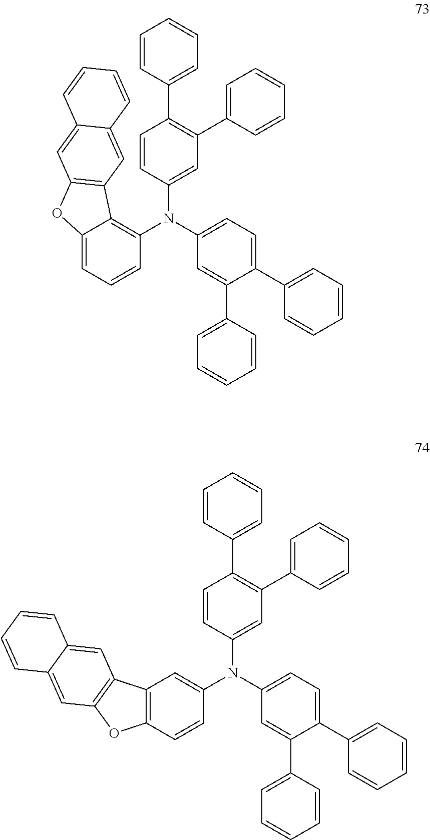 [SMILES: c1ccc(-c2ccc(N(c3ccc(-c4ccccc4)c(-c4ccccc4)c3)c3cccc4oc5cc6ccccc6cc5c34)cc2-c2ccccc2)cc1 ; c1ccc(-c2ccc(N(c3ccc(-c4ccccc4)c(-c4ccccc4)c3)c3ccc4oc5cc6ccccc6cc5c4c3)cc2-c2ccccc2)cc1]

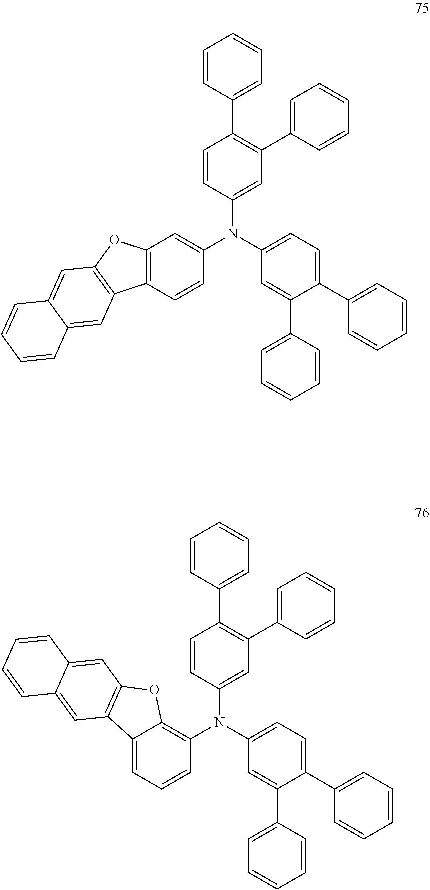 [SMILES: c1ccc(-c2ccc(N(c3ccc(-c4ccccc4)c(-c4ccccc4)c3)c3ccc4c(c3)oc3cc5ccccc5cc34)cc2-c2ccccc2)cc1 ; c1ccc(-c2ccc(N(c3ccc(-c4ccccc4)c(-c4ccccc4)c3)c3cccc4c3oc3cc5ccccc5cc34)cc2-c2ccccc2)cc1]

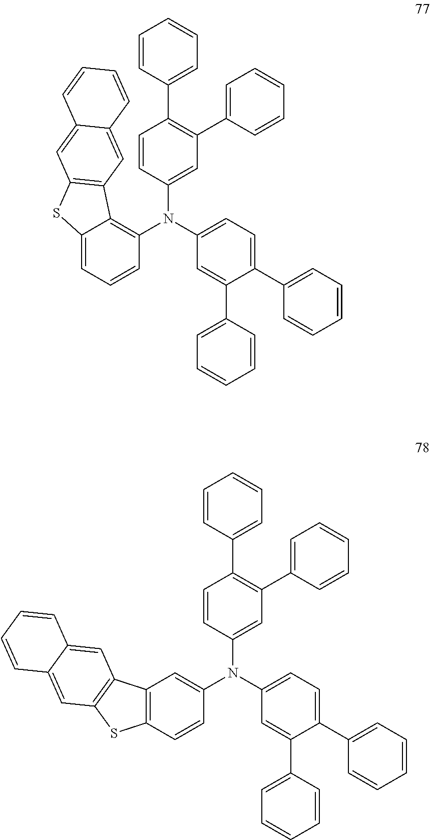 [SMILES: c1ccc(-c2ccc(N(c3ccc(-c4ccccc4)c(-c4ccccc4)c3)c3cccc4sc5cc6ccccc6cc5c34)cc2-c2ccccc2)cc1 ; c1ccc(-c2ccc(N(c3ccc(-c4ccccc4)c(-c4ccccc4)c3)c3ccc4sc5cc6ccccc6cc5c4c3)cc2-c2ccccc2)cc1]

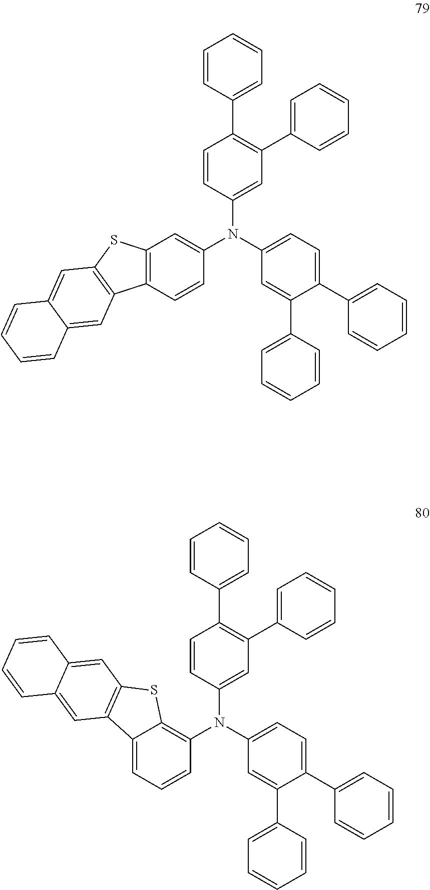 [SMILES: c1ccc(-c2ccc(N(c3ccc(-c4ccccc4)c(-c4ccccc4)c3)c3ccc4c(c3)sc3cc5ccccc5cc34)cc2-c2ccccc2)cc1 ; c1ccc(-c2ccc(N(c3ccc(-c4ccccc4)c(-c4ccccc4)c3)c3cccc4c3sc3cc5ccccc5cc34)cc2-c2ccccc2)cc1]

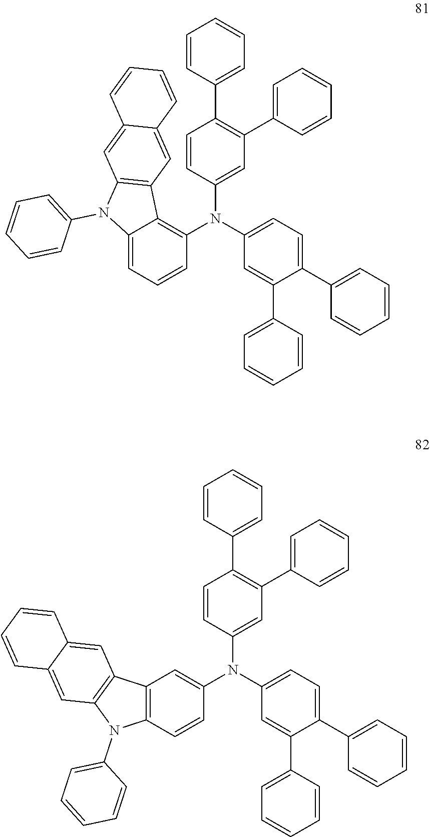 [SMILES: c1ccc(-c2ccc(N(c3ccc(-c4ccccc4)c(-c4ccccc4)c3)c3cccc4c3c3cc5ccccc5cc3n4-c3ccccc3)cc2-c2ccccc2)cc1 ; c1ccc(-c2ccc(N(c3ccc(-c4ccccc4)c(-c4ccccc4)c3)c3ccc4c(c3)c3cc5ccccc5cc3n4-c3ccccc3)cc2-c2ccccc2)cc1]

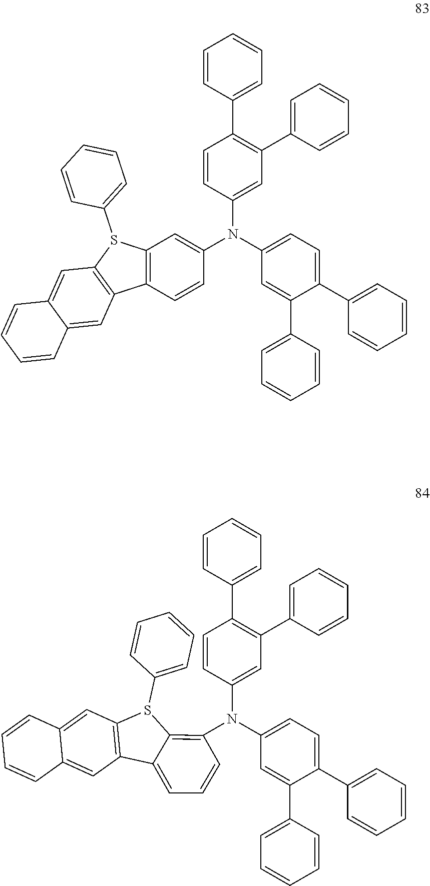

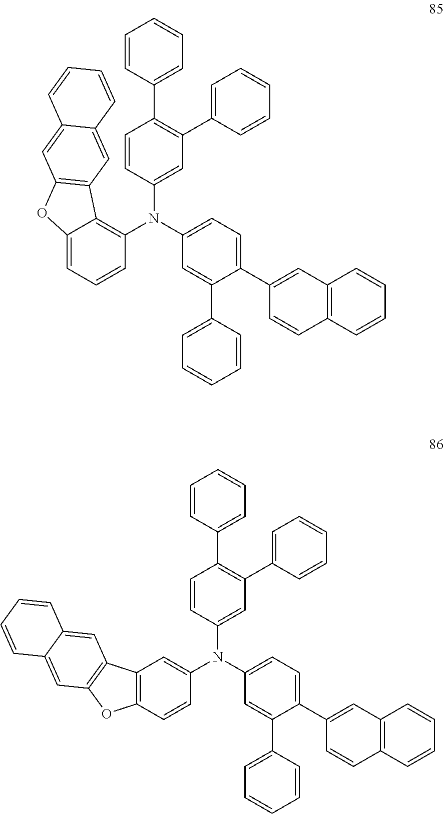 [SMILES: c1ccc(-c2ccc(N(c3ccc(-c4ccc5ccccc5c4)c(-c4ccccc4)c3)c3cccc4oc5cc6ccccc6cc5c34)cc2-c2ccccc2)cc1 ; c1ccc(-c2ccc(N(c3ccc(-c4ccc5ccccc5c4)c(-c4ccccc4)c3)c3ccc4oc5cc6ccccc6cc5c4c3)cc2-c2ccccc2)cc1]

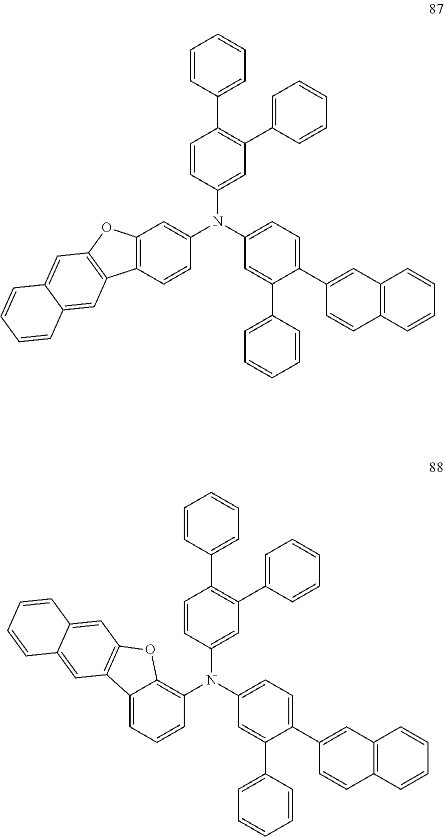 [SMILES: c1ccc(-c2ccc(N(c3ccc(-c4ccc5ccccc5c4)c(-c4ccccc4)c3)c3ccc4c(c3)oc3cc5ccccc5cc34)cc2-c2ccccc2)cc1 ; c1ccc(-c2ccc(N(c3ccc(-c4ccc5ccccc5c4)c(-c4ccccc4)c3)c3cccc4c3oc3cc5ccccc5cc34)cc2-c2ccccc2)cc1]

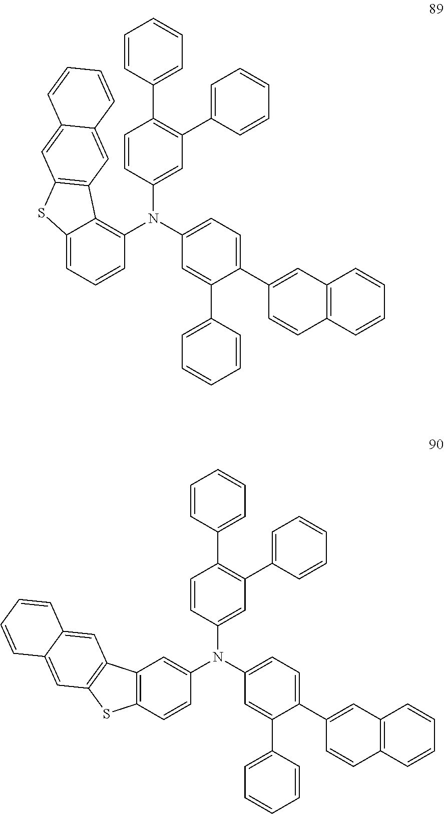 [SMILES: c1ccc(-c2ccc(N(c3ccc(-c4ccc5ccccc5c4)c(-c4ccccc4)c3)c3cccc4sc5cc6ccccc6cc5c34)cc2-c2ccccc2)cc1 ; c1ccc(-c2ccc(N(c3ccc(-c4ccc5ccccc5c4)c(-c4ccccc4)c3)c3ccc4sc5cc6ccccc6cc5c4c3)cc2-c2ccccc2)cc1]

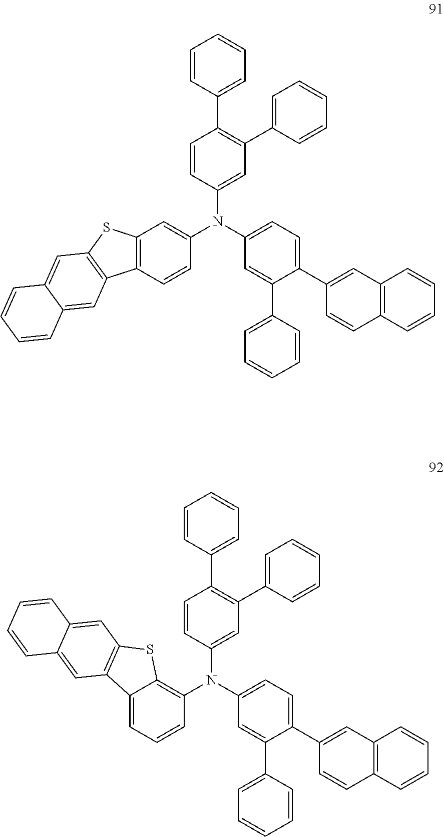 [SMILES: c1ccc(-c2ccc(N(c3ccc(-c4ccc5ccccc5c4)c(-c4ccccc4)c3)c3ccc4c(c3)sc3cc5ccccc5cc34)cc2-c2ccccc2)cc1 ; c1ccc(-c2ccc(N(c3ccc(-c4ccc5ccccc5c4)c(-c4ccccc4)c3)c3cccc4c3sc3cc5ccccc5cc34)cc2-c2ccccc2)cc1]

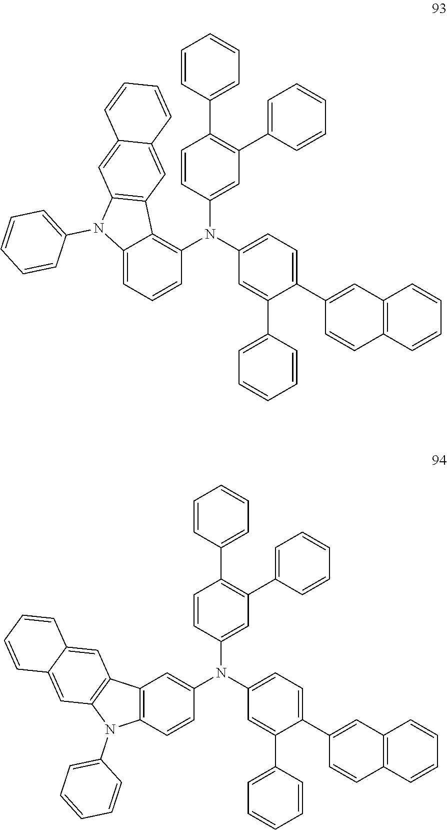 [SMILES: c1ccc(-c2ccc(N(c3ccc(-c4ccc5ccccc5c4)c(-c4ccccc4)c3)c3cccc4c3c3cc5ccccc5cc3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3cc5ccccc5cc3n4-c3ccccc3)cc2-c2ccccc2)cc1]

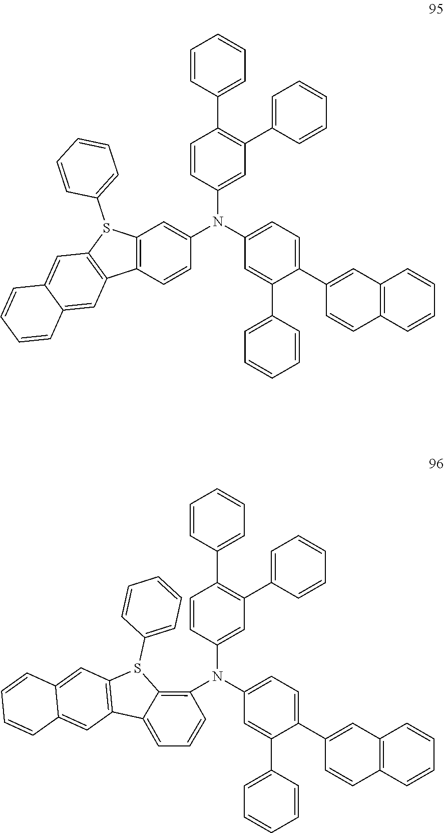

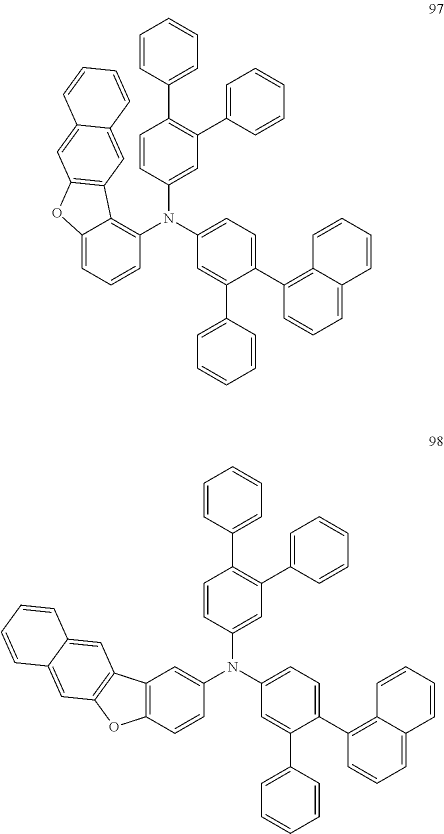 [SMILES: c1ccc(-c2ccc(N(c3ccc(-c4cccc5ccccc45)c(-c4ccccc4)c3)c3cccc4oc5cc6ccccc6cc5c34)cc2-c2ccccc2)cc1 ; c1ccc(-c2ccc(N(c3ccc(-c4cccc5ccccc45)c(-c4ccccc4)c3)c3ccc4oc5cc6ccccc6cc5c4c3)cc2-c2ccccc2)cc1]

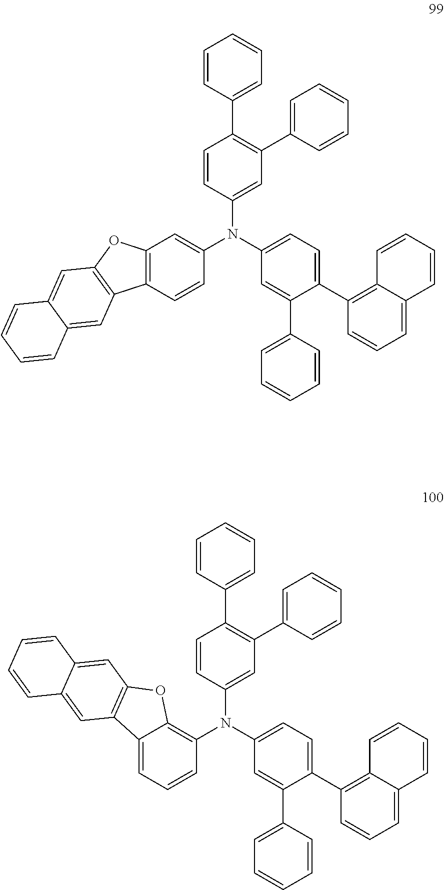 [SMILES: c1ccc(-c2ccc(N(c3ccc(-c4cccc5ccccc45)c(-c4ccccc4)c3)c3ccc4c(c3)oc3cc5ccccc5cc34)cc2-c2ccccc2)cc1 ; c1ccc(-c2ccc(N(c3ccc(-c4cccc5ccccc45)c(-c4ccccc4)c3)c3cccc4c3oc3cc5ccccc5cc34)cc2-c2ccccc2)cc1]

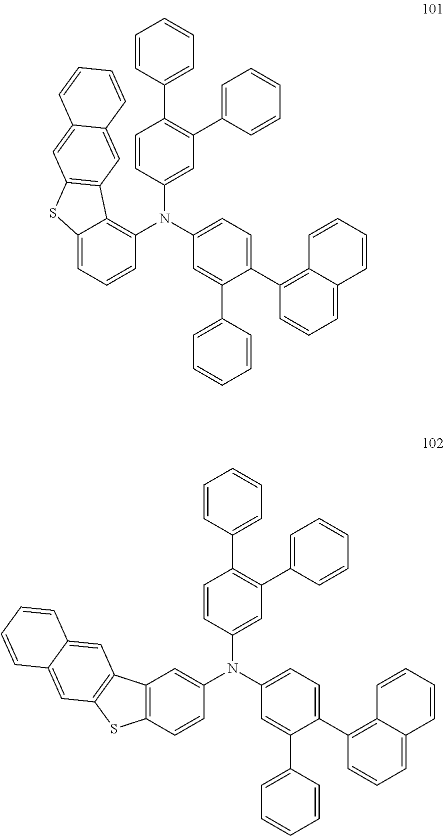 [SMILES: c1ccc(-c2ccc(N(c3ccc(-c4cccc5ccccc45)c(-c4ccccc4)c3)c3cccc4sc5cc6ccccc6cc5c34)cc2-c2ccccc2)cc1 ; c1ccc(-c2ccc(N(c3ccc(-c4cccc5ccccc45)c(-c4ccccc4)c3)c3ccc4sc5cc6ccccc6cc5c4c3)cc2-c2ccccc2)cc1]

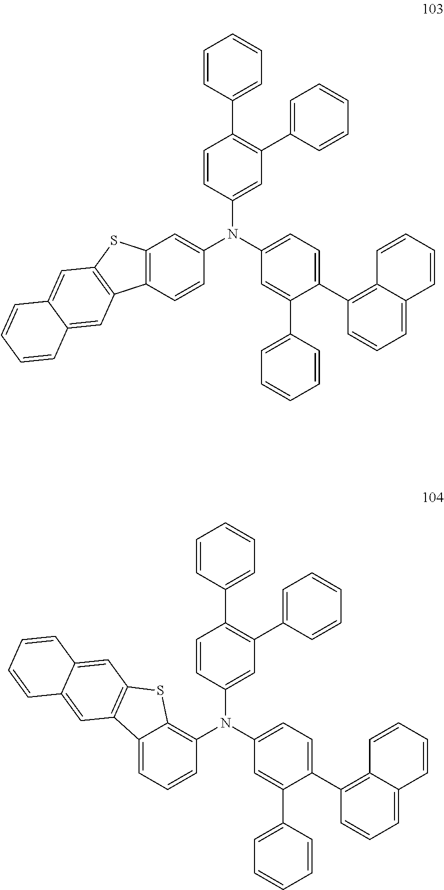 [SMILES: c1ccc(-c2ccc(N(c3ccc(-c4cccc5ccccc45)c(-c4ccccc4)c3)c3ccc4c(c3)sc3cc5ccccc5cc34)cc2-c2ccccc2)cc1 ; c1ccc(-c2ccc(N(c3ccc(-c4cccc5ccccc45)c(-c4ccccc4)c3)c3cccc4c3sc3cc5ccccc5cc34)cc2-c2ccccc2)cc1]

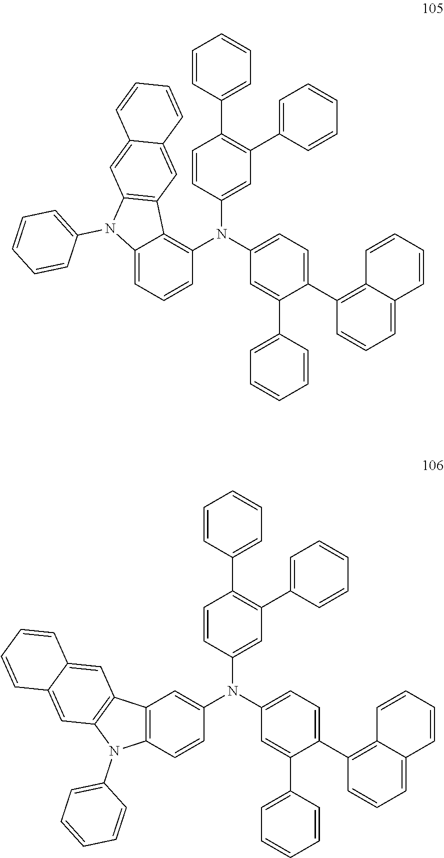 [SMILES: c1ccc(-c2ccc(N(c3ccc(-c4cccc5ccccc45)c(-c4ccccc4)c3)c3cccc4c3c3cc5ccccc5cc3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3cc5ccccc5cc3n4-c3ccccc3)cc2-c2ccccc2)cc1]

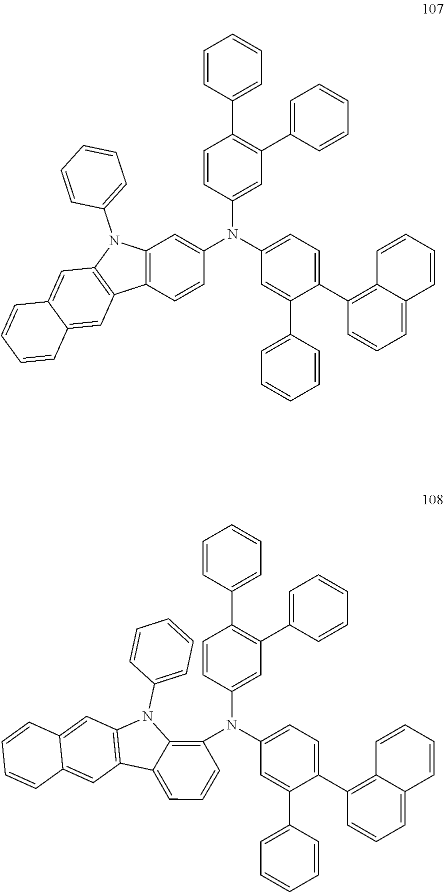 [SMILES: c1ccc(-c2ccc(N(c3ccc(-c4cccc5ccccc45)c(-c4ccccc4)c3)c3ccc4c5cc6ccccc6cc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5cc6ccccc6cc5n(-c5ccccc5)c34)cc2-c2ccccc2)cc1]

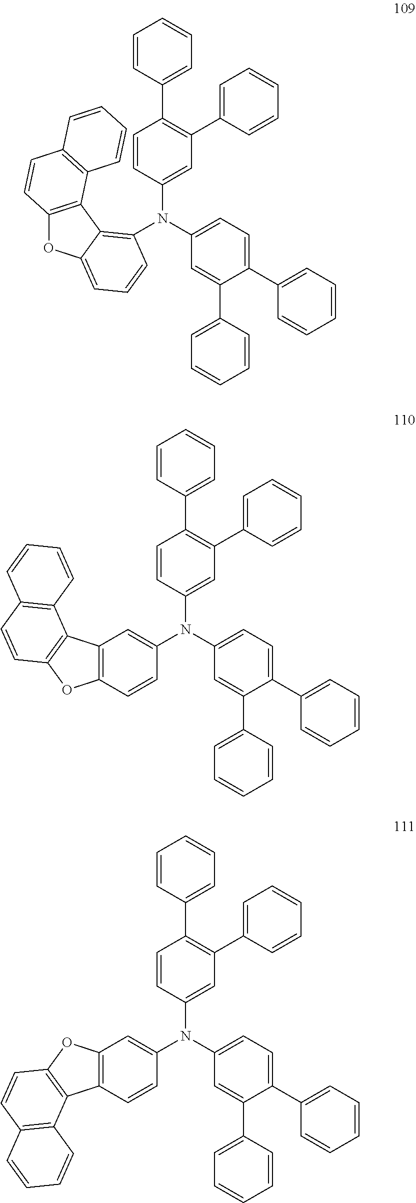 [SMILES: c1ccc(-c2ccc(N(c3ccc(-c4ccccc4)c(-c4ccccc4)c3)c3cccc4oc5ccc6ccccc6c5c34)cc2-c2ccccc2)cc1 ; c1ccc(-c2ccc(N(c3ccc(-c4ccccc4)c(-c4ccccc4)c3)c3ccc4oc5ccc6ccccc6c5c4c3)cc2-c2ccccc2)cc1 ; c1ccc(-c2ccc(N(c3ccc(-c4ccccc4)c(-c4ccccc4)c3)c3ccc4c(c3)oc3ccc5ccccc5c34)cc2-c2ccccc2)cc1]

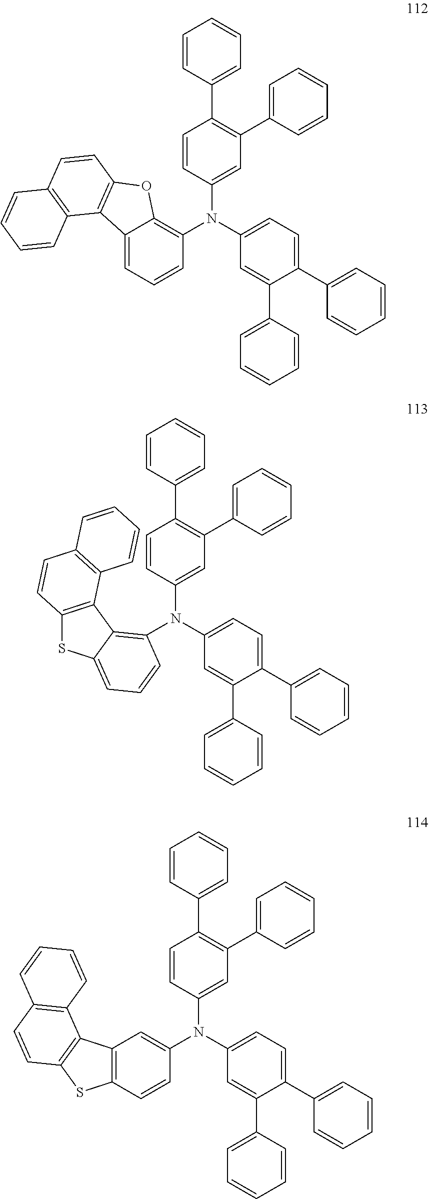 [SMILES: c1ccc(-c2ccc(N(c3ccc(-c4ccccc4)c(-c4ccccc4)c3)c3cccc4c3oc3ccc5ccccc5c34)cc2-c2ccccc2)cc1 ; c1ccc(-c2ccc(N(c3ccc(-c4ccccc4)c(-c4ccccc4)c3)c3cccc4sc5ccc6ccccc6c5c34)cc2-c2ccccc2)cc1 ; c1ccc(-c2ccc(N(c3ccc(-c4ccccc4)c(-c4ccccc4)c3)c3ccc4sc5ccc6ccccc6c5c4c3)cc2-c2ccccc2)cc1]

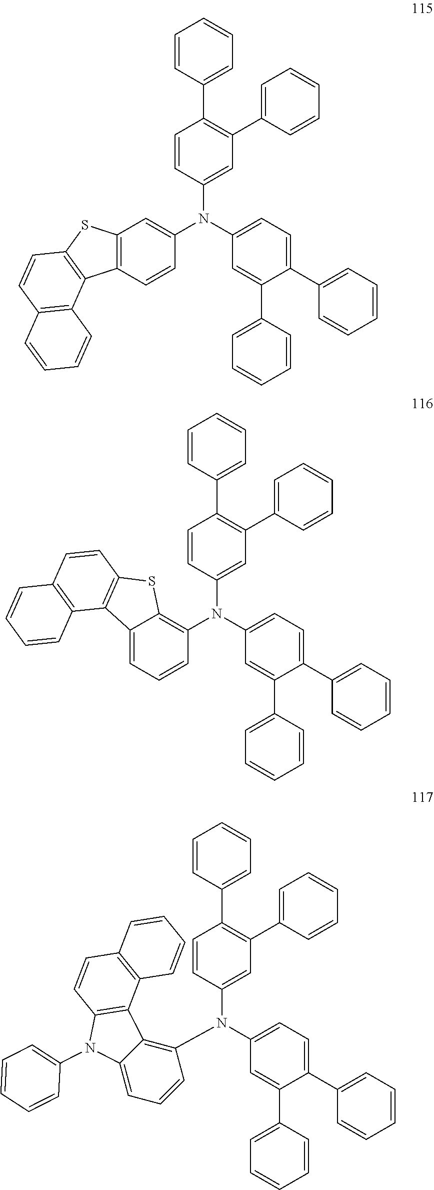 [SMILES: c1ccc(-c2ccc(N(c3ccc(-c4ccccc4)c(-c4ccccc4)c3)c3ccc4c(c3)sc3ccc5ccccc5c34)cc2-c2ccccc2)cc1 ; c1ccc(-c2ccc(N(c3ccc(-c4ccccc4)c(-c4ccccc4)c3)c3cccc4c3sc3ccc5ccccc5c34)cc2-c2ccccc2)cc1 ; c1ccc(-c2ccc(N(c3ccc(-c4ccccc4)c(-c4ccccc4)c3)c3cccc4c3c3c5ccccc5ccc3n4-c3ccccc3)cc2-c2ccccc2)cc1]

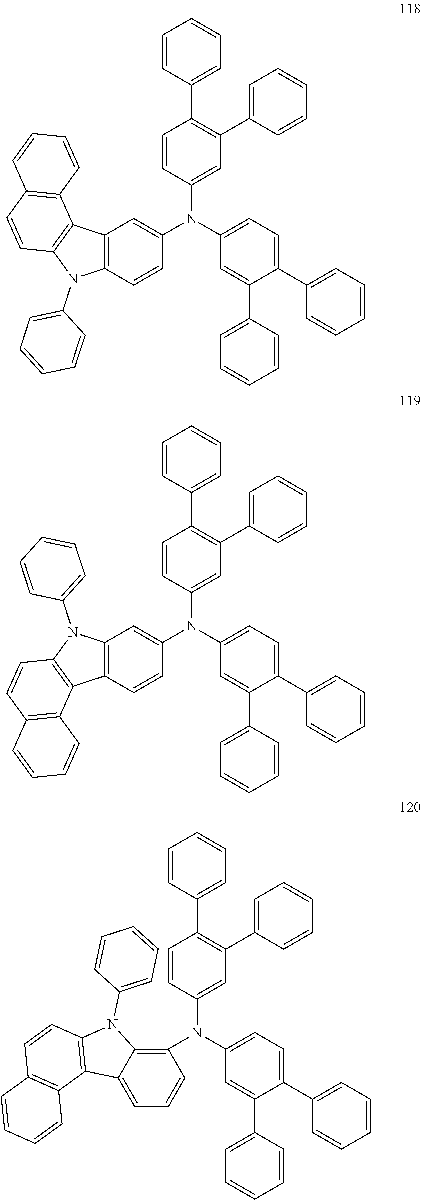 [SMILES: c1ccc(-c2ccc(N(c3ccc(-c4ccccc4)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4ccccc4)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4ccccc4)c(-c4ccccc4)c3)c3cccc4c5c6ccccc6ccc5n(-c5ccccc5)c34)cc2-c2ccccc2)cc1]

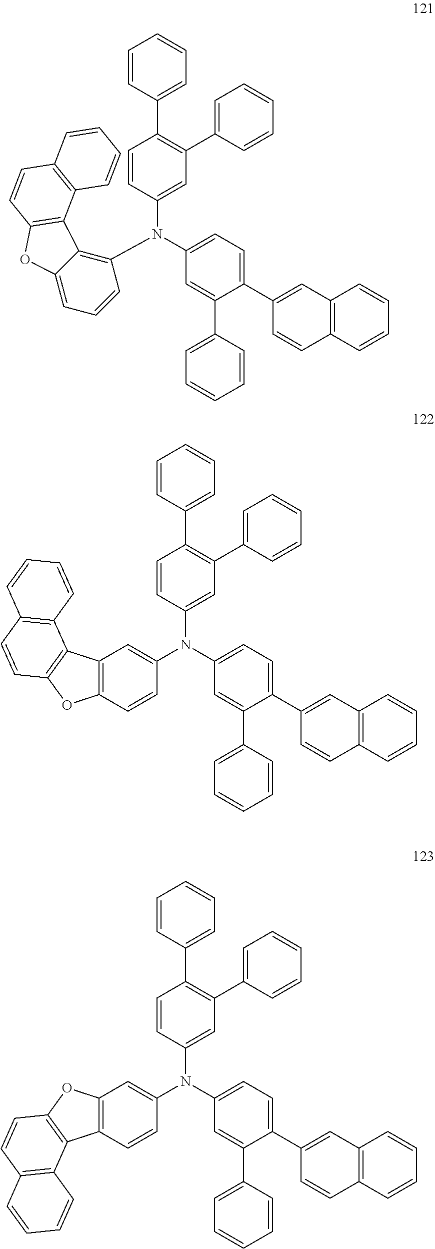 [SMILES: c1ccc(-c2ccc(N(c3ccc(-c4ccc5ccccc5c4)c(-c4ccccc4)c3)c3cccc4oc5ccc6ccccc6c5c34)cc2-c2ccccc2)cc1 ; c1ccc(-c2ccc(N(c3ccc(-c4ccc5ccccc5c4)c(-c4ccccc4)c3)c3ccc4oc5ccc6ccccc6c5c4c3)cc2-c2ccccc2)cc1 ; c1ccc(-c2ccc(N(c3ccc(-c4ccc5ccccc5c4)c(-c4ccccc4)c3)c3ccc4c(c3)oc3ccc5ccccc5c34)cc2-c2ccccc2)cc1]

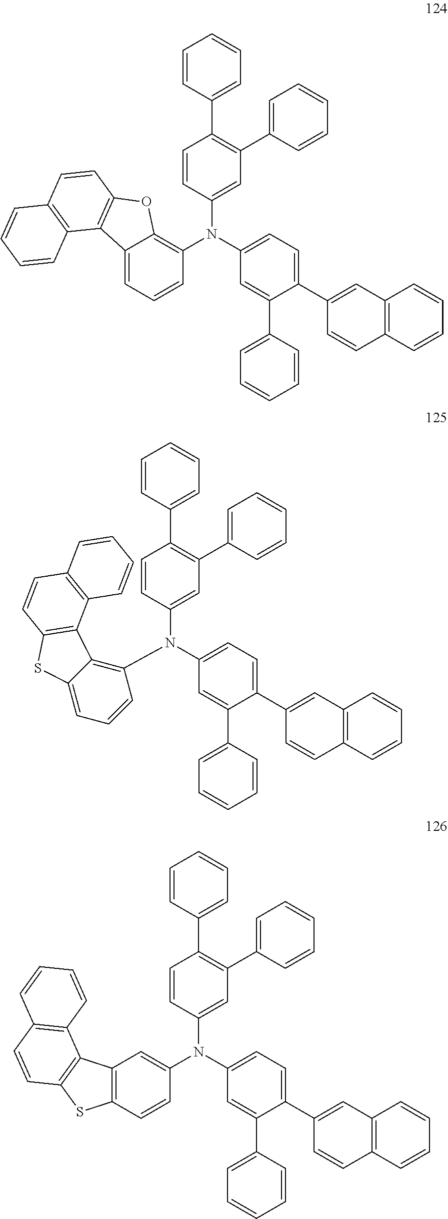 [SMILES: c1ccc(-c2ccc(N(c3ccc(-c4ccc5ccccc5c4)c(-c4ccccc4)c3)c3cccc4c3oc3ccc5ccccc5c34)cc2-c2ccccc2)cc1 ; c1ccc(-c2ccc(N(c3ccc(-c4ccc5ccccc5c4)c(-c4ccccc4)c3)c3cccc4sc5ccc6ccccc6c5c34)cc2-c2ccccc2)cc1 ; c1ccc(-c2ccc(N(c3ccc(-c4ccc5ccccc5c4)c(-c4ccccc4)c3)c3ccc4sc5ccc6ccccc6c5c4c3)cc2-c2ccccc2)cc1]

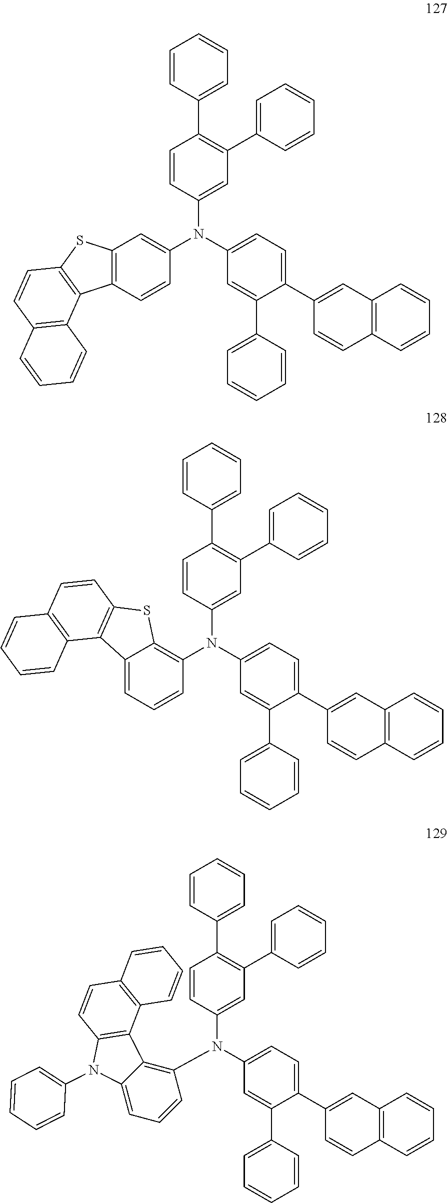 [SMILES: c1ccc(-c2ccc(N(c3ccc(-c4ccc5ccccc5c4)c(-c4ccccc4)c3)c3ccc4c(c3)sc3ccc5ccccc5c34)cc2-c2ccccc2)cc1 ; c1ccc(-c2ccc(N(c3ccc(-c4ccc5ccccc5c4)c(-c4ccccc4)c3)c3cccc4c3sc3ccc5ccccc5c34)cc2-c2ccccc2)cc1 ; c1ccc(-c2ccc(N(c3ccc(-c4ccc5ccccc5c4)c(-c4ccccc4)c3)c3cccc4c3c3c5ccccc5ccc3n4-c3ccccc3)cc2-c2ccccc2)cc1]

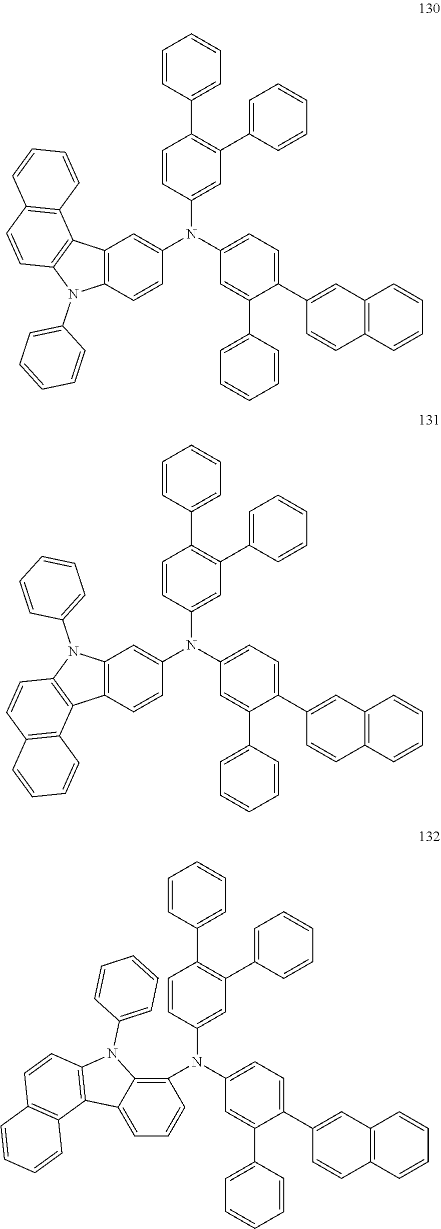 [SMILES: c1ccc(-c2ccc(N(c3ccc(-c4ccc5ccccc5c4)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4ccc5ccccc5c4)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5c6ccccc6ccc5n(-c5ccccc5)c34)cc2-c2ccccc2)cc1]

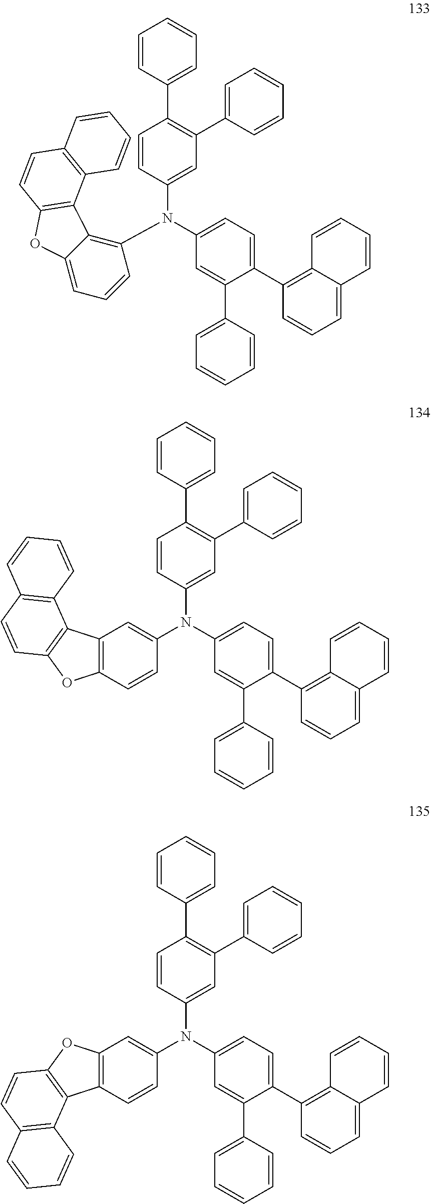 [SMILES: c1ccc(-c2ccc(N(c3ccc(-c4cccc5ccccc45)c(-c4ccccc4)c3)c3cccc4oc5ccc6ccccc6c5c34)cc2-c2ccccc2)cc1 ; c1ccc(-c2ccc(N(c3ccc(-c4cccc5ccccc45)c(-c4ccccc4)c3)c3ccc4oc5ccc6ccccc6c5c4c3)cc2-c2ccccc2)cc1 ; c1ccc(-c2ccc(N(c3ccc(-c4cccc5ccccc45)c(-c4ccccc4)c3)c3ccc4c(c3)oc3ccc5ccccc5c34)cc2-c2ccccc2)cc1]

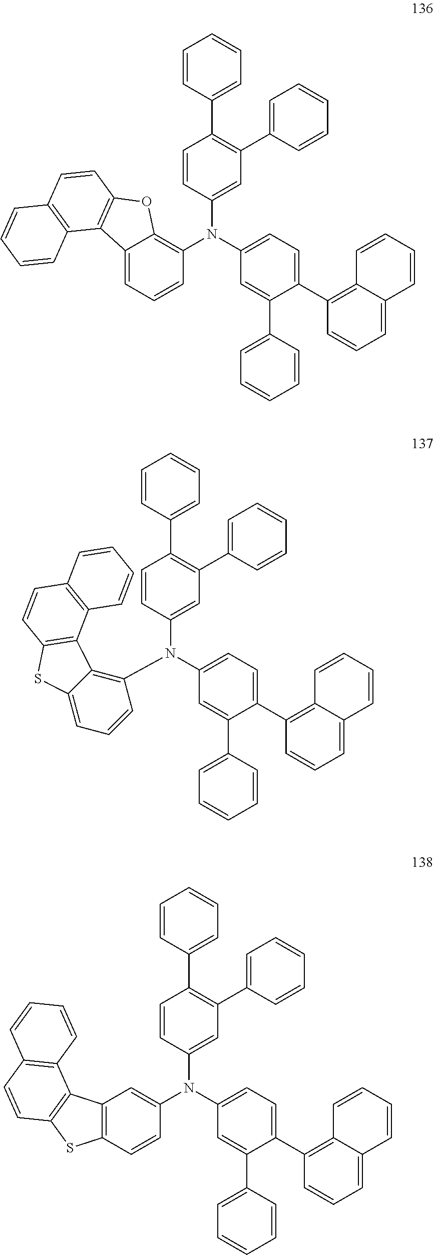 [SMILES: c1ccc(-c2ccc(N(c3ccc(-c4cccc5ccccc45)c(-c4ccccc4)c3)c3cccc4c3oc3ccc5ccccc5c34)cc2-c2ccccc2)cc1 ; c1ccc(-c2ccc(N(c3ccc(-c4cccc5ccccc45)c(-c4ccccc4)c3)c3cccc4sc5ccc6ccccc6c5c34)cc2-c2ccccc2)cc1 ; c1ccc(-c2ccc(N(c3ccc(-c4cccc5ccccc45)c(-c4ccccc4)c3)c3ccc4sc5ccc6ccccc6c5c4c3)cc2-c2ccccc2)cc1]

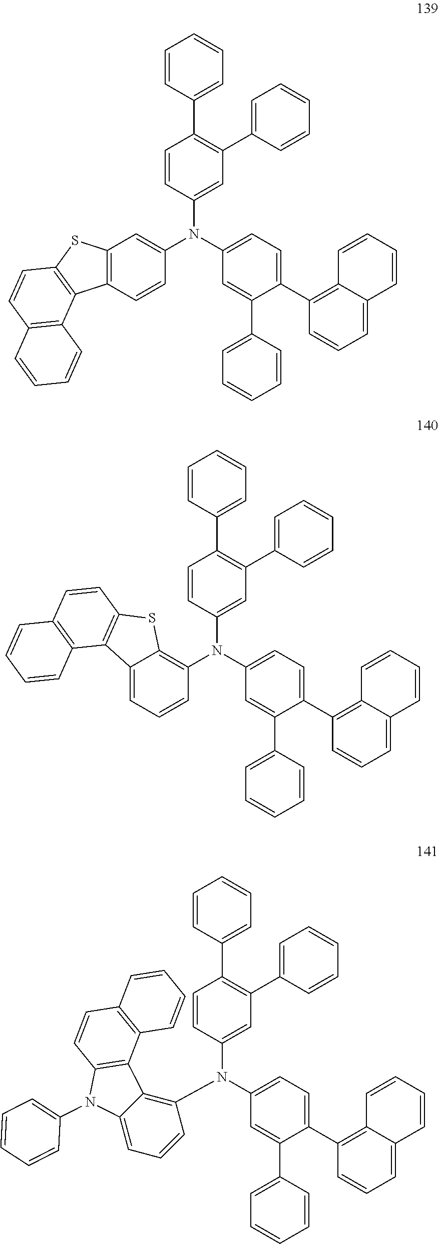 [SMILES: c1ccc(-c2ccc(N(c3ccc(-c4cccc5ccccc45)c(-c4ccccc4)c3)c3ccc4c(c3)sc3ccc5ccccc5c34)cc2-c2ccccc2)cc1 ; c1ccc(-c2ccc(N(c3ccc(-c4cccc5ccccc45)c(-c4ccccc4)c3)c3cccc4c3sc3ccc5ccccc5c34)cc2-c2ccccc2)cc1 ; c1ccc(-c2ccc(N(c3ccc(-c4cccc5ccccc45)c(-c4ccccc4)c3)c3cccc4c3c3c5ccccc5ccc3n4-c3ccccc3)cc2-c2ccccc2)cc1]

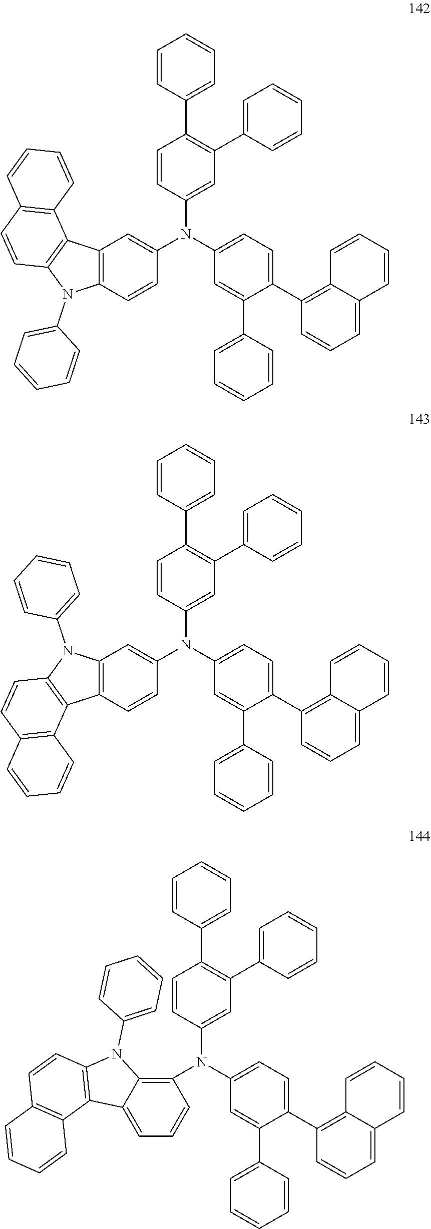 [SMILES: c1ccc(-c2ccc(N(c3ccc(-c4cccc5ccccc45)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4cccc5ccccc45)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5c6ccccc6ccc5n(-c5ccccc5)c34)cc2-c2ccccc2)cc1]

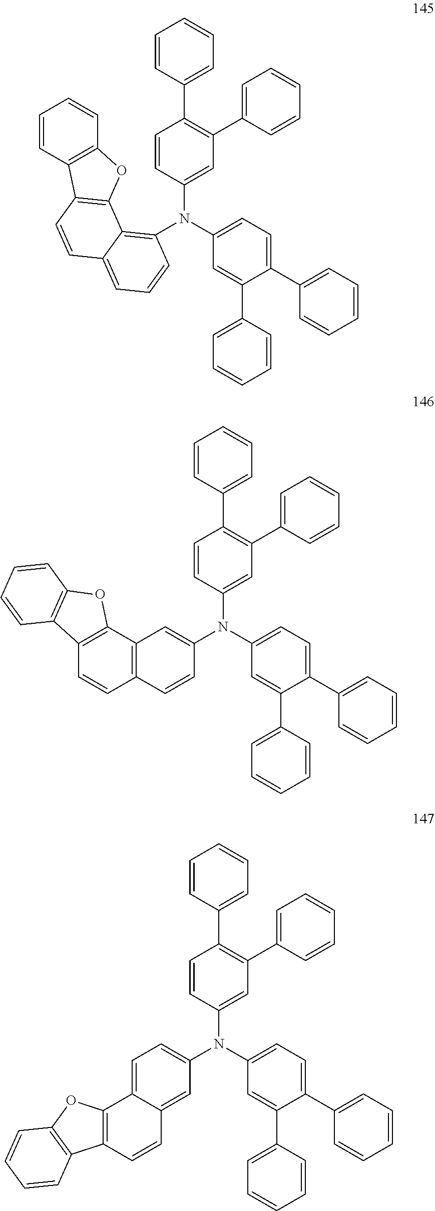

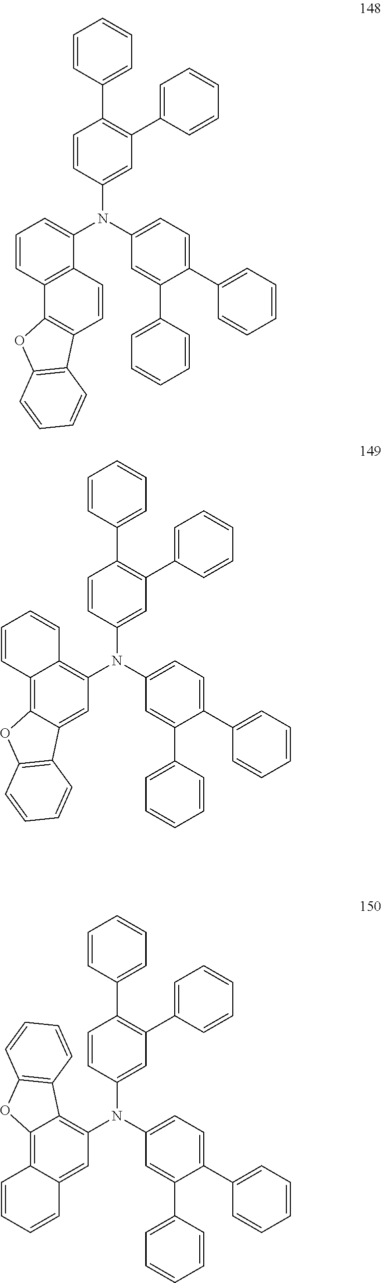 [SMILES: c1ccc(-c2ccc(N(c3ccc(-c4ccccc4)c(-c4ccccc4)c3)c3cccc4c3ccc3c5ccccc5oc43)cc2-c2ccccc2)cc1 ; c1ccc(-c2ccc(N(c3ccc(-c4ccccc4)c(-c4ccccc4)c3)c3cc4c5ccccc5oc4c4ccccc34)cc2-c2ccccc2)cc1 ; c1ccc(-c2ccc(N(c3ccc(-c4ccccc4)c(-c4ccccc4)c3)c3cc4ccccc4c4oc5ccccc5c34)cc2-c2ccccc2)cc1]

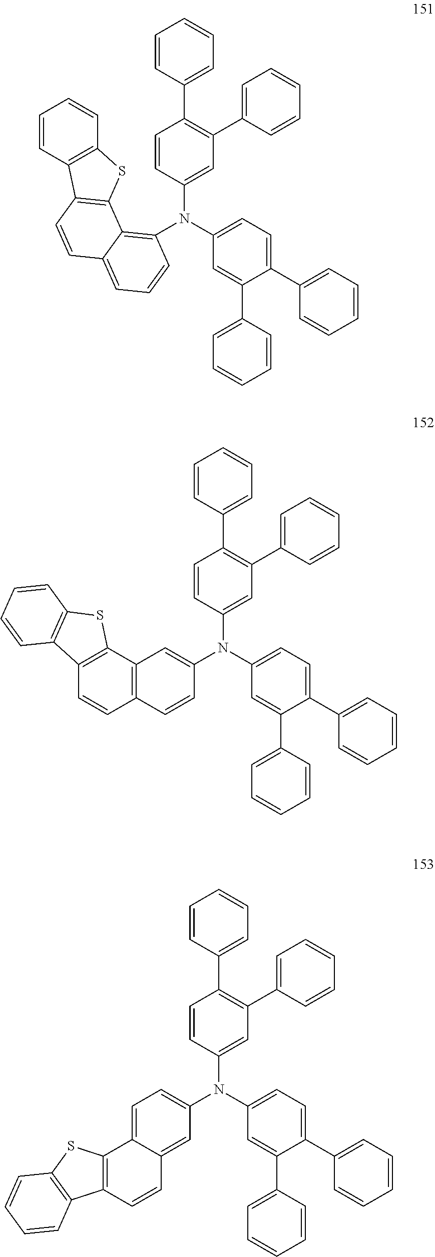 [SMILES: c1ccc(-c2ccc(N(c3ccc(-c4ccccc4)c(-c4ccccc4)c3)c3cccc4ccc5c6ccccc6sc5c34)cc2-c2ccccc2)cc1 ; c1ccc(-c2ccc(N(c3ccc(-c4ccccc4)c(-c4ccccc4)c3)c3ccc4ccc5c6ccccc6sc5c4c3)cc2-c2ccccc2)cc1 ; c1ccc(-c2ccc(N(c3ccc(-c4ccccc4)c(-c4ccccc4)c3)c3ccc4c(ccc5c6ccccc6sc45)c3)cc2-c2ccccc2)cc1]

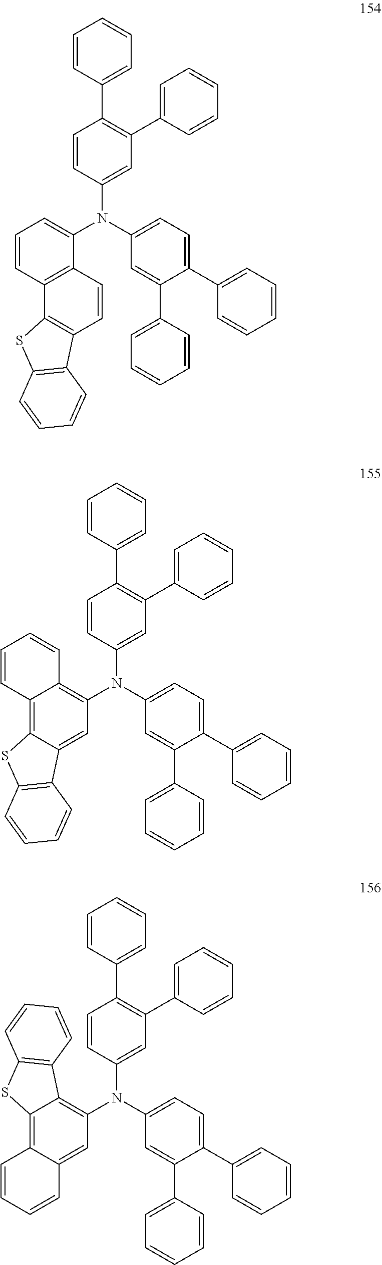 [SMILES: c1ccc(-c2ccc(N(c3ccc(-c4ccccc4)c(-c4ccccc4)c3)c3cccc4c3ccc3c5ccccc5sc43)cc2-c2ccccc2)cc1 ; c1ccc(-c2ccc(N(c3ccc(-c4ccccc4)c(-c4ccccc4)c3)c3cc4c5ccccc5sc4c4ccccc34)cc2-c2ccccc2)cc1 ; c1ccc(-c2ccc(N(c3ccc(-c4ccccc4)c(-c4ccccc4)c3)c3cc4ccccc4c4sc5ccccc5c34)cc2-c2ccccc2)cc1]

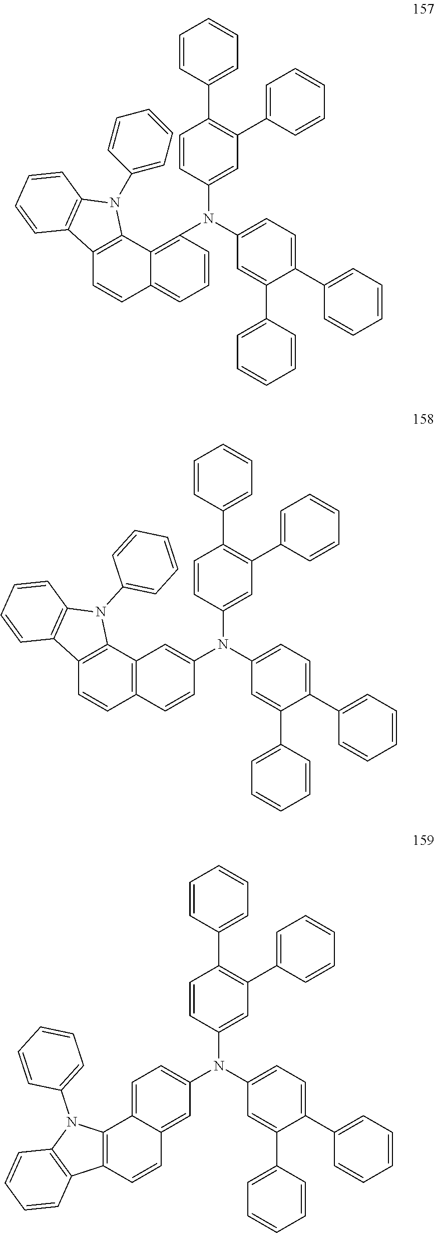 [SMILES: c1ccc(-c2ccc(N(c3ccc(-c4ccccc4)c(-c4ccccc4)c3)c3cccc4ccc5c6ccccc6n(-c6ccccc6)c5c34)cc2-c2ccccc2)cc1 ; c1ccc(-c2ccc(N(c3ccc(-c4ccccc4)c(-c4ccccc4)c3)c3ccc4ccc5c6ccccc6n(-c6ccccc6)c5c4c3)cc2-c2ccccc2)cc1 ; c1ccc(-c2ccc(N(c3ccc(-c4ccccc4)c(-c4ccccc4)c3)c3ccc4c(ccc5c6ccccc6n(-c6ccccc6)c45)c3)cc2-c2ccccc2)cc1]

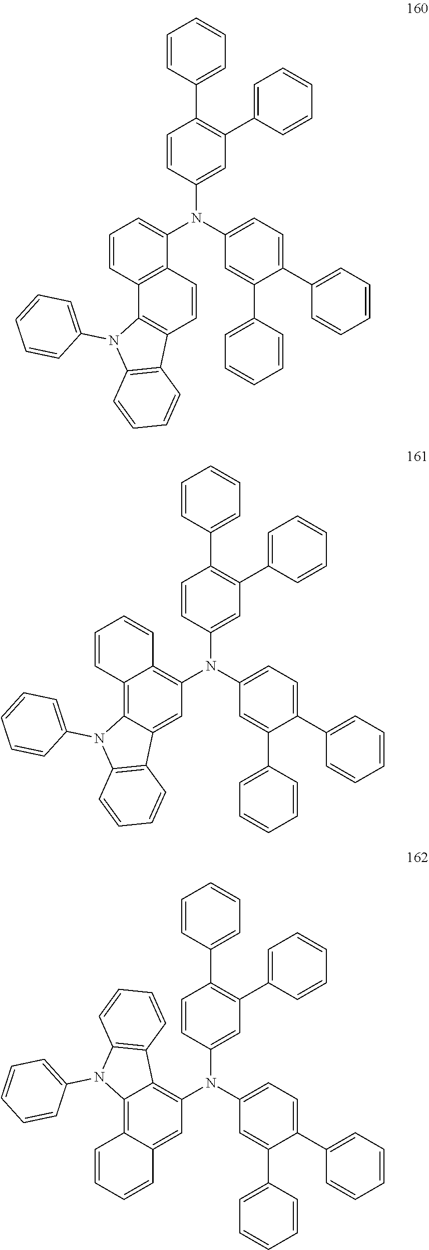 [SMILES: c1ccc(-c2ccc(N(c3ccc(-c4ccccc4)c(-c4ccccc4)c3)c3cccc4c3ccc3c5ccccc5n(-c5ccccc5)c43)cc2-c2ccccc2)cc1 ; c1ccc(-c2ccc(N(c3ccc(-c4ccccc4)c(-c4ccccc4)c3)c3cc4c5ccccc5n(-c5ccccc5)c4c4ccccc34)cc2-c2ccccc2)cc1 ; c1ccc(-c2ccc(N(c3ccc(-c4ccccc4)c(-c4ccccc4)c3)c3cc4ccccc4c4c3c3ccccc3n4-c3ccccc3)cc2-c2ccccc2)cc1]

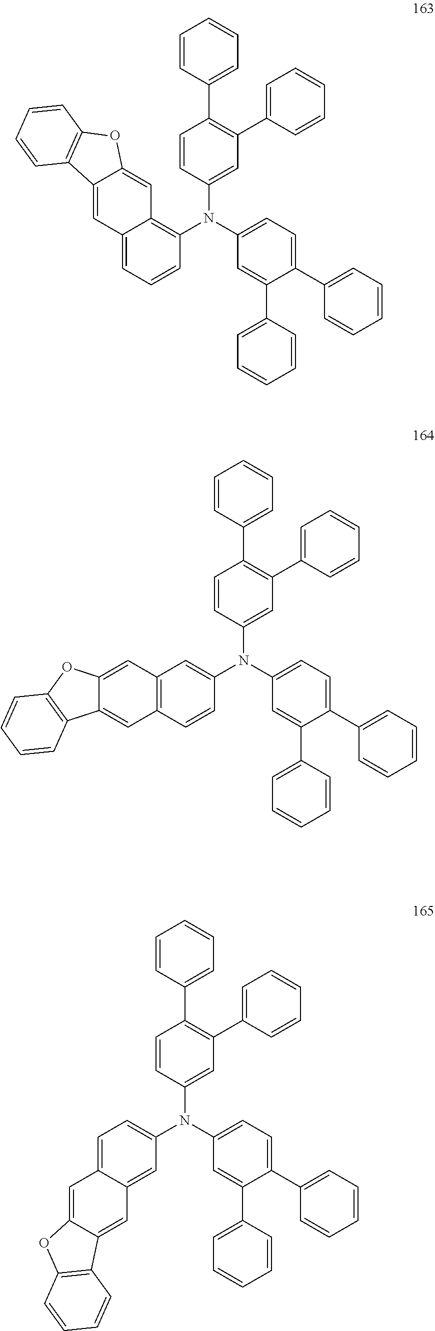 [SMILES: c1ccc(-c2ccc(N(c3ccc(-c4ccccc4)c(-c4ccccc4)c3)c3cccc4cc5c(cc34)oc3ccccc35)cc2-c2ccccc2)cc1 ; c1ccc(-c2ccc(N(c3ccc(-c4ccccc4)c(-c4ccccc4)c3)c3ccc4cc5c(cc4c3)oc3ccccc35)cc2-c2ccccc2)cc1 ; c1ccc(-c2ccc(N(c3ccc(-c4ccccc4)c(-c4ccccc4)c3)c3ccc4cc5oc6ccccc6c5cc4c3)cc2-c2ccccc2)cc1]

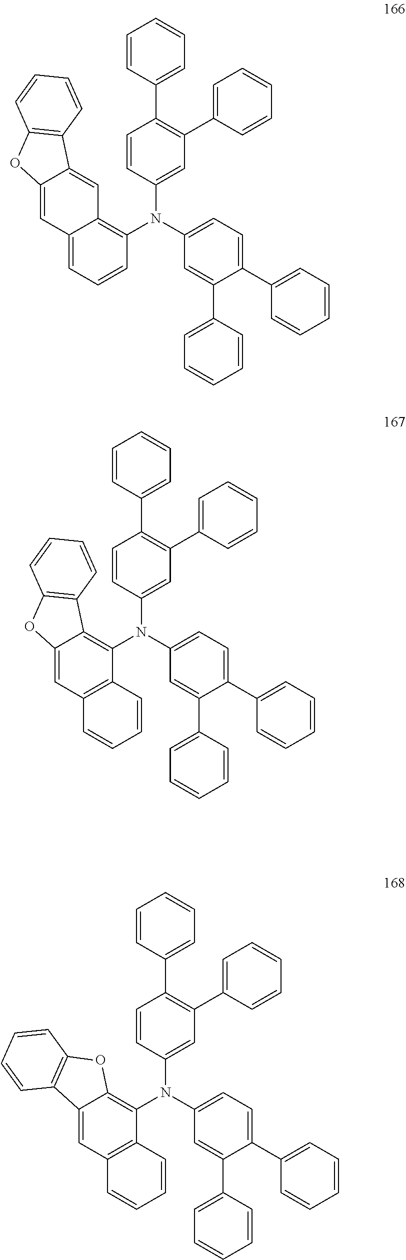 [SMILES: c1ccc(-c2ccc(N(c3ccc(-c4ccccc4)c(-c4ccccc4)c3)c3cccc4cc5oc6ccccc6c5cc34)cc2-c2ccccc2)cc1 ; c1ccc(-c2ccc(N(c3ccc(-c4ccccc4)c(-c4ccccc4)c3)c3c4ccccc4cc4oc5ccccc5c34)cc2-c2ccccc2)cc1 ; c1ccc(-c2ccc(N(c3ccc(-c4ccccc4)c(-c4ccccc4)c3)c3c4ccccc4cc4c3oc3ccccc34)cc2-c2ccccc2)cc1]

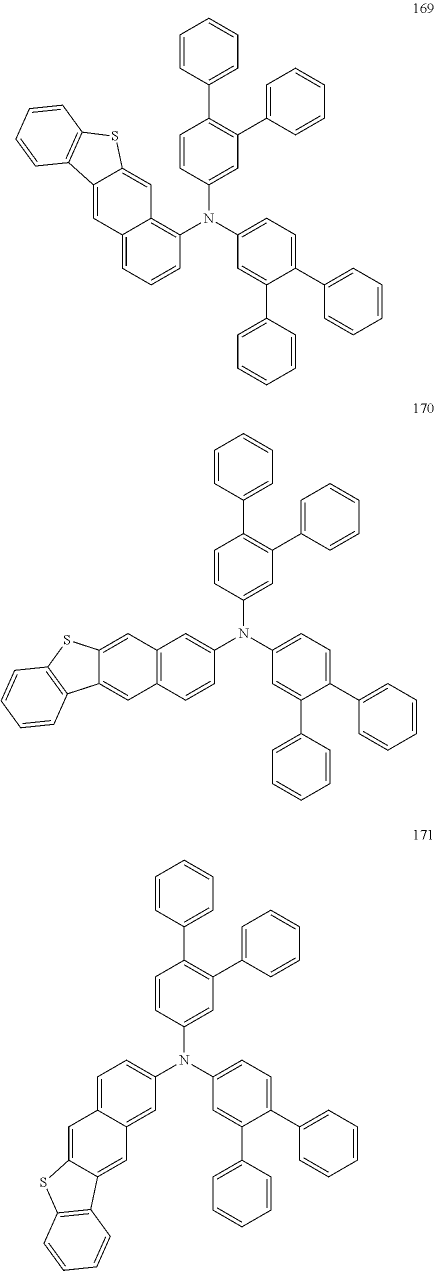 [SMILES: c1ccc(-c2ccc(N(c3ccc(-c4ccccc4)c(-c4ccccc4)c3)c3cccc4cc5c(cc34)sc3ccccc35)cc2-c2ccccc2)cc1 ; c1ccc(-c2ccc(N(c3ccc(-c4ccccc4)c(-c4ccccc4)c3)c3ccc4cc5c(cc4c3)sc3ccccc35)cc2-c2ccccc2)cc1 ; c1ccc(-c2ccc(N(c3ccc(-c4ccccc4)c(-c4ccccc4)c3)c3ccc4cc5sc6ccccc6c5cc4c3)cc2-c2ccccc2)cc1]

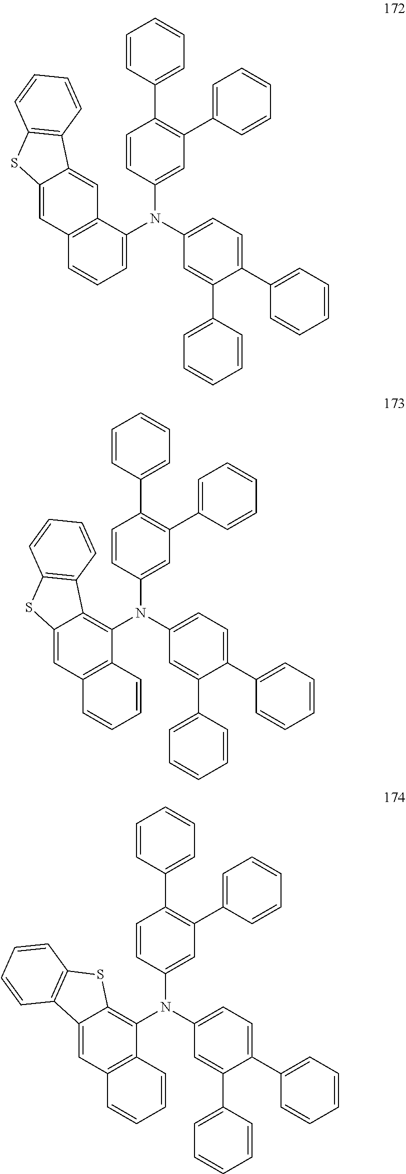

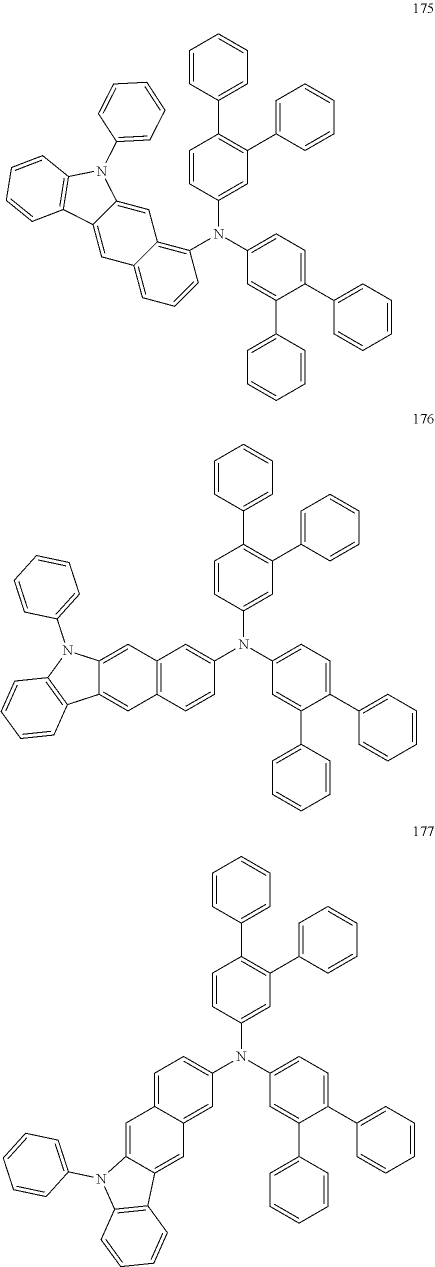 [SMILES: c1ccc(-c2ccc(N(c3ccc(-c4ccccc4)c(-c4ccccc4)c3)c3cccc4cc5c6ccccc6n(-c6ccccc6)c5cc34)cc2-c2ccccc2)cc1 ; c1ccc(-c2ccc(N(c3ccc(-c4ccccc4)c(-c4ccccc4)c3)c3ccc4cc5c6ccccc6n(-c6ccccc6)c5cc4c3)cc2-c2ccccc2)cc1 ; c1ccc(-c2ccc(N(c3ccc(-c4ccccc4)c(-c4ccccc4)c3)c3ccc4cc5c(cc4c3)c3ccccc3n5-c3ccccc3)cc2-c2ccccc2)cc1]

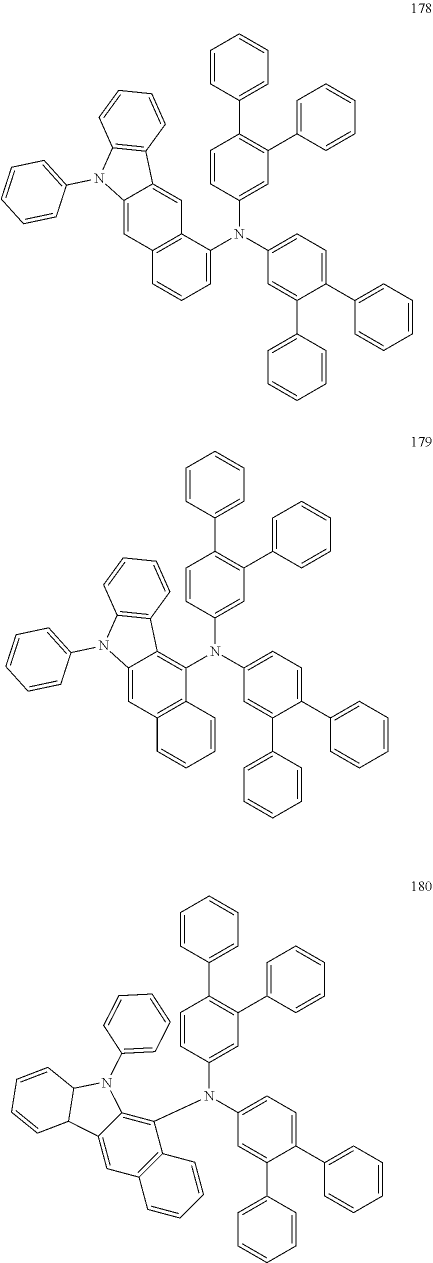 [SMILES: c1ccc(-c2ccc(N(c3ccc(-c4ccccc4)c(-c4ccccc4)c3)c3cccc4cc5c(cc34)c3ccccc3n5-c3ccccc3)cc2-c2ccccc2)cc1 ; c1ccc(-c2ccc(N(c3ccc(-c4ccccc4)c(-c4ccccc4)c3)c3c4ccccc4cc4c3c3ccccc3n4-c3ccccc3)cc2-c2ccccc2)cc1 ; C1=CC2c3cc4ccccc4c(N(c4ccc(-c5ccccc5)c(-c5ccccc5)c4)c4ccc(-c5ccccc5)c(-c5ccccc5)c4)c3N(c3ccccc3)C2C=C1]

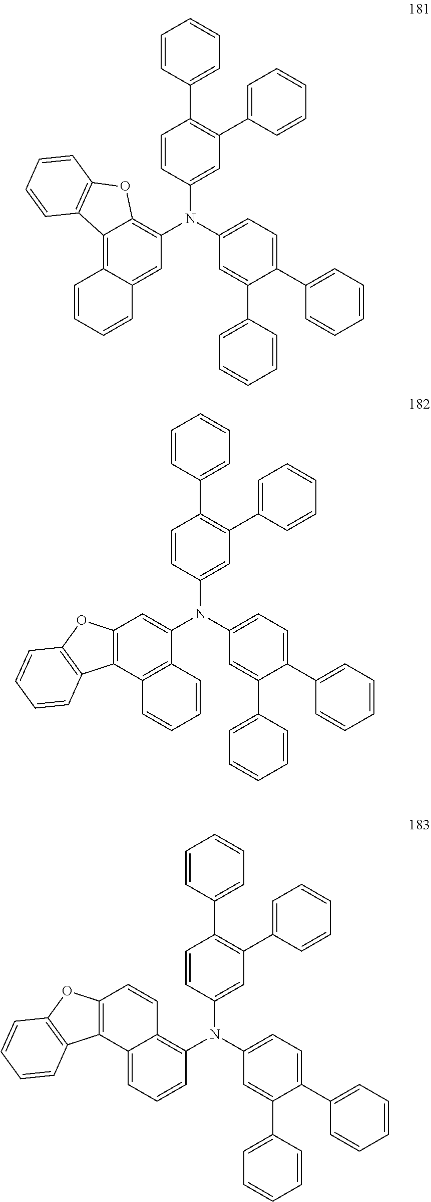 [SMILES: c1ccc(-c2ccc(N(c3ccc(-c4ccccc4)c(-c4ccccc4)c3)c3cc4ccccc4c4c3oc3ccccc34)cc2-c2ccccc2)cc1 ; c1ccc(-c2ccc(N(c3ccc(-c4ccccc4)c(-c4ccccc4)c3)c3cc4oc5ccccc5c4c4ccccc34)cc2-c2ccccc2)cc1 ; c1ccc(-c2ccc(N(c3ccc(-c4ccccc4)c(-c4ccccc4)c3)c3cccc4c3ccc3oc5ccccc5c34)cc2-c2ccccc2)cc1]

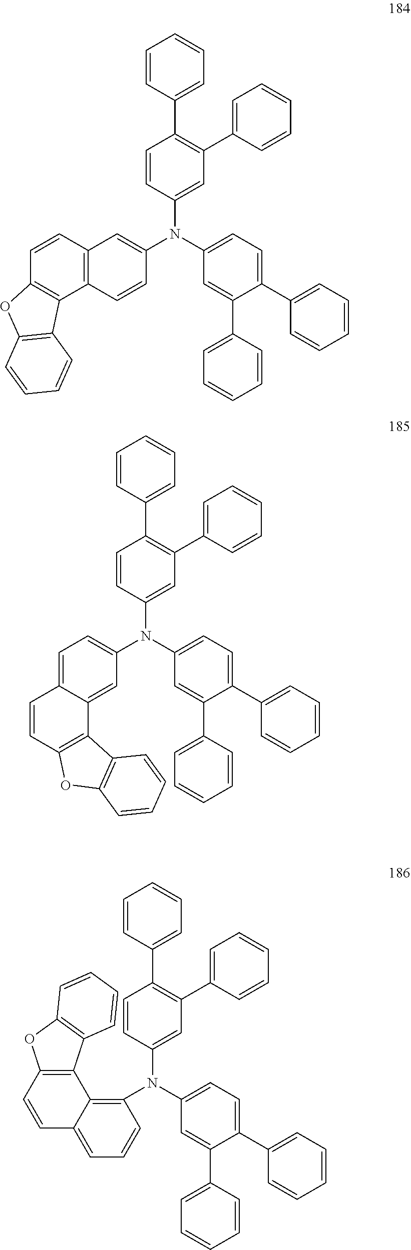 [SMILES: c1ccc(-c2ccc(N(c3ccc(-c4ccccc4)c(-c4ccccc4)c3)c3ccc4c(ccc5oc6ccccc6c54)c3)cc2-c2ccccc2)cc1 ; c1ccc(-c2ccc(N(c3ccc(-c4ccccc4)c(-c4ccccc4)c3)c3ccc4ccc5oc6ccccc6c5c4c3)cc2-c2ccccc2)cc1 ; c1ccc(-c2ccc(N(c3ccc(-c4ccccc4)c(-c4ccccc4)c3)c3cccc4ccc5oc6ccccc6c5c34)cc2-c2ccccc2)cc1]

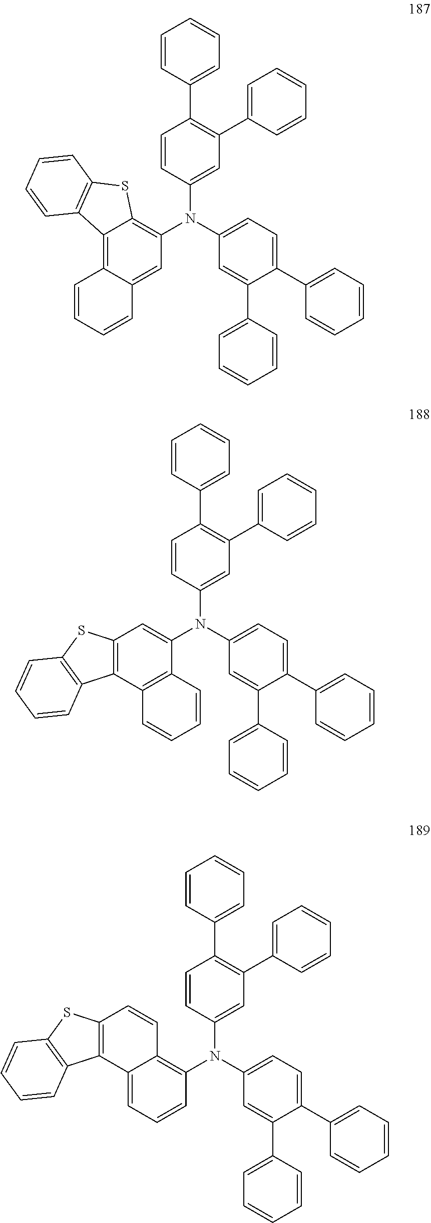 [SMILES: c1ccc(-c2ccc(N(c3ccc(-c4ccccc4)c(-c4ccccc4)c3)c3cc4ccccc4c4c3sc3ccccc34)cc2-c2ccccc2)cc1 ; c1ccc(-c2ccc(N(c3ccc(-c4ccccc4)c(-c4ccccc4)c3)c3cc4sc5ccccc5c4c4ccccc34)cc2-c2ccccc2)cc1 ; c1ccc(-c2ccc(N(c3ccc(-c4ccccc4)c(-c4ccccc4)c3)c3cccc4c3ccc3sc5ccccc5c34)cc2-c2ccccc2)cc1]

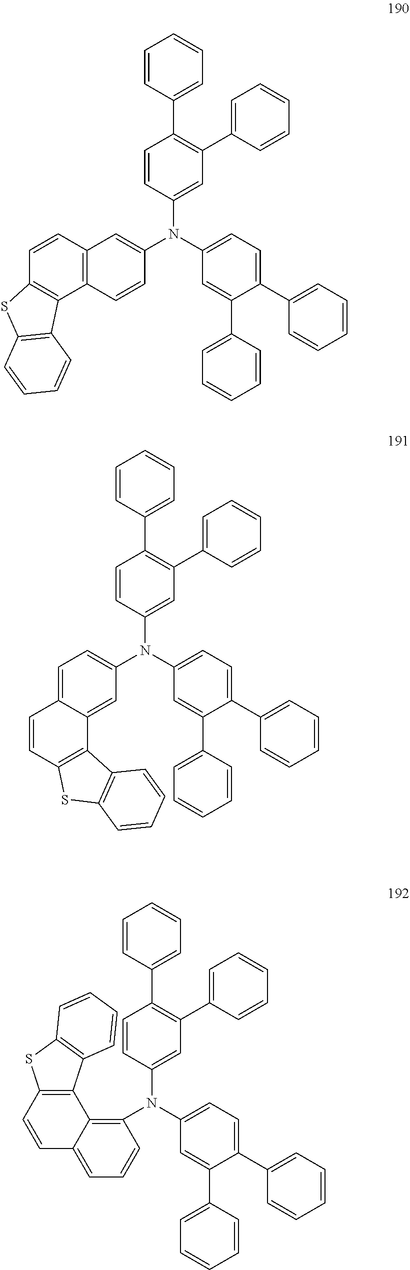 [SMILES: c1ccc(-c2ccc(N(c3ccc(-c4ccccc4)c(-c4ccccc4)c3)c3ccc4c(ccc5sc6ccccc6c54)c3)cc2-c2ccccc2)cc1 ; c1ccc(-c2ccc(N(c3ccc(-c4ccccc4)c(-c4ccccc4)c3)c3ccc4ccc5sc6ccccc6c5c4c3)cc2-c2ccccc2)cc1 ; c1ccc(-c2ccc(N(c3ccc(-c4ccccc4)c(-c4ccccc4)c3)c3cccc4ccc5sc6ccccc6c5c34)cc2-c2ccccc2)cc1]

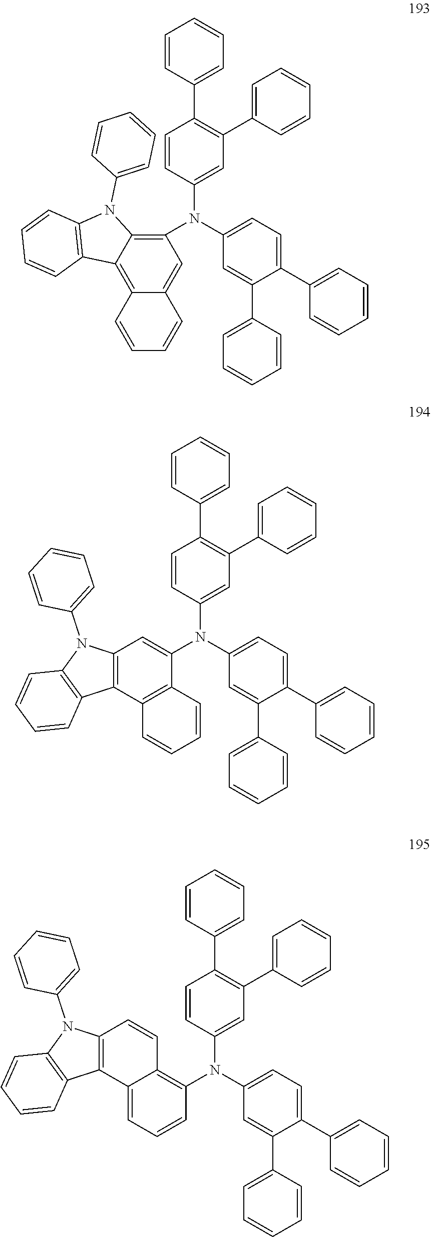 [SMILES: c1ccc(-c2ccc(N(c3ccc(-c4ccccc4)c(-c4ccccc4)c3)c3cc4ccccc4c4c5ccccc5n(-c5ccccc5)c34)cc2-c2ccccc2)cc1 ; c1ccc(-c2ccc(N(c3ccc(-c4ccccc4)c(-c4ccccc4)c3)c3cc4c(c5ccccc35)c3ccccc3n4-c3ccccc3)cc2-c2ccccc2)cc1 ; c1ccc(-c2ccc(N(c3ccc(-c4ccccc4)c(-c4ccccc4)c3)c3cccc4c3ccc3c4c4ccccc4n3-c3ccccc3)cc2-c2ccccc2)cc1]

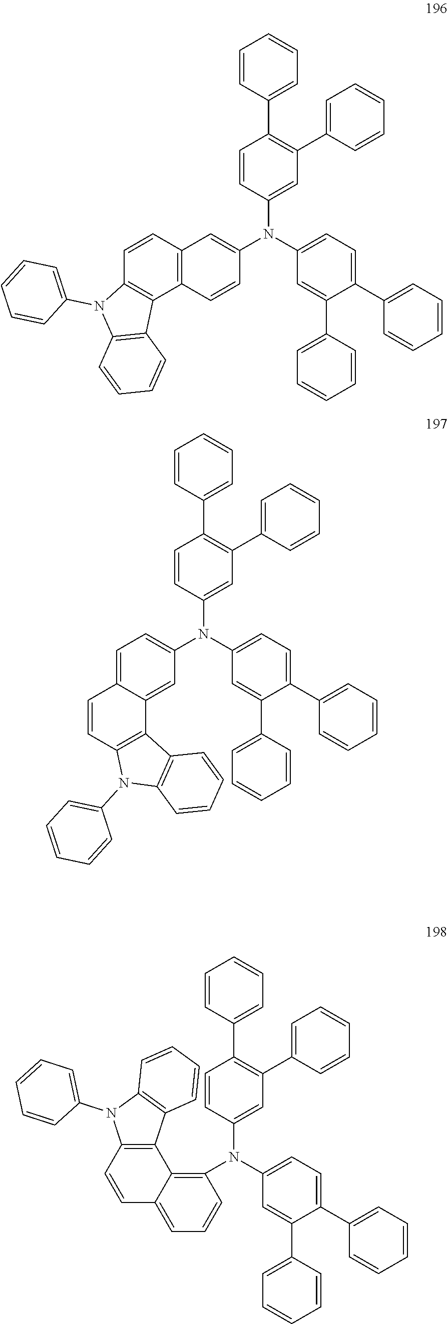 [SMILES: c1ccc(-c2ccc(N(c3ccc(-c4ccccc4)c(-c4ccccc4)c3)c3ccc4c(ccc5c4c4ccccc4n5-c4ccccc4)c3)cc2-c2ccccc2)cc1 ; c1ccc(-c2ccc(N(c3ccc(-c4ccccc4)c(-c4ccccc4)c3)c3ccc4ccc5c(c4c3)c3ccccc3n5-c3ccccc3)cc2-c2ccccc2)cc1 ; c1ccc(-c2ccc(N(c3ccc(-c4ccccc4)c(-c4ccccc4)c3)c3cccc4ccc5c(c6ccccc6n5-c5ccccc5)c34)cc2-c2ccccc2)cc1]

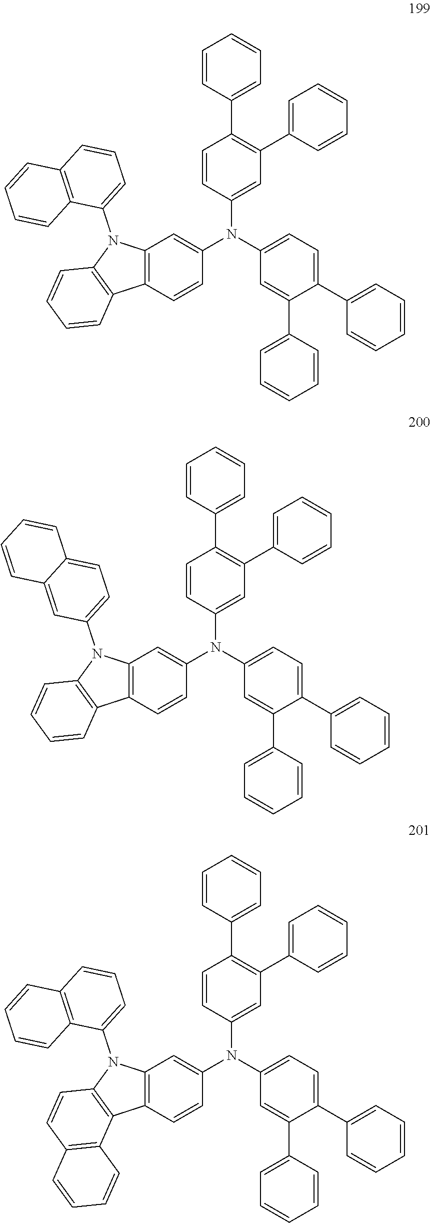 [SMILES: c1ccc(-c2ccc(N(c3ccc(-c4ccccc4)c(-c4ccccc4)c3)c3ccc4c5ccccc5n(-c5cccc6ccccc56)c4c3)cc2-c2ccccc2)cc1 ; c1ccc(-c2ccc(N(c3ccc(-c4ccccc4)c(-c4ccccc4)c3)c3ccc4c5ccccc5n(-c5ccc6ccccc6c5)c4c3)cc2-c2ccccc2)cc1 ; c1ccc(-c2ccc(N(c3ccc(-c4ccccc4)c(-c4ccccc4)c3)c3ccc4c5c6ccccc6ccc5n(-c5cccc6ccccc56)c4c3)cc2-c2ccccc2)cc1]

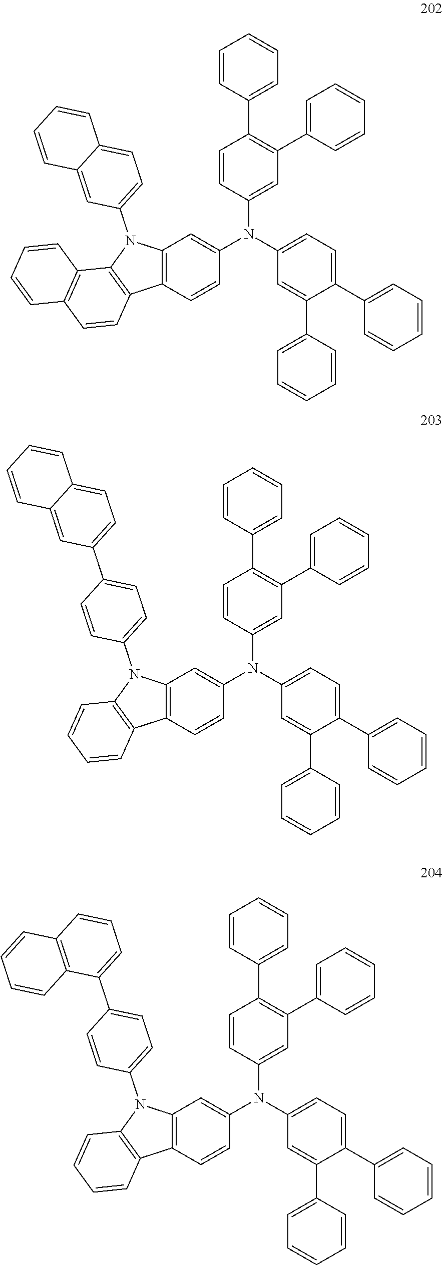 [SMILES: c1ccc(-c2ccc(N(c3ccc(-c4ccccc4)c(-c4ccccc4)c3)c3ccc4c5ccc6ccccc6c5n(-c5ccc6ccccc6c5)c4c3)cc2-c2ccccc2)cc1 ; c1ccc(-c2ccc(N(c3ccc(-c4ccccc4)c(-c4ccccc4)c3)c3ccc4c5ccccc5n(-c5ccc(-c6ccc7ccccc7c6)cc5)c4c3)cc2-c2ccccc2)cc1 ; c1ccc(-c2ccc(N(c3ccc(-c4ccccc4)c(-c4ccccc4)c3)c3ccc4c5ccccc5n(-c5ccc(-c6cccc7ccccc67)cc5)c4c3)cc2-c2ccccc2)cc1]

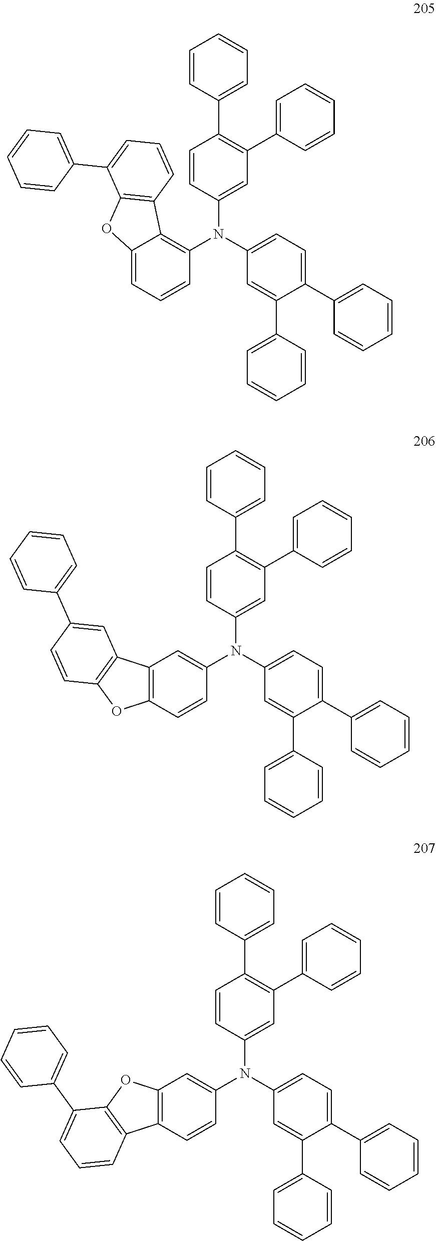 [SMILES: c1ccc(-c2ccc(N(c3ccc(-c4ccccc4)c(-c4ccccc4)c3)c3cccc4oc5c(-c6ccccc6)cccc5c34)cc2-c2ccccc2)cc1 ; c1ccc(-c2ccc3oc4ccc(N(c5ccc(-c6ccccc6)c(-c6ccccc6)c5)c5ccc(-c6ccccc6)c(-c6ccccc6)c5)cc4c3c2)cc1 ; c1ccc(-c2ccc(N(c3ccc(-c4ccccc4)c(-c4ccccc4)c3)c3ccc4c(c3)oc3c(-c5ccccc5)cccc34)cc2-c2ccccc2)cc1]

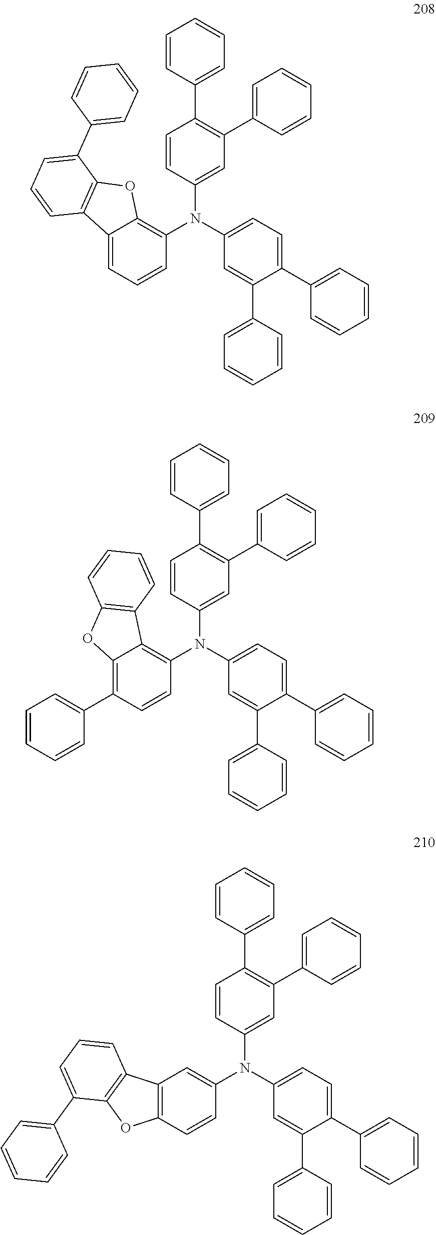 [SMILES: c1ccc(-c2ccc(N(c3ccc(-c4ccccc4)c(-c4ccccc4)c3)c3cccc4c3oc3c(-c5ccccc5)cccc34)cc2-c2ccccc2)cc1 ; c1ccc(-c2ccc(N(c3ccc(-c4ccccc4)c(-c4ccccc4)c3)c3ccc(-c4ccccc4)c4oc5ccccc5c34)cc2-c2ccccc2)cc1 ; c1ccc(-c2ccc(N(c3ccc(-c4ccccc4)c(-c4ccccc4)c3)c3ccc4oc5c(-c6ccccc6)cccc5c4c3)cc2-c2ccccc2)cc1]

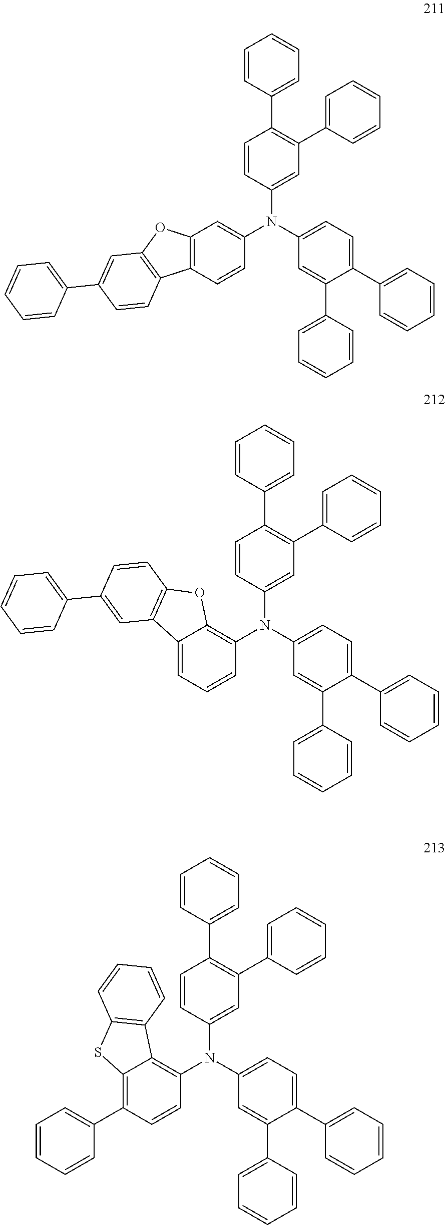 [SMILES: c1ccc(-c2ccc3c(c2)oc2cc(N(c4ccc(-c5ccccc5)c(-c5ccccc5)c4)c4ccc(-c5ccccc5)c(-c5ccccc5)c4)ccc23)cc1 ; c1ccc(-c2ccc3oc4c(N(c5ccc(-c6ccccc6)c(-c6ccccc6)c5)c5ccc(-c6ccccc6)c(-c6ccccc6)c5)cccc4c3c2)cc1 ; c1ccc(-c2ccc(N(c3ccc(-c4ccccc4)c(-c4ccccc4)c3)c3ccc(-c4ccccc4)c4sc5ccccc5c34)cc2-c2ccccc2)cc1]

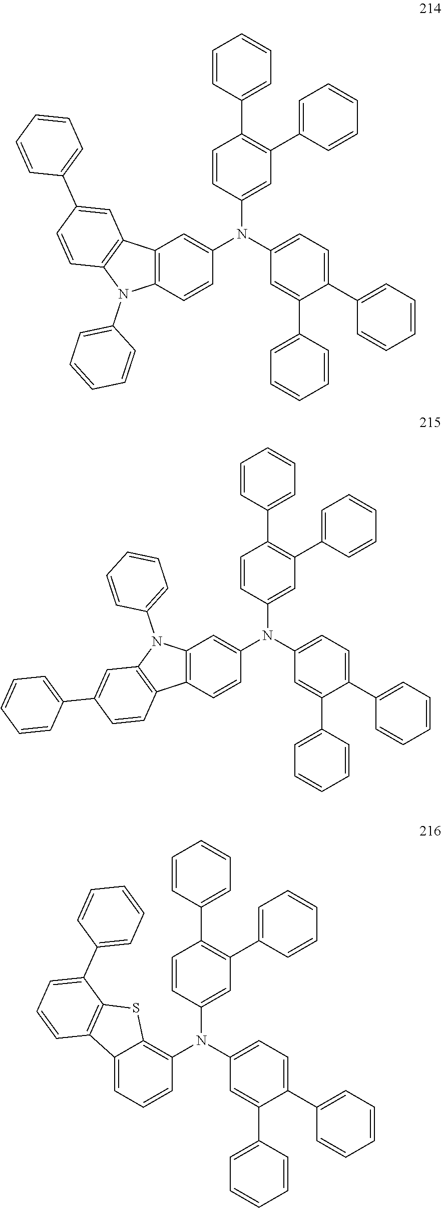 [SMILES: c1ccc(-c2ccc3c(c2)c2cc(N(c4ccc(-c5ccccc5)c(-c5ccccc5)c4)c4ccc(-c5ccccc5)c(-c5ccccc5)c4)ccc2n3-c2ccccc2)cc1 ; c1ccc(-c2ccc3c4ccc(N(c5ccc(-c6ccccc6)c(-c6ccccc6)c5)c5ccc(-c6ccccc6)c(-c6ccccc6)c5)cc4n(-c4ccccc4)c3c2)cc1 ; c1ccc(-c2ccc(N(c3ccc(-c4ccccc4)c(-c4ccccc4)c3)c3cccc4c3sc3c(-c5ccccc5)cccc34)cc2-c2ccccc2)cc1]

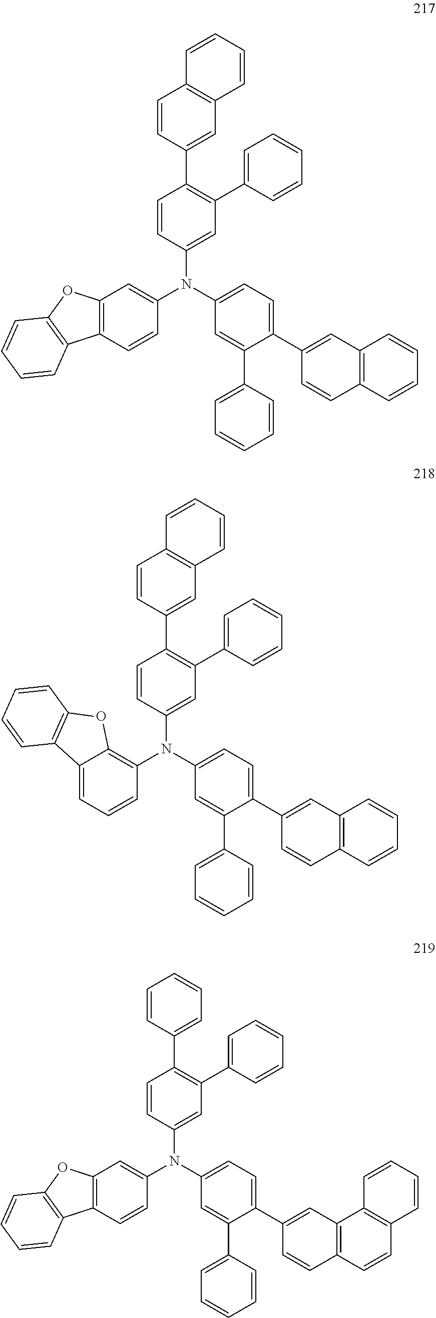 [SMILES: c1ccc(-c2cc(N(c3ccc(-c4ccc5ccccc5c4)c(-c4ccccc4)c3)c3ccc4c(c3)oc3ccccc34)ccc2-c2ccc3ccccc3c2)cc1 ; c1ccc(-c2cc(N(c3ccc(-c4ccc5ccccc5c4)c(-c4ccccc4)c3)c3cccc4c3oc3ccccc34)ccc2-c2ccc3ccccc3c2)cc1 ; c1ccc(-c2ccc(N(c3ccc(-c4ccc5ccc6ccccc6c5c4)c(-c4ccccc4)c3)c3ccc4c(c3)oc3ccccc34)cc2-c2ccccc2)cc1]

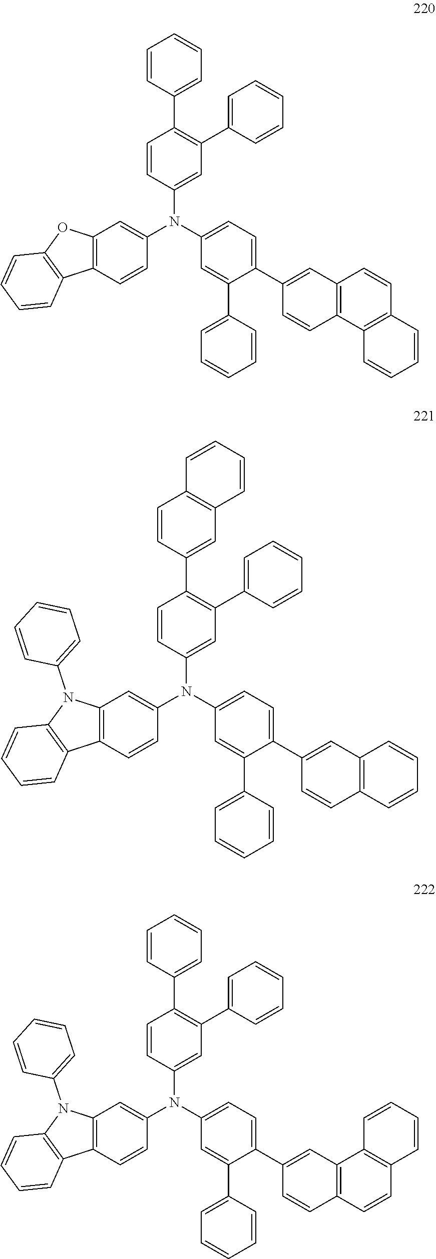 [SMILES: c1ccc(-c2ccc(N(c3ccc(-c4ccc5c(ccc6ccccc65)c4)c(-c4ccccc4)c3)c3ccc4c(c3)oc3ccccc34)cc2-c2ccccc2)cc1 ; c1ccc(-c2cc(N(c3ccc(-c4ccc5ccccc5c4)c(-c4ccccc4)c3)c3ccc4c5ccccc5n(-c5ccccc5)c4c3)ccc2-c2ccc3ccccc3c2)cc1 ; c1ccc(-c2ccc(N(c3ccc(-c4ccc5ccc6ccccc6c5c4)c(-c4ccccc4)c3)c3ccc4c5ccccc5n(-c5ccccc5)c4c3)cc2-c2ccccc2)cc1]

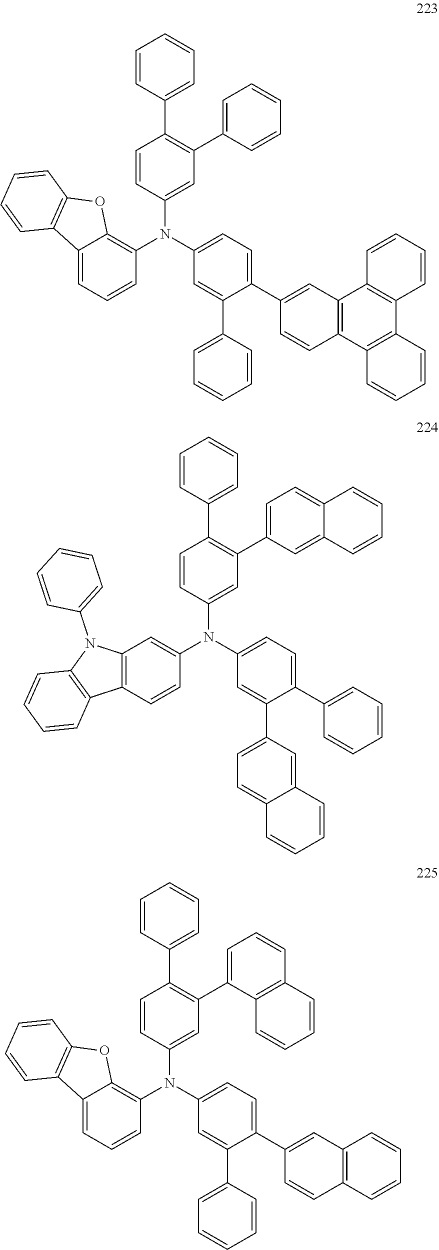

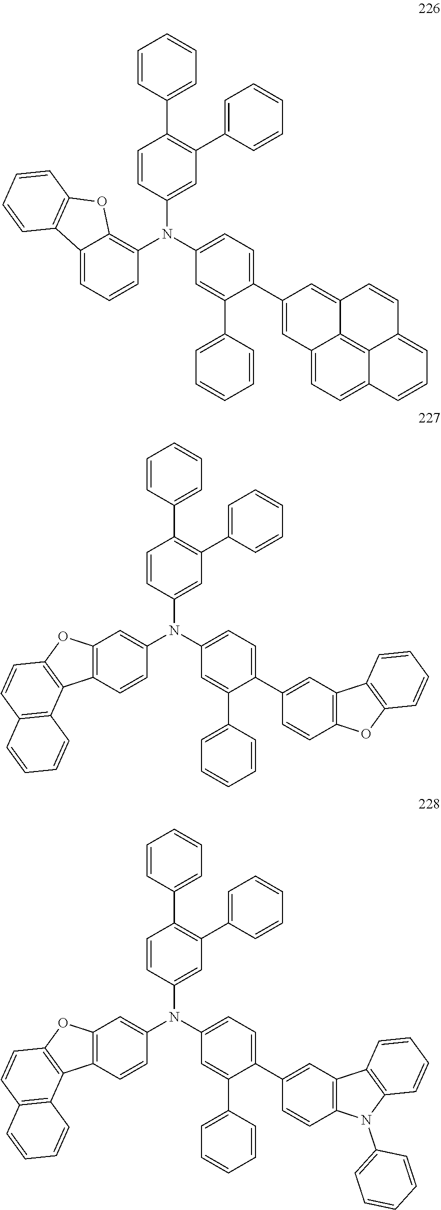 [SMILES: c1ccc(-c2ccc(N(c3ccc(-c4cc5ccc6cccc7ccc(c4)c5c67)c(-c4ccccc4)c3)c3cccc4c3oc3ccccc34)cc2-c2ccccc2)cc1 ; c1ccc(-c2ccc(N(c3ccc(-c4ccc5oc6ccccc6c5c4)c(-c4ccccc4)c3)c3ccc4c(c3)oc3ccc5ccccc5c34)cc2-c2ccccc2)cc1 ; c1ccc(-c2ccc(N(c3ccc(-c4ccc5c(c4)c4ccccc4n5-c4ccccc4)c(-c4ccccc4)c3)c3ccc4c(c3)oc3ccc5ccccc5c34)cc2-c2ccccc2)cc1]

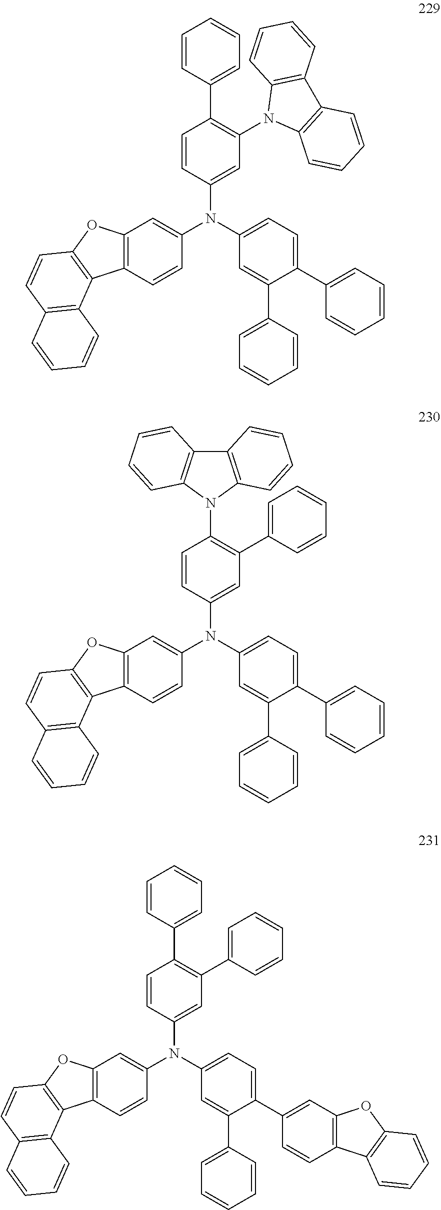

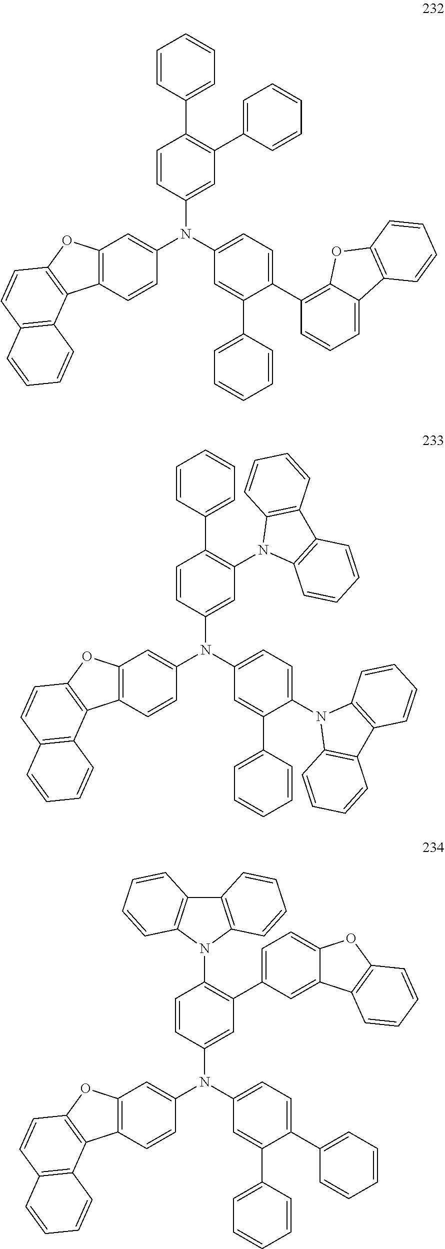 [SMILES: c1ccc(-c2ccc(N(c3ccc(-c4cccc5c4oc4ccccc45)c(-c4ccccc4)c3)c3ccc4c(c3)oc3ccc5ccccc5c34)cc2-c2ccccc2)cc1 ; c1ccc(-c2cc(N(c3ccc(-c4ccccc4)c(-n4c5ccccc5c5ccccc54)c3)c3ccc4c(c3)oc3ccc5ccccc5c34)ccc2-n2c3ccccc3c3ccccc32)cc1 ; c1ccc(-c2ccc(N(c3ccc(-n4c5ccccc5c5ccccc54)c(-c4ccc5oc6ccccc6c5c4)c3)c3ccc4c(c3)oc3ccc5ccccc5c34)cc2-c2ccccc2)cc1]

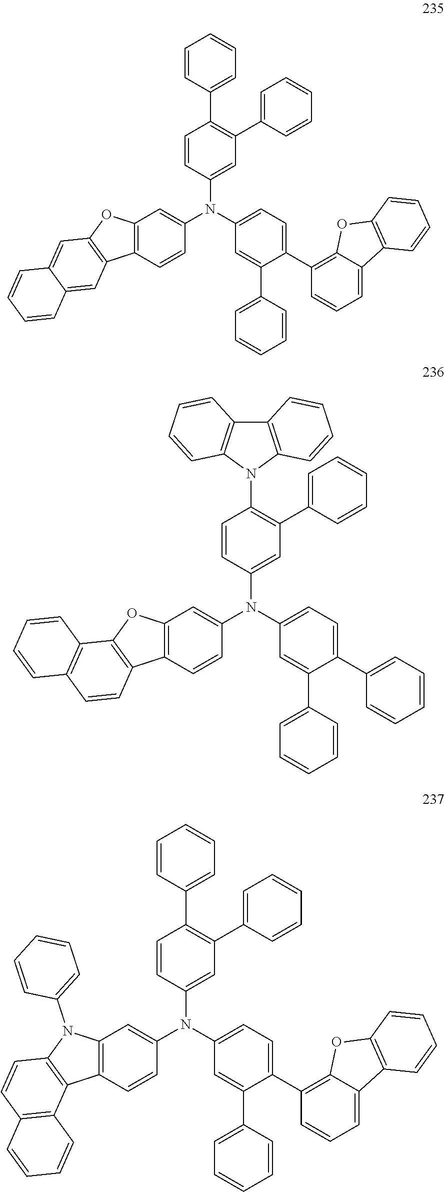 [SMILES: c1ccc(-c2ccc(N(c3ccc(-c4cccc5c4oc4ccccc45)c(-c4ccccc4)c3)c3ccc4c(c3)oc3cc5ccccc5cc34)cc2-c2ccccc2)cc1 ; c1ccc(-c2ccc(N(c3ccc(-n4c5ccccc5c5ccccc54)c(-c4ccccc4)c3)c3ccc4c(c3)oc3c5ccccc5ccc43)cc2-c2ccccc2)cc1 ; c1ccc(-c2ccc(N(c3ccc(-c4cccc5c4oc4ccccc45)c(-c4ccccc4)c3)c3ccc4c5c6ccccc6ccc5n(-c5ccccc5)c4c3)cc2-c2ccccc2)cc1]

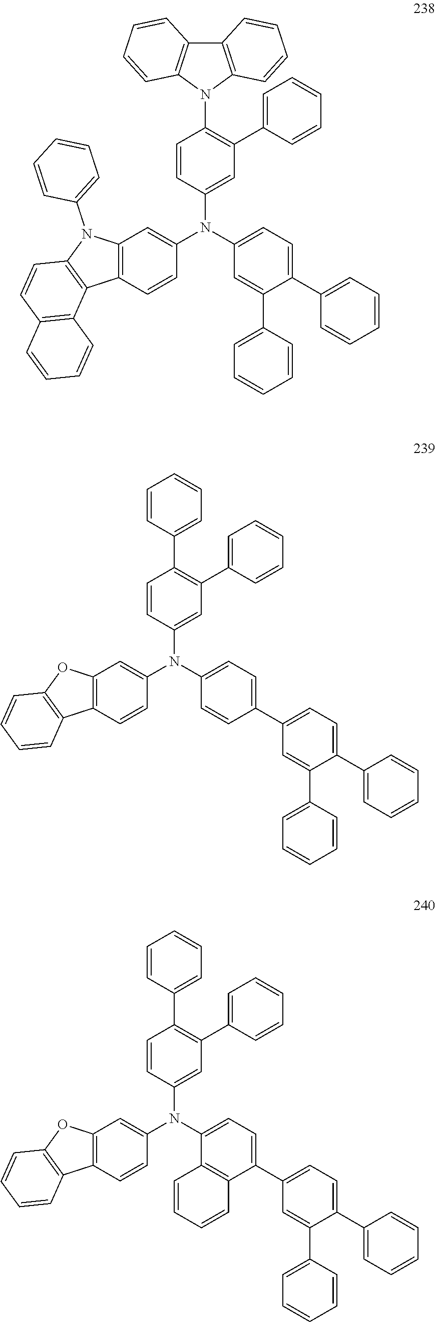 [SMILES: c1ccc(-c2ccc(N(c3ccc(-n4c5ccccc5c5ccccc54)c(-c4ccccc4)c3)c3ccc4c5c6ccccc6ccc5n(-c5ccccc5)c4c3)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)cc3)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4ccccc34)cc2-c2ccccc2)cc1]

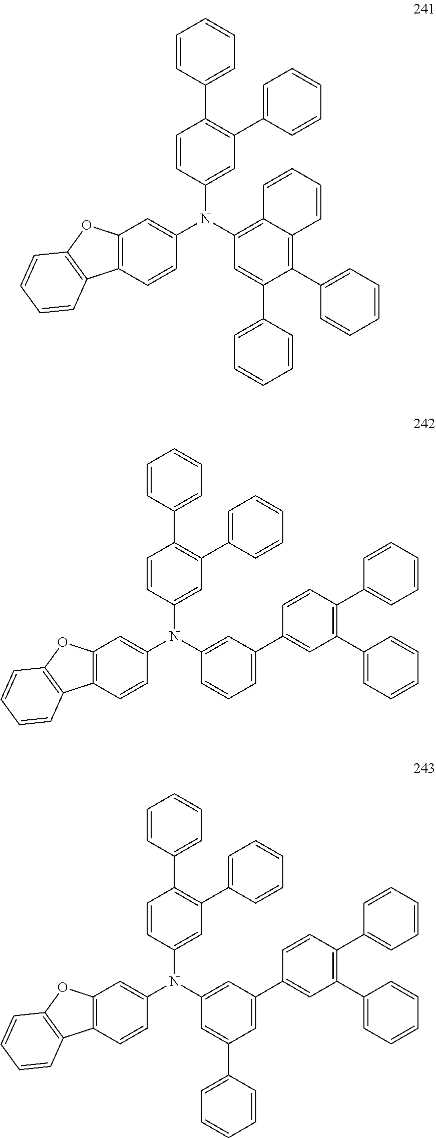 [SMILES: c1ccc(-c2ccc(N(c3ccc4c(c3)oc3ccccc34)c3cc(-c4ccccc4)c(-c4ccccc4)c4ccccc34)cc2-c2ccccc2)cc1 ; c1ccc(-c2ccc(-c3cccc(N(c4ccc(-c5ccccc5)c(-c5ccccc5)c4)c4ccc5c(c4)oc4ccccc45)c3)cc2-c2ccccc2)cc1 ; c1ccc(-c2cc(-c3ccc(-c4ccccc4)c(-c4ccccc4)c3)cc(N(c3ccc(-c4ccccc4)c(-c4ccccc4)c3)c3ccc4c(c3)oc3ccccc34)c2)cc1]

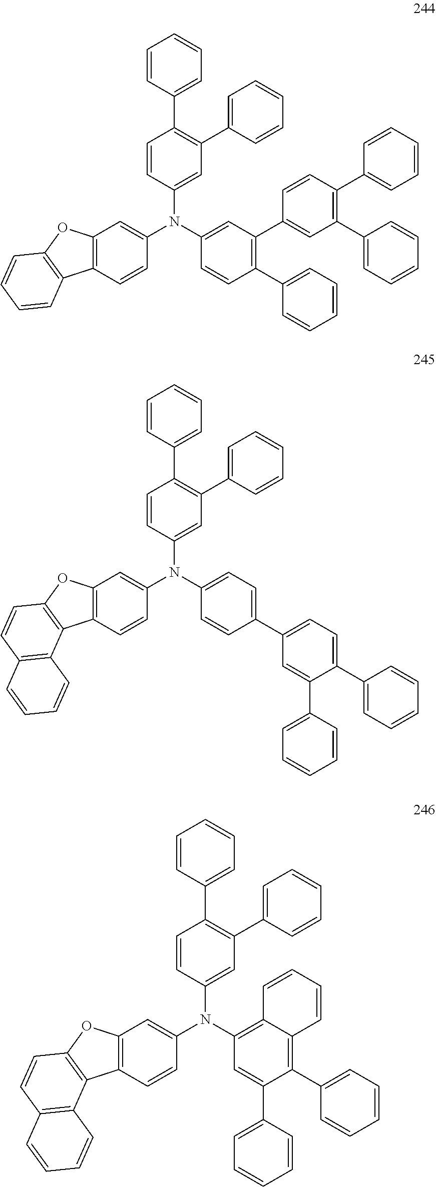 [SMILES: c1ccc(-c2ccc(-c3cc(N(c4ccc(-c5ccccc5)c(-c5ccccc5)c4)c4ccc5c(c4)oc4ccccc45)ccc3-c3ccccc3)cc2-c2ccccc2)cc1 ; c1ccc(-c2ccc(-c3ccc(N(c4ccc(-c5ccccc5)c(-c5ccccc5)c4)c4ccc5c(c4)oc4ccc6ccccc6c45)cc3)cc2-c2ccccc2)cc1 ; c1ccc(-c2ccc(N(c3ccc4c(c3)oc3ccc5ccccc5c34)c3cc(-c4ccccc4)c(-c4ccccc4)c4ccccc34)cc2-c2ccccc2)cc1]

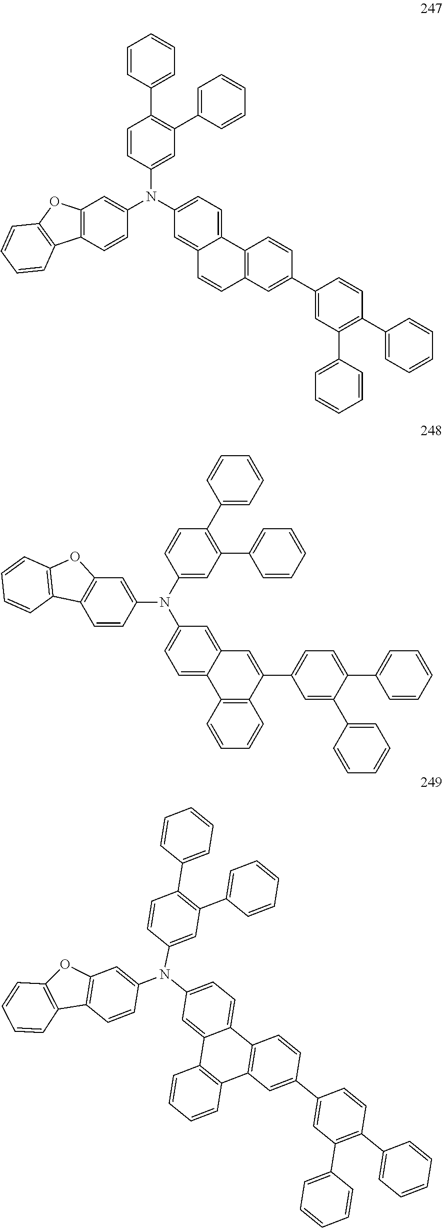 [SMILES: c1ccc(-c2ccc(-c3ccc4c(ccc5cc(N(c6ccc(-c7ccccc7)c(-c7ccccc7)c6)c6ccc7c(c6)oc6ccccc67)ccc54)c3)cc2-c2ccccc2)cc1 ; c1ccc(-c2ccc(-c3cc4cc(N(c5ccc(-c6ccccc6)c(-c6ccccc6)c5)c5ccc6c(c5)oc5ccccc56)ccc4c4ccccc34)cc2-c2ccccc2)cc1 ; c1ccc(-c2ccc(-c3ccc4c5ccc(N(c6ccc(-c7ccccc7)c(-c7ccccc7)c6)c6ccc7c(c6)oc6ccccc67)cc5c5ccccc5c4c3)cc2-c2ccccc2)cc1]

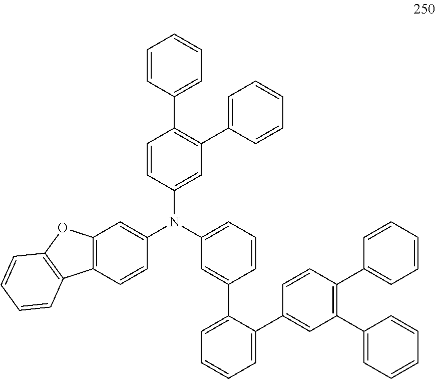 [SMILES: c1ccc(-c2ccc(-c3ccccc3-c3cccc(N(c4ccc(-c5ccccc5)c(-c5ccccc5)c4)c4ccc5c(c4)oc4ccccc45)c3)cc2-c2ccccc2)cc1]

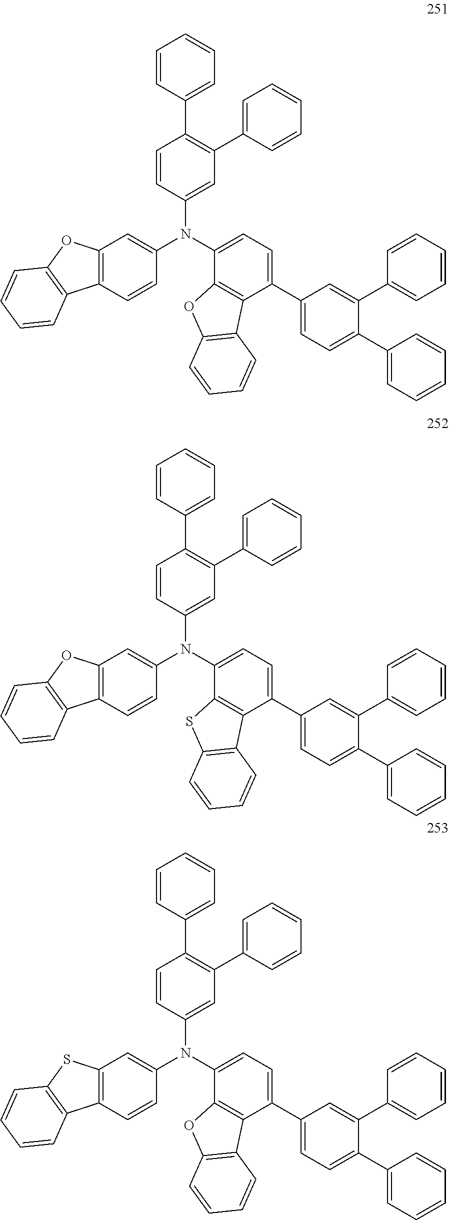 [SMILES: c1ccc(-c2ccc(-c3ccc(N(c4ccc(-c5ccccc5)c(-c5ccccc5)c4)c4ccc5c(c4)oc4ccccc45)c4oc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4sc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)sc4ccccc45)c4oc5ccccc5c34)cc2-c2ccccc2)cc1]

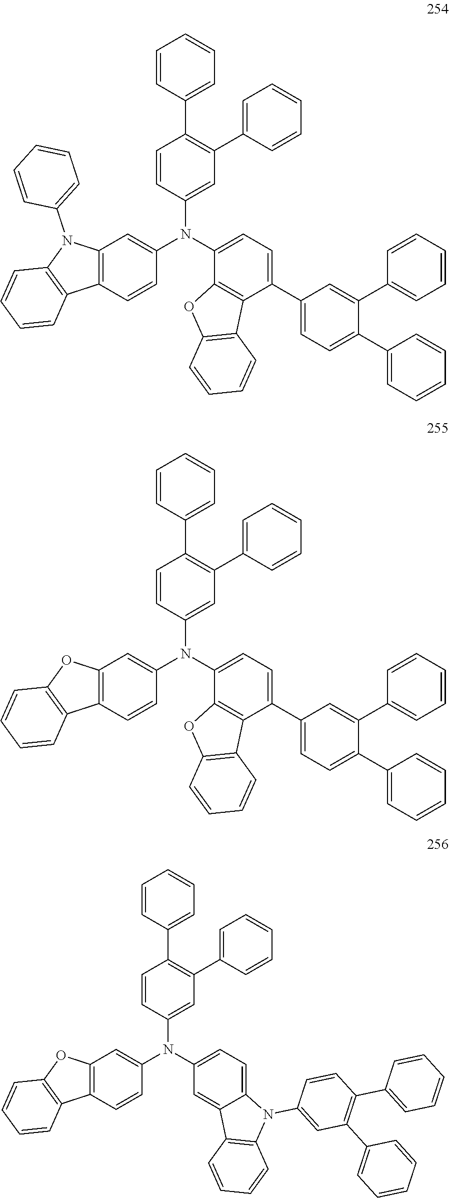 [SMILES: c1ccc(-c2ccc(-c3ccc(N(c4ccc(-c5ccccc5)c(-c5ccccc5)c4)c4ccc5c6ccccc6n(-c6ccccc6)c5c4)c4oc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4oc5ccccc5c34)cc2-c2ccccc2)cc1 ; c1ccc(-c2ccc(N(c3ccc4c(c3)oc3ccccc34)c3ccc4c(c3)c3ccccc3n4-c3ccc(-c4ccccc4)c(-c4ccccc4)c3)cc2-c2ccccc2)cc1]

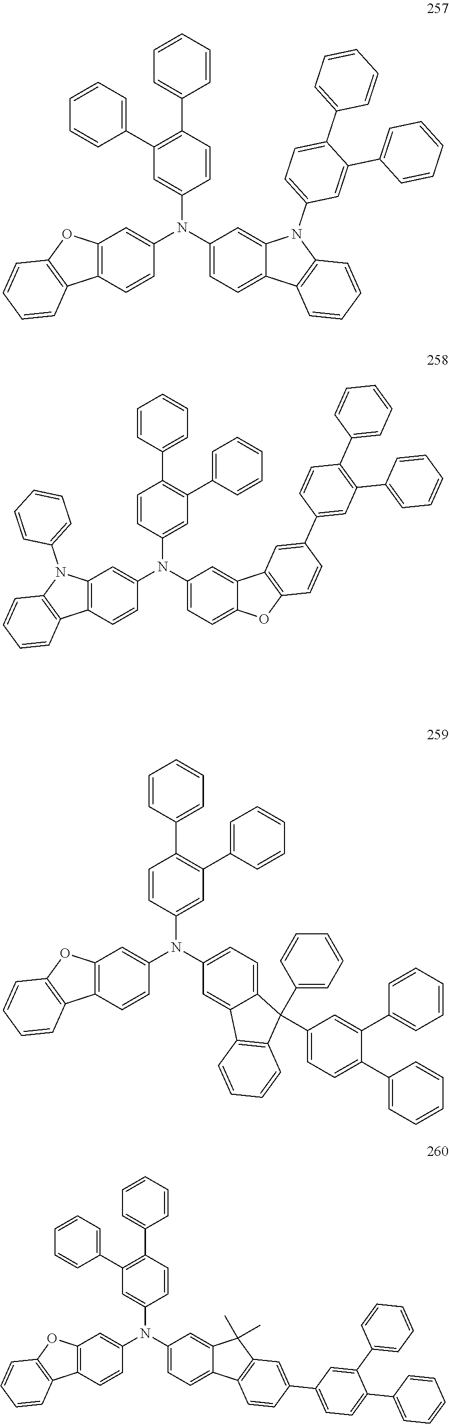 [SMILES: c1ccc(-c2ccc(N(c3ccc4c(c3)oc3ccccc34)c3ccc4c5ccccc5n(-c5ccc(-c6ccccc6)c(-c6ccccc6)c5)c4c3)cc2-c2ccccc2)cc1 ; c1ccc(-c2ccc(-c3ccc4oc5ccc(N(c6ccc(-c7ccccc7)c(-c7ccccc7)c6)c6ccc7c8ccccc8n(-c8ccccc8)c7c6)cc5c4c3)cc2-c2ccccc2)cc1 ; c1ccc(-c2ccc(N(c3ccc4c(c3)-c3ccccc3C4(c3ccccc3)c3ccc(-c4ccccc4)c(-c4ccccc4)c3)c3ccc4c(c3)oc3ccccc34)cc2-c2ccccc2)cc1 ; CC1(C)c2cc(-c3ccc(-c4ccccc4)c(-c4ccccc4)c3)ccc2-c2ccc(N(c3ccc(-c4ccccc4)c(-c4ccccc4)c3)c3ccc4c(c3)oc3ccccc34)cc21]

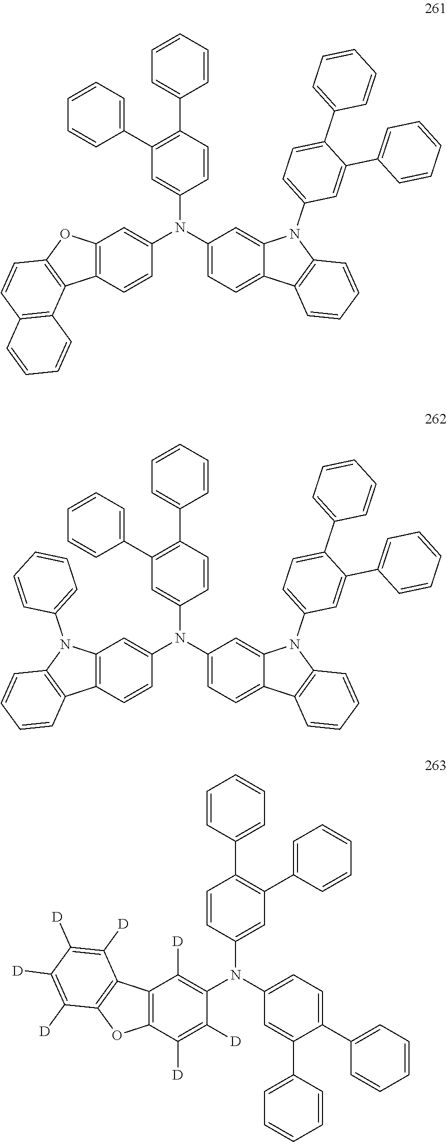 [SMILES: c1ccc(-c2ccc(N(c3ccc4c(c3)oc3ccc5ccccc5c34)c3ccc4c5ccccc5n(-c5ccc(-c6ccccc6)c(-c6ccccc6)c5)c4c3)cc2-c2ccccc2)cc1 ; c1ccc(-c2ccc(N(c3ccc4c5ccccc5n(-c5ccccc5)c4c3)c3ccc4c5ccccc5n(-c5ccc(-c6ccccc6)c(-c6ccccc6)c5)c4c3)cc2-c2ccccc2)cc1 ; [2H]c1c([2H])c([2H])c2c(oc3c([2H])c([2H])c(N(c4ccc(-c5ccccc5)c(-c5ccccc5)c4)c4ccc(-c5ccccc5)c(-c5ccccc5)c4)c([2H])c32)c1[2H]]

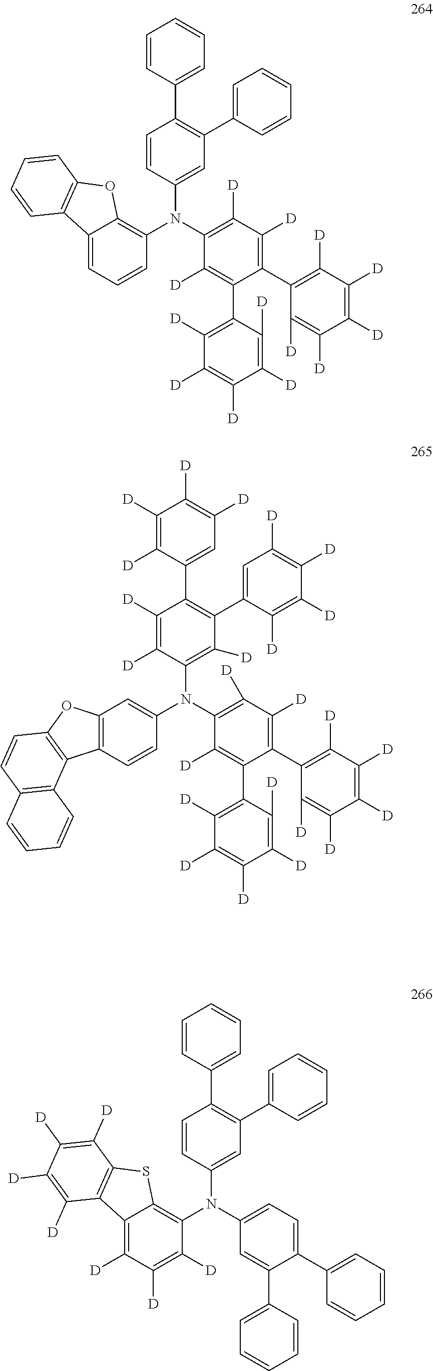

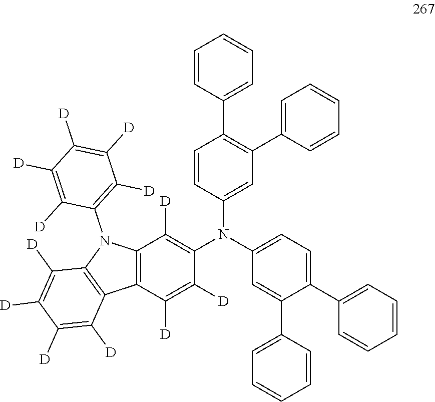 [SMILES: [2H]c1c([2H])c([2H])c(-n2c3c([2H])c([2H])c([2H])c([2H])c3c3c([2H])c([2H])c(N(c4ccc(-c5ccccc5)c(-c5ccccc5)c4)c4ccc(-c5ccccc5)c(-c5ccccc5)c4)c([2H])c32)c([2H])c1[2H]]

The amine compound represented by Formula 1 may include a dibenzoheterole derivative, and two 3,4-substituted phenyl groups (ortho-terphenyl derivative), in which the dibenzoheterole derivative may be directly bonded to a nitrogen atom of the amine compound, and the 3,4-substituted phenyl groups may be each bonded to the nitrogen atom at specific positions. The amine compound according to an embodiment may have excellent electrical stability and high charge transportability due to an introduction of such substituents and a bonding position of a substituent. Therefore, the amine compound according to an embodiment may have an improved lifespan. The light-emitting element according to an embodiment including the amine compound may have improved luminous efficiency and lifespan.

In the light-emitting element ED according to an embodiment, the hole transport region HTR may further include a compound represented by Formula H-1. For example, the light-emitting element ED may include a compound represented by Formula H-1 in another layer of the hole transport region HTR that does not include the amine compound represented by Formula 1. However, embodiments are not limited thereto.

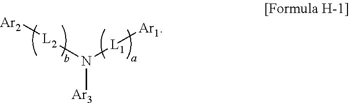

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is 2 or greater, multiple L₁ groups or multiple L² groups may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In Formula H-1, Ar₃ may be a substituted or unsubstituted aryl group having 6 to 30 ring-forming carbon atoms or a substituted or unsubstituted heteroaryl group having 2 to 30 ring-forming carbon atoms.

In an embodiment, the compound represented by Formula H-1 may be a monoamine compound. In another embodiment, the compound represented by Formula H-1 may be a diamine compound in which at least one of Ar₁ to Ar₃ includes an amine group as a substituent. In an embodiment, the compound represented by Formula H-1 may be a carbazole-based compound in which at least one of Ar₁ and Ar₂ includes a substituted or unsubstituted carbazole group, or may be a fluorene-based compound in which at least one of Ar₁ and Ar₂ includes a substituted or unsubstituted fluorene group.

The compound represented by Formula H-1 may be any compound selected from Compound Group H. However, the compounds listed in Compound Group H are only examples, and the compounds represented by Formula H-1 are not limited to Compound Group H:

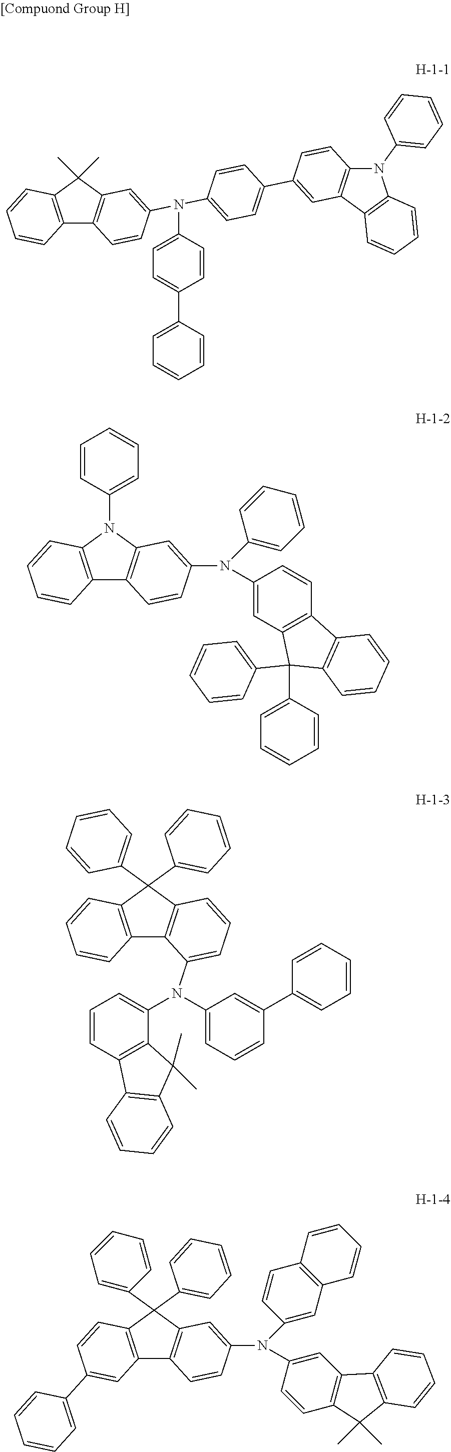 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

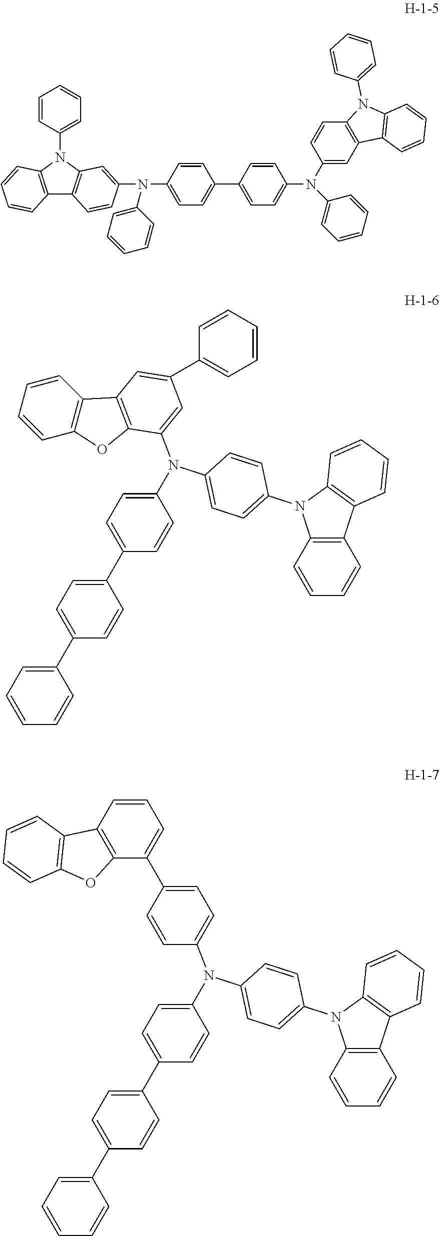 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

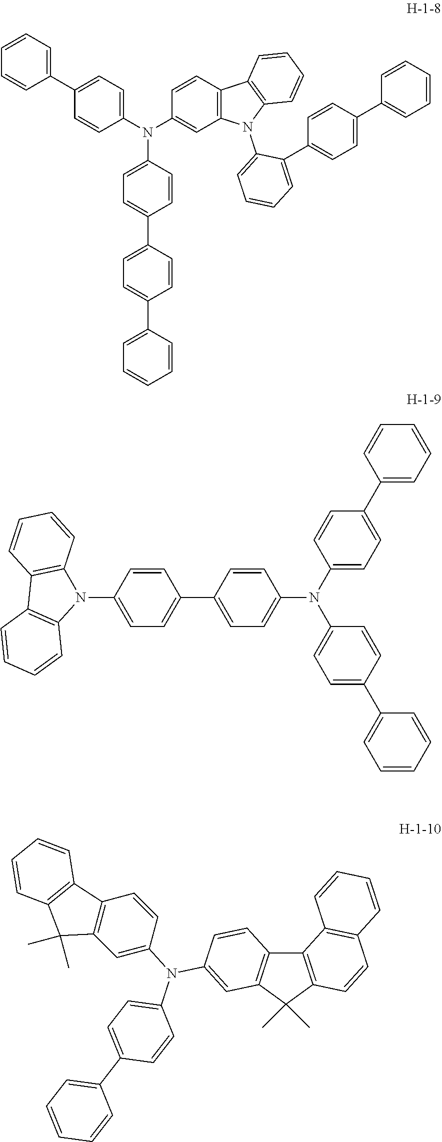 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

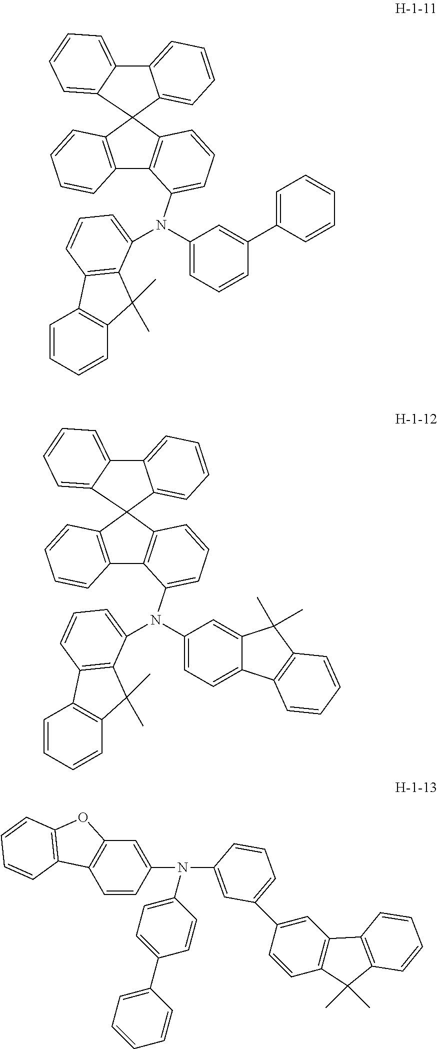 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

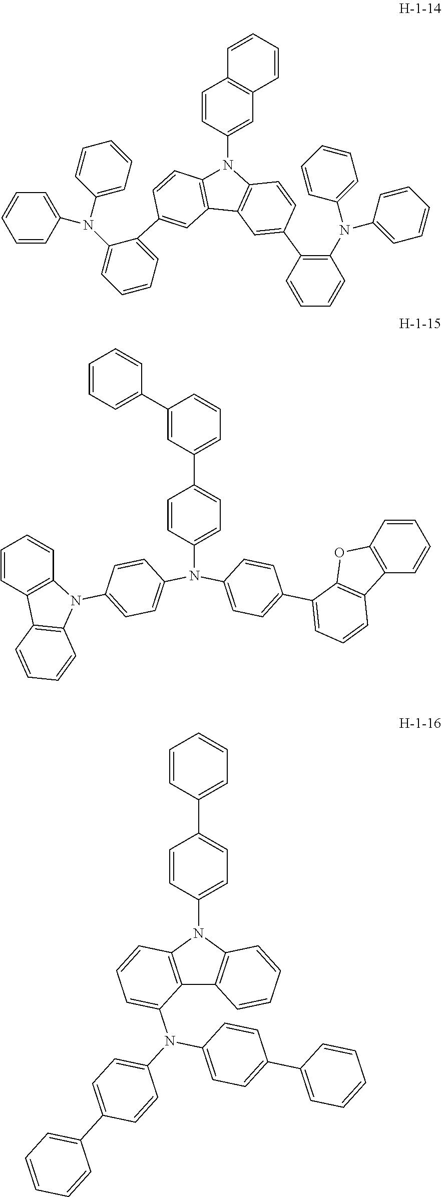 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

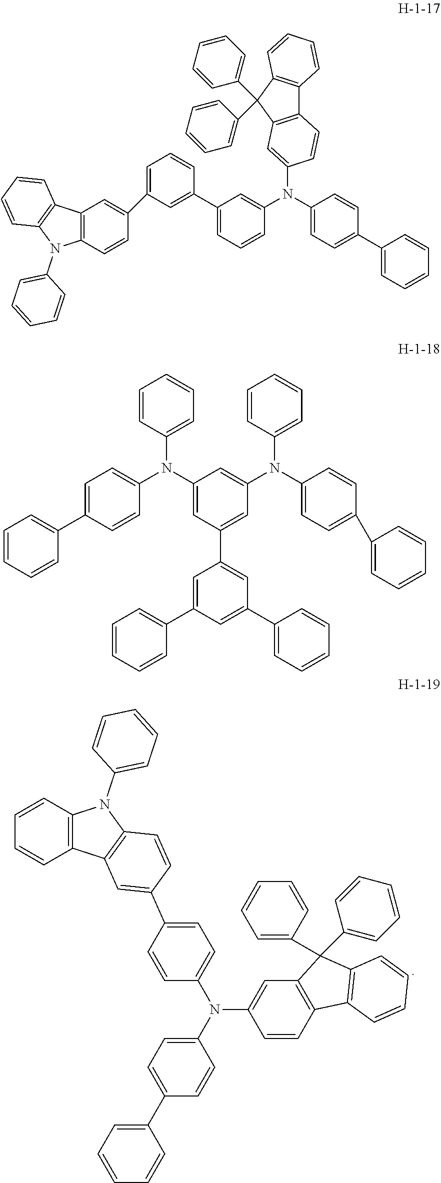

The hole transport region HTR may further include a phthalocyanine compound such as copper phthalocyanine; N1,N1-([1,1′-biphenyl]-4,4′-diyl)bis(N1-phenyl-N4,N4-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris [N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzene sulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl) borate], dipyrazino[2,3-f: 2′,3′-h]quinoxaline-2,3,6,7,10,11-hexacarbonitrile (HAT-CN), etc.

The hole transport region HTR may further include a carbazole-based derivative such as N-phenyl carbazole and polyvinyl carbazole, a fluorene-based derivative, N,N′-bis(3-methylphenyl)-N,N′-diphenyl-[1,1-biphenyl]-4,4′-diamine (TPD), a triphenylamine-based derivative such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzenamine](TAPC), 4,4′-bis [N,N′-(3-tolyl)amino]-3,3′-dimethylbiphenyl (HMTPD), 1,3-bis(N-carbazolyl)benzene (mCP), etc.

In an embodiment, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region HTR in at least one of a hole injection layer HIL, a hole transport layer HTL, an emission auxiliary layer EAL, and an electron blocking layer EBL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have a thickness in a range of about 30 Å to about 1,000 Å. When the hole transport region HTR includes a hole transport layer HTL, the hole transport layer HTL may have a thickness in a range of about 250 Å to about 1,000 Å. When the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a metal halide, a quinone derivative, a metal oxide, and a cyano group-containing compound, but embodiments are not limited thereto. For example, the p-dopant may include a metal halide such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis [cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include at least one of an emission auxiliary layer EAL and an electron blocking layer EBL, in addition to a hole injection layer HIL and a hole transport layer HTL. The emission auxiliary layer EAL may compensate for a resonance distance according to a wavelength of light emitted from an emission layer EML, and may increase light emission efficiency by adjusting hole charge balance in the emission layer EML. The emission auxiliary layer EAL may also prevent electron injection into the hole transport region HTR. Materials which may be included in the hole transport region HTR may be included in the emission auxiliary layer EAL. The electron blocking layer EBL may prevent electron injection from the electron transport region ETR to the hole transport region HTR.

According to an embodiment, the emission layer EML may be provided on the hole transport region HTR. The emission layer EML may have a thickness in a range of about 100 Å to about 1,000 Å. For example, the emission layer EML may have a thickness in a range of about 100 Å to about 300 Å. The emission layer EML may have a structure consisting of a layer consisting of a single material, a structure consisting of a layer including different materials, or a structure including multiple layers including different materials.

In the light-emitting element ED, the emission layer EML may emit blue light. The light-emitting element ED may include the amine compound according to an embodiment in a hole transport region HTR and may show high efficiency and long-life characteristics in a blue emission region. The light-emitting element ED may include the amine compound in the hole transport region HTR, and the emission layer EML may emit blue fluorescence. However, embodiments are not limited thereto.

In the light-emitting element ED, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light-emitting elements ED according to embodiments as shown in each of FIG. 3 to FIG. 7, the emission layer EML may include a host and a dopant, and the emission layer EML may include a compound represented by Formula E-1. The compound represented by Formula E-1 may be used as a fluorescence host material.

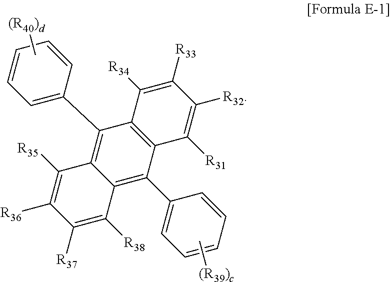

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

In an embodiment, the compound represented by Formula E-1 may be any compound selected from Compound El to Compound E19:

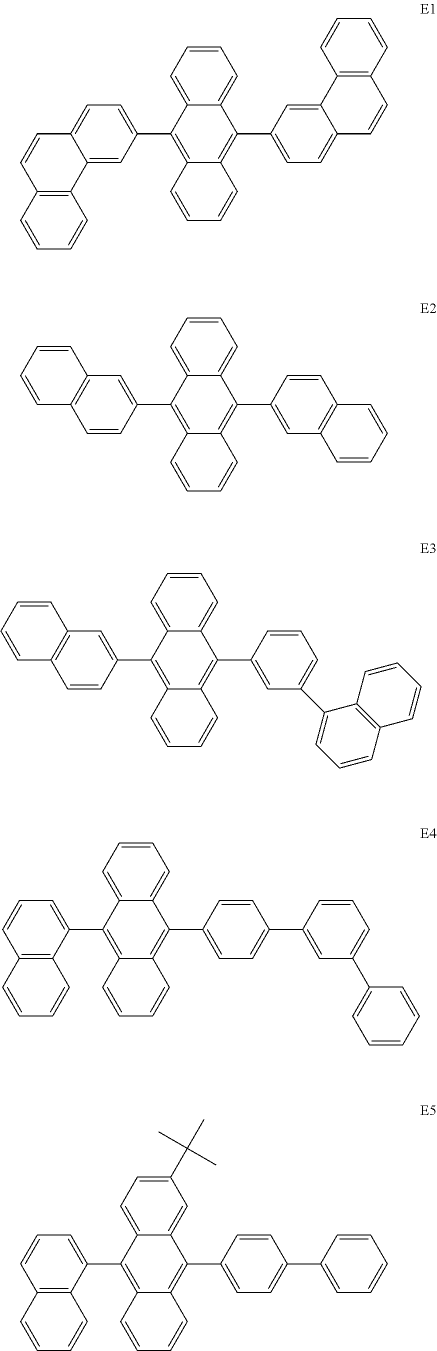

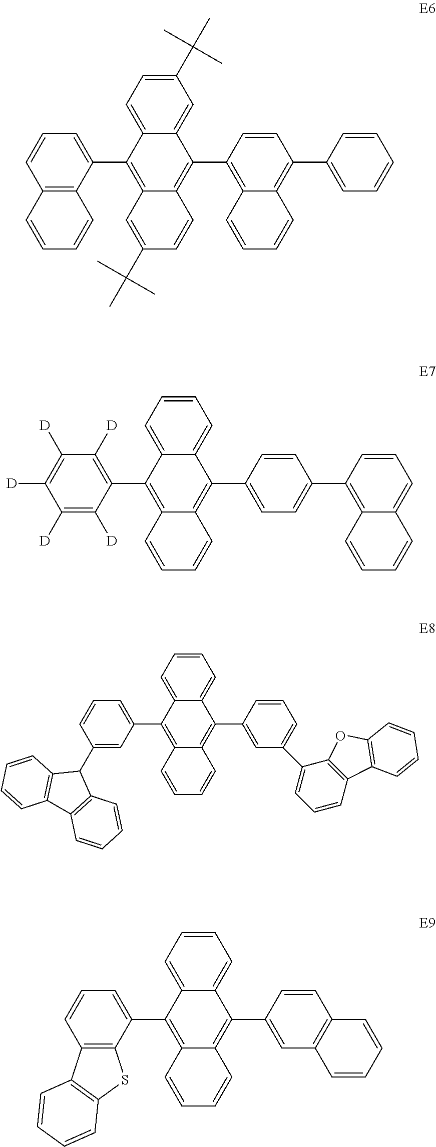

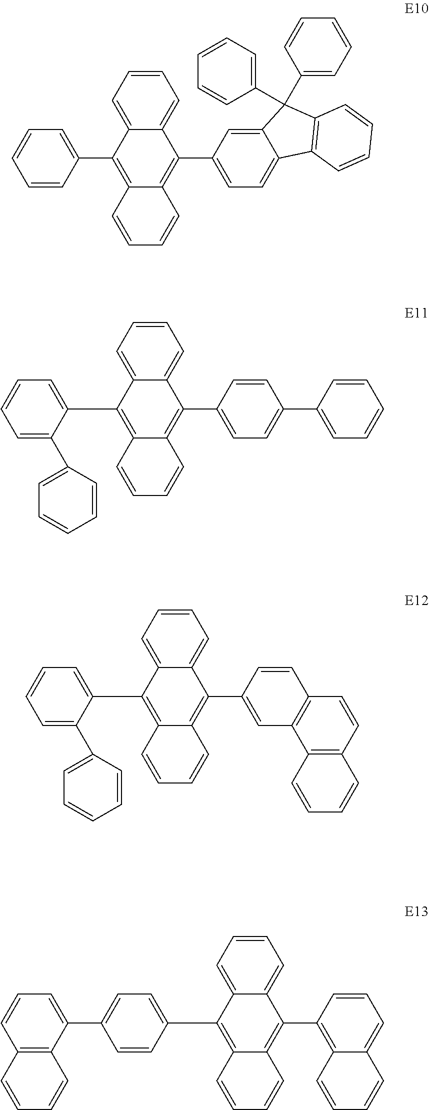

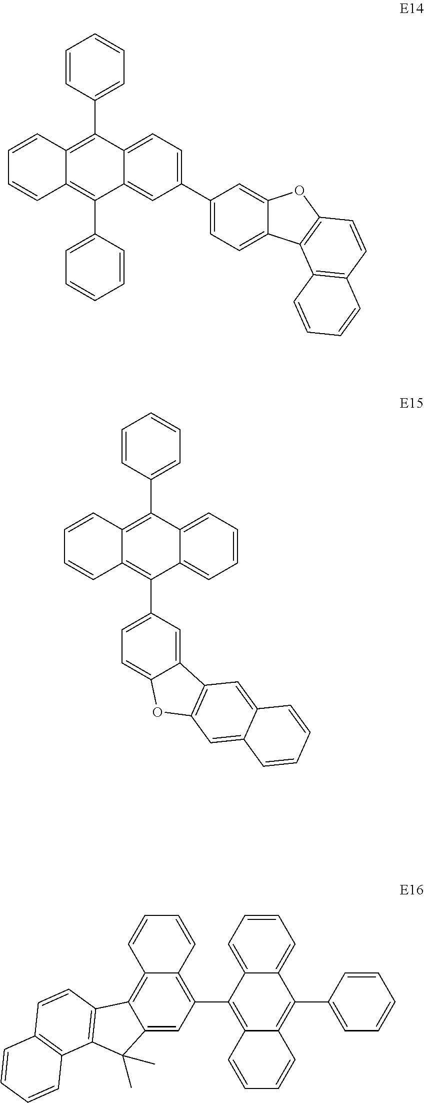

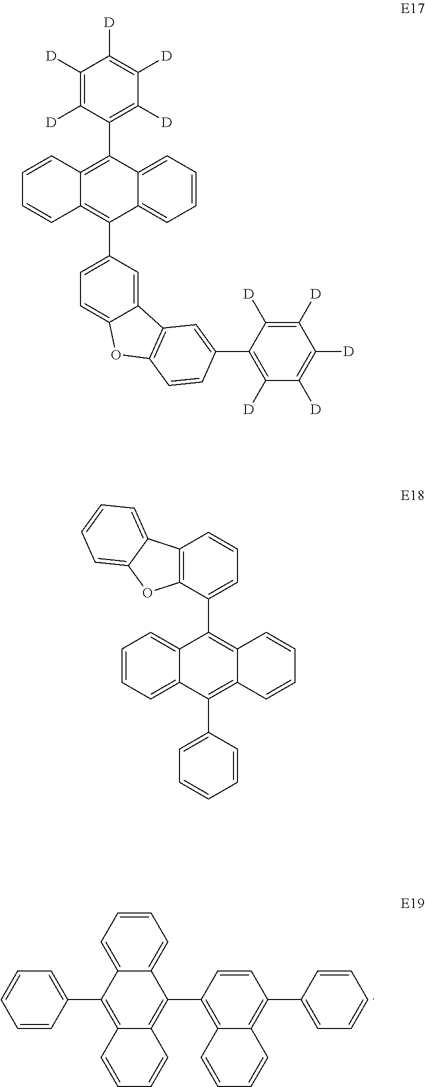

In an embodiment, the emission layer EML may include a compound represented by Formula E-2a or Formula E-2b. The compound represented by Formula E-2a or Formula E-2b may be used as a host material for a phosphorescence element.

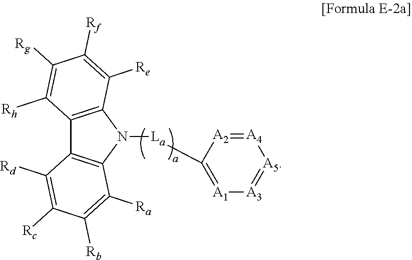

In Formula E-2a, a may be an integer from 0 to 10; and Lₐ may be a direct linkage, a substituted or unsubstituted arylene group of 6 to 30 ring-forming carbon atoms, or a substituted or unsubstituted heteroarylene group of 2 to 30 ring-forming carbon atoms. If a is 2 or more, multiple Lₐ groups may each independently be a substituted or unsubstituted arylene group of 6 to 30 ring-forming carbon atoms or a substituted or unsubstituted heteroarylene group of 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, Rₐ to Rᵢ may be combined with an adjacent group to form a hydrocarbon ring or a heterocycle that includes N, O, S, etc. as a ring-forming atom.

In Formula E-2a, two or three of A₁ to A₅ may each be N, and the remainder of A₁ to A₅ may each independently be C(Rᵢ).

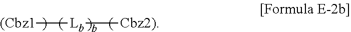

In Formula E-2b, Cbz1 and Cbz2 may each independently be an unsubstituted carbazole group or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10. If b is 2 or more, multiple L_{b} groups may each independently be a substituted or unsubstituted arylene group of 6 to 30 ring-forming carbon atoms or a substituted or unsubstituted heteroarylene group of 2 to 30 ring-forming carbon atoms.

In an embodiment, the compound represented by Formula E-2a or Formula E-2b may be any compound selected from Compound Group E-2. However, the compounds listed in Compound Group E-2 are only examples, and the compound represented by Formula E-2a or Formula E-2b is not limited to Compound Group E-2:

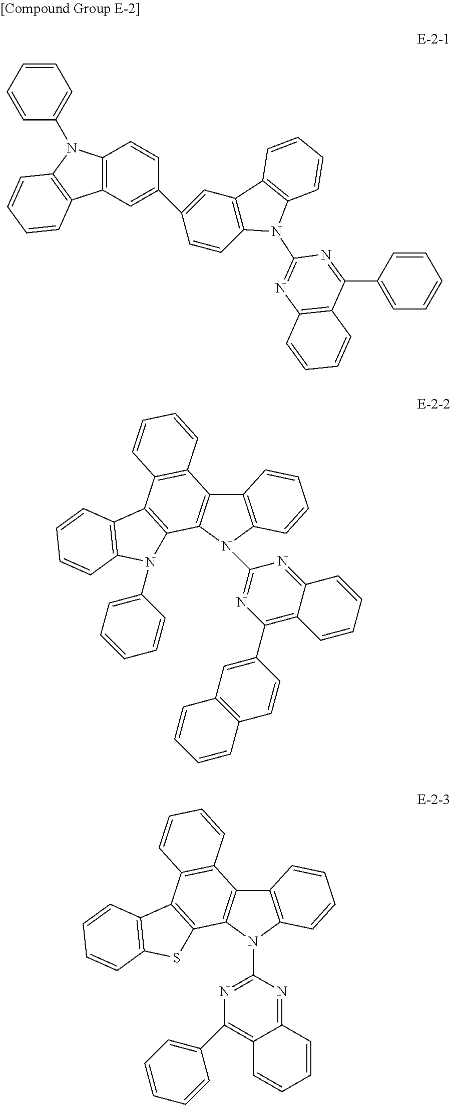

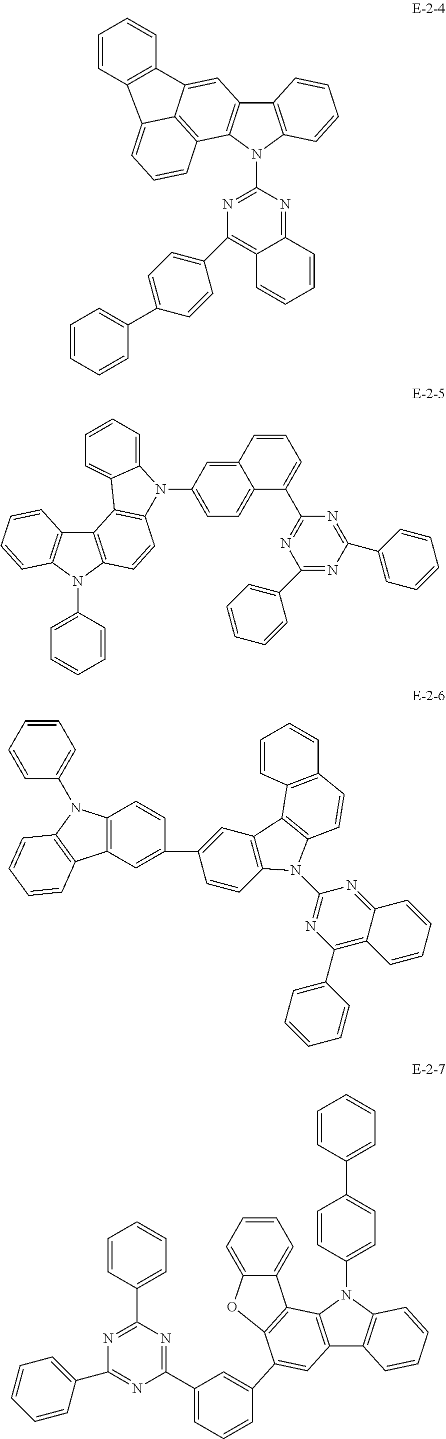

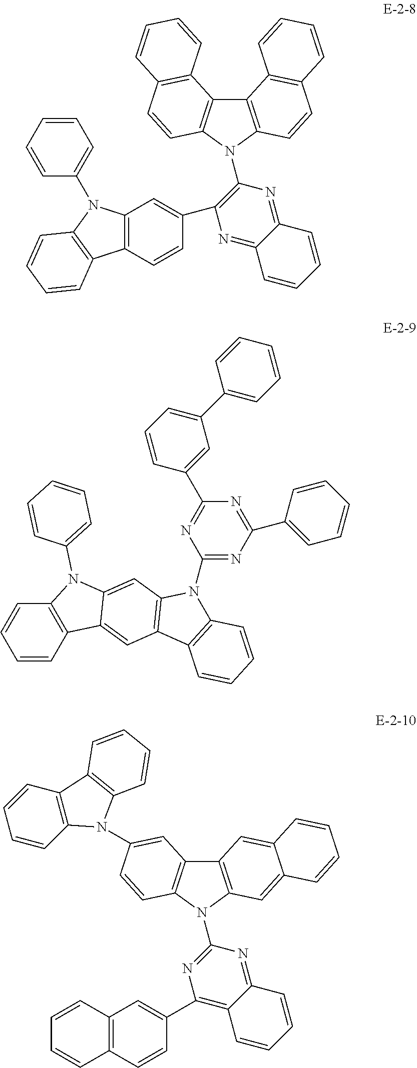 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

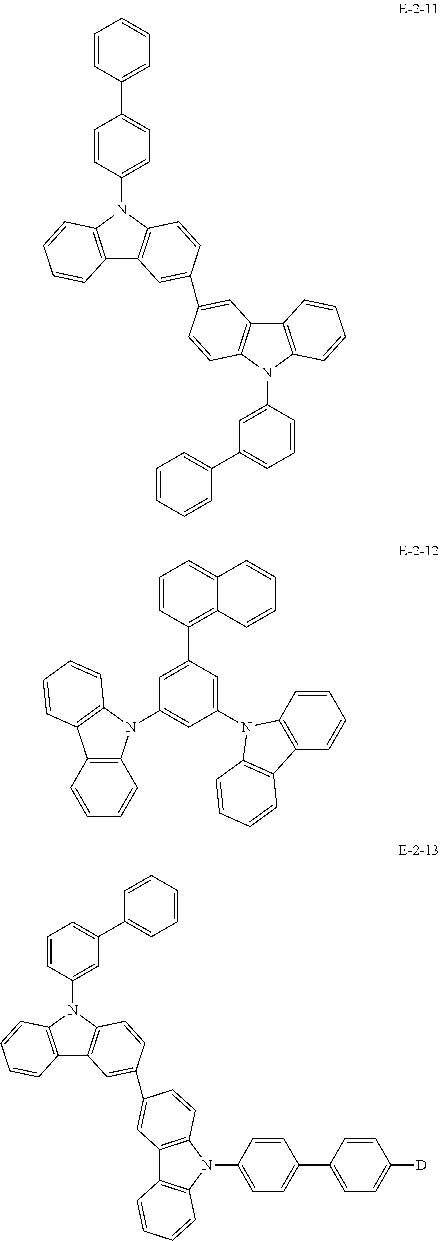

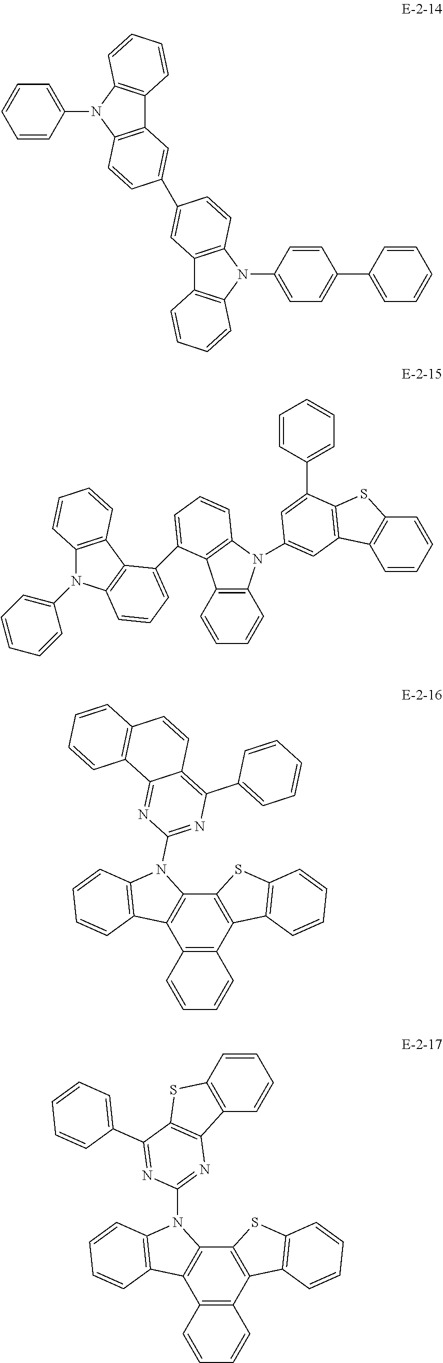 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

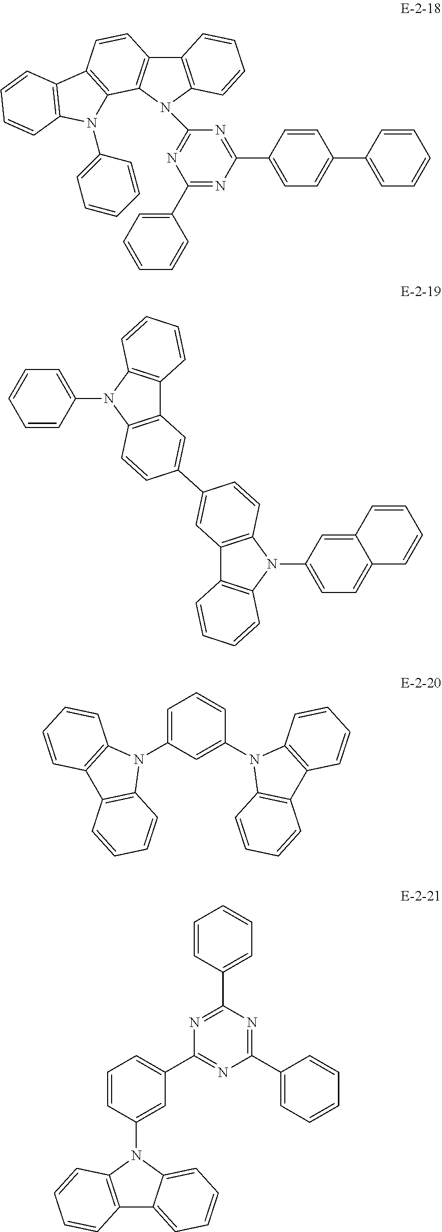 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

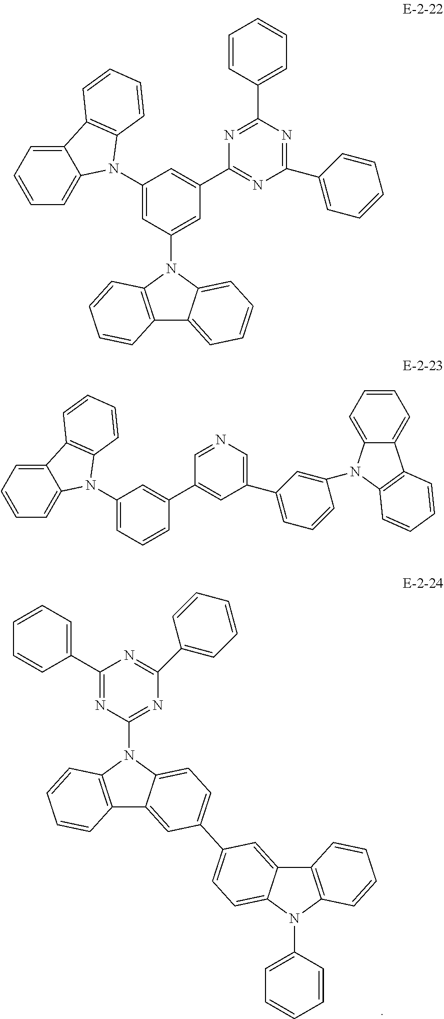 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis [2-(diphenylphosphino)phenyl]ether oxide (DPEPO), 4,4′-bis(carbazol-9-yl) biphenyl (CBP), 1,3-bis(carbazol-9-yl)benzene (mCP), 2,8-bis(diphenylphosphoryl) dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments not limited are thereto. For example, tris(8-hydroxyquinolino)aluminum (Alq₃), 9,10-di(naphthalene-2-yl) anthracene (ADN), 2-tert-butyl-9,10-di(naphth-2-yl) anthracene (TBADN), distyrylarylene (DSA), 4,4′-bis(9-carbazolyl)-2,2′-dimethyl-biphenyl (CDBP), 2-methyl-9,10-bis(naphthalen-2-yl) anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

In an embodiment, the emission layer EML may include a compound represented by Formula M-a or Formula M-b. The compound represented by Formula M-a or Formula M-b may be used as a host material for phosphorescent element.

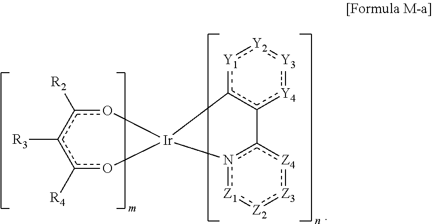

In Formula M-a, Y₁ to Y₄ and Z₁ to Z₄ may each independently be C(R₁) or N; and R¹ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. In Formula M-a, m may be 0 or 1, and n may be 2 or 3. In Formula M-a, if m is 0, n may be 3, and if m is 1, n may be 2.

In an embodiment, the compound represented by Formula M-a may be any compound selected from Compounds M-a1 to M-a25. However, Compounds M-a1 to M-a25 are only examples, and the compound represented by Formula M-a is not limited to Compounds M-a1 to M-a25:

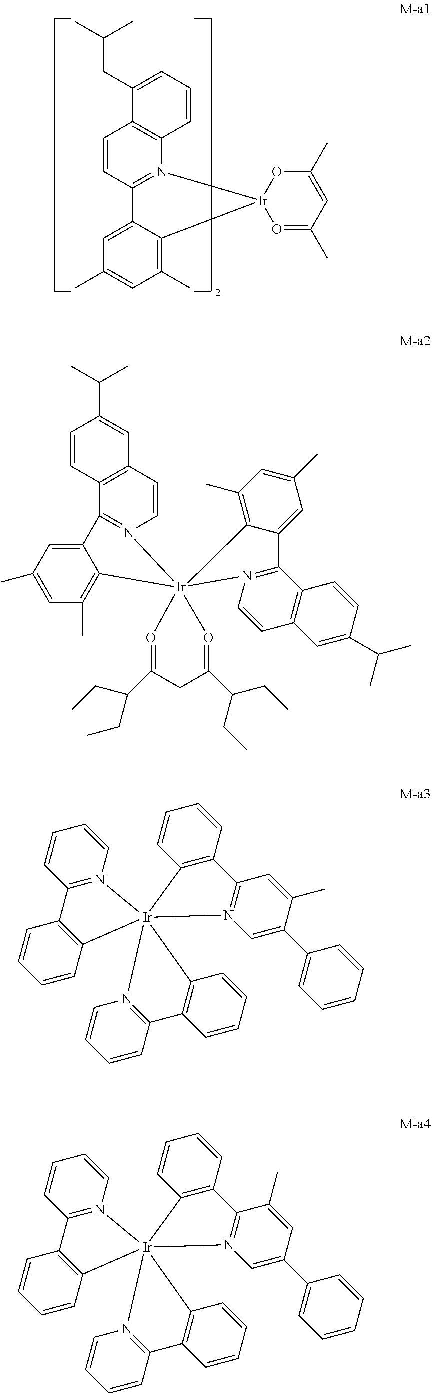

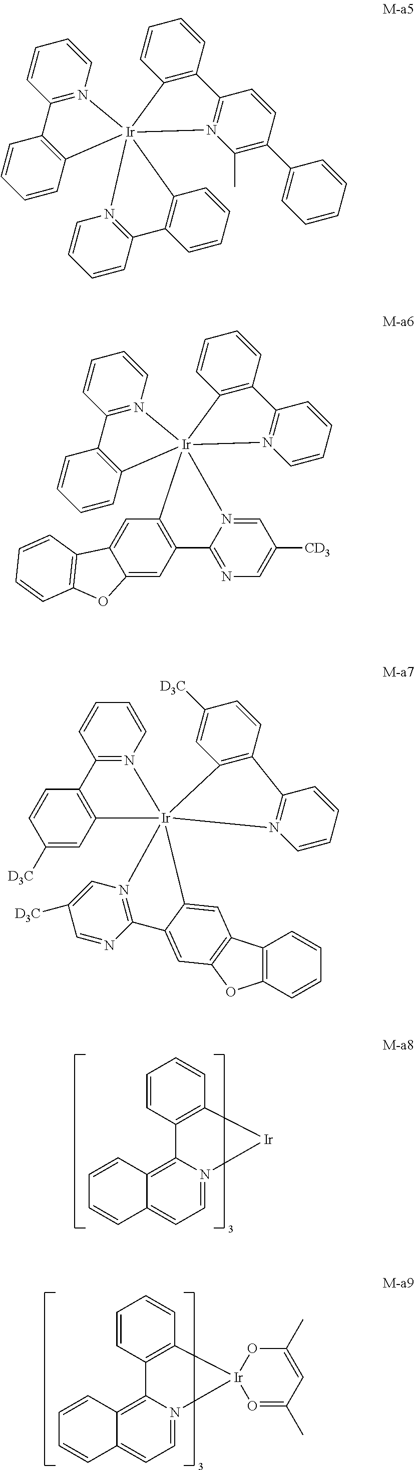

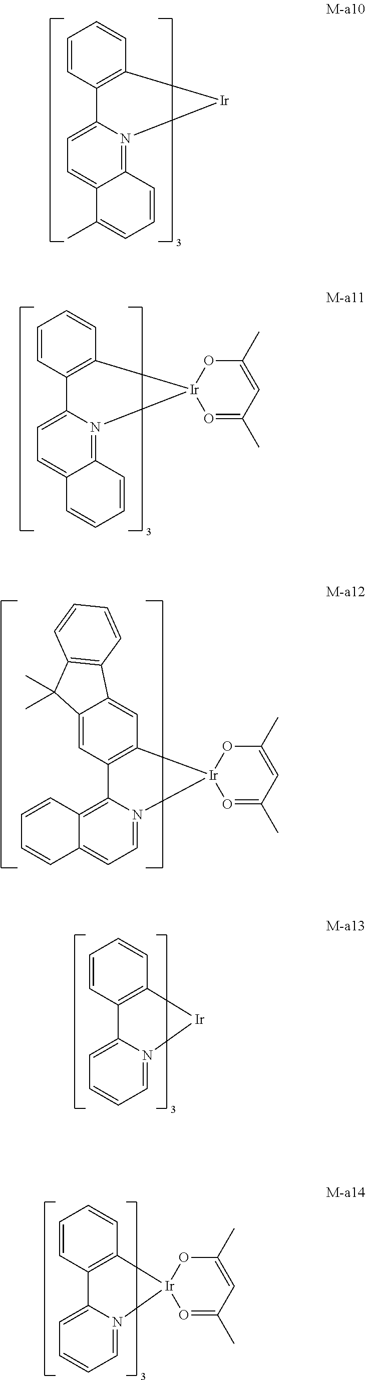

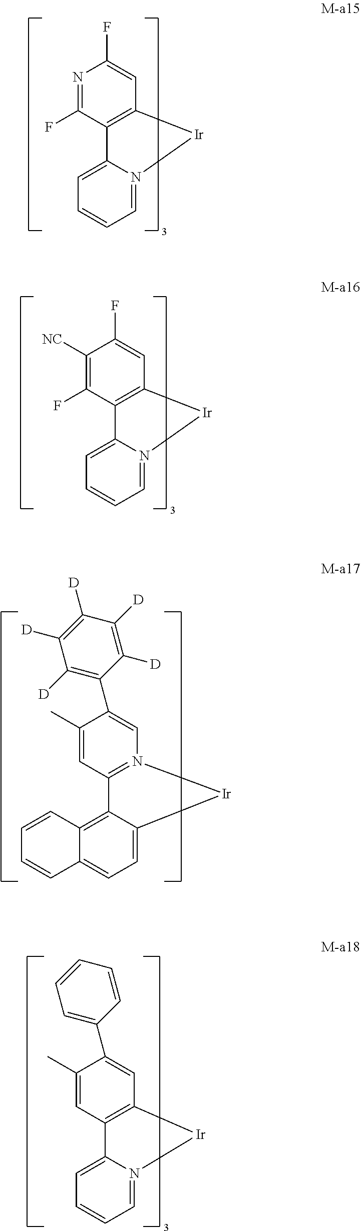

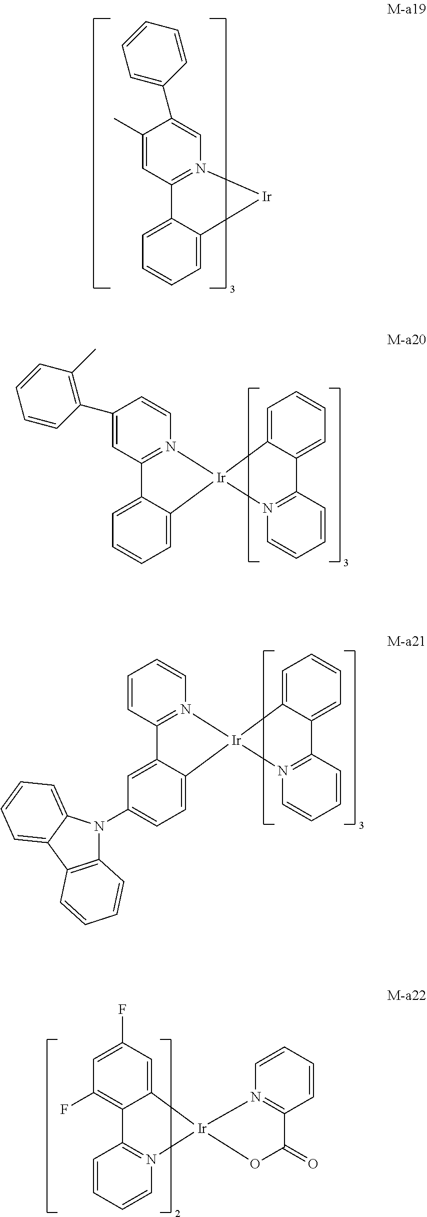

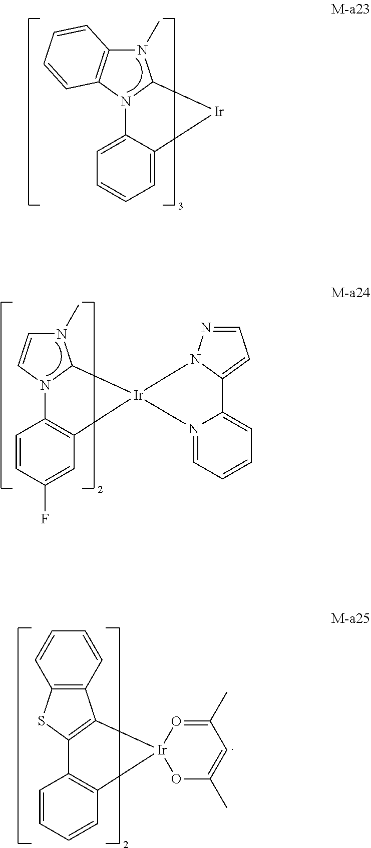

Compound M-a1 and Compound M-a2 may each be used as a red dopant material, and Compound M-a3 to Compound M-a5 may each be used as a green dopant material.

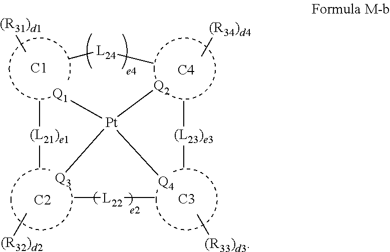

In Formula M-b, Q₁ to Q₄ may each independently be C or N; and C1 to C4 may each independently be a substituted or unsubstituted hydrocarbon ring of 5 to 30 ring-forming carbon atoms or a substituted or unsubstituted heterocycle of 2 to 30 ring-forming carbon atoms. In Formula M-b, L₂₁ to L₂₄ may each independently be a direct linkage,

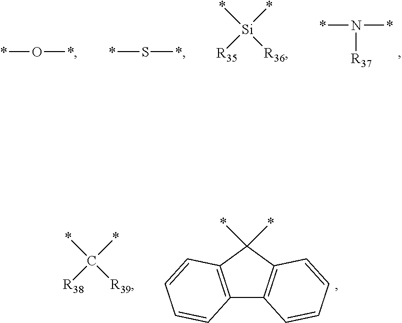

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms; and d1 to e4 may each independently be 0 or 1.

In Formula M-b, R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring; and d1 to d4 may each independently be an integer from 0 to 4. If d1 is greater than or equal to 2, multiple R₃₁ groups may be the same as each other or at least one thereof may be different from the remainder. If d2 is greater than or equal to 2, multiple R₃₂ groups may be the same as each other or at least one thereof may be different from the remainder. If d3 is greater than or equal to 2, multiple R₃₃ groups may be the same as each other or at least one thereof may be different from the remainder. If d4 is greater than or equal to 2, multiple R₃₄ groups may be the same as each other or at least one thereof may be different from the remainder.

The compound represented by Formula M-b may be used as a blue phosphorescent dopant or as a green phosphorescent dopant. In an embodiment, the compound represented by Formula M-b may further be included in the light-emitting layer EML as an auxiliary dopant.

In an embodiment, the compound represented by Formula M-b may be any compound selected from Compounds M-b-1 to M-b-11. However, Compounds M-b-1 to M-b-11 are only examples, and the compound represented by Formula M-b is not limited to Compounds M-b-1 to M-b-11:

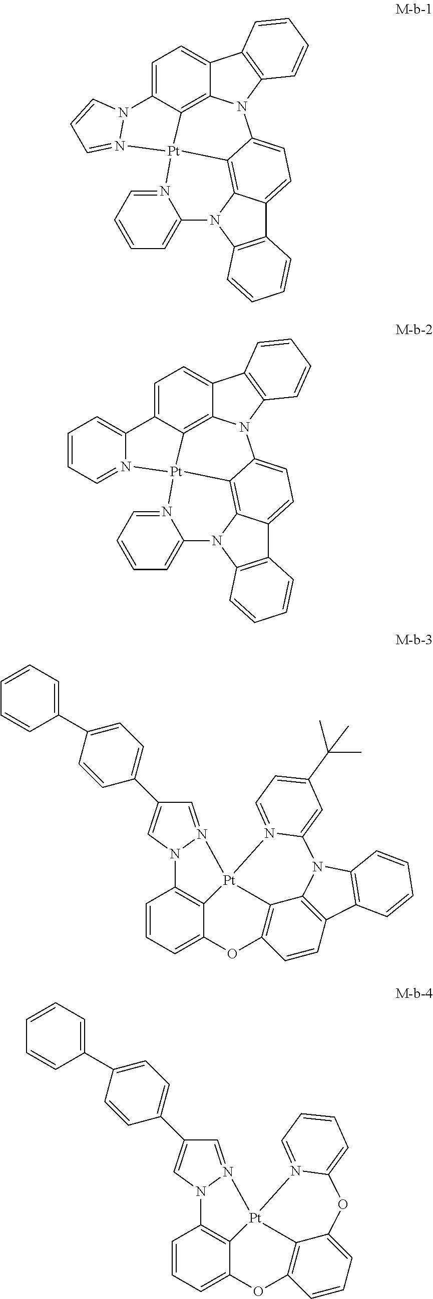

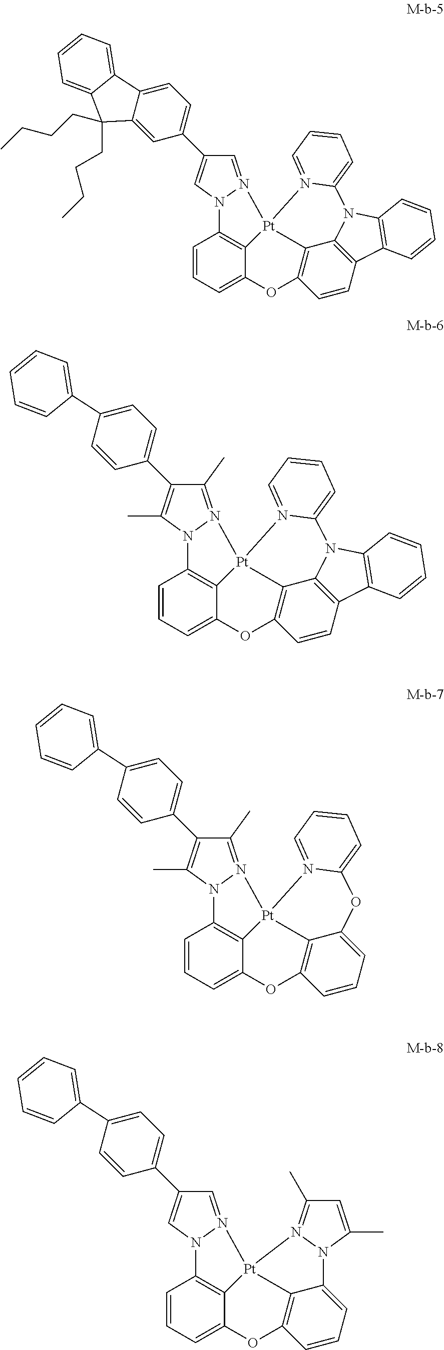

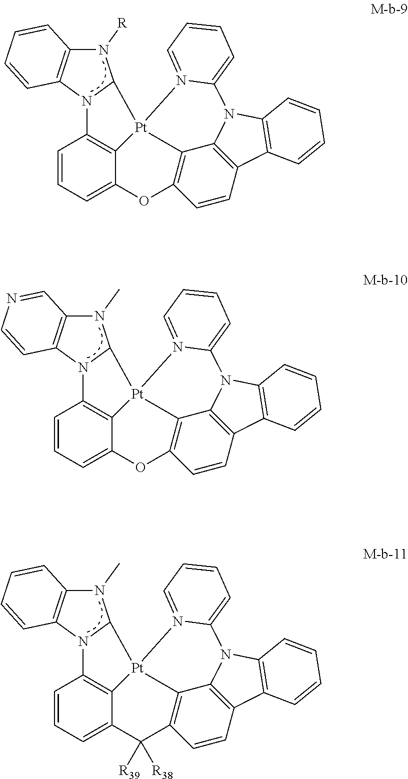

In Compounds M-b-1 to M-b-11, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

In an embodiment, the emission layer EML may include a compound represented by one of Formula F-a to Formula F-c. The compound represented by one of Formula F-a to Formula F-c may be used as a fluorescence dopant material.

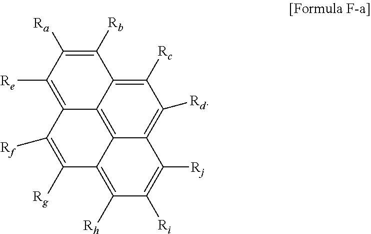

In Formula F-a, two of Rₐ to Rⱼ may each independently be substituted with a group represented by *—NAr₁Ar₂. The remainder of Rₐ to Rⱼ that are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In the group represented by *—NAr₁Ar₂, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. For example, at least one of Ar₁ and Ar² may each independently be a heteroaryl group containing O or S as a ring-forming atom.

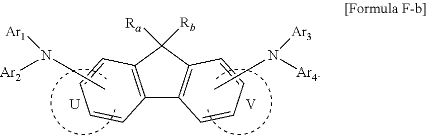

In Formula F-b, R^{a} and R^{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. In Formula F-b, Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms. For example, at least one of Ar₁ to Ar₄ may each independently be a heteroaryl group containing O or S as a ring-forming atom.

In Formula F-b, U and V may each independently be a substituted or unsubstituted hydrocarbon ring having 5 to 30 ring-forming carbon atoms or a substituted or unsubstituted heterocycle having 2 to 30 ring-forming carbon atoms.

In Formula F-b, the number of rings represented by U and V may each independently be 0 or 1. When the number of U or V is 1, a fused ring may be present at a portion respectively indicated by U or V, and when the number of U or V is 0, a fused ring may not be present at the portion respectively indicated by U or V. When the number of U is 0 and the number of V is 1, or when the number of U is 1 and the number of V is 0, a fused ring having a fluorene core of Formula F-b may be a cyclic compound having four rings. When the number of U and V is each 0, a fused ring having a fluorene core of Formula F-b may be a cyclic compound having three rings. When the number of U and V is each 1, a fused ring having a fluorene core of Formula F-b may be a cyclic compound having five rings.

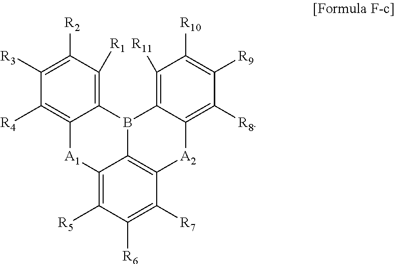

In Formula F-c, A1 and A2 may each independently be O, S, Se, or N(Rₘ); and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

In Formula F-c, A₁ and A₂ may each independently be bonded to a substituent of an adjacent ring to form a fused ring. For example, when A₁ and A₂ are each independently N(Rₘ), A₁ may be bonded to R₄ or R₅ to form a fused ring, and/or A₂ may be bonded to R₇ or R₈ to form a fused ring.

In an embodiment, the emission layer EML may further include, as a dopant material of the related art, a styryl derivative (e.g., 1,4-bis [2-(3-N-ethylcarbazolyl)vinyl]benzene (BCzVB), 4-(di-p-tolylamino)-4′-[(di-p-tolylamino)styryl]stilbene(DPAVB), and N-(4-((E)-2-(6-((E)-4-(diphenylamino) styryl) naphthalen-2-yl) vinyl)phenyl)-N-phenylbenzenamine (N-BDAVBi), 4,4′-bis [2-(4-(N,N-diphenylamino)phenyl)vinyl]biphenyl(DPAVBi), perylene or a derivative thereof (e.g., 2,5,8, 11-tetra-t-butylperylene (TBP)), pyrene or a derivative thereof (e.g., 1,1-dipyrene, 1,4-dipyrenylbenzene, 1,4-bis(N,N-diphenylamino) pyrene), etc.

The emission layer EML may further include a phosphorescence dopant material of the related art. For example, a metal complex containing iridium (Ir), platinum (Pt), osmium (Os), gold (Au), titanium (Ti), zirconium (Zr), hafnium (Hf), curopium (Eu), terbium (Tb), or thulium (Tm) may be used as a phosphorescent dopant. For example, iridium (III) bis(4,6-difluorophenylpyridinato-N,C2′)picolinato (FIrpic), bis(2,4-difluorophenylpyridinato)-tetrakis(1-pyrazolyl) borate iridium (III) (Flr6), or platinum octaethyl porphyrin (PtOEP) may be used as a phosphorescent dopant. However, embodiments are not limited thereto. In an embodiment, the emission layer EML may be a delayed fluorescence emission layer that includes a host and a dopant. For example, the emission layer EML may cmit thermally activated delayed fluorescence (TADF). In an embodiment, the emission layer EML may include a thermally activated delayed fluorescence dopant of the related art.

In an embodiment, the emission layer EML may include host materials, thermally activated delayed fluorescence dopants, phosphorescent sensitizers, and the like.

In an embodiment, the emission layer EML may include a quantum dot material. The quantum dot may include a Group II-VI compound, a Group III-VI compound, a Group I-III-VI compound, a Group III-V compound, a Group III—II-V compound, a Group IV-VI compound, a Group IV element, a Group IV compound, or a combination thereof.

Examples of a Group II-VI compound may include: a binary compound such as CdSe, CdTe, CdS, ZnS, ZnSc, ZnTe, ZnO, HgS, HgSe, HgTe, MgSe, MgS, and a mixture thereof; a ternary compound such as CdSeS, CdSeTe, CdSTe, ZnSeS, ZnSeTe, ZnSTe, HgSeS, HgSeTc, HgSTc, CdZnS, CdZnSc, CdZnTe, CdHgS, CdHgSc, CdHgTe, HgZnS, HgZnSc, HgZnTc, MgZnSe, MgZnS, and a mixture thereof; a quaternary compound such as HgZnTeS, CdZnSeS, CdZnSeTe, CdZnSTe, CdHgSeS, CdHgSeTe, CdHgSTe, HgZnSeS, HgZnSeTe, and a mixture thereof; and any combination thereof.

Examples of a Group III-VI compound may include: a binary compound such as In₂S₃ or In₂Se₃; a ternary compound such as InGaS₃ or InGaSe₃; and any combination thereof.

Examples of a Group I-III-VI compound may include: a ternary compound such as AgInS, AgInS₂, CuInS, CuInS₂, AgGaS₂, CuGaS₂, CuGaO₂, AgGaO₂, AgAlO₂, and a mixture thereof; a quaternary compound such as AgInGaS, AgInGaS2, CulnGaS₂, AgInGaSe, AgInGaSe₂, or CuInGaS; and any combination thereof.

Examples of a Group III-V compound may include: a binary compound such as GaN, GaP, GaAs, GaSb, AlN, AlP, AlAs, AlSb, InN, InP, InAs, InSb, and a mixture thereof; a ternary compound such as GaNP, GaNAs, GaNSb, GaPAs, GaPSb, AlNP, AlNAs, AlNSb, AIPAS, AlPSb, InGaP, InAlP, InNP, InNAs, InNSb, InPAs, InPSb, and a mixture thereof; a quaternary compound such as GaAINP, GaAINAs, GaAINSb, GaAlPAs, GaAlPSb, GalnNP, GalnNAs, GalnNSb, GalnPAs, GalnPSb, InAINP, InAINAs, InAINSb, InAlPAs, InAlPSb, and a mixture thereof; and any combination thereof. In an embodiment, a Group III-V compound may further include a Group II element. Examples of a Group III-II-V compound may include InZnP, etc.

Examples of a Group IV-VI compound may include: a binary compound such as SnS, SnSe, SnTe, PbS, PbSe, PbTe, and a mixture thereof; a ternary compound such as SnSeS, SnSeTe, SnSTe, PbSeS, PbSeTe, PbSTe, SnPbS, SnPbSe, SnPbTe, and a mixture thereof; a quaternary compound such as SnPbSSe, SnPbSeTe, SnPbSTe, and a mixture thereof; and any combination thereof. Examples of a Group IV element may include Si, Ge, and a mixture thereof. Examples of a Group IV compound may include a binary compound such as SiC, SiGe, and a mixture thereof.

Each element included in a compound, such as a binary compound, a ternary compound, or a quaternary compound, may be present in a particle at a uniform concentration distribution or at a non-uniform concentration distribution. For example, a formula may indicate the elements that are included in a compound, but an elemental ratio of the compound may vary. For example, AgInGaS2 may indicate AgInₓGa₁₋ₓS₂ (where x may be a real number between 0 and 1).

In an embodiment, a quantum dot may have a single structure in which the concentration of each element included in the quantum dot is uniform. In an embodiment, a quantum dot may have a core-shell structure in which a quantum dot material surrounds another quantum dot. For example, a material included in the core may be different from a material included in the shell.

The shell of a quantum dot may serve as a protection layer that prevents chemical deformation of the core to maintain semiconductor properties, and/or may serve as a charging layer that imparts electrophoretic properties to the quantum dot. The shell may have a single-layered structure or a multilayered structure. An interface between the core and the shell may have a concentration gradient in which the concentration of an element that is present in the shell decreases towards the center.

In embodiments, the quantum dot may have the above-described core/shell structure that includes a core containing nanocrystals and a shell surrounding the core. Examples of a shell of a quantum dot may include a metal oxide, a non-metal oxide, a semiconductor compound, or a combination thereof.

Examples of a metal oxide or a non-metal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; or a ternary compound such as MgAl₂O₄, CoFe₂O₄, NiFe₂O₄, or CoMn₂O₄, but embodiments are not limited thereto.

Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width at half maximum (FWHM) of an emission spectrum equal to or less than about 45 nm. For example, the quantum dot may have an FWHM of an emission spectrum equal to or less than about 40 nm. For example, the quantum dot may have an FWHM of an emission spectrum equal to or less than about 30 nm. Color purity or color reproducibility may be improved in any of the above ranges. Light emitted through a quantum dot may be emitted in all directions, so that a wide viewing angle may be improved.

The form of a quantum dot is not particularly limited and may be any form used in the related art. For example, a quantum dot may have a spherical shape, a pyramidal shape, a multi-arm shape, or a cubic shape, or a quantum dot may be in the form of nanoparticles, nanotubes, nanowires, nanofibers, nanoplate particles, etc.

As a size of a quantum dot is adjusted or an elemental ratio in a quantum dot compound is adjusted, an energy band gap may be changed accordingly, so that light in various wavelength ranges may be emitted by a quantum dot emission layer. Therefore, by utilizing a quantum dot as described above (for example, using different sizes of quantum dots or having different elemental ratios in a quantum dot compound), a light-emitting element that emits light of various wavelengths may be implemented. For example, the size of the quantum dot or the elemental ratio of the quantum dot compound may be adjusted to emit red light, green light, and/or blue light. For example, quantum dots may be configured to emit white light by combining various colors of light.

In the light-emitting elements ED according to embodiments as shown in each of FIGS. 3 to 7, the electron transport region ETR may be provided on the emission layer EML. The electron transport region ETR may include at least one of a hole blocking layer HBL, an electron transport layer ETL, and an electron injection layer EIL, but embodiments are not limited thereto.

The electron transport region ETR may have a structure consisting of a layer consisting of a single material, a structure consisting of a layer including different materials, or a structure including multiple layers including different materials.

In embodiments, the electron transport region ETR may have a single-layered structure of an electron injection layer EIL or an electron transport layer ETL, or may have a single-layered structure that includes an electron injection material and an electron transport material. In embodiments, the electron transport region ETR may have a single-layered structure formed of different materials, or may have a structure in which an electron transport layer ETL/electron injection layer EIL, or a hole blocking layer HBL/electron transport layer ETL/electron injection layer EIL are stacked in its respective stated order from an emission layer EML, but embodiments are not limited thereto. The electron transport region ETR may have a thickness in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light emitting element ED according to an embodiment, the electron transport region ETR may include a compound represented by Formula ET-2:

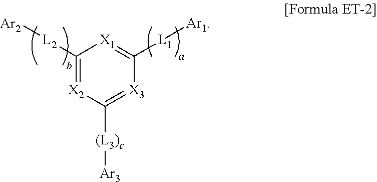

In Formula ET-2, at least one of X¹ to X³ may each be N; and the remainder of X¹ to X³ may each independently be C(Rₐ). In Formula ET-2, R^{a} may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-2, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer from 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are each 2 or more, multiple groups of each of L₁ to L₃ may each independently be a substituted or unsubstituted arylene group having 6 to 30 ring-forming carbon atoms or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzoimidazol-1-yl)phenyl)-9,10-dinaphthylanthracene, 1,3,5-tri (1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), beryllium bis(benzoquinolin-10-olate) (Bebq2), 9,10-di(naphthalene-2-yl) anthracene (ADN), 1,3-bis[3,5-di(pyridin-3-yl)phenyl]benzene (BmPyPhB), or a mixture thereof.

In an embodiment, the electron transport region ETR may include at least one compound selected from Compound ET1 to Compound ET36:

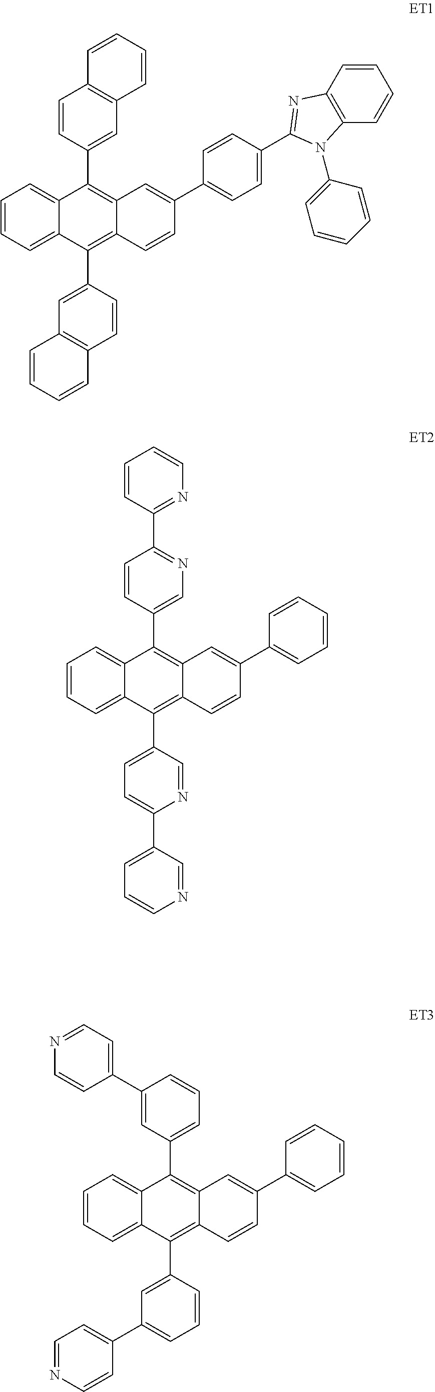 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5cccnc5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

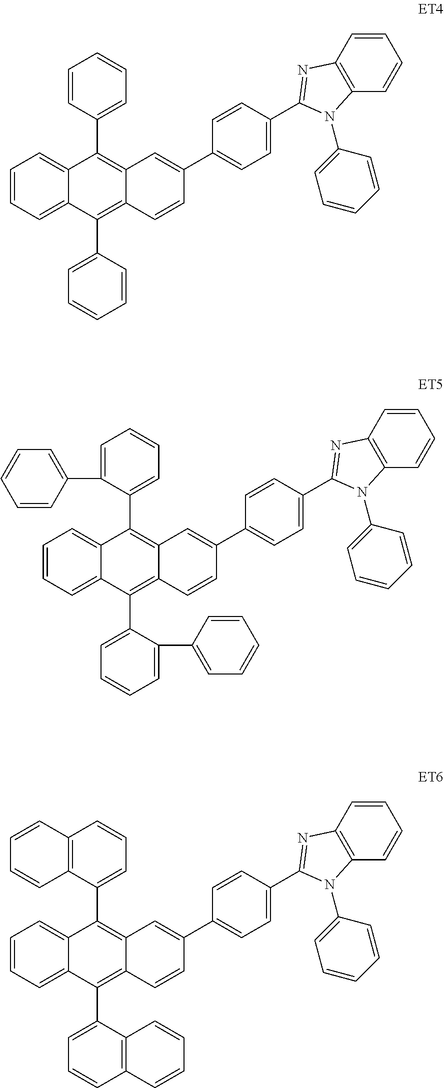 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

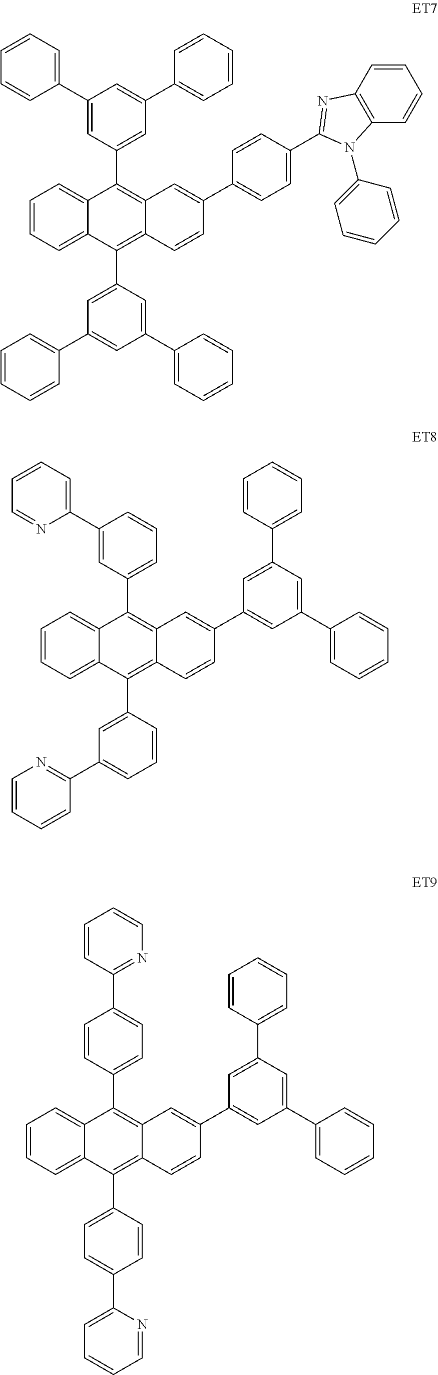 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

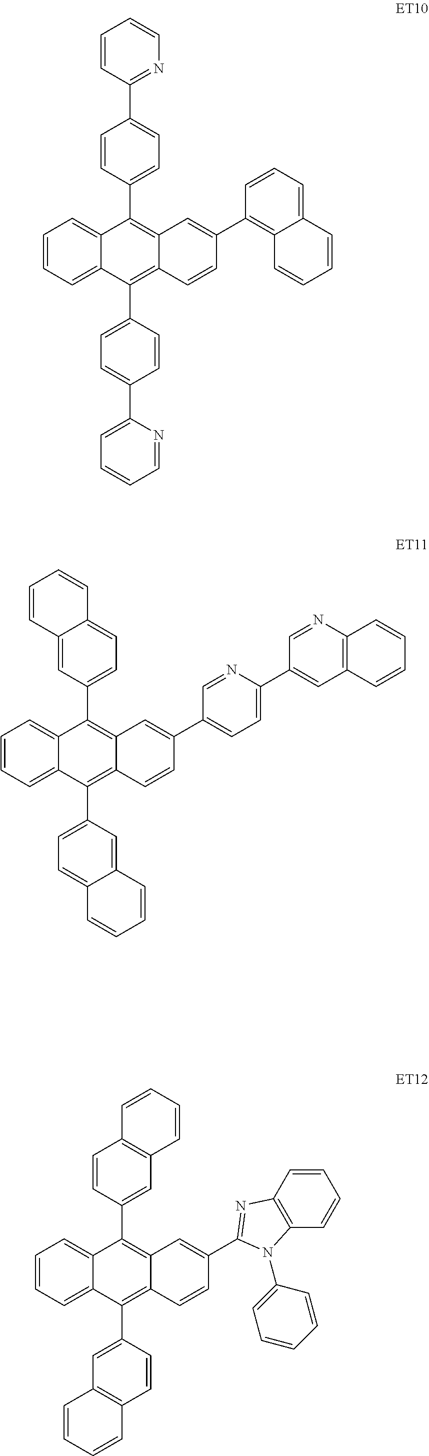 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

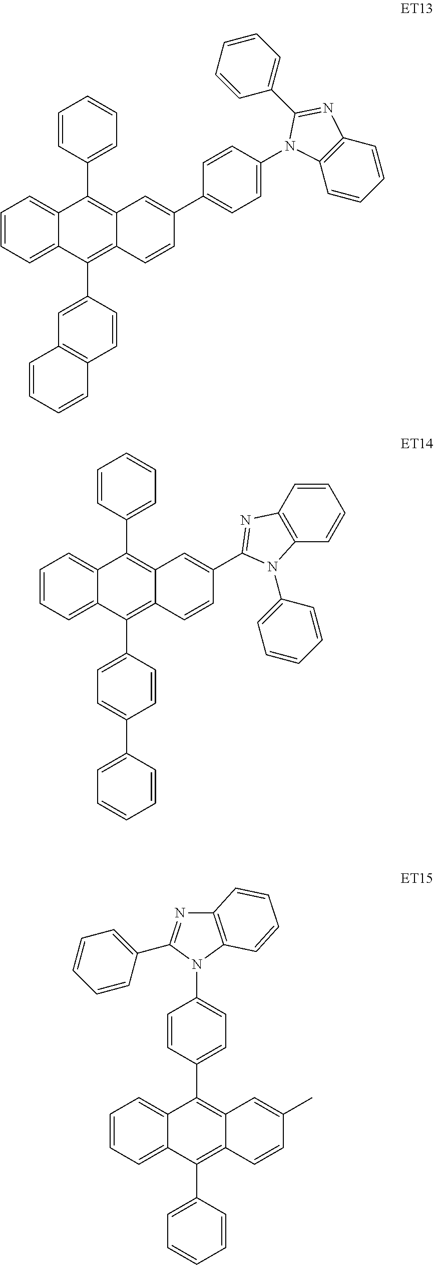 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

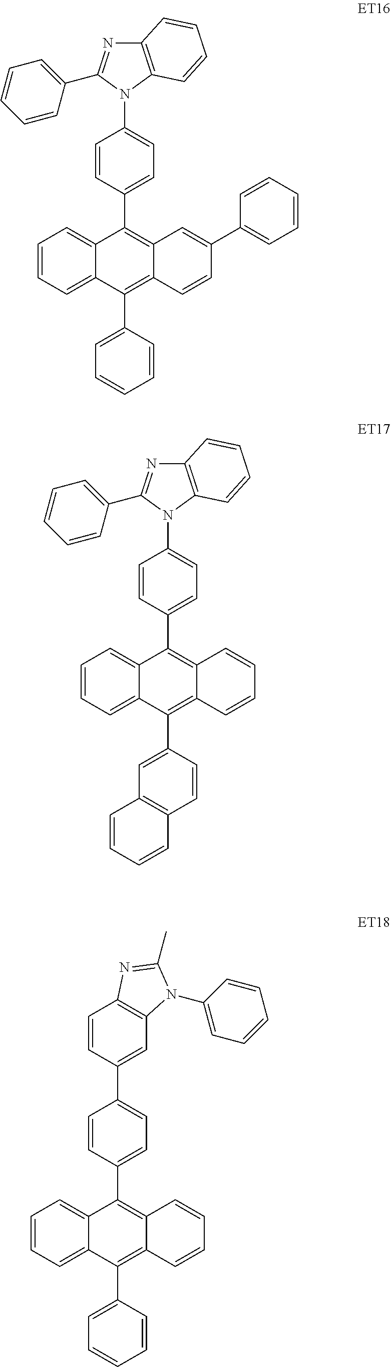 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

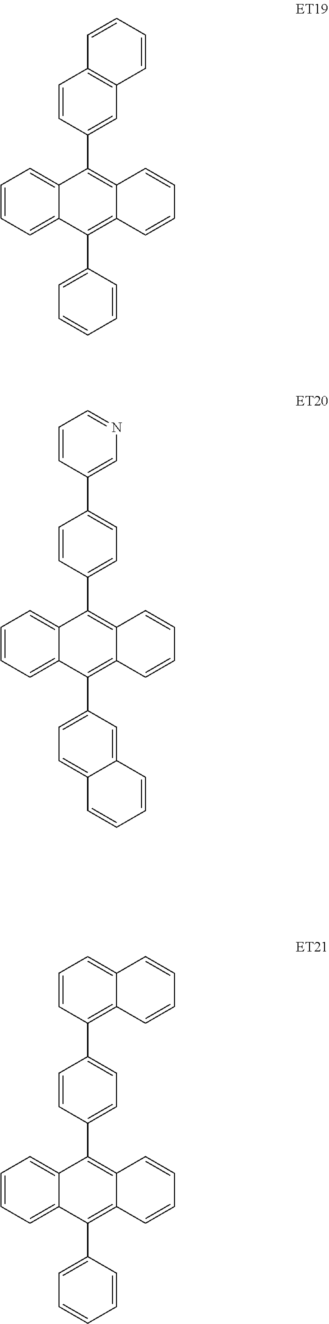

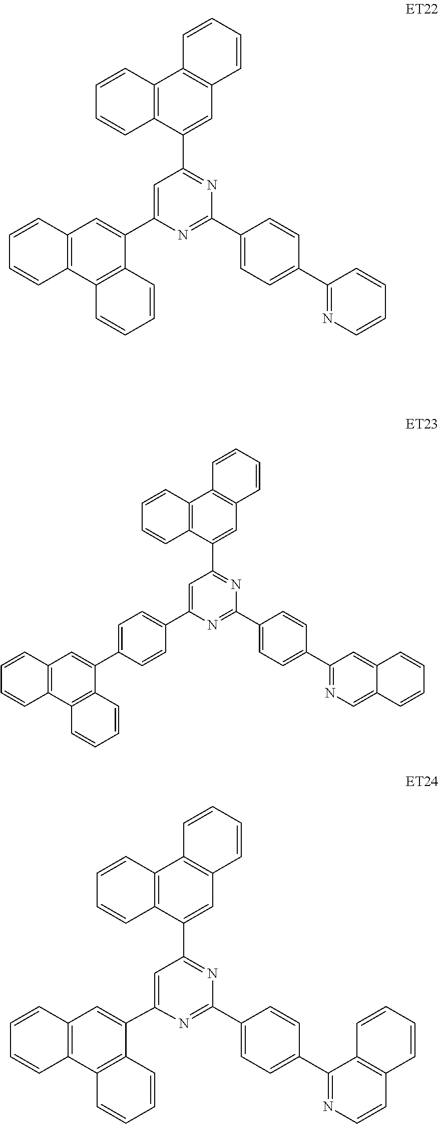 [SMILES: c1ccc(-c2ccc(-c3nc(-c4cc5ccccc5c5ccccc45)cc(-c4cc5ccccc5c5ccccc45)n3)cc2)nc1 ; c1ccc2cc(-c3ccc(-c4nc(-c5ccc(-c6cc7ccccc7c7ccccc67)cc5)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1]

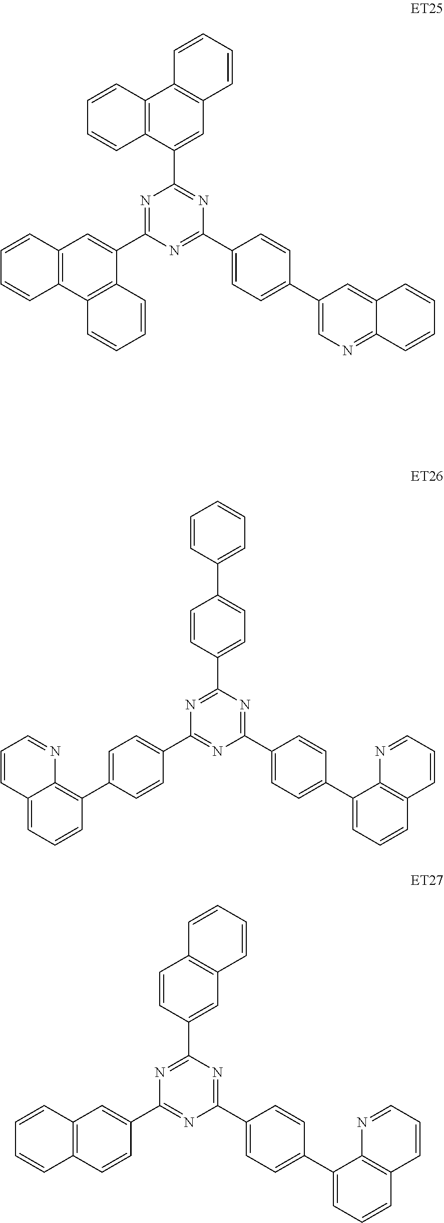 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

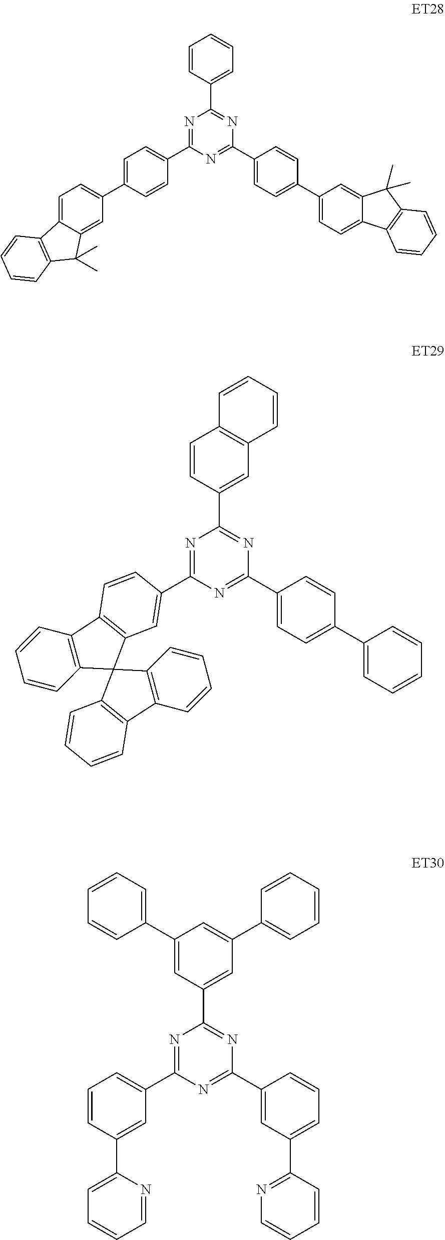 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

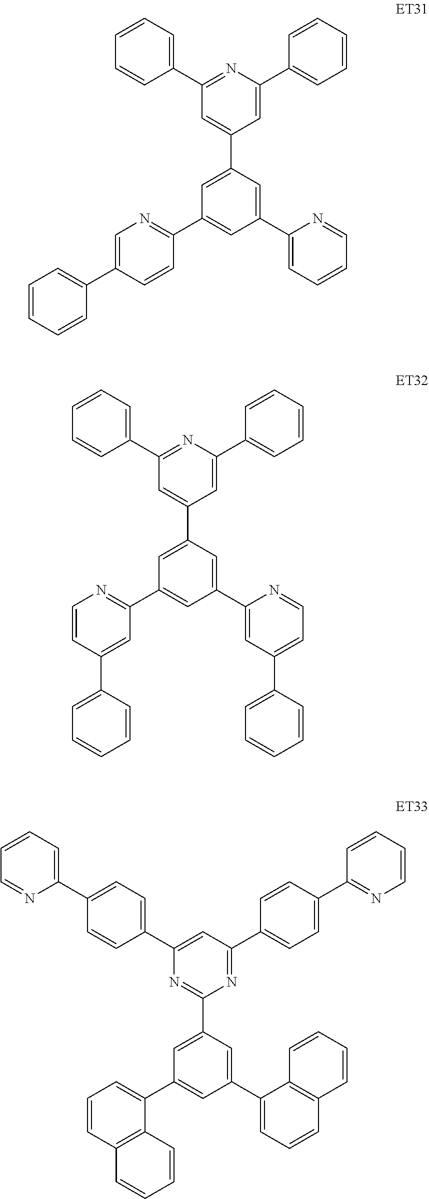 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

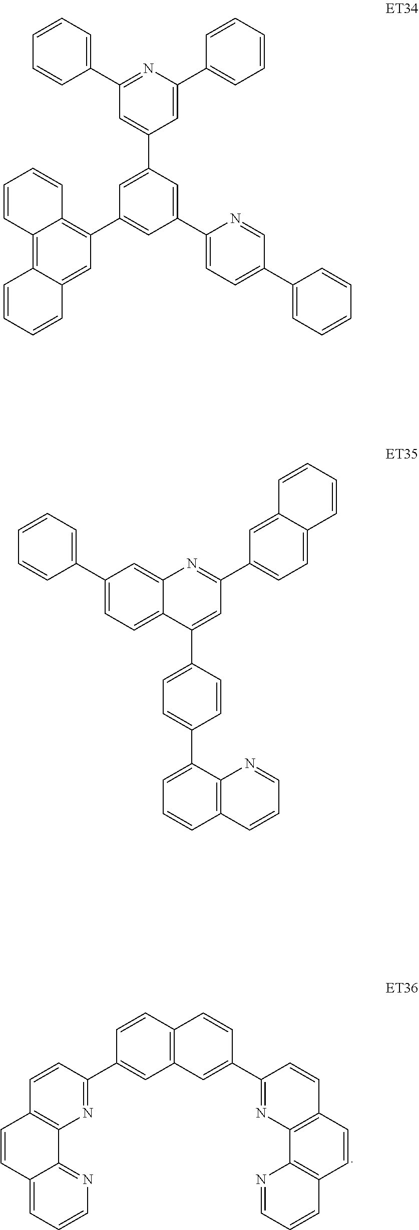 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In an embodiment, the electron transport region ETR may include a metal halide such as LiF, NaCl, CsF, RbCl, RbI, CuI, and KI; a lanthanide such as Yb; or a co-deposited material of the metal halide and the lanthanide. For example, the electron transport region ETR may include KI:Yb, RbI:Yb, LiF:Yb, etc., as a co-deposited material. The electron transport region ETR may include a metal oxide such as Li₂O or BaO, or 8-hydroxyl-lithium quinolate (Liq), etc., but embodiments are not limited thereto. The electron transport region ETR may also be formed of a mixture of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen), in addition to the above-described materials, but embodiments are not limited thereto.

The electron transport region ETR may include the above-described compounds of the electron transport region ETR in at least one of an electron injection layer EIL, an electron transport layer ETL, and a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies the aforementioned range, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies the above-described range, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In an embodiment, the second electrode EL2 may have a multilayered structure that includes a reflective film or a transflective film formed of the above-described materials, and a transparent conductive film formed of ITO, IZO, ZnO, ITZO, etc. For example, the second electrode EL2 may include the above-described metal materials, combinations of at least two of the above-described metal materials, oxides of the above-described metal materials, or the like.

Although not shown in the drawings, the second electrode EL2 may be electrically connected to an auxiliary electrode. If the second electrode EL2 is electrically connected to an auxiliary electrode, resistance of the second electrode EL2 may decrease.

In an embodiment, the light emitting element ED may further include a capping layer CPL disposed on the second electrode EL2. The capping layer CPL may have a multilayered structure or a single-layered structure.

In an embodiment, the capping layer CPL may include an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNₓ, SiO_{y}, etc.

For example, when the capping layer CPL includes an organic material, the organic material may include a-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or may include an epoxy resin, or an acrylate such as methacrylate. However, embodiments are not limited thereto, and the capping layer CPL may include at least one of Compounds P1 to P5:

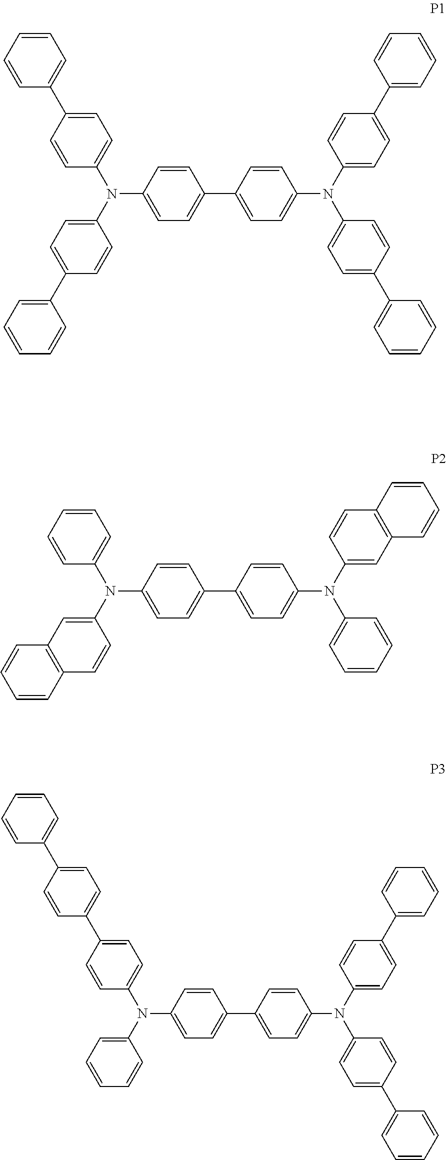 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

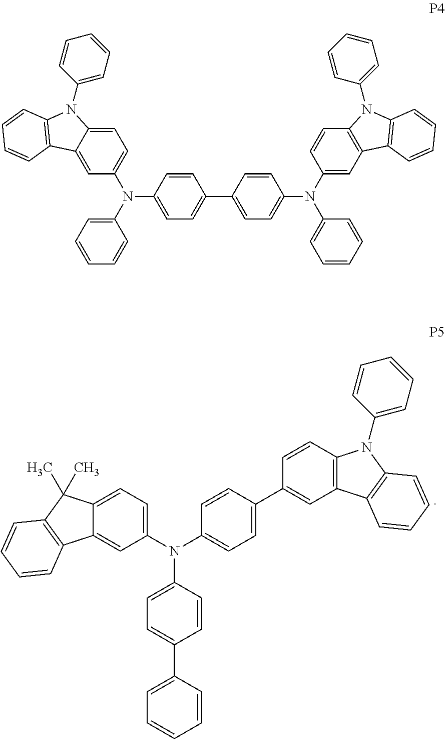 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6, with respect to light in a wavelength range of about 550 nm to about 660 nm.

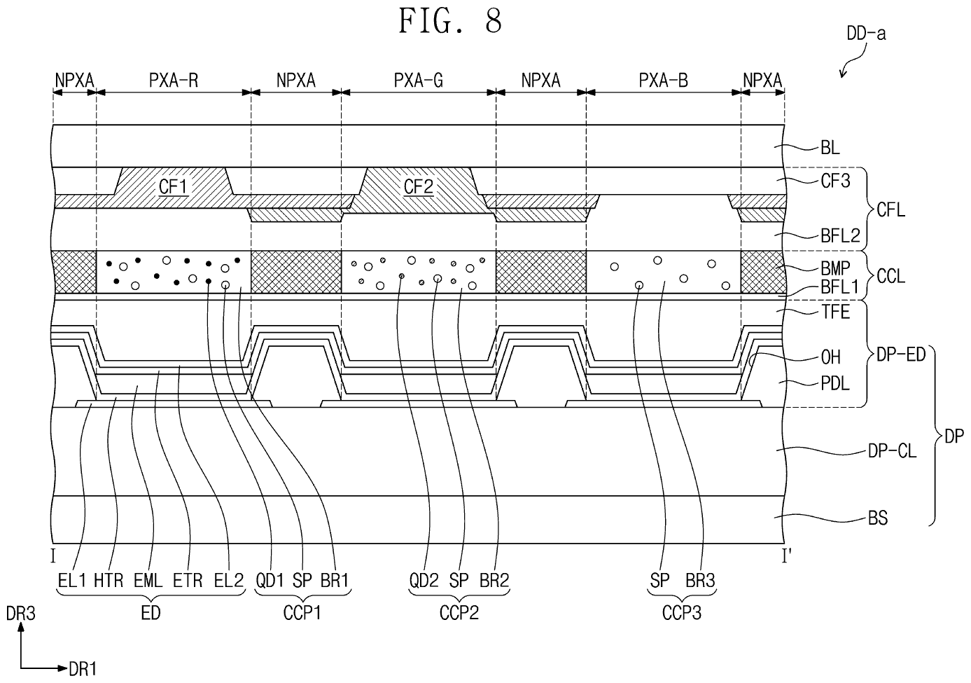

FIGS. 8 to 11 are each a schematic cross-sectional view of a display device according to an embodiment. In the descriptions of the display devices according to embodiments as shown in FIGS. 8 to 11, the features which have been previously described above with respect to FIGS. 1 to 7 will not be described again, and the differing features will be described.

Referring to FIG. 8, the display device DD-a according to an embodiment may include a display panel DP including a display element layer DP-ED, a light control layer CCL disposed on the display panel DP, and a color filter layer CFL. In an embodiment shown in FIG. 8, the display panel DP may include a base layer BS, a circuit layer DP-CL provided on the base layer BS, and the display element layer DP-ED, and the display element layer DP-ED may include a light-emitting element ED.

The light-emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In embodiments, a structure of the light emitting element ED shown in FIG. 8 may be the same as a structure of a light-emitting element ED according to one of FIG. 3 to FIG. 7 as described above.

The light-emitting element ED shown in FIG. 8 may include the amine compound according to an embodiment in the hole transport region HTR. Therefore, the light-emitting element ED may exhibit characteristics of high efficiency and long lifespan. The light-emitting element ED according to an embodiment may exhibit high luminous efficiency and improved lifespan characteristics in a blue light emission region. The light-emitting element ED according to an embodiment includes the amine compound according to an embodiment in the hole transport region HTR, thereby exhibiting characteristics of high efficiency and long lifespan, and thus the display device DD-a may exhibit excellent display quality.

Referring to FIG. 8, the emission layer EML may be disposed in an opening OH defined in a pixel defining film PDL. For example, the emission layer EML, which is separated by the pixel defining film PDL and correspondingly provided to each of the light-emitting regions PXA-R, PXA-G, and PXA-B, may emit light in a same wavelength range. In the display device DD-a, the emission layer EML may emit blue light. Although not shown in the drawings, in an embodiment, the emission layer EML may be provided as a common layer throughout the light-emitting regions PXA-R, PXA-G, and PXA-B.

The light control layer CCL may be disposed on the display panel DP. Although the light control layer CCL is shown as being disposed of the display element layer DP-ED, embodiments are not limited thereto, and the light control layer CCL may be disposed below the display element layer DP-ED. The light control layer CCL may include a light conversion body. The light conversion body may be a quantum dot, a phosphor, or the like. The light conversion body may convert the wavelength of a provided light and transmit the resulting light. For example, the light control layer CCL may be a layer that includes a quantum dot or a layer that includes a phosphor.

The light control layer CCL may include light control parts CCP1, CCP2, and CCP3. The light control parts CCP1, CCP2, and CCP3 may be spaced apart from each other.

Referring to FIG. 8, divided patterns BMP may be disposed between the light control parts CCP1, CCP2, and CCP3, which are spaced apart from each other, but embodiments are not limited thereto. In FIG. 8, it is shown that the divided patterns BMP do not overlap the light control parts CCP1, CCP2, and CCP3, but the edges of the light control parts CCP1, CCP2, and CCP3 may overlap at least a portion of the divided patterns BMP.

The light control layer CCL may include a first light control part CCP1 containing a first quantum dot QD1 that converts first color light provided from the light-emitting element ED into second color light, a second light control part CCP2 containing a second quantum dot QD2 that converts the first color light into third color light, and a third light control part CCP3 that transmits the first color light.

In an embodiment, the first light control part CCP1 may provide red light, which is the second color light, and the second light control part CCP2 may provide green light, which is the third color light. The third light control part CCP3 may provide blue light by transmitting the blue light that is the first color light provided from the light-emitting element ED. For example, the first quantum dot QD1 may be a red quantum dot, and the second quantum dot QD2 may be a green quantum dot. The quantum dots QD1 and QD2 may each be a quantum dot as described above.

The light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include any quantum dot but may include the scatterer SP.

The scatterer SP may be inorganic particles. For example, the scatterer SP may include at least one of TiO₂, ZnO, Al₂O₃, SiO₂, and hollow silica. The scatterer SP may include one of TiO₂, ZnO, Al₂O₃, SiO₂, and hollow silica, or may be a mixture of at least two materials selected from TiO₂, ZnO, Al₂O₃, SiO₂, and hollow silica.

The first light control part CCP1, the second light control part CCP2, and the third light control part CCP3 may respectively include base resins BR1, BR2, and BR3, in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may include various resin compositions, which may be referred to as a binder. For example, the base resins BR1, BR2, and BR3 may be acrylic-based resins, urethane-based resins, silicone-based resins, epoxy-based resins, etc. The base resins BR1, BR2, and BR3 may each be a transparent resin. In an embodiment, the first base resin BR1, the second base resin BR2, and the third base resin BR3 may be the same as or different from each other.

The light control layer CCL may include a barrier layer BFL1. The barrier layer BFL1 may prevent the penetration of moisture and/or oxygen (hereinafter, referred to as ‘moisture/oxygen’). The barrier layer BFL1 may block the light control parts CCP1, CCP2, and CCP3 from exposure to moisture/oxygen. The barrier layer BFL1 may cover the light control parts CCP1, CCP2, and CCP3. In an embodiment, the barrier layer BFL2 may be provided between the light control parts CCP1, CCP2, and CCP3 and the filters CF1, CF2, and CF3.

The barrier layers BFL1 and BFL2 may each independently include at least one inorganic layer. For example, the barrier layers BFL1 and BFL2 may each independently include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film that secures light transmittance, etc. The barrier layers BFL1 and BFL2 may each independently further include an organic film. The barrier layers BFL1 and BFL2 may be single-layered or multilayered.

In the display device DD-a, the color filter layer CFL may be disposed on the light control layer CCL. In an embodiment, the color filter layer CFL may be directly disposed on the light control layer CCL. For example, the barrier layer BFL2 may be omitted.

The color filter layer CFL may include filters CF1, CF2, and CF3. The color filter layer CFL may include a first filter CFI that transmits the second color light, a second filter CF2 that transmits the third color light, and a third filter CF3 that transmits the first color light. For example, the first filter CFI may be a red filter, the second filter CF2 may be a green filter, and the third filter CF3 may be a blue filter. The filters CF1, CF2, and CF3 may each include a polymeric photosensitive resin and a pigment or dye. The first filter CFI may include a red pigment or dye, the second filter CF2 may include a green pigment or dye, and the third filter CF3 may include a blue pigment or dye.

However, embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be provided as separate filters and may be provided as a unitary filter.

Although not shown in the drawings, in an embodiment, the color filter layer CFL may further include a light shielding part (not shown). The light shielding part (not shown) may be a black matrix. The light shielding part (not shown) may include an organic light shielding material or an inorganic light shielding material, each including a black pigment or a black dye. The light shielding part (not shown) may prevent light leakage, and may separate boundaries between adjacent filters CF1, CF2, and CF3. In an embodiment, the light shielding part (not shown) may be formed of a blue filter.

The first filter CF1, the second filter CF2, and the third filter CF3 may be respectively disposed to correspond to the red light-emitting region PXA-R, the green light-emitting region PXA-G, and the blue light-emitting region PXA-B.

A base substrate BL may be disposed on the color filter layer CFL. The base substrate BL may provide a base surface on which the color filter layer CFL, the light control layer CCL, and the like are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

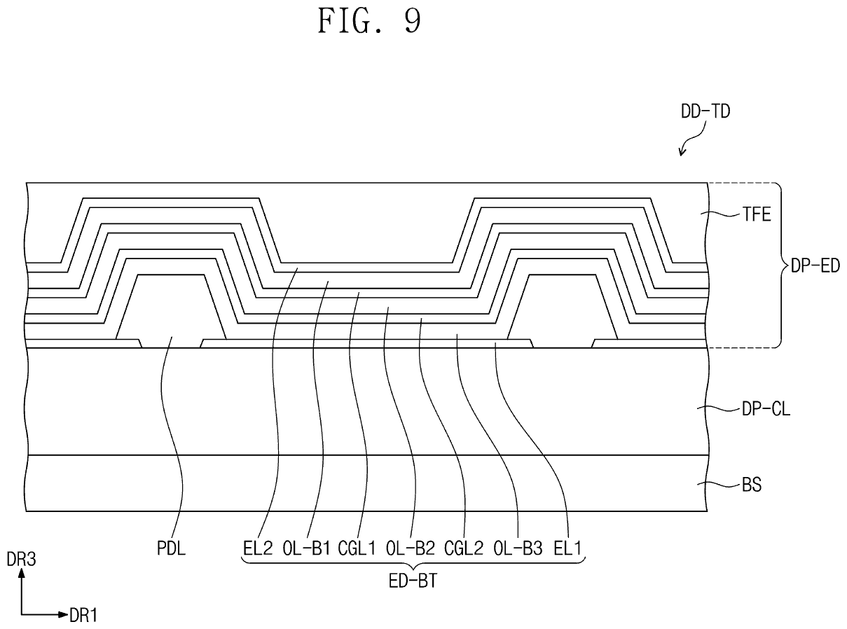

FIG. 9 is a schematic cross-sectional view of a portion of a display device according to an embodiment. In a display device DD-TD according to an embodiment, a light-emitting element ED-BT may include light-emitting structures OL-B1, OL-B2, and OL-B3.

The light-emitting element ED-BT may include a first electrode EL1 and a second electrode EL2 which face each other, and light-emitting structures OL-B1, OL-B2, and OL-B3 stacked in a thickness direction between the first electrode EL1 and the second electrode EL2. The light-emitting structures OL-B1, OL-B2, and OL-B3 may each include a hole transport region HTR, an emission layer EML (FIG. 8), and an electron transport region ETR, which may be disposed in that order between the first electrode EL1 and the second electrode EL2. For example, the light-emitting element ED-BT included in the display device DD-TD may be a light-emitting element having a tandem structure that includes multiple emission layers EML.

In an embodiment illustrated in FIG. 9, light emitted from the light-emitting structures OL-B1, OL-B2, and OL-B3 may each be blue light. However, embodiments are not limited thereto, and the light emitted from each of the light-emitting structures OL-B1, OL-B2, and OL-B3 may have wavelength ranges that are different from each other. For example, the light-emitting element ED-BT that includes the light-emitting structures OL-B1, OL-B2, and OL-B3, which emit light having wavelength ranges that are different from each other, may emit white light.

Charge generating layers CGL1 and CGL2 may each be disposed between two adjacent light emitting structures among the light-emitting structures OL-B1, OL-B2, and OL-B3. Charge generating layer CGL1 and CGL2 may each independently include a p-type charge generating layer and/or an n-type charge generating layer.

In an embodiment illustrated in FIG. 9, at least one among the light-emitting structures OL-B1, OL-B2, and OL-B3 may include the amine compound according to an embodiment. Therefore, at least one of the light-emitting structures OL-B1, OL-B2, and OL-B3 may exhibit characteristics of high efficiency and long lifespan, and a light-emitting element ED-BT may exhibit characteristics of high efficiency and long lifespan.

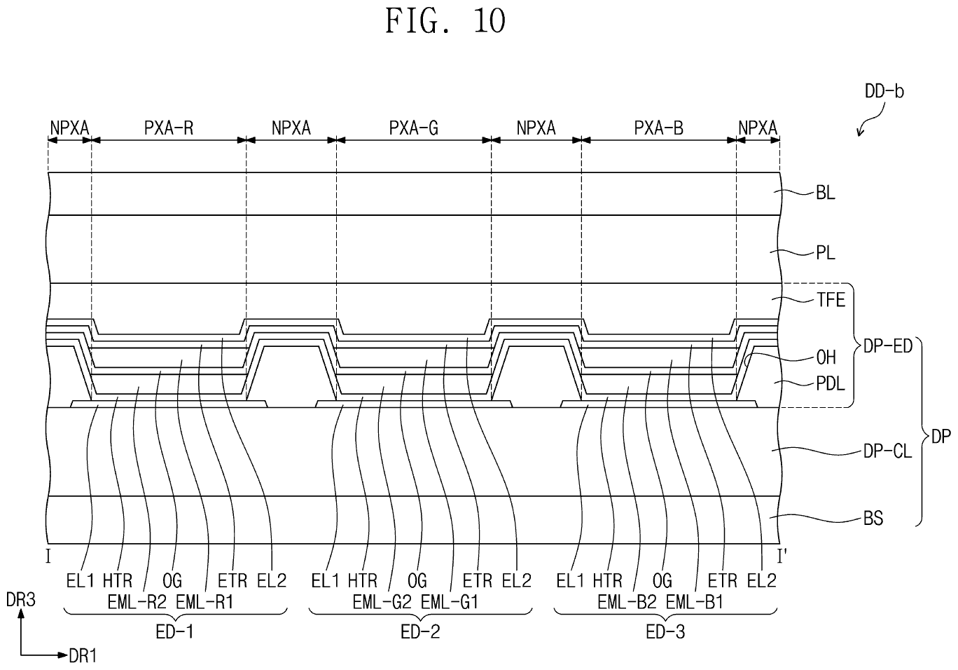

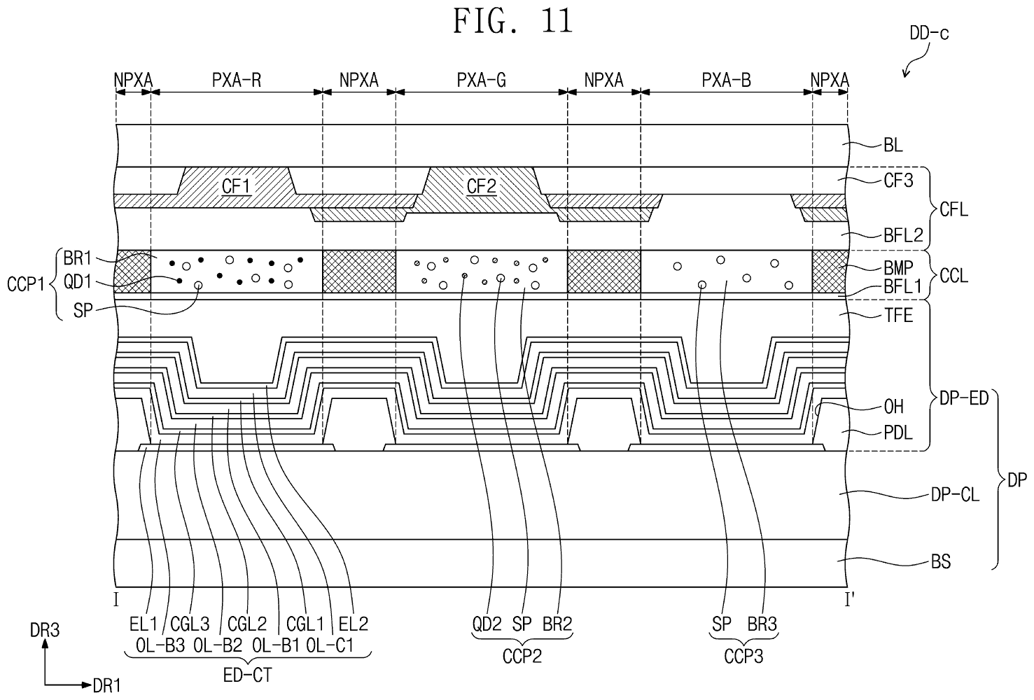

FIG. 10 is a schematic cross-sectional view of a display device DD-b according to an embodiment. FIG. 11 is a schematic cross-sectional view of a display device DD-c according to an embodiment.

Referring to FIG. 10, a display device DD-b according to an embodiment may include light-emitting elements ED-1, ED-2, and ED-3, in which two emission layers are stacked. At least one of the light-emitting elements ED-1, ED-2, and ED-3 may include the amine compound according to an embodiment. Therefore, the light-emitting elements ED-1, ED-2, and ED-3 may exhibit characteristics of high efficiency and long lifespan. In an embodiment, a light-emitting element ED-3 that includes the amine compound according to an embodiment may exhibit characteristics of high efficiency and improved lifespan in a blue light emission region.

In comparison to the display device DD shown in FIG. 2, the embodiment illustrated in FIG. 10 is different at least in that the first to third light-emitting elements ED-1, ED-2, and ED-3 each include two emission layers that are stacked in a thickness direction. In each of the first to third light-emitting elements ED-1, ED-2, and ED-3, the two emission layers may emit light in a same wavelength region.

The first light-emitting element ED-1 may include a first red emission layer EML-R1 and a second red emission layer EML-R². The second light-emitting element ED-2 may include a first green emission layer EML-G1 and a second green emission layer EML-G2. The third light-emitting element ED-3 may include a first blue emission layer EML-B1 and a second blue emission layer EML-B2. An emission auxiliary part OG may be disposed between the first red emission layer EML-R1 and the second red emission layer EML-R², between the first green emission layer EML-G1 and the second green emission layer EML-G2, and between the first blue emission layer EML-BI and the second blue emission layer EML-B2.

The emission auxiliary part OG may have a single layered structure or a multilayered structure. The emission auxiliary part OG may include a charge generation layer. For example, the emission auxiliary part OG may include an electron transport region, a charge generation layer, and a hole transport region, which may be stacked in that order. The emission auxiliary part OG may be provided as a common layer for each of the first to third light-emitting elements ED-1, ED-2, and ED-3. However, embodiments are not limited thereto, and the emission auxiliary part OG may be provided by being patterned within the openings OH defined in the pixel defining film PDL.

The first red emission layer EML-R¹, the first green emission layer EML-G1, and the first blue emission layer EML-B1 may each be disposed between the electron transport region ETR and the emission auxiliary part OG. The second red emission layer EML-R², the second green emission layer EML-G2, and the second blue emission layer EML-B2 may each be disposed between the emission auxiliary part OG and the hole transport region HTR.

For example, the first light-emitting element ED-1 may include a first electrode EL1, a hole transport region HTR, a second red emission layer EML-R², an emission auxiliary part OG, a first red emission layer EML-R1, an electron transport region ETR, and a second electrode EL2, which are stacked in that order. The second light-emitting element ED-2 may include a first electrode EL1, a hole transport region HTR, a second green emission layer EML-G2, an emission auxiliary part OG, a first green emission layer EML-G1, an electron transport region ETR, and a second electrode EL2, which are stacked in that order. The third light-emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, which are stacked in that order.

The light-emitting elements ED-1, ED-2, and ED-3 may each include the amine compound according to an embodiment in the hole transport region HTR to exhibit high efficiency and long service life characteristics, and thus the display device DD-b may exhibit excellent display quality.

An optical auxiliary layer PL may be disposed on the display element layer DP-ED. The optical auxiliary layer PL may include a polarizing layer. The optical auxiliary layer PL may be disposed on the display panel DP and may control light that is reflected at the display panel DP from an external light. Although not shown in the drawings, in an embodiment, the optical auxiliary layer PL may be omitted from the display device DD-b.

In contrast to FIGS. 9 and 10, FIG. 11 shows a display device DD-c that is different at least in that it includes four light-emitting structures OL-B1, OL-B2, OL-B3, and OL-C1. A light-emitting element ED-CT may include a first electrode EL1 and a second electrode EL2 that face each other, and first to fourth light-emitting structures OL-B1, OL-B2, OL-B3, and OL-C1 that are stacked in a thickness direction between the first electrode EL1 and the second electrode EL2.

At least one of the first to fourth light-emitting structures OL-B1, OL-B2, OL-B3, and OL-C1 may each independently include the amine compound according to an embodiment. Therefore, a light-emitting element ED-CT may exhibit characteristics of high efficiency and long lifespan, and the display device DD-c according to an embodiment may exhibit excellent display quality.

The light emitting structures OL-C1, OL-B1, OL-B2, and OL-B3 may be sequentially stacked in this order; charge generation layer CGL1 may be disposed between light emitting structures OL-B1 and OL-C1; charge generation layer CGL2 may be disposed between light emitting structures OL-B1 and OL-B2; and charge generation layer CGL3 may be disposed between light emitting structures OL-B2 and OL-B3The charge generation layers CGL1, CGL2, and CGL3 may each independently include a p-type charge generation layer and/or an n-type charge generation layer.

Among the four light-emitting structures, the first to third light-emitting structures OL-B1, OL-B2, and OL-B3 may each emit blue light, and the fourth light-emitting structure OL-C1 may emit green light. However, embodiments are not limited thereto, and the first to fourth light-emitting structures OL-B1, OL-B2, OL-B3, and OL-C1 may emit light in different wavelength regions.

In an embodiment, an electronic apparatus may include a display device that includes multiple light-emitting elements, and a control part that controls the display device. The electronic apparatus may be a device that is activated by an electrical signal. The electronic apparatus may include display devices according to various embodiments. Examples of an electronic apparatus may include large, medium-sized, and small apparatuses, such as a television set, a monitor, a billboard, a personal computer, a laptop computer, a personal digital terminal, a display device for a vehicle, a game console, a portable electronic device, or a camera.

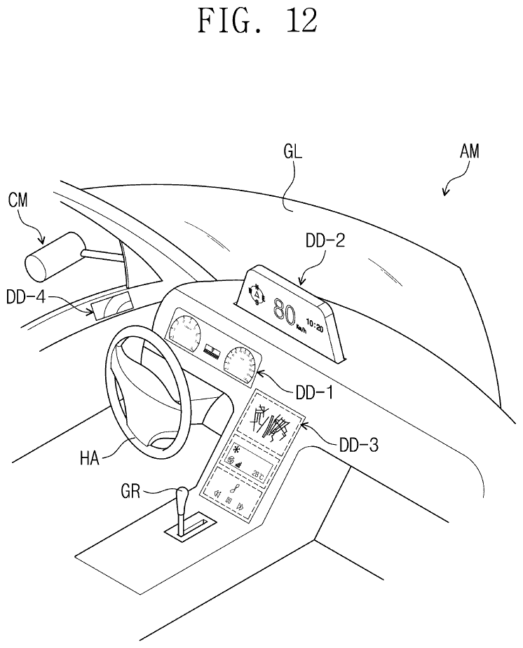

FIG. 12 is a schematic perspective view of a vehicle AM that includes first to fourth display devices DD-1, DD-2, DD-3, and DD-4. At least one of the first to fourth display devices DD-1, DD-2, DD-3, and DD-4 may have a structure according to one of display devices DD, DD-TD, DD-a, DD-b, and DD-c, as described above with reference to FIGS. 1, 2, and 8 to 11.

FIG. 12 illustrates a vehicle AM, but this is only an example, and the first to fourth display devices DD-1, DD-2, DD-3, and DD-4 may be disposed in various transportation means, such as a bicycle, a motorcycle, a train, a ship, and an airplane. In an embodiment, at least one of the first to fourth display devices DD-1, DD-2, DD-3, and DD-4 each having a structure according to one of display devices DD, DD-TD, DD-a, DD-b, and DD-c, may be included in a personal computer, a laptop computer, a personal digital terminal, a game console, a portable electronic device, a television, a monitor, a billboard, or the like. However, these are merely provided as examples, and the display device may be included in other electronic apparatuses.

At least one of the first to fourth display devices DD-1, DD-2, DD-3, and DD-4 may each independently include a light-emitting element ED according to an embodiment as described with reference to any of FIGS. 3 to 7.

At least one of the first to fourth display devices DD-1, DD-2, DD-3, and DD-4 may each independently include the amine compound according to an embodiment. Accordingly, the first to fourth display devices DD-1, DD-2, DD-3, and DD-4 including the amine compound may have an improvement in display efficiency and a display service life. The first to fourth display devices DD-1, DD-2, DD-3, and DD-4 including the amine compound may exhibit excellent display quality.

Referring to FIG. 12, the vehicle AM may include a steering wheel HA and a gearshift GR for operating the vehicle AM. The vehicle AM may include a front window GL that is disposed so as to face the driver.

The first display device DD-1 may be disposed in a first region that overlaps the steering wheel HA. For example, the first display device DD-1 may be a digital cluster that displays first information of the vehicle AM. The first information may include a first scale that indicates a driving speed of the vehicle AM, a second scale that indicates an engine speed (for example, as revolutions per minute (RPM)), an image that represents a fuel gauge, etc. The first scale and the second scale may each be represented by a digital image

The second display device DD-2 may be disposed in a second region facing the driver's seat that overlaps the front window GL. The driver's seat may be a seat where the steering wheel HA is disposed. For example, the second display device DD-2 may be a head up display (HUD) that shows second information of the vehicle AM. The second display device DD-2 may be optically transparent. The second information may include digital numbers that indicate a driving speed and may further include information such as the current time. Although not shown in the drawings, in an embodiment, the second information of the second display device DD-2 may be displayed by being projected onto the front window GL.

The third display device DD-3 may be disposed in a third region that is adjacent to the gearshift GR. For example, the third display device DD-3 may be disposed between a driver's seat and a passenger seat and may be a center information display (CID) for the vehicle AM that displays third information. The passenger seat may be a seat that is spaced apart from the driver's seat, and the gearshift GR may be disposed between the driver's seat and the passenger seat. The third information may include information about traffic (e.g., navigation information), about music or radio that is playing, about a video (or an image) that is displayed, about temperatures inside the vehicle AM, etc.

The fourth display device DD-4 may be disposed in a fourth region that is spaced apart from the steering wheel HA and the gearshift GR, and may be adjacent to a side of the vehicle AM. For example, the fourth display device DD-4 may be a digital side-view mirror that displays fourth information. The fourth display device DD-4 may display an image that is external to the vehicle AM, which may be taken by a camera module CM disposed on the exterior of the vehicle AM. The fourth information may include an exterior image of the vehicle AM.

The first to fourth information as described above are only provided as examples, and the first to fourth display devices DD-1, DD-2, DD-3, and DD-4 may further display information about the interior and exterior of the vehicle AM. The first to fourth information may include information that is different from each other. However, embodiments are not limited thereto, and a portion of the first to fourth information may include a same information.

Hereinafter, an amine compound according to an embodiment and a light-emitting element according to an embodiment will be described in detail with reference to the Examples and the Comparative Examples. The Examples shown below are only provided to facilitate the understanding of the disclosure, and thus, the scope of the disclosure is not limited thereto.

### EXAMPLES

### 1. Synthesis of Amine Compound According to Embodiment

A synthesis method of an amine compound according to an embodiment will be explained in detail by describing synthesis methods for Compounds 8, 11, 40, 44, 111, 131, 135, 218, 235, 239, and 261. In the following descriptions, the synthesis method for the amine compound is only provided as an example, and the synthesis method for the amine compound according to an embodiment is not limited to the Examples below.

In the synthesis methods of the amine compounds, the molecular weight of the compounds was measured by FAB-MS using JMS-700V (JEOL Ltd.).

### (1) Synthesis of Compound 8

Compound 8 according to an embodiment may be synthesized by, for example, Reaction Scheme 1:

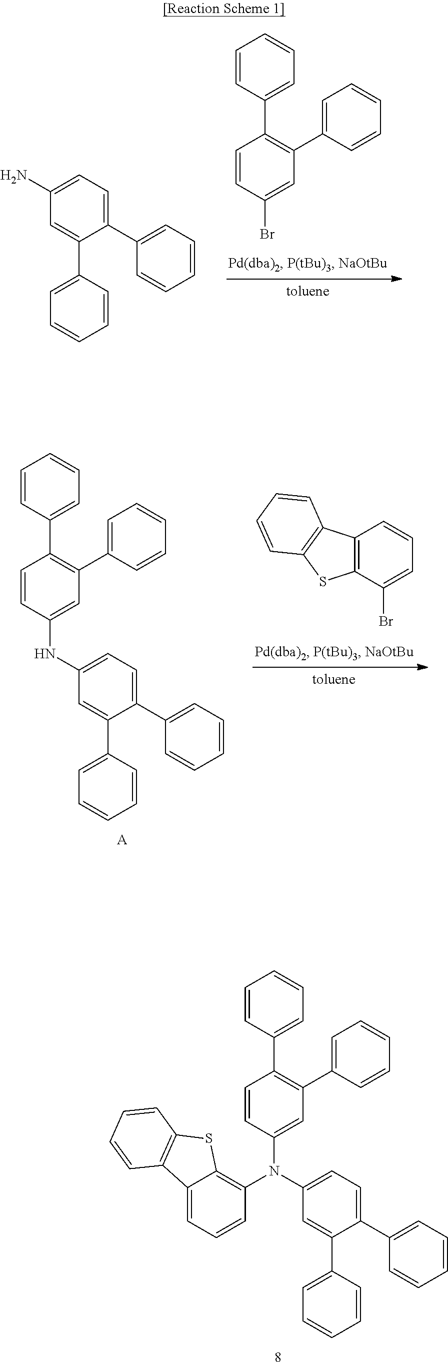

### Synthesis of Compound 8

In an argon (Ar) atmosphere, [1,1′: 2′,1″-terphenyl]-4′-amine (20.0 g), 4′-bromo-1,1′: 2′,1″-terphenyl (25.2 g), bis(dibenzylideneacetone) palladium (0) (Pd (dba) 2, 2.3 g), and sodium tert-butoxide (NaOtBu, 11.7 g) were put into a 1 L three-neck flask and dissolved in toluene (400 mL). Tri-tert-butylphosphine (P(tBu)₃, 2.0 M in toluene, 2.0 mL) was added, and the mixture was stirred at room temperature for 8 hours. Water was added, the resulting product was extracted with CH₂Cl₂, an organic layer was collected and dried over MgSO₄, and the solvent was removed by distillation under reduced pressure. The obtained crude product was purified by silica gel column chromatography to obtain Intermediate Compound A of 22.3 g (yield of 58%). A molecular weight of Intermediate Compound A was 473, as measured by FAB-MS measurement.

In an argon atmosphere, Intermediate Compound A (5.0 g), 4-bromodibenzo[b,d]thiophene (2.8 g), Pd(dba)₂ (0.30 g), and NaOtBu (1.5 g) were put into a 300 mL three-neck flask, dissolved in toluene (100 mL), P(tBu)₃ (2.0 M in toluene, 0.5 mL) was added, and the mixture was stirred at 100° C. for 4 hours. Water was added, the resulting product was extracted with CH₂Cl₂, an organic layer was collected and dried over MgSO₄, and the solvent was removed by distillation under reduced pressure. The obtained crude product was purified by silica gel column chromatography to obtain Compound 8 of 5.5 g (yield of 80%). A molecular weight of Compound 8 was 655, as measured by FAB-MS measurement.

### (2) Synthesis of Compound 11

Compound 11 according to an embodiment may be synthesized by, for example, Reaction Scheme 2:

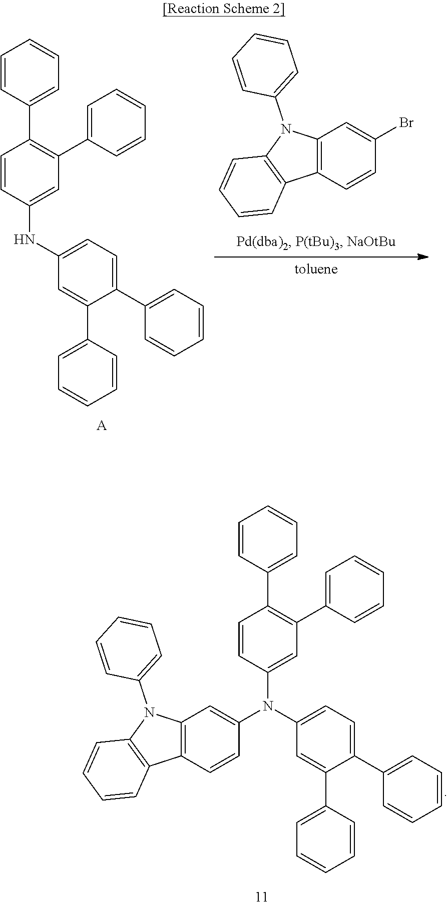

In the same manner as the synthesis of Compound 8 described above, Compound 11 of 6.4 g (yield of 85%) was obtained from Intermediate Compound A (5.0 g), and 2-bromo-9-phenyl-9H-carbazole (3.4 g). A molecular weight of Compound 11 was 714, as measured by FAB-MS measurement.

### (3) Synthesis of Compound 40

Compound 40 according to an embodiment may be synthesized by, for example, Reaction Scheme 3:

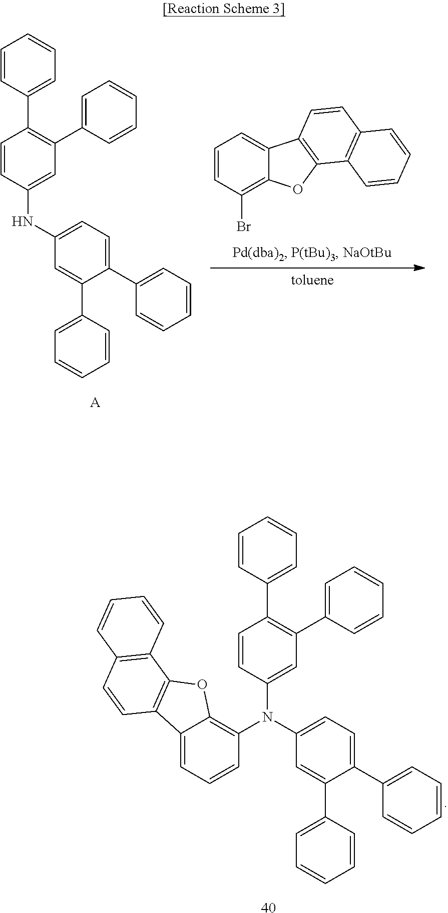

In the same manner as the synthesis of Compound 8 described above, Compound 40 of 5.4 g (yield of 75%) was obtained from Intermediate Compound A (5.0 g), and 10-bromonaphtho[1,2-b]benzofuran (3.2 g). The molecular weight of Compound 40 was 689, as measured by FAB-MS measurement.

### (4) Synthesis of Compound 44

Compound 44 according to an embodiment may be synthesized by, for example, Reaction Scheme 4:

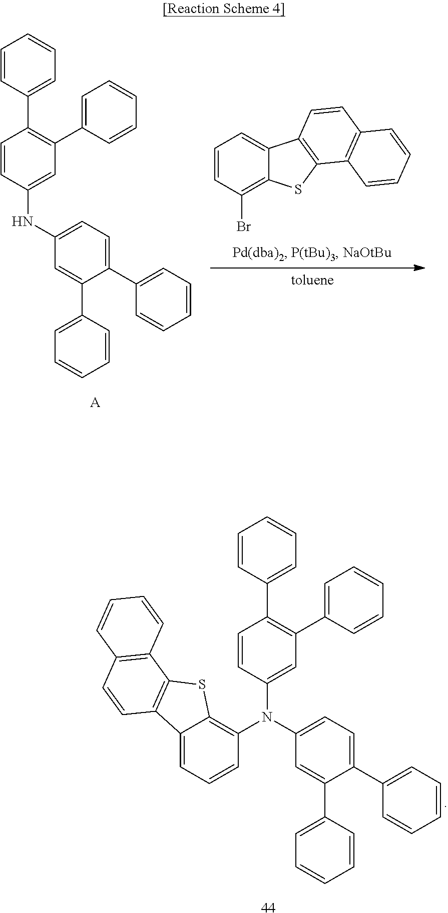

In the same manner as the synthesis of Compound 8 described above, Compound 44 of 6.0 g (yield of 81%) was obtained from Intermediate Compound A (5.0 g), and 10-bromobenzo[b]naphtho[2,1-d]thiophene (3.3 g). A molecular weight of Compound 44 was 705, as measured by FAB-MS measurement.

### (5) Synthesis of Compound 111

Compound 111 according to an embodiment may be synthesized by, for example,

### Reaction Scheme 5:

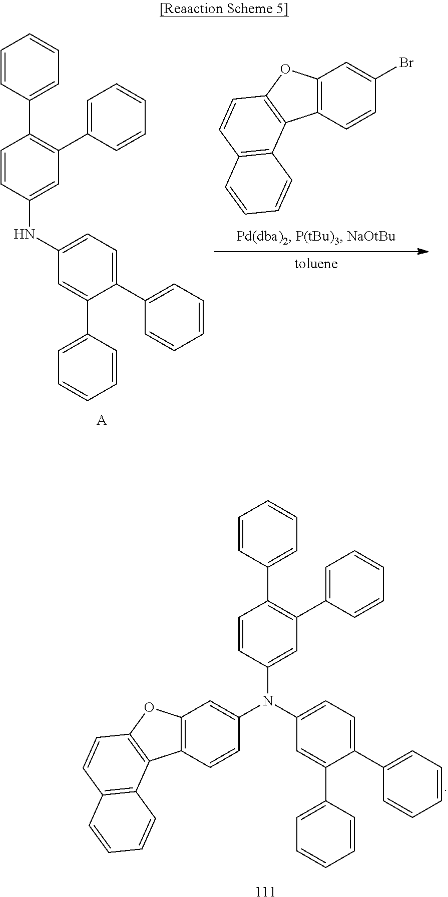

In the same manner as the synthesis of Compound 8 described above, Compound 111 of 5.2 g (yield of 82%) was obtained from Intermediate Compound A (5.0 g), and 9-bromonaphtho[2,1-b]benzofuran (3.2 g). A molecular weight of Compound 111 was 689, as measured by FAB-MS measurement.

### (6) Synthesis of Compound 131

Compound 131 according to an embodiment may be synthesized by, for example, Reaction Scheme 6:

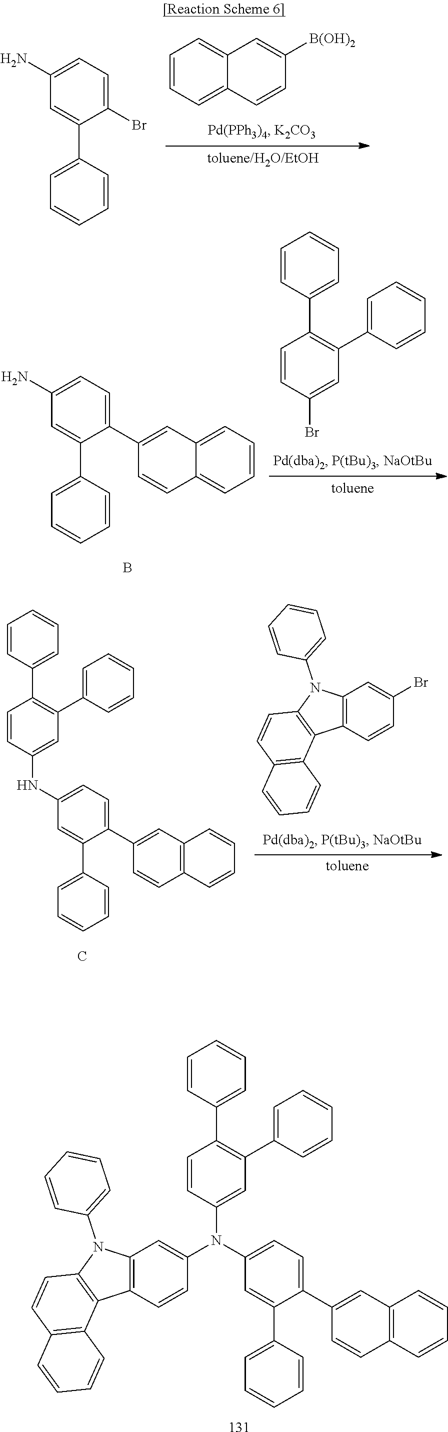

In an argon atmosphere, 6-bromo-[1,1′-biphenyl]-3-amine (30.0 g), Naphthalen-2-ylboronic acid (20.8 g), Tetrakis(triphenylphosphine) palladium (0) (Pd(PPh₃)₄, 6.9 g), and potassium carbonate (K₂CO₃, 33.4 g) were put into a 500 mL three-neck flask, dissolved in a mixed solvent of toluene, water, and ethanol (10:2:1, 200 mL), and the mixture was heated and stirred at 80° C. for 8 hours. Water was added, the resulting product was extracted with CH₂Cl₂, an organic layer was collected and dried over MgSO₄, and the solvent was removed by distillation under reduced pressure. The obtained crude product was purified by silica gel column chromatography to obtain Intermediate Compound B of 20.0 g (yield of 56%). A molecular weight of Intermediate Compound B was 295, as measured by FAB-MS measurement.

In the same manner as the synthesis of Intermediate Compound A described above, Intermediate Compound C of 18.0 g (yield of 68%) was obtained from Intermediate Compound B (15.0 g), and 4′-bromo-1,1′: 2′,1″-terphenyl (15.7 g). A molecular weight of Intermediate Compound C was 523, as measured by FAB-MS measurement.

In the same manner as the synthesis of Compound 8 described above, Compound 131 of 5.4 g (yield of 70%) was obtained from Intermediate Compound C(5.0 g), and 9-bromo-7-phenyl-7H-benzo[c]carbazole (3.6 g). A molecular weight of Compound 131 was 815, as measured by FAB-MS measurement.

### (7) Synthesis of Compound 135

Compound 135 according to an embodiment may be synthesized by, for example, Reaction Scheme 7:

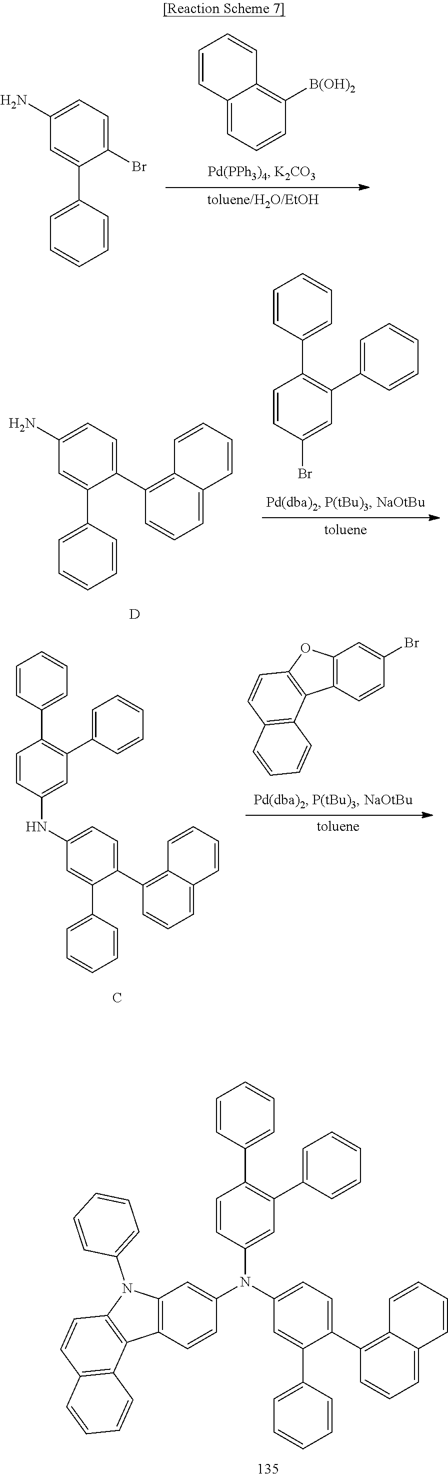

In the same manner as the synthesis of Intermediate Compound B described above, Intermediate Compound D of 14.0 g (yield of 59%) was obtained from 6-bromo-[1,1′-biphenyl]-3-amine (20.0 g) and naphthalene-1-ylboronic acid (13.8 g). A molecular weight of Intermediate Compound D was 295, as measured by FAB-MS measurement.

In the same manner as the synthesis of Intermediate Compound A described above, Intermediate Compound E of 11.8 g (yield of 67%) was obtained from Intermediate Compound D (10.0 g), and 4′-bromo-1,1′: 2′,1″-terphenyl (10.4 g). A molecular weight of Intermediate Compound E was 523, as measured by FAB-MS measurement.

In the same manner as the synthesis of Compound 8 described above, Compound 135 of 5.3 g (yield of 75%) was obtained from Intermediate Compound E (5.0 g), and 9-bromonaphtho[2,1-b]benzofuran (2.9 g). A molecular weight of Compound 135 was 739, as measured by FAB-MS measurement.

### (8) Synthesis of Compound 218

Compound 218 according to an embodiment may be synthesized by, for example, Reaction Scheme 8:

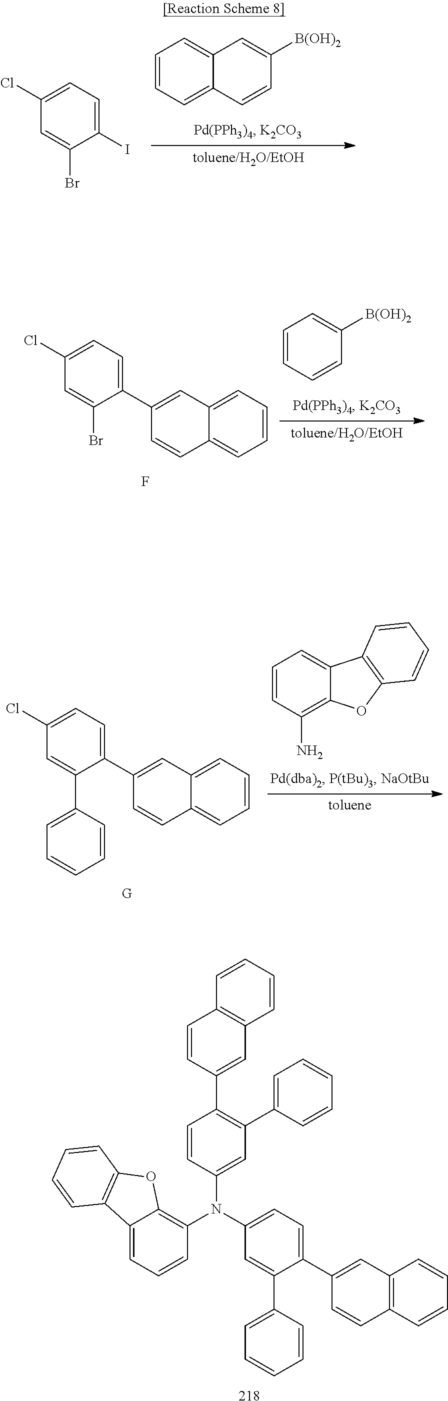

In the same manner as the synthesis of Intermediate Compound B described above, Intermediate Compound F of 16.8 g (yield of 84%) was obtained from 2-bromo-4-chloro-1-iodobenzene (20.0 g) and naphthalen-2-ylboronic acid (10.8 g). A molecular weight of Intermediate Compound F was 317, as measured by FAB-MS measurement.

In the same manner as the synthesis of Intermediate Compound B described above, Intermediate Compound G of 12.1 g (yield of 82%) was obtained from Intermediate Compound F (15.0 g), and phenylboronic acid (5.8 g). A molecular weight of Intermediate Compound G was 314, as measured by FAB-MS measurement.

In an argon atmosphere, Intermediate Compound G (7.0 g), dibenzo[b,d]furan-4-amine (2.0 g), Pd (dba) 2 (0.30 g), and NaOtBu (3.2 g) were put into a 300 mL three-neck flask and dissolved in toluene (100 mL), P (tBu) 3 (2.0 M in toluene, 0.5 mL) was added, and the mixture was heated and stirred at 100° C. for 4 hours. Water was added, the resulting product was extracted with CH₂Cl₂, an organic layer was collected and dried over MgSO₄, and the solvent was removed by distillation under reduced pressure. The obtained crude product was purified by silica gel column chromatography to obtain Compound 218 of 5.8 g (yield of 71%). A molecular weight of Compound 218 was 739, as measured by FAB-MS measurement.

### (9) Synthesis of Compound 235

Compound 235 according to an embodiment may be synthesized by, for example, Reaction Scheme 9:

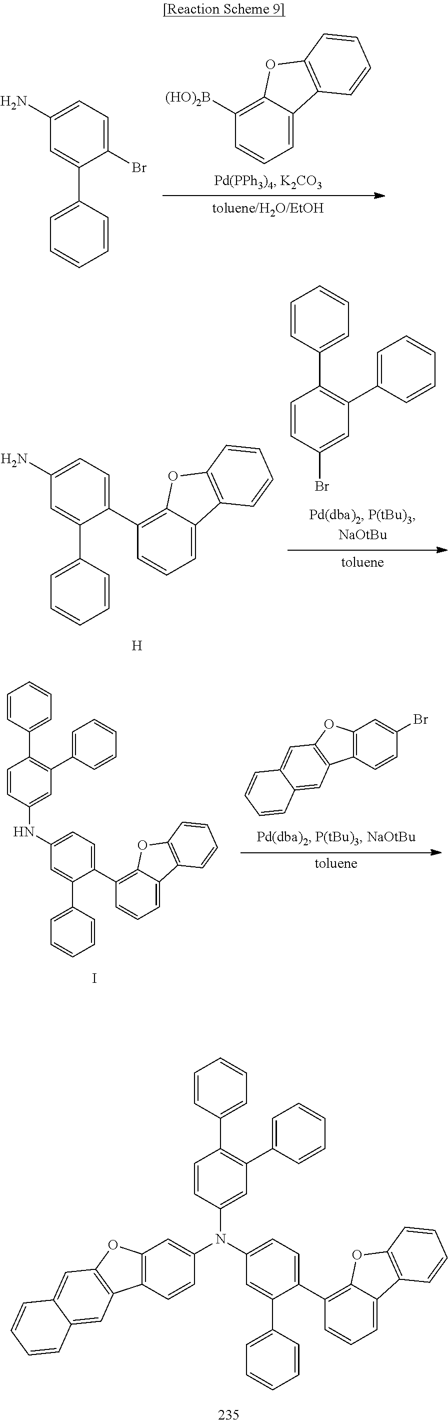

In the same manner as the synthesis of Intermediate Compound B described above, Intermediate Compound H of 7.4 g (yield of 55%) was obtained from 6-bromo-[1,1′-biphenyl]-3-amine (10.0 g) and dibenzo[b,d]furan-4-ylboronic acid (8.5 g). The molecular weight of Intermediate Compound H was 335, as measured by FAB-MS measurement.

In the same manner as the synthesis of Intermediate Compound A described above, Intermediate Compound I of 7.5 g (yield of 64%) was obtained from Intermediate Compound H(7.0 g) and 4′-bromo-1,1′: 2′,1″-terphenyl (6.4 g). A molecular weight of Intermediate Compound I was 563, as measured by FAB-MS measurement.

In the same manner as the synthesis of Compound 8 described above, Compound 235 of 5.5 g (yield of 80%) was obtained from Intermediate Compound I (5.0 g) and 3-bromonaphtho[2,3-b]benzofuran (2.7 g). The molecular weight of Compound 235 was 779, as measured by FAB-MS measurement.

### (10) Synthesis of Compound 239

Compound 239 according to an embodiment may be synthesized by, for example, Reaction Scheme 10:

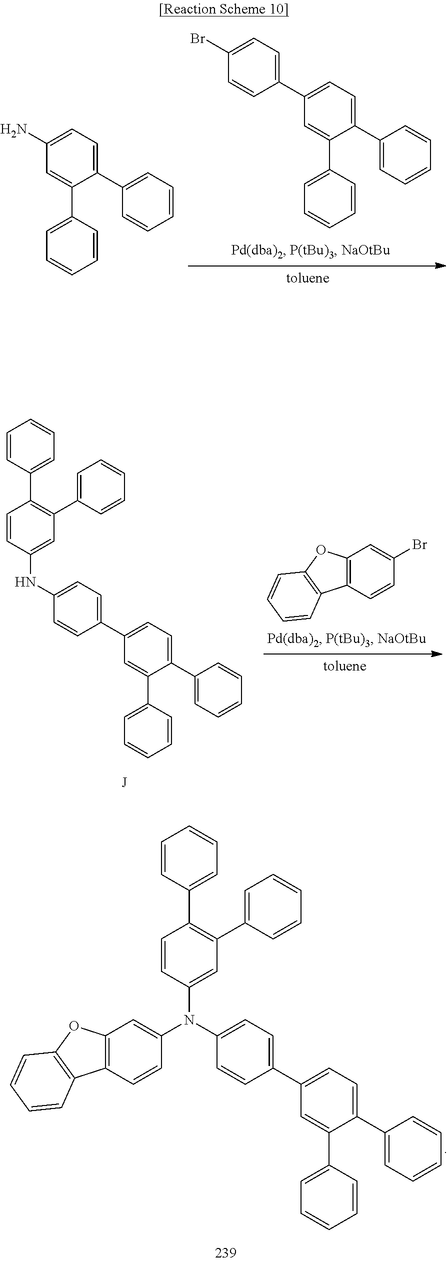 [SMILES: Nc1ccc(-c2ccccc2)c(-c2ccccc2)c1 ; Brc1ccc(-c2ccc(-c3ccccc3)c(-c3ccccc3)c2)cc1 ; c1ccc(-c2ccc(Nc3ccc(-c4ccc(-c5ccccc5)c(-c5ccccc5)c4)cc3)cc2-c2ccccc2)cc1 ; Brc1ccc2c(c1)oc1ccccc12 ; c1ccc(-c2ccc(-c3ccc(N(c4ccc(-c5ccccc5)c(-c5ccccc5)c4)c4ccc5c(c4)oc4ccccc45)cc3)cc2-c2ccccc2)cc1]

In the same manner as the synthesis of Intermediate Compound A described above, Intermediate Compound J of 13.0 g (yield of 58%) was obtained from [1,1′: 2′,1″-terphenyl]-4′-amine (10.0 g) and 4′-(4-bromophenyl)-1,1′: 2′,1″-terphenyl (15.7 g). A molecular weight of Intermediate Compound J was 549, as measured by FAB-MS measurement.

In the same manner as the synthesis of Compound 8 described above, Compound 239 of 3.7 g (yield of 58%) was obtained from Intermediate Compound J (5.0 g), and 3-bromodibenzo[b,d]furan (2.3 g). A molecular weight of Compound 239 was 715, as measured by FAB-MS measurement.

### (11) Synthesis of Compound 261

Compound 261 according to an embodiment may be synthesized by, for example, Reaction Scheme 11:

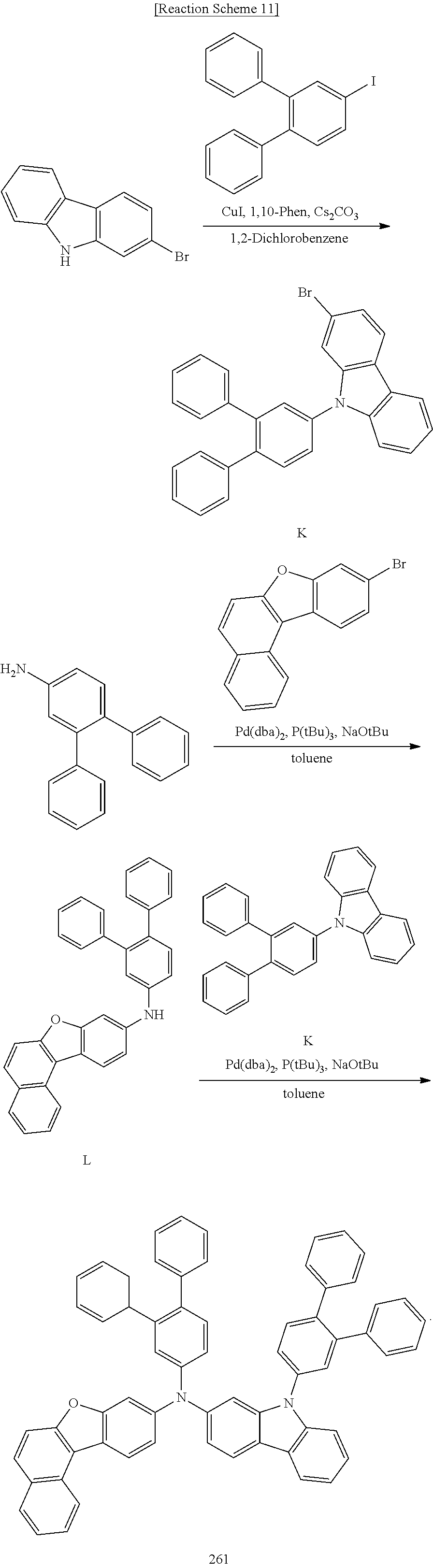

In an argon atmosphere, 2-bromocarbazole (10.0 g), 4′-iodo-1,1′: 2′,1″-terphenyl (14.5 g), copper (I) iodide (CuI, 0.8 g), 1,10-phenanthroline (1,10-Phen, 1.5 g), and cesium carbonate (Cs₂CO₃, 26.4 g) were put into a 1 L three-neck flask and dissolved in 400 mL of toluene, tri-tert-butylphosphine (P (tBu) 3 of 2.0 M was added, and was dissolved in 1,2-dichlorobenzene (200 mL), and the mixture was heated and stirred at 150° C. for 12 hours. Water was added, the resulting product was extracted with CH₂Cl₂, an organic layer was collected and dried over MgSO₄, and the solvent was removed by distillation under reduced pressure. The obtained crude product was purified by silica gel column chromatography and recrystallization to obtain Intermediate Compound K of 10.6 g (yield of 55%). A molecular weight of Intermediate Compound K was 474, as measured by FAB-MS measurement.

In the same manner as the synthesis of Intermediate Compound A described above, Intermediate Compound L of 14.6 g (yield of 78%) was obtained from [1,l′: 2′,1″-terphenyl]-4′-amine (10.0 g) and 9-bromonaphtho[2,1-b]benzofuran (12.1 g). A molecular weight of Intermediate Compound L was 461, as measured by FAB-MS measurement.

In the same manner as the synthesis of Compound 8 described above, Compound 261 of 6.1 g (yield of 66%) was obtained from Intermediate Compound L (5.0 g) and Intermediate Compound K (5.1 g). A molecular weight of Compound 261 was 855, as observed by FAB-MS measurement.

### 2. Manufacture and Evaluation of Light-Emitting Elements

### (1) Manufacture of Light-Emitting Elements

A light-emitting element according to an embodiment, including an amine compound according to an embodiment or a comparative example compound in a hole transport layer, was manufactured as follows. Light-emitting elements according to Example I to Example 11 were respectively manufactured using, as materials for a hole transport layer, Compounds 8, 11, 40, 44, 111, 131, 135, 218, 235, 239, and 261, which are amine compounds according to embodiments. Light-emitting elements according to Comparative Examples 1 to 13 were respectively manufactured using, as materials for a hole transport layer, Comparative Example Compounds X-1 to X-13.

A glass substrate, on which 150 nm of ITO was patterned as a first electrode, was cleansed with isopropyl alcohol and pure water, using ultrasonic waves for about 5 minutes each. After ultrasonic wave cleansing, the glass substrate was irradiated by UV for about 30 minutes and treated with ozone. 2-TNATA was deposited at a thickness of about 60 nm to form a hole injection layer. An Example Compound or a Comparative Example Compound was deposited at a thickness of about 30 nm to form a hole transport layer.

On the hole transport layer, TBP and ADN were co-deposited at a thickness of about 25 nm to form an emission layer. TBP and ADN were co-deposited at a weight ratio of 3:97. Alq₃ at a thickness of about 25 nm, and LiF at a thickness of about 1 nm were sequentially deposited to form an electron transport region. Al was deposited at a thickness of about 100 nm to form a second electrode. In the manufacture of the light-emitting element, the hole injection layer, the hole transport layer, the emission layer, the electron transport region, and the second electrode were each formed using vacuum deposition equipment.

The compounds used in the manufacture of the light-emitting elements are as follows:

### <Materials Used in Manufacture of Light-Emitting Elements>

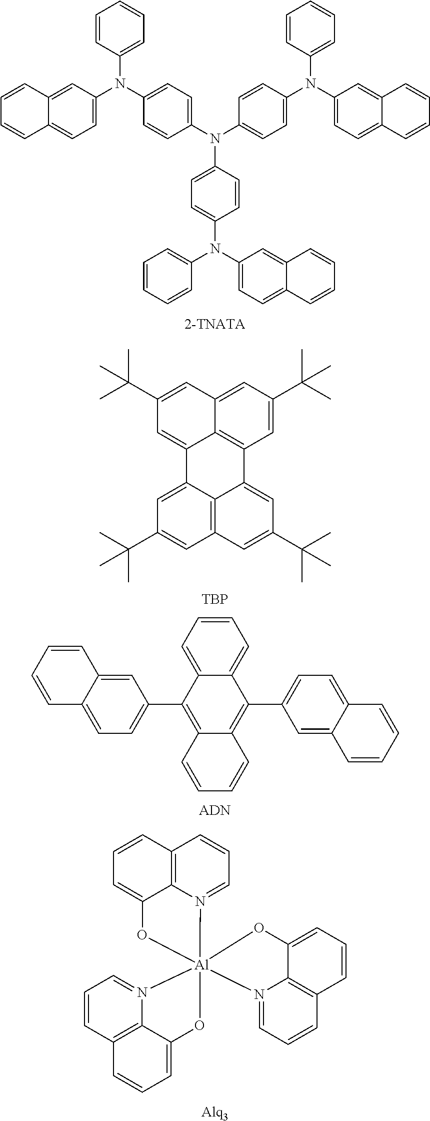

### <Example Compounds>

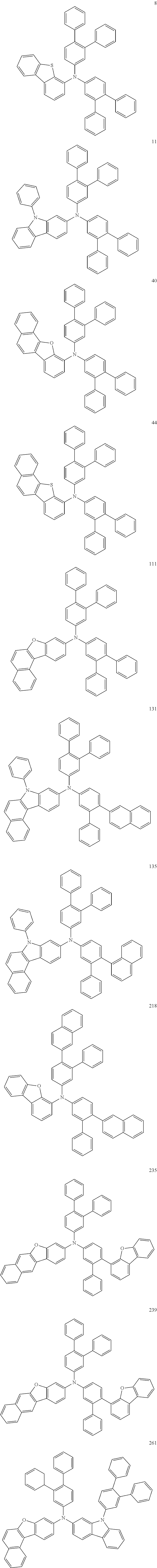

### <Comparative Example Compounds>

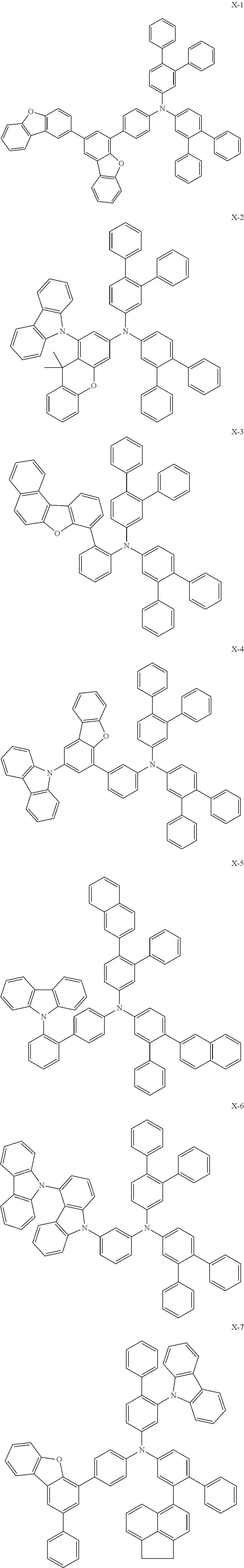

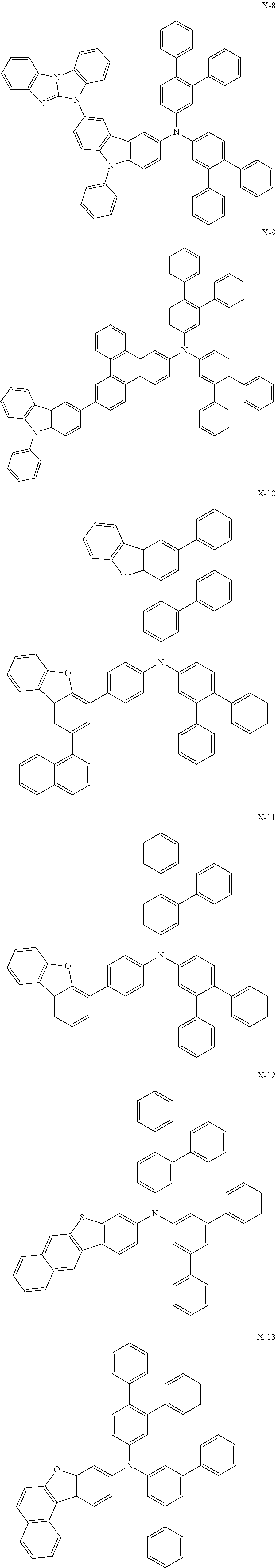

### (2) Evaluation of Light-Emitting Elements

The light-emitting elements according to the Examples and the Comparative Examples were evaluated and the results are listed in Table 1. Luminous efficiency and element lifespan of the light-emitting elements according to the Examples and the Comparative Examples were listed in Table 1. The evaluations of luminous efficiency and element lifespan were performed using luminance orientation characteristic measurement equipment C9920-11 made by Hamamatsu Photonics K.K.

The luminous efficiency of the light-emitting element was relatively expressed with respect to the luminous efficiency of the light-emitting element according to Comparative Example 1 at 10 mA/cm². The time taken for luminance to decrease to 95% of an initial luminance was measured for an element lifespan of the light-emitting elements, and relative element lifespans were expressed in Table 1 with respect to the element lifespan of the light-emitting element according to Comparative Example 1.

TABLE 1

| Element | | Luminous | Element |
|---|---|---|---|
| manufacture | Hole transport layer | Efficiency | Lifespan |
| examples | material | (%) | (LT50) (%) |
| Example 1 | Compound 8 | 105% | 150% |
| Example 2 | Compound 11 | 102% | 140% |
| Example 3 | Compound 40 | 104% | 130% |
| Example 4 | Compound 44 | 105% | 130% |
| Example 5 | Compound 111 | 103% | 150% |
| Example 6 | Compound 131 | 102% | 160% |
| Example 7 | Compound 135 | 104% | 140% |
| Example 8 | Compound 218 | 103% | 150% |
| Example 9 | Compound 235 | 103% | 130% |
| Example 10 | Compound 239 | 102% | 150% |
| Example 11 | Compound 261 | 102% | 140% |
| Comparative | Comparative Example | 100% | 100% |
| Example 1 | Compound X-1 | | |
| Comparative | Comparative Example | 100% | 65% |
| Example 2 | Compound X-2 | | |
| Comparative | Comparative Example | 101% | 70% |
| Example 3 | Compound X-3 | | |
| Comparative | Comparative Example | 101% | 85% |
| Example 4 | Compound X-4 | | |
| Comparative | Comparative Example | 95% | 90% |
| Example 5 | Compound X-5 | | |
| Comparative | Comparative Example | 98% | 100% |
| Example 6 | Compound X-6 | | |
| Comparative | Comparative Example | 95% | 50% |
| Example 7 | Compound X-7 | | |
| Comparative | Comparative Example | 90% | 50% |
| Example 8 | Compound X-8 | | |
| Comparative | Comparative Example | 100% | 75% |
| Example 9 | Compound X-9 | | |
| Comparative | Comparative Example | 101% | 60% |
| Example 10 | Compound X-10 | | |
| Comparative | Comparative Example | 97% | 100% |
| Example 11 | Compound X-11 | | |
| Comparative | Comparative Example | 99% | 80% |
| Example 12 | Compound X-12 | | |
| Comparative | Comparative Example | 100% | 80% |
| Example 13 | Compound X-13 | | |

Referring to Table 1, the light-emitting elements according to Example 1 to Example 11 exhibit characteristics of higher efficiency and longer lifespan than the light-emitting elements according to Comparative Example 1 to Comparative Example 13.

As compared to the Example Compounds, Comparative Example Compounds X-1, X-4, and X-6 each include two 3,4-substituted phenyl groups (ortho-terphenyl derivatives), but a third substituent of a dibenzoheterole group is substituted with a heteroaryl group, and the third substituent is connected to a nitrogen atom of an amine group via a phenylene group as a linking moiety. As compared to the light-emitting elements according to Comparative Examples 1, 4, and 6 respectively containing Comparative Example Compounds X-1, X-4, and X-6, the light-emitting elements according to the Examples containing the amine Example Compounds exhibited characteristics of high luminous efficiency and long lifespan.

As compared to the Example Compounds, Comparative Example Compound X-2 includes two 3,4-substituted phenyl groups but does not include a dibenzoheterole group or a benzonaphtoheterole group. As compared to the light-emitting element according to Comparative Example 2 containing Comparative Example Compound X-2, the light-emitting elements according the Examples containing the amine Example Compounds exhibited characteristics of high luminous efficiency and long lifespan.

As compared to the Example Compounds, Comparative Example Compounds X-3, X-5, X-7, X-9, X-10, and X-11 each include two 3,4-substituted phenyl groups, but the third substituent is linked to a nitrogen atom of an amine group via a phenylene group as a linking moiety. As compared to the light-emitting elements according to Comparative Examples 3, 5, 7, 9, 10, and 11 respectively containing Comparative Example Compounds X-3, X-5, X-7, X-9, X-10, and X-11, the light-emitting elements according to the Examples containing the amine Example Compounds exhibited characteristics of high luminous efficiency and long lifespan.

As compared to the Example Compounds, Comparative Example Compound X-8 includes two 3,4-substituted phenyl groups, but the third substituent of the dibenzoheterole group is substituted with a heteroaryl group. As compared to the light-emitting element according to Comparative Example 8 containing Comparative Example Compound X-8, the light-emitting elements according to the Examples containing the amine Example Compounds exhibited characteristics of high luminous efficiency and long lifespan.

As compared to the Example Compounds, Comparative Example Compounds X-12 and X-13 each include one 3,4-substituted phenyl group (an ortho-terphenyl derivative). As compared to the light-emitting elements according to Comparative Examples 12 and 13 respectively containing Comparative Example Compounds X-12 and X-13, the light-emitting elements according to the Examples containing the amine Example Compounds exhibited characteristics of high luminous efficiency and long lifespan.

Therefore, it can be confirmed that the Example Compounds have excellent charge transportability and contribute to charge balance due to a structural characteristic thereof, which is distinguished from the structural characteristics of the Comparative Example Compounds, and thus the light-emitting elements according to the Examples each including an amine Example Compound in a hole transport layer may exhibit characteristics of high efficiency and long lifespan.

The amine compound according to an embodiment has a structure that includes a dibenzoheterole derivative and two ortho-terphenyl derivatives, wherein the dibenzoheterole derivative is directly bonded to a nitrogen atom of an amine group, and the ortho-terphenyl derivatives are each bonded to the nitrogen atom at a specific position via a direct linkage or a linking moiety. Therefore, the amine compound according to an embodiment may exhibit excellent material stability and high charge transportability. The light-emitting element according to an embodiment containing the amine compound according to an embodiment in a hole transport region may exhibit characteristics of excellent luminous efficiency and long lifespan. A light-emitting element according to an embodiment, may emit blue light and contains the amine compound according to an embodiment in the hole transport region, and thus may exhibit characteristics of high efficiency and long lifespan.

The light-emitting element according to an embodiment includes the amine compound according to an embodiment in the hole transport region and thus may exhibit characteristics of high efficiency and long lifespan.

The amine compound according to an embodiment may be used as a material for achieving improved characteristics in high efficiency and long lifespan of the light-emitting element.

The display device according to an embodiment may exhibit excellent display quality.

Embodiments have been disclosed herein, and although terms are employed, they are used and are to be interpreted in a generic and descriptive sense only and not for the purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light-emitting element comprising:
a first electrode;
a second electrode disposed on the first electrode;
an emission layer disposed between the first electrode and the second electrode; and
a hole transport region disposed between the first electrode and the emission layer, wherein
the hole transport region includes an amine compound represented by Formula 1: 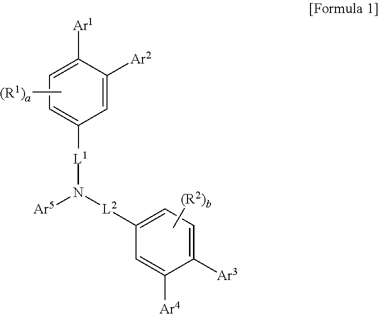
wherein in Formula 1,
L¹ and L² are each independently a direct linkage, a substituted or unsubstituted arylene group having 6 to 30 ring-forming carbon atoms, or a substituted or unsubstituted heteroarylene group having 5 to 30 ring-forming carbon atoms,
a and b are each independently an integer from 0 to 3,
R¹ and R² are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or bonded to an adjacent group to form a ring,
Ar¹ to Art are each independently a substituted or unsubstituted aryl group having 6 to 30 ring-forming carbon atoms or a substituted or unsubstituted heteroaryl group having 5 to 30 ring-forming carbon atoms, and
Ar⁵ is a group represented by one of Formula 2-1 to Formula 2-4: 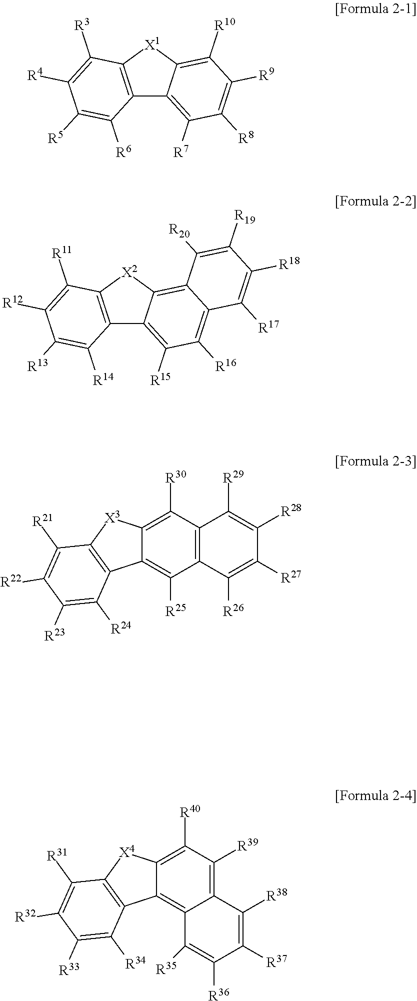
wherein in Formula 2-1 to Formula 2-4,
X¹ to X⁴ are each independently O, S, or N(R⁴¹),
R³ to R¹⁰ are each independently a hydrogen atom, a deuterium atom, or a substituted or unsubstituted aryl group having 6 to 30 ring-forming carbon atoms, and
R¹¹ to R⁴¹ are each independently a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms, except that:
in Formula 2-1, one of R³ to R¹⁰ and R⁴¹ is a position connected to Formula 1,
in Formula 2-2, one of R¹¹ to R²⁰ and R⁴¹ is a position connected to Formula 1,
in Formula 2-3, one of R²¹ to R³⁰ and R⁴¹ is a position connected to Formula 1, and
in Formula 2-4, one of R³¹ to R⁴¹ is a position connected to Formula 1.

2. The light-emitting element of claim 1, wherein
the hole transport region comprises at least one of a hole injection layer, a hole transport layer, an electron blocking layer, and an emission auxiliary layer, and
the amine compound is included in at least one of the hole injection layer, the hole transport layer, the electron blocking layer, and the emission auxiliary layer.

3. The light-emitting element of claim 1, wherein
the hole transport region comprises:
a hole injection layer disposed on the first electrode; and
a hole transport layer disposed on the hole injection layer, and
the hole transport layer includes the amine compound.

4. The light-emitting element of claim 1, wherein the amine compound is represented by Formula 3-1 or Formula 3-2: 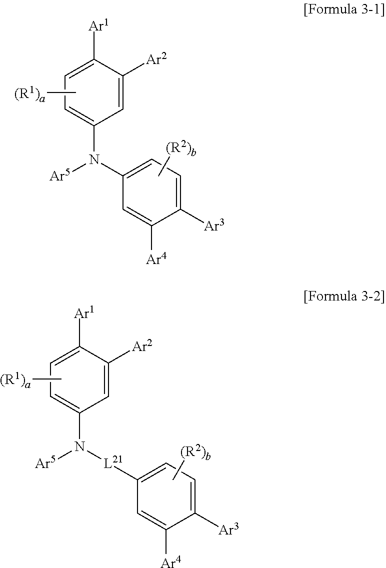
wherein in Formula 3-2,
L²¹ is a substituted or unsubstituted phenylene group, a substituted or unsubstituted biphenylene group, a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group, and
wherein in Formula 3-1 and Formula 3-2,
Ar¹ to Ar⁵, R¹, R², a, and b are the same as defined in Formula 1.

5. The light-emitting element of claim 1, wherein the amine compound is represented by Formula 4: 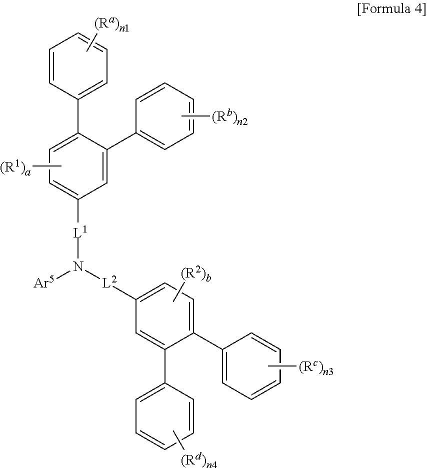
wherein in Formula 4,
n1 to n4 are each independently an integer from 0 to 5,
R^{a} to R^{d} are each independently a hydrogen atom, a deuterium atom, a halogen atom, a substituted or unsubstituted alkyl group having 1 to 15 ring-forming carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring, and
Ar⁵, R¹, R², L′, L², a, and b are the same as defined in Formula 1.

6. The light-emitting element of claim 1, wherein Ar¹ to Ar⁴ are each independently a substituted or unsubstituted phenyl group, a substituted or unsubstituted naphthyl group, a substituted or unsubstituted phenanthryl group, a substituted or unsubstituted pyrenyl group, a substituted or unsubstituted triphenylene group, a substituted or unsubstituted carbazole group, a substituted or unsubstituted dibenzothiophene group, or a substituted or unsubstituted dibenzofuran group.

7. The light-emitting element of claim 1, wherein at least one hydrogen atom of the amine compound is substituted with a deuterium atom.

8. The light-emitting element of claim 1, wherein when Ar⁵ is a group represented by Formula 2-1, Ar¹ to Ar are each independently a substituted or unsubstituted aryl group having 6 to 30 ring-forming carbon atoms.

9. The light-emitting element of claim 1, wherein the amine compound is selected from Compound Group 1: 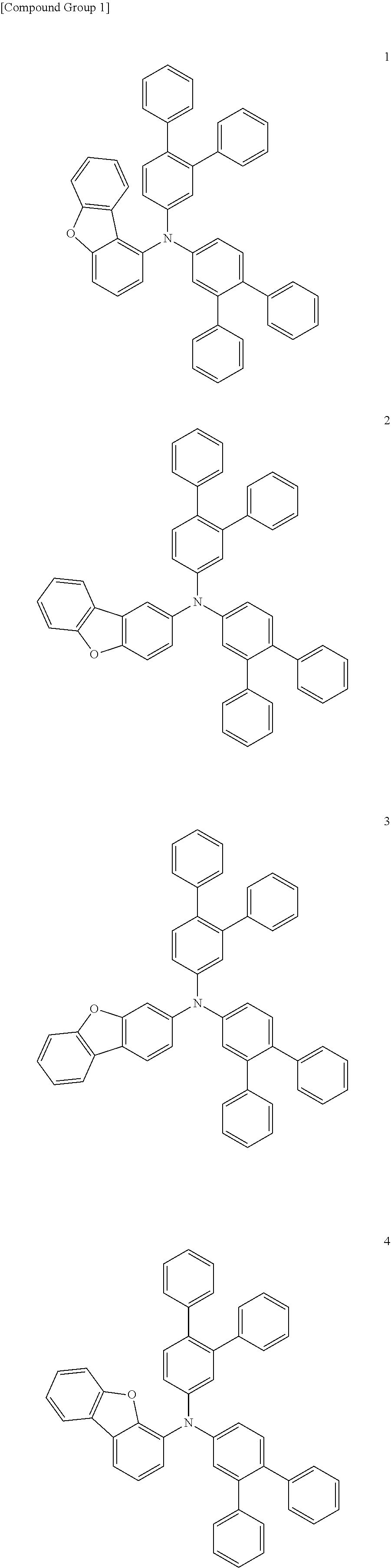 [SMILES: c1ccc(-c2ccc(N(c3ccc(-c4ccccc4)c(-c4ccccc4)c3)c3cccc4oc5ccccc5c34)cc2-c2ccccc2)cc1 ; c1ccc(-c2ccc(N(c3ccc(-c4ccccc4)c(-c4ccccc4)c3)c3ccc4oc5ccccc5c4c3)cc2-c2ccccc2)cc1 ; c1ccc(-c2ccc(N(c3ccc(-c4ccccc4)c(-c4ccccc4)c3)c3ccc4c(c3)oc3ccccc34)cc2-c2ccccc2)cc1 ; c1ccc(-c2ccc(N(c3ccc(-c4ccccc4)c(-c4ccccc4)c3)c3cccc4c3oc3ccccc34)cc2-c2ccccc2)cc1] 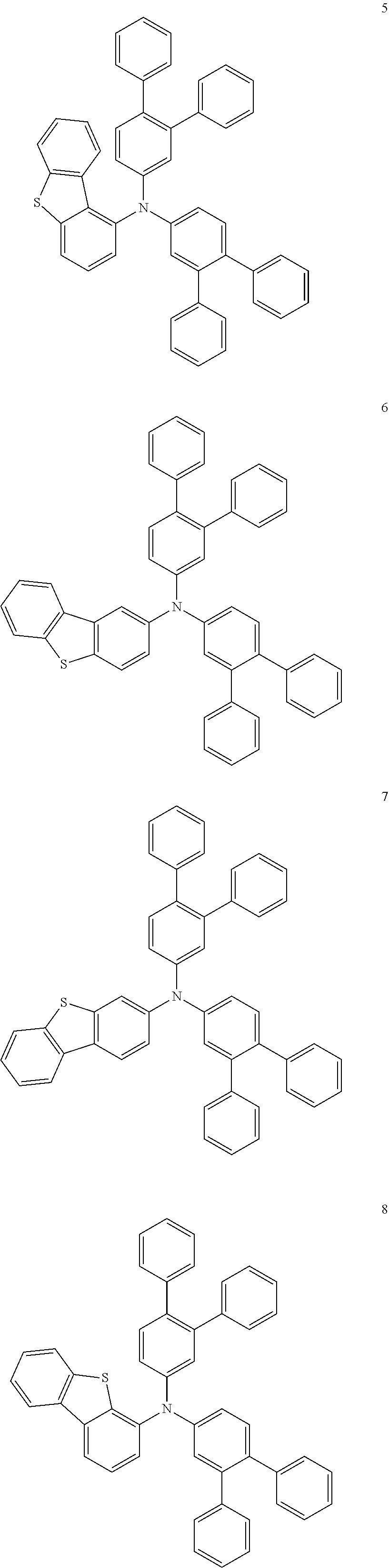 [SMILES: c1ccc(-c2ccc(N(c3ccc(-c4ccccc4)c(-c4ccccc4)c3)c3cccc4sc5ccccc5c34)cc2-c2ccccc2)cc1 ; c1ccc(-c2ccc(N(c3ccc(-c4ccccc4)c(-c4ccccc4)c3)c3ccc4sc5ccccc5c4c3)cc2-c2ccccc2)cc1 ; c1ccc(-c2ccc(N(c3ccc(-c4ccccc4)c(-c4ccccc4)c3)c3ccc4c(c3)sc3ccccc34)cc2-c2ccccc2)cc1 ; c1ccc(-c2ccc(N(c3ccc(-c4ccccc4)c(-c4ccccc4)c3)c3cccc4c3sc3ccccc34)cc2-c2ccccc2)cc1] 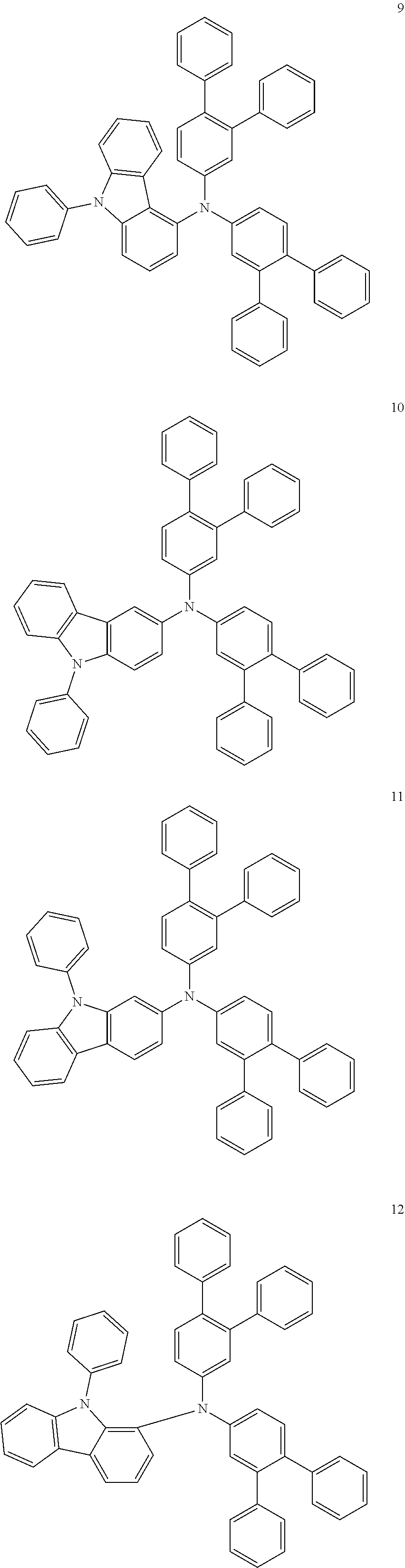 [SMILES: c1ccc(-c2ccc(N(c3ccc(-c4ccccc4)c(-c4ccccc4)c3)c3cccc4c3c3ccccc3n4-c3ccccc3)cc2-c2ccccc2)cc1 ; c1ccc(-c2ccc(N(c3ccc(-c4ccccc4)c(-c4ccccc4)c3)c3ccc4c(c3)c3ccccc3n4-c3ccccc3)cc2-c2ccccc2)cc1 ; c1ccc(-c2ccc(N(c3ccc(-c4ccccc4)c(-c4ccccc4)c3)c3ccc4c5ccccc5n(-c5ccccc5)c4c3)cc2-c2ccccc2)cc1 ; c1ccc(-c2ccc(N(c3ccc(-c4ccccc4)c(-c4ccccc4)c3)c3cccc4c5ccccc5n(-c5ccccc5)c34)cc2-c2ccccc2)cc1] 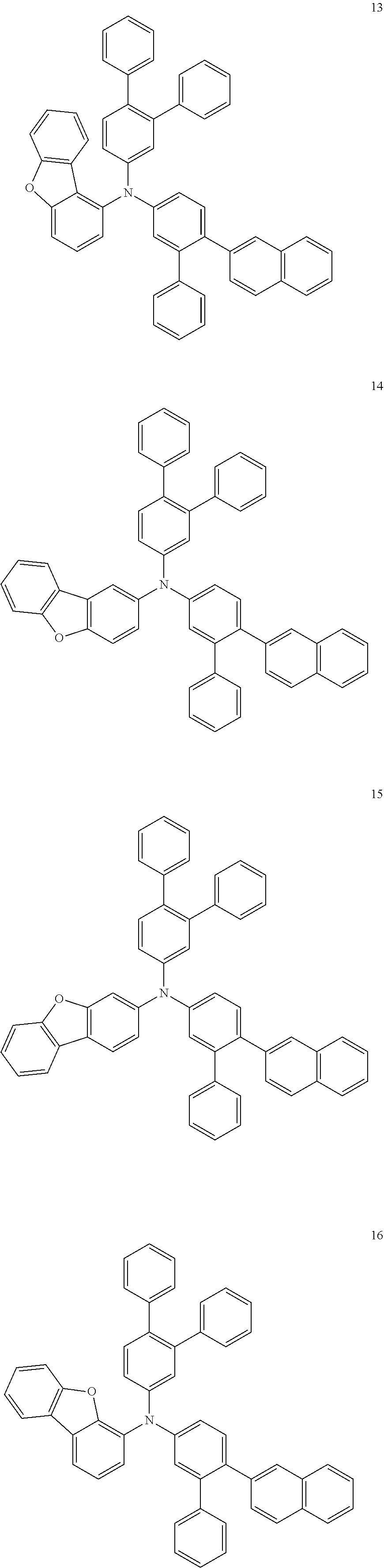 [SMILES: c1ccc(-c2ccc(N(c3ccc(-c4ccc5ccccc5c4)c(-c4ccccc4)c3)c3cccc4oc5ccccc5c34)cc2-c2ccccc2)cc1 ; c1ccc(-c2ccc(N(c3ccc(-c4ccc5ccccc5c4)c(-c4ccccc4)c3)c3ccc4oc5ccccc5c4c3)cc2-c2ccccc2)cc1 ; c1ccc(-c2ccc(N(c3ccc(-c4ccc5ccccc5c4)c(-c4ccccc4)c3)c3ccc4c(c3)oc3ccccc34)cc2-c2ccccc2)cc1 ; c1ccc(-c2ccc(N(c3ccc(-c4ccc5ccccc5c4)c(-c4ccccc4)c3)c3cccc4c3oc3ccccc34)cc2-c2ccccc2)cc1] 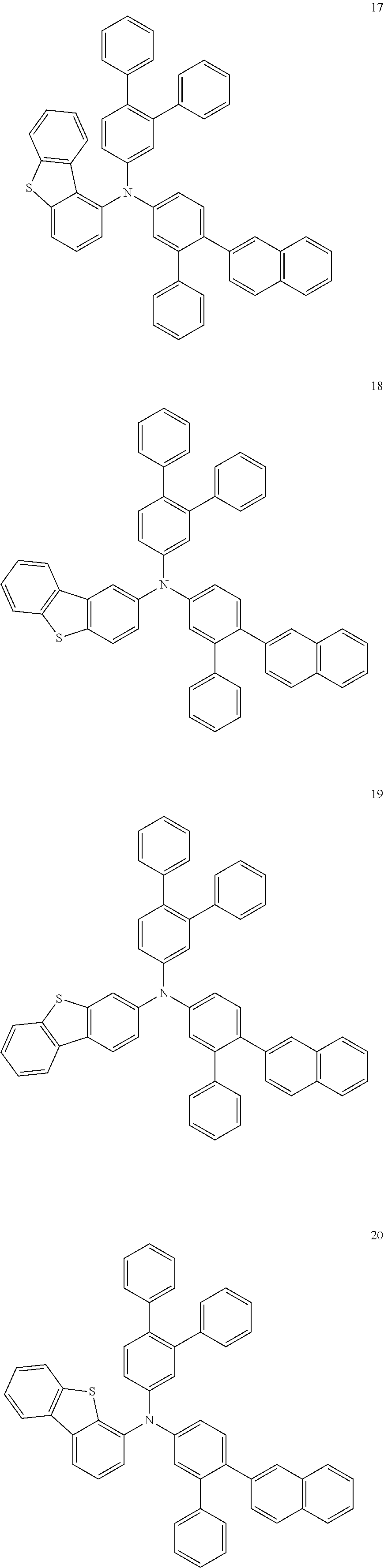 [SMILES: c1ccc(-c2ccc(N(c3ccc(-c4ccc5ccccc5c4)c(-c4ccccc4)c3)c3cccc4sc5ccccc5c34)cc2-c2ccccc2)cc1 ; c1ccc(-c2ccc(N(c3ccc(-c4ccc5ccccc5c4)c(-c4ccccc4)c3)c3ccc4sc5ccccc5c4c3)cc2-c2ccccc2)cc1 ; c1ccc(-c2ccc(N(c3ccc(-c4ccc5ccccc5c4)c(-c4ccccc4)c3)c3ccc4c(c3)sc3ccccc34)cc2-c2ccccc2)cc1 ; c1ccc(-c2ccc(N(c3ccc(-c4ccc5ccccc5c4)c(-c4ccccc4)c3)c3cccc4c3sc3ccccc34)cc2-c2ccccc2)cc1] 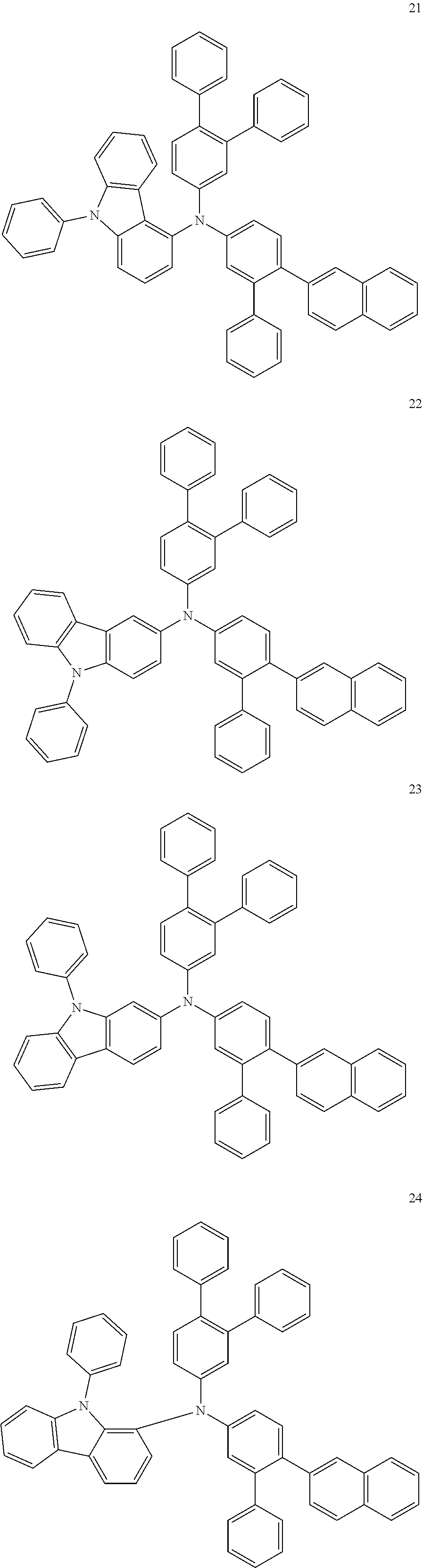 [SMILES: c1ccc(-c2ccc(N(c3ccc(-c4ccc5ccccc5c4)c(-c4ccccc4)c3)c3cccc4c3c3ccccc3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3ccccc3n4-c3ccccc3)cc2-c2ccccc2)cc1 ; c1ccc(-c2ccc(N(c3ccc(-c4ccc5ccccc5c4)c(-c4ccccc4)c3)c3ccc4c5ccccc5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5ccccc5n(-c5ccccc5)c34)cc2-c2ccccc2)cc1] 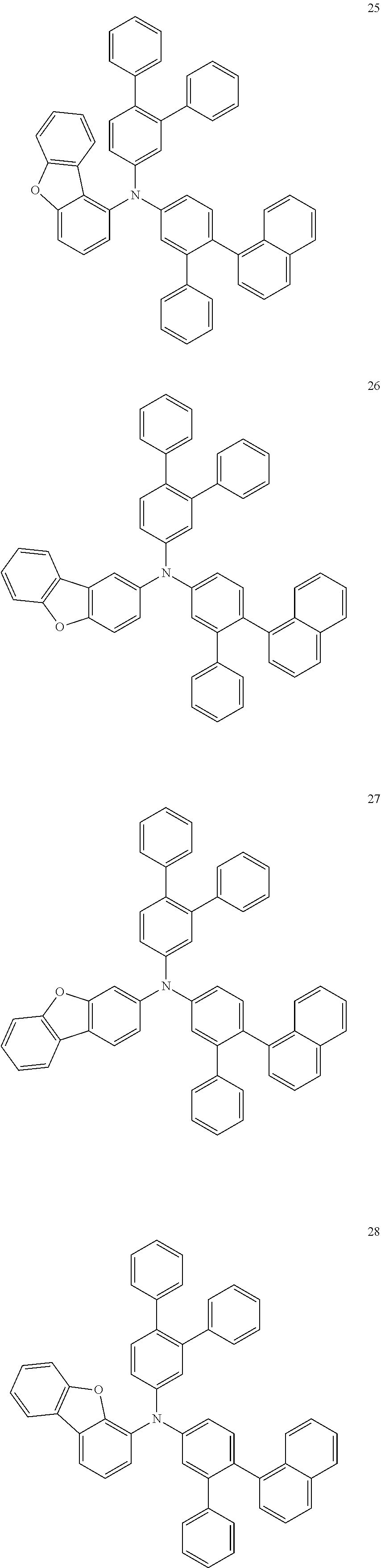 [SMILES: c1ccc(-c2ccc(N(c3ccc(-c4cccc5ccccc45)c(-c4ccccc4)c3)c3cccc4oc5ccccc5c34)cc2-c2ccccc2)cc1 ; c1ccc(-c2ccc(N(c3ccc(-c4cccc5ccccc45)c(-c4ccccc4)c3)c3ccc4oc5ccccc5c4c3)cc2-c2ccccc2)cc1 ; c1ccc(-c2ccc(N(c3ccc(-c4cccc5ccccc45)c(-c4ccccc4)c3)c3ccc4c(c3)oc3ccccc34)cc2-c2ccccc2)cc1 ; c1ccc(-c2ccc(N(c3ccc(-c4cccc5ccccc45)c(-c4ccccc4)c3)c3cccc4c3oc3ccccc34)cc2-c2ccccc2)cc1] 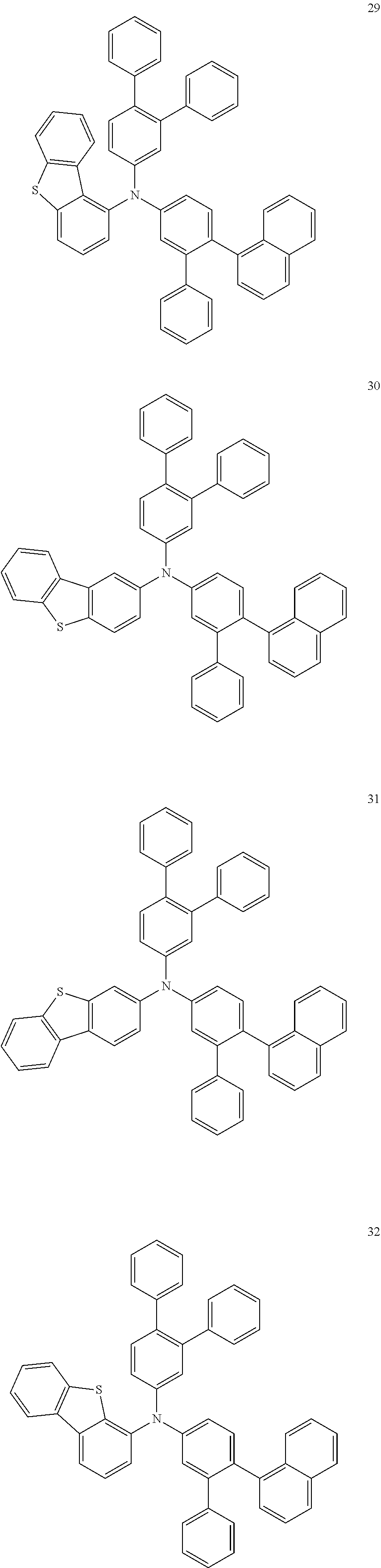 [SMILES: c1ccc(-c2ccc(N(c3ccc(-c4cccc5ccccc45)c(-c4ccccc4)c3)c3cccc4sc5ccccc5c34)cc2-c2ccccc2)cc1 ; c1ccc(-c2ccc(N(c3ccc(-c4cccc5ccccc45)c(-c4ccccc4)c3)c3ccc4sc5ccccc5c4c3)cc2-c2ccccc2)cc1 ; c1ccc(-c2ccc(N(c3ccc(-c4cccc5ccccc45)c(-c4ccccc4)c3)c3ccc4c(c3)sc3ccccc34)cc2-c2ccccc2)cc1 ; c1ccc(-c2ccc(N(c3ccc(-c4cccc5ccccc45)c(-c4ccccc4)c3)c3cccc4c3sc3ccccc34)cc2-c2ccccc2)cc1] 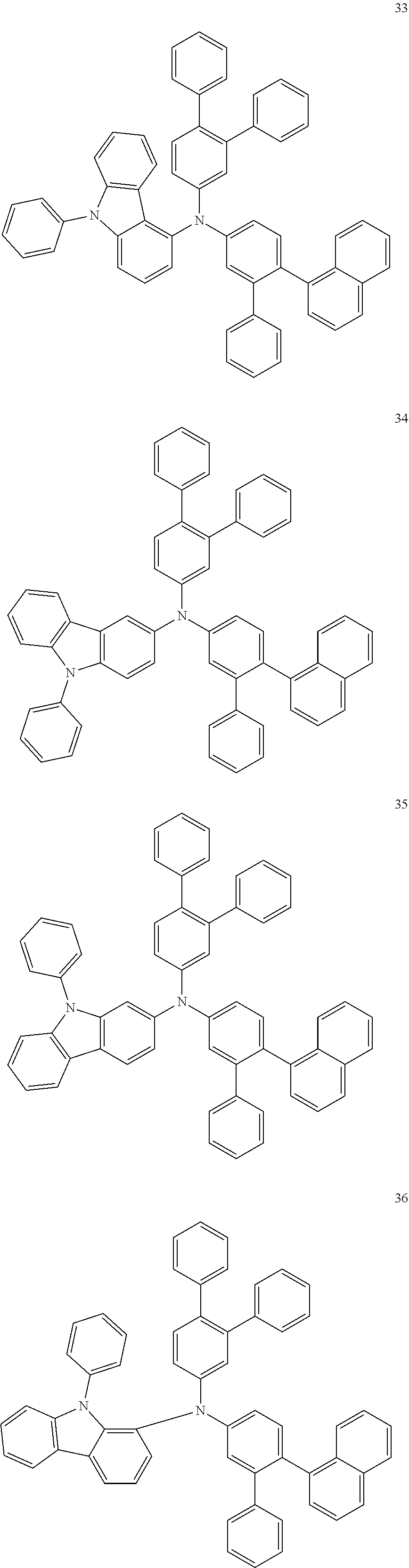 [SMILES: c1ccc(-c2ccc(N(c3ccc(-c4cccc5ccccc45)c(-c4ccccc4)c3)c3cccc4c3c3ccccc3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3ccccc3n4-c3ccccc3)cc2-c2ccccc2)cc1 ; c1ccc(-c2ccc(N(c3ccc(-c4cccc5ccccc45)c(-c4ccccc4)c3)c3ccc4c5ccccc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5ccccc5n(-c5ccccc5)c34)cc2-c2ccccc2)cc1] 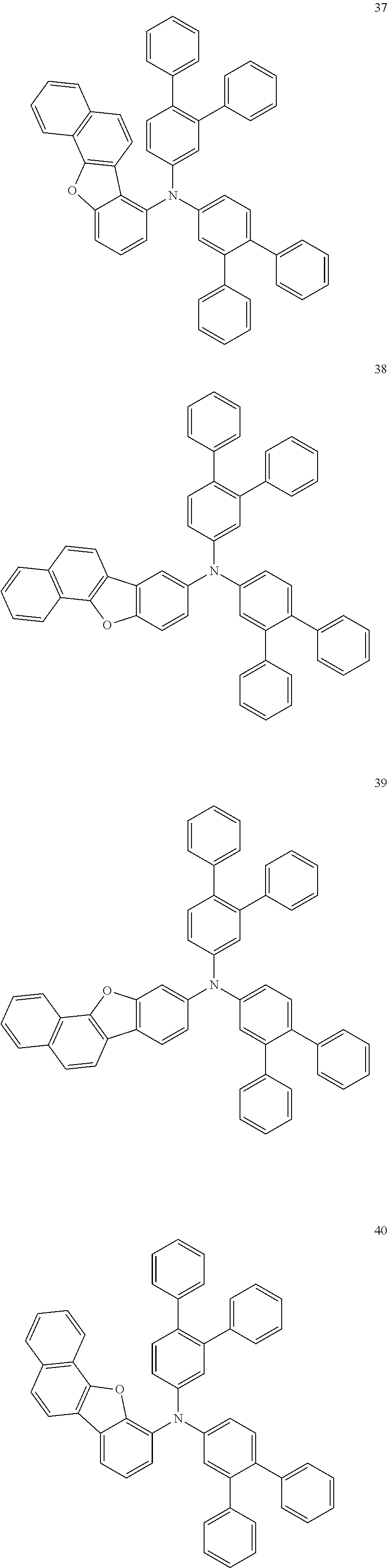 [SMILES: c1ccc(-c2ccc(N(c3ccc(-c4ccccc4)c(-c4ccccc4)c3)c3cccc4oc5c6ccccc6ccc5c34)cc2-c2ccccc2)cc1 ; c1ccc(-c2ccc(N(c3ccc(-c4ccccc4)c(-c4ccccc4)c3)c3ccc4oc5c6ccccc6ccc5c4c3)cc2-c2ccccc2)cc1 ; c1ccc(-c2ccc(N(c3ccc(-c4ccccc4)c(-c4ccccc4)c3)c3ccc4c(c3)oc3c5ccccc5ccc43)cc2-c2ccccc2)cc1 ; c1ccc(-c2ccc(N(c3ccc(-c4ccccc4)c(-c4ccccc4)c3)c3cccc4c3oc3c5ccccc5ccc43)cc2-c2ccccc2)cc1] 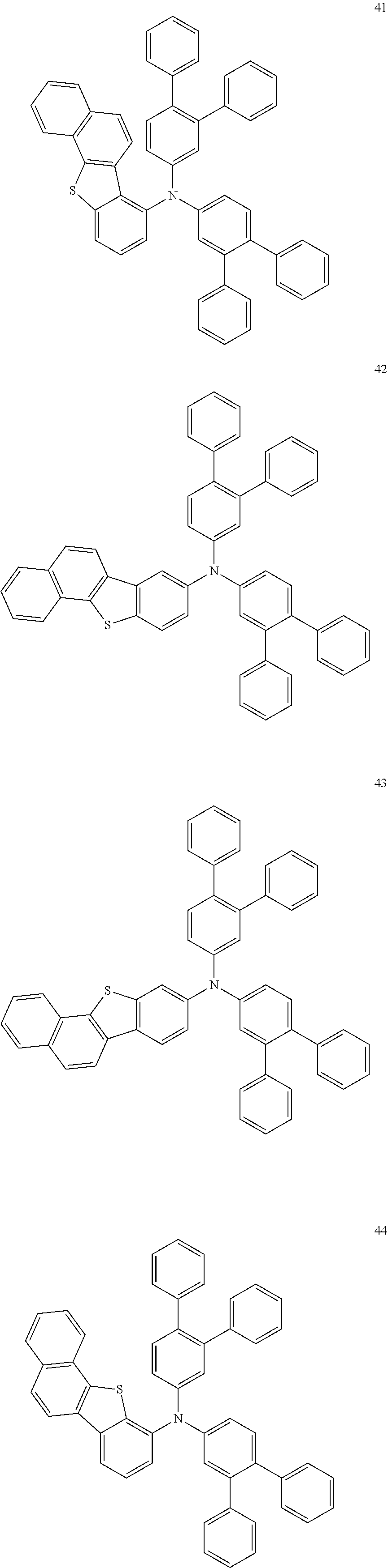 [SMILES: c1ccc(-c2ccc(N(c3ccc(-c4ccccc4)c(-c4ccccc4)c3)c3cccc4sc5c6ccccc6ccc5c34)cc2-c2ccccc2)cc1 ; c1ccc(-c2ccc(N(c3ccc(-c4ccccc4)c(-c4ccccc4)c3)c3ccc4sc5c6ccccc6ccc5c4c3)cc2-c2ccccc2)cc1 ; c1ccc(-c2ccc(N(c3ccc(-c4ccccc4)c(-c4ccccc4)c3)c3ccc4c(c3)sc3c5ccccc5ccc43)cc2-c2ccccc2)cc1 ; c1ccc(-c2ccc(N(c3ccc(-c4ccccc4)c(-c4ccccc4)c3)c3cccc4c3sc3c5ccccc5ccc43)cc2-c2ccccc2)cc1] 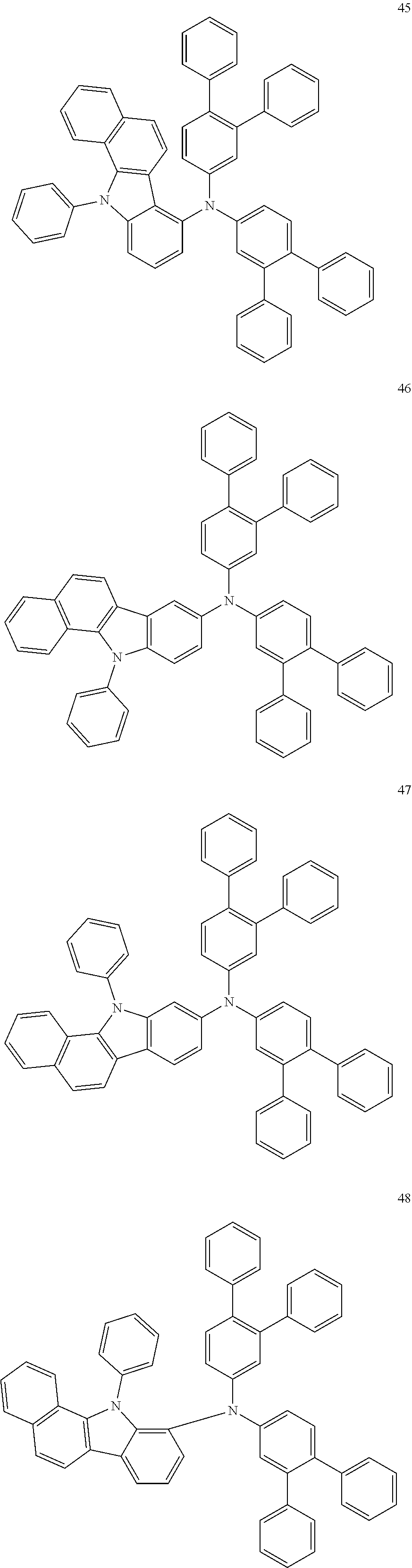 [SMILES: c1ccc(-c2ccc(N(c3ccc(-c4ccccc4)c(-c4ccccc4)c3)c3cccc4c3c3ccc5ccccc5c3n4-c3ccccc3)cc2-c2ccccc2)cc1 ; c1ccc(-c2ccc(N(c3ccc(-c4ccccc4)c(-c4ccccc4)c3)c3ccc4c(c3)c3ccc5ccccc5c3n4-c3ccccc3)cc2-c2ccccc2)cc1 ; c1ccc(-c2ccc(N(c3ccc(-c4ccccc4)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4ccccc4)c(-c4ccccc4)c3)c3cccc4c5ccc6ccccc6c5n(-c5ccccc5)c34)cc2-c2ccccc2)cc1] 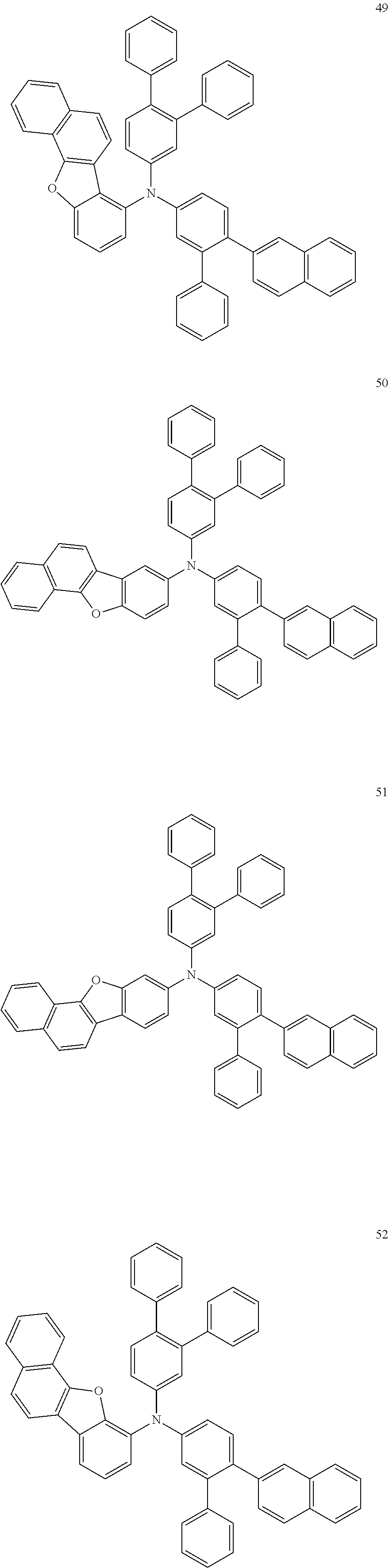 [SMILES: c1ccc(-c2ccc(N(c3ccc(-c4ccc5ccccc5c4)c(-c4ccccc4)c3)c3cccc4oc5c6ccccc6ccc5c34)cc2-c2ccccc2)cc1 ; c1ccc(-c2ccc(N(c3ccc(-c4ccc5ccccc5c4)c(-c4ccccc4)c3)c3ccc4oc5c6ccccc6ccc5c4c3)cc2-c2ccccc2)cc1 ; c1ccc(-c2ccc(N(c3ccc(-c4ccc5ccccc5c4)c(-c4ccccc4)c3)c3ccc4c(c3)oc3c5ccccc5ccc43)cc2-c2ccccc2)cc1 ; c1ccc(-c2ccc(N(c3ccc(-c4ccc5ccccc5c4)c(-c4ccccc4)c3)c3cccc4c3oc3c5ccccc5ccc43)cc2-c2ccccc2)cc1] 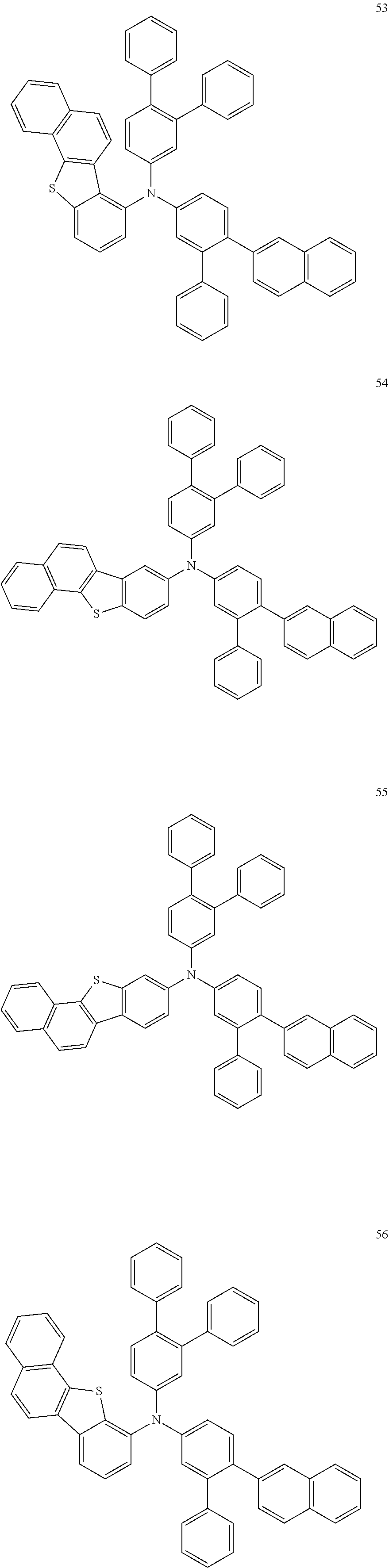 [SMILES: c1ccc(-c2ccc(N(c3ccc(-c4ccc5ccccc5c4)c(-c4ccccc4)c3)c3cccc4sc5c6ccccc6ccc5c34)cc2-c2ccccc2)cc1 ; c1ccc(-c2ccc(N(c3ccc(-c4ccc5ccccc5c4)c(-c4ccccc4)c3)c3ccc4sc5c6ccccc6ccc5c4c3)cc2-c2ccccc2)cc1 ; c1ccc(-c2ccc(N(c3ccc(-c4ccc5ccccc5c4)c(-c4ccccc4)c3)c3ccc4c(c3)sc3c5ccccc5ccc43)cc2-c2ccccc2)cc1 ; c1ccc(-c2ccc(N(c3ccc(-c4ccc5ccccc5c4)c(-c4ccccc4)c3)c3cccc4c3sc3c5ccccc5ccc43)cc2-c2ccccc2)cc1] 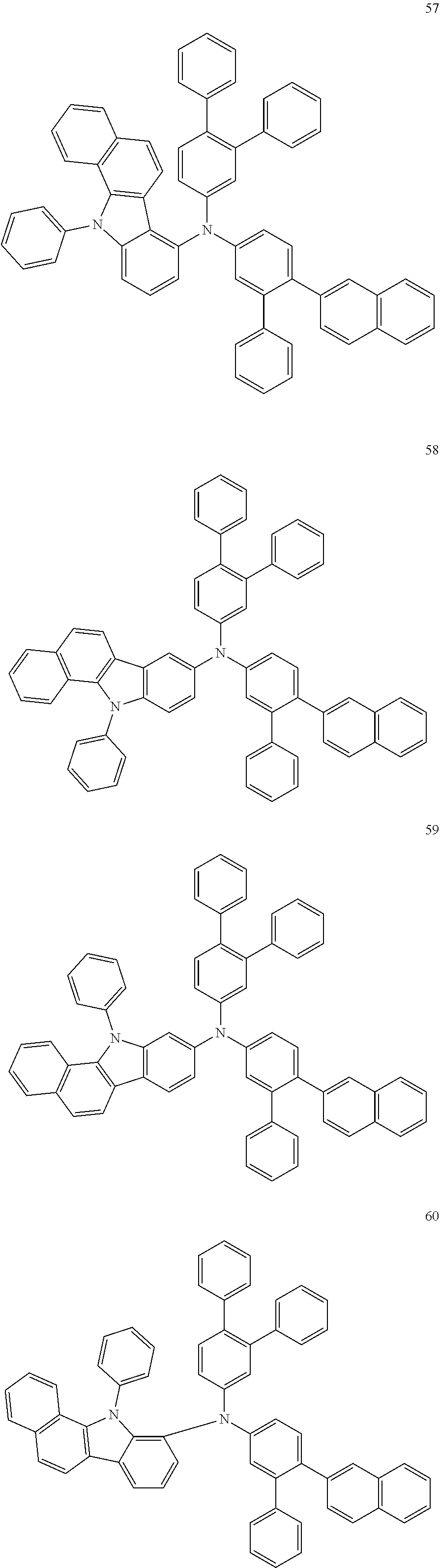 [SMILES: c1ccc(-c2ccc(N(c3ccc(-c4ccc5ccccc5c4)c(-c4ccccc4)c3)c3cccc4c3c3ccc5ccccc5c3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3ccc5ccccc5c3n4-c3ccccc3)cc2-c2ccccc2)cc1 ; c1ccc(-c2ccc(N(c3ccc(-c4ccc5ccccc5c4)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5ccc6ccccc6c5n(-c5ccccc5)c34)cc2-c2ccccc2)cc1] 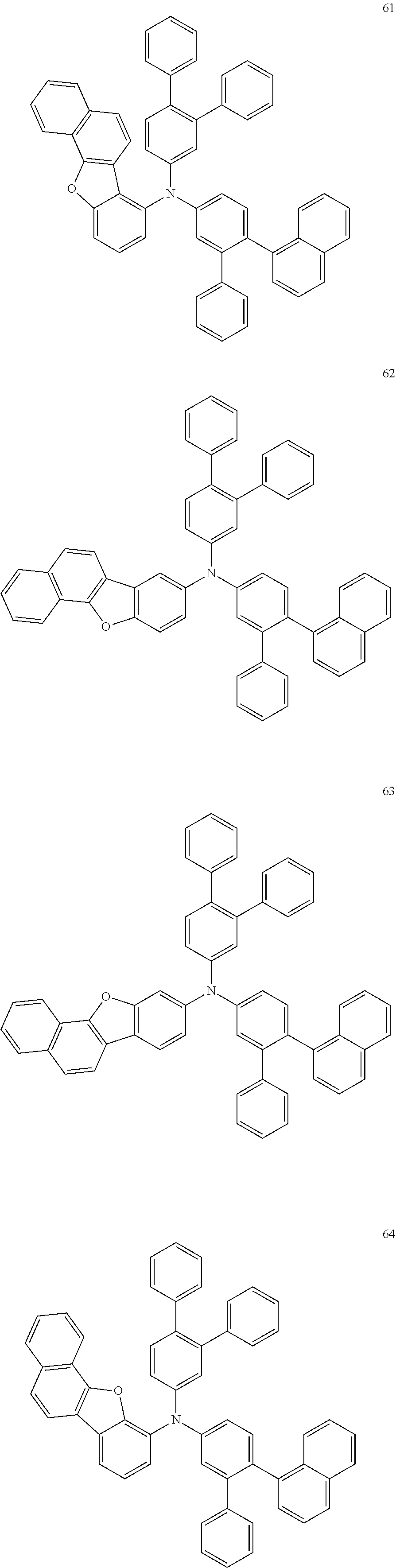 [SMILES: c1ccc(-c2ccc(N(c3ccc(-c4cccc5ccccc45)c(-c4ccccc4)c3)c3cccc4oc5c6ccccc6ccc5c34)cc2-c2ccccc2)cc1 ; c1ccc(-c2ccc(N(c3ccc(-c4cccc5ccccc45)c(-c4ccccc4)c3)c3ccc4oc5c6ccccc6ccc5c4c3)cc2-c2ccccc2)cc1 ; c1ccc(-c2ccc(N(c3ccc(-c4cccc5ccccc45)c(-c4ccccc4)c3)c3ccc4c(c3)oc3c5ccccc5ccc43)cc2-c2ccccc2)cc1 ; c1ccc(-c2ccc(N(c3ccc(-c4cccc5ccccc45)c(-c4ccccc4)c3)c3cccc4c3oc3c5ccccc5ccc43)cc2-c2ccccc2)cc1] 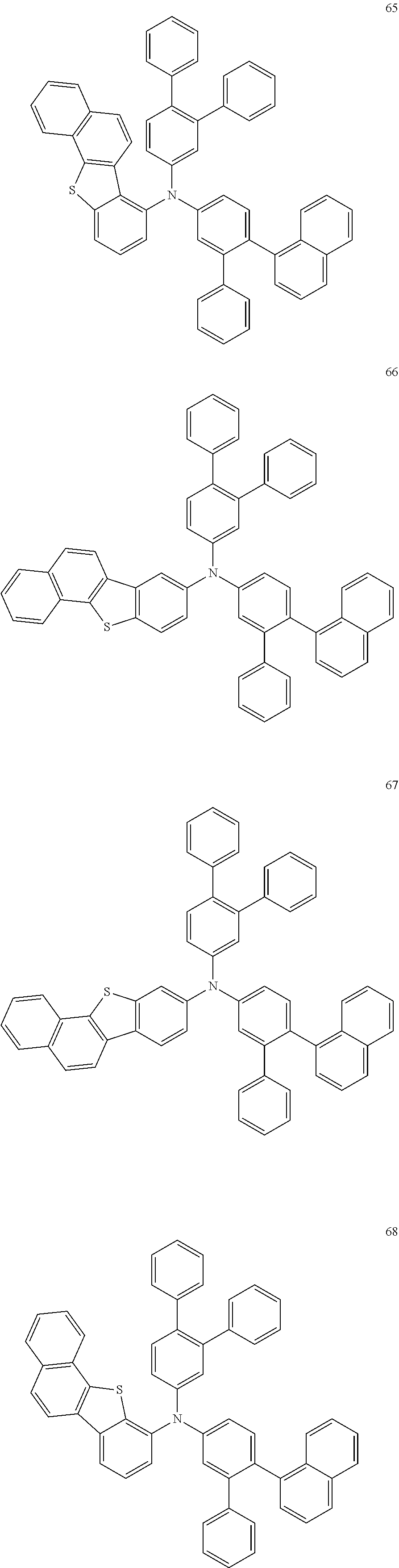 [SMILES: c1ccc(-c2ccc(N(c3ccc(-c4cccc5ccccc45)c(-c4ccccc4)c3)c3cccc4sc5c6ccccc6ccc5c34)cc2-c2ccccc2)cc1 ; c1ccc(-c2ccc(N(c3ccc(-c4cccc5ccccc45)c(-c4ccccc4)c3)c3ccc4sc5c6ccccc6ccc5c4c3)cc2-c2ccccc2)cc1 ; c1ccc(-c2ccc(N(c3ccc(-c4cccc5ccccc45)c(-c4ccccc4)c3)c3ccc4c(c3)sc3c5ccccc5ccc43)cc2-c2ccccc2)cc1 ; c1ccc(-c2ccc(N(c3ccc(-c4cccc5ccccc45)c(-c4ccccc4)c3)c3cccc4c3sc3c5ccccc5ccc43)cc2-c2ccccc2)cc1] 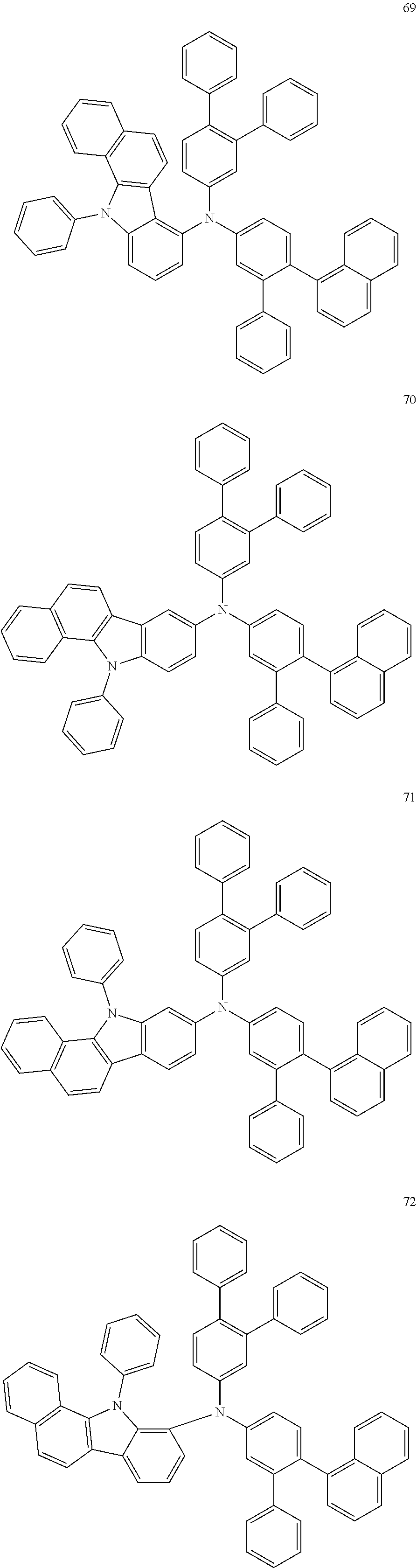 [SMILES: c1ccc(-c2ccc(N(c3ccc(-c4cccc5ccccc45)c(-c4ccccc4)c3)c3cccc4c3c3ccc5ccccc5c3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3ccc5ccccc5c3n4-c3ccccc3)cc2-c2ccccc2)cc1 ; c1ccc(-c2ccc(N(c3ccc(-c4cccc5ccccc45)c(-c4ccccc4)c3)c3ccc4c5ccc6ccccc6c5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5ccc6ccccc6c5n(-c5ccccc5)c34)cc2-c2ccccc2)cc1] 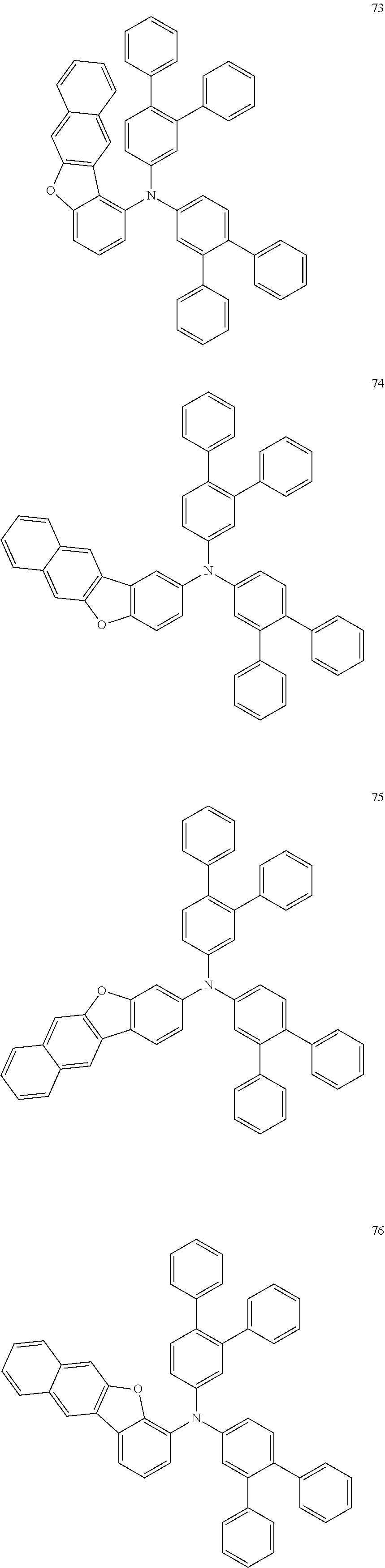 [SMILES: c1ccc(-c2ccc(N(c3ccc(-c4ccccc4)c(-c4ccccc4)c3)c3cccc4oc5cc6ccccc6cc5c34)cc2-c2ccccc2)cc1 ; c1ccc(-c2ccc(N(c3ccc(-c4ccccc4)c(-c4ccccc4)c3)c3ccc4oc5cc6ccccc6cc5c4c3)cc2-c2ccccc2)cc1 ; c1ccc(-c2ccc(N(c3ccc(-c4ccccc4)c(-c4ccccc4)c3)c3ccc4c(c3)oc3cc5ccccc5cc34)cc2-c2ccccc2)cc1 ; c1ccc(-c2ccc(N(c3ccc(-c4ccccc4)c(-c4ccccc4)c3)c3cccc4c3oc3cc5ccccc5cc34)cc2-c2ccccc2)cc1] 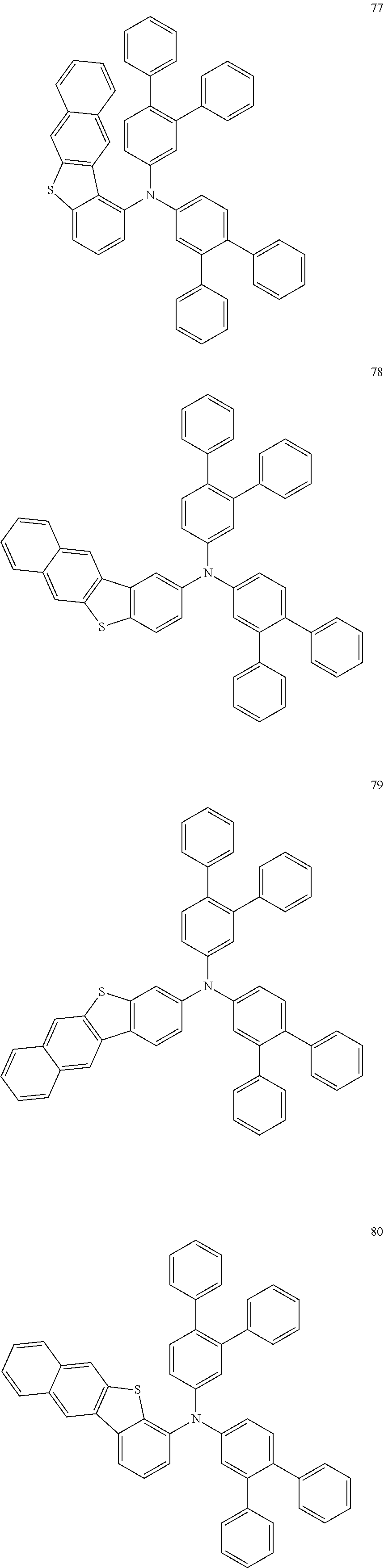 [SMILES: c1ccc(-c2ccc(N(c3ccc(-c4ccccc4)c(-c4ccccc4)c3)c3cccc4sc5cc6ccccc6cc5c34)cc2-c2ccccc2)cc1 ; c1ccc(-c2ccc(N(c3ccc(-c4ccccc4)c(-c4ccccc4)c3)c3ccc4sc5cc6ccccc6cc5c4c3)cc2-c2ccccc2)cc1 ; c1ccc(-c2ccc(N(c3ccc(-c4ccccc4)c(-c4ccccc4)c3)c3ccc4c(c3)sc3cc5ccccc5cc34)cc2-c2ccccc2)cc1 ; c1ccc(-c2ccc(N(c3ccc(-c4ccccc4)c(-c4ccccc4)c3)c3cccc4c3sc3cc5ccccc5cc34)cc2-c2ccccc2)cc1] 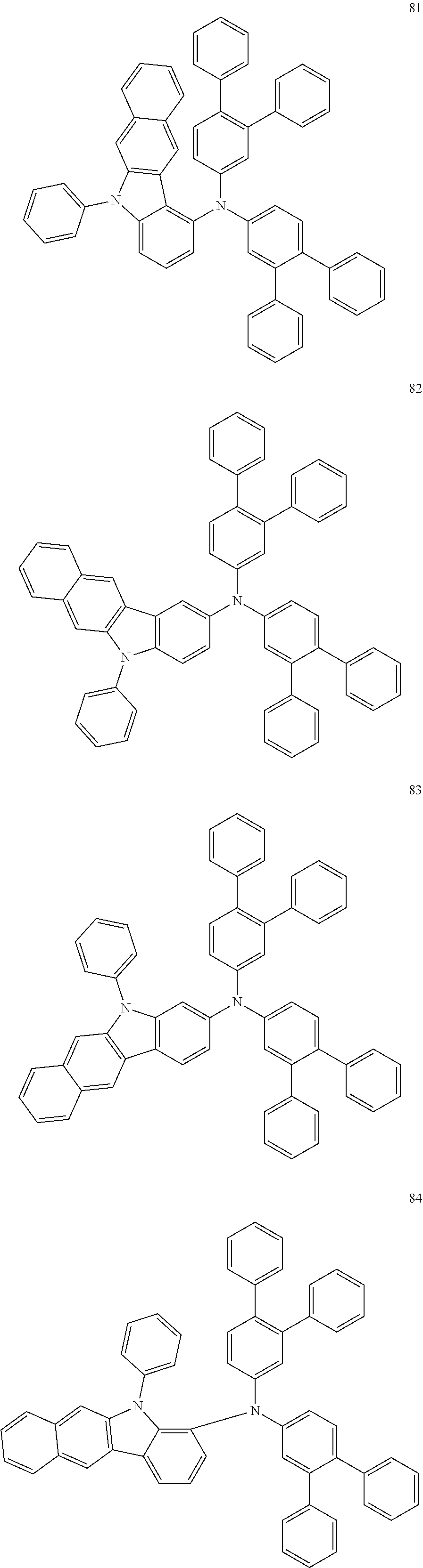 [SMILES: c1ccc(-c2ccc(N(c3ccc(-c4ccccc4)c(-c4ccccc4)c3)c3cccc4c3c3cc5ccccc5cc3n4-c3ccccc3)cc2-c2ccccc2)cc1 ; c1ccc(-c2ccc(N(c3ccc(-c4ccccc4)c(-c4ccccc4)c3)c3ccc4c(c3)c3cc5ccccc5cc3n4-c3ccccc3)cc2-c2ccccc2)cc1 ; c1ccc(-c2ccc(N(c3ccc(-c4ccccc4)c(-c4ccccc4)c3)c3ccc4c5cc6ccccc6cc5n(-c5ccccc5)c4c3)cc2-c2ccccc2)cc1 ; c1ccc(-c2ccc(N(c3ccc(-c4ccccc4)c(-c4ccccc4)c3)c3cccc4c5cc6ccccc6cc5n(-c5ccccc5)c34)cc2-c2ccccc2)cc1] 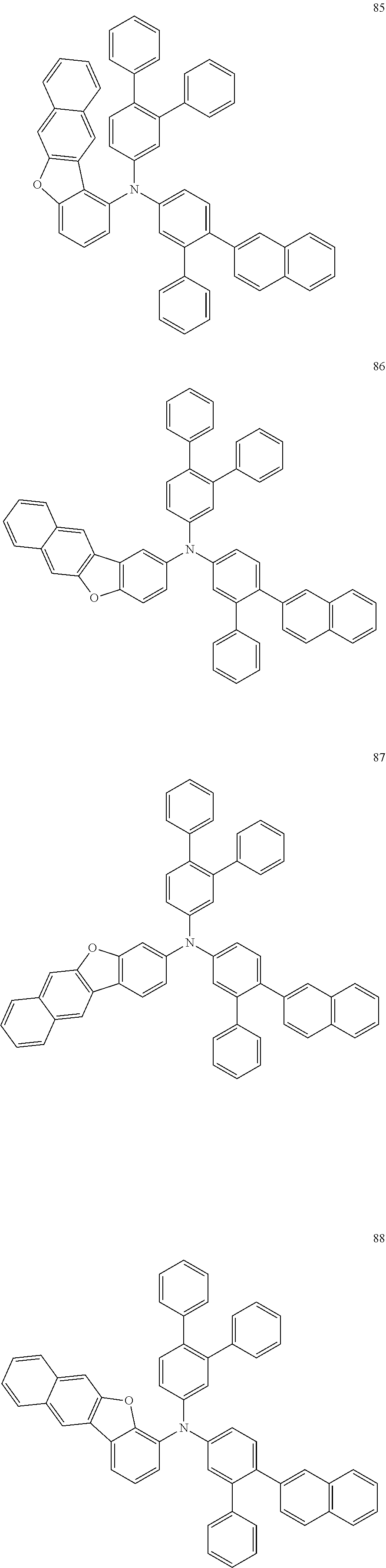 [SMILES: c1ccc(-c2ccc(N(c3ccc(-c4ccc5ccccc5c4)c(-c4ccccc4)c3)c3cccc4oc5cc6ccccc6cc5c34)cc2-c2ccccc2)cc1 ; c1ccc(-c2ccc(N(c3ccc(-c4ccc5ccccc5c4)c(-c4ccccc4)c3)c3ccc4oc5cc6ccccc6cc5c4c3)cc2-c2ccccc2)cc1 ; c1ccc(-c2ccc(N(c3ccc(-c4ccc5ccccc5c4)c(-c4ccccc4)c3)c3ccc4c(c3)oc3cc5ccccc5cc34)cc2-c2ccccc2)cc1 ; c1ccc(-c2ccc(N(c3ccc(-c4ccc5ccccc5c4)c(-c4ccccc4)c3)c3cccc4c3oc3cc5ccccc5cc34)cc2-c2ccccc2)cc1] 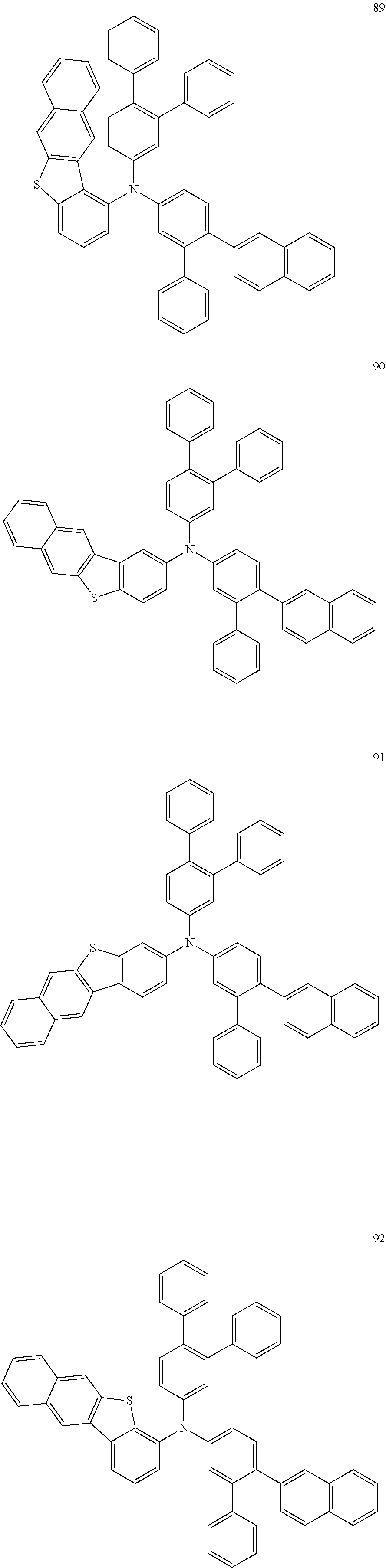 [SMILES: c1ccc(-c2ccc(N(c3ccc(-c4ccc5ccccc5c4)c(-c4ccccc4)c3)c3cccc4sc5cc6ccccc6cc5c34)cc2-c2ccccc2)cc1 ; c1ccc(-c2ccc(N(c3ccc(-c4ccc5ccccc5c4)c(-c4ccccc4)c3)c3ccc4sc5cc6ccccc6cc5c4c3)cc2-c2ccccc2)cc1 ; c1ccc(-c2ccc(N(c3ccc(-c4ccc5ccccc5c4)c(-c4ccccc4)c3)c3ccc4c(c3)sc3cc5ccccc5cc34)cc2-c2ccccc2)cc1 ; c1ccc(-c2ccc(N(c3ccc(-c4ccc5ccccc5c4)c(-c4ccccc4)c3)c3cccc4c3sc3cc5ccccc5cc34)cc2-c2ccccc2)cc1] 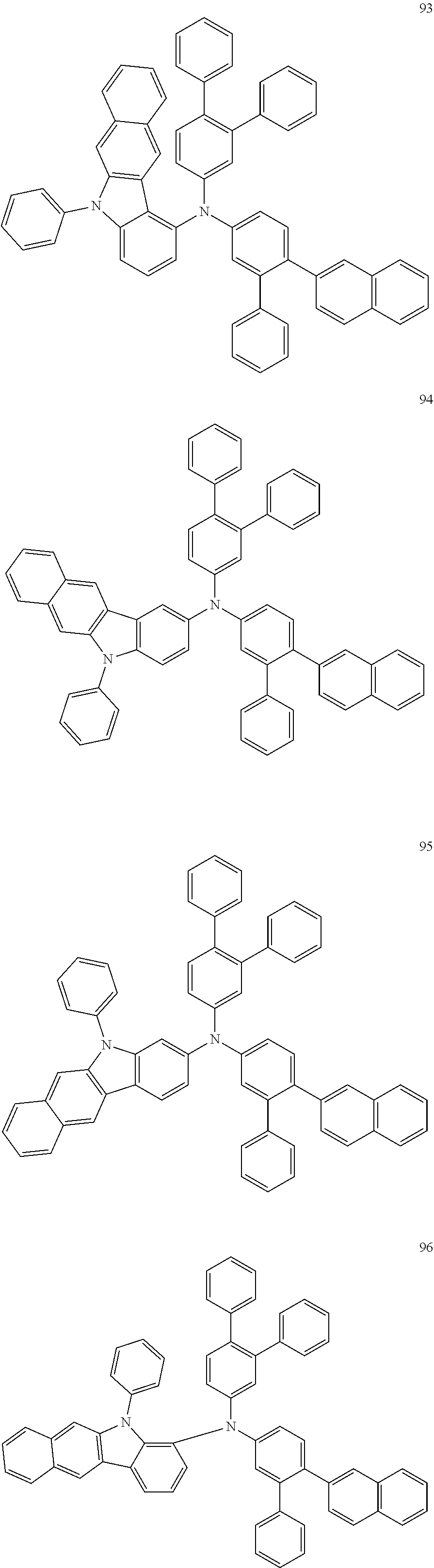 [SMILES: c1ccc(-c2ccc(N(c3ccc(-c4ccc5ccccc5c4)c(-c4ccccc4)c3)c3cccc4c3c3cc5ccccc5cc3n4-c3ccccc3)cc2-c2ccccc2)cc1 ; c1ccc(-c2ccc(N(c3ccc(-c4ccc5ccccc5c4)c(-c4ccccc4)c3)c3ccc4c(c3)c3cc5ccccc5cc3n4-c3ccccc3)cc2-c2ccccc2)cc1 ; c1ccc(-c2ccc(N(c3ccc(-c4ccc5ccccc5c4)c(-c4ccccc4)c3)c3ccc4c5cc6ccccc6cc5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5cc6ccccc6cc5n(-c5ccccc5)c34)cc2-c2ccccc2)cc1] 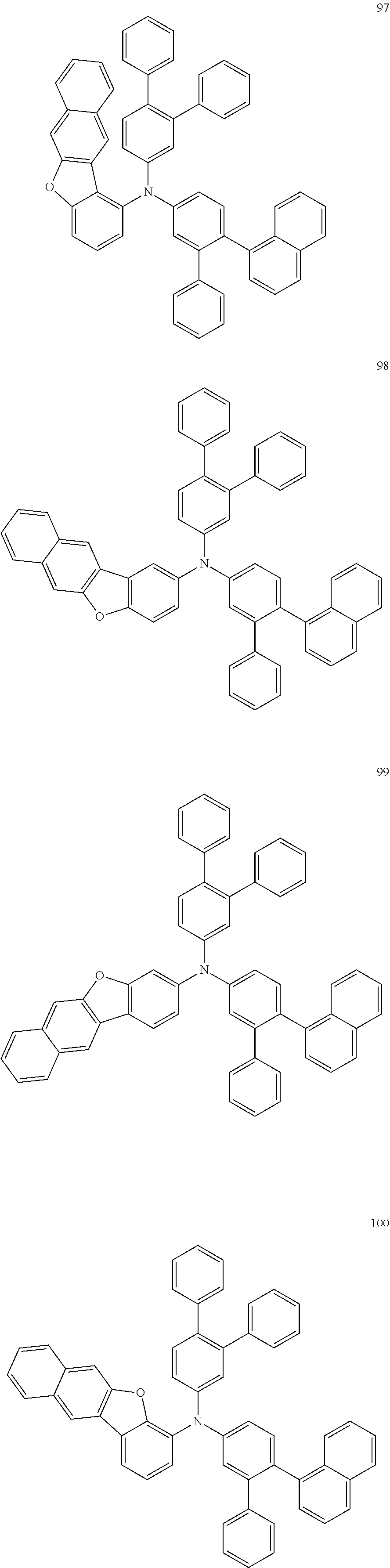 [SMILES: c1ccc(-c2ccc(N(c3ccc(-c4cccc5ccccc45)c(-c4ccccc4)c3)c3cccc4oc5cc6ccccc6cc5c34)cc2-c2ccccc2)cc1 ; c1ccc(-c2ccc(N(c3ccc(-c4cccc5ccccc45)c(-c4ccccc4)c3)c3ccc4oc5cc6ccccc6cc5c4c3)cc2-c2ccccc2)cc1 ; c1ccc(-c2ccc(N(c3ccc(-c4cccc5ccccc45)c(-c4ccccc4)c3)c3ccc4c(c3)oc3cc5ccccc5cc34)cc2-c2ccccc2)cc1 ; c1ccc(-c2ccc(N(c3ccc(-c4cccc5ccccc45)c(-c4ccccc4)c3)c3cccc4c3oc3cc5ccccc5cc34)cc2-c2ccccc2)cc1] 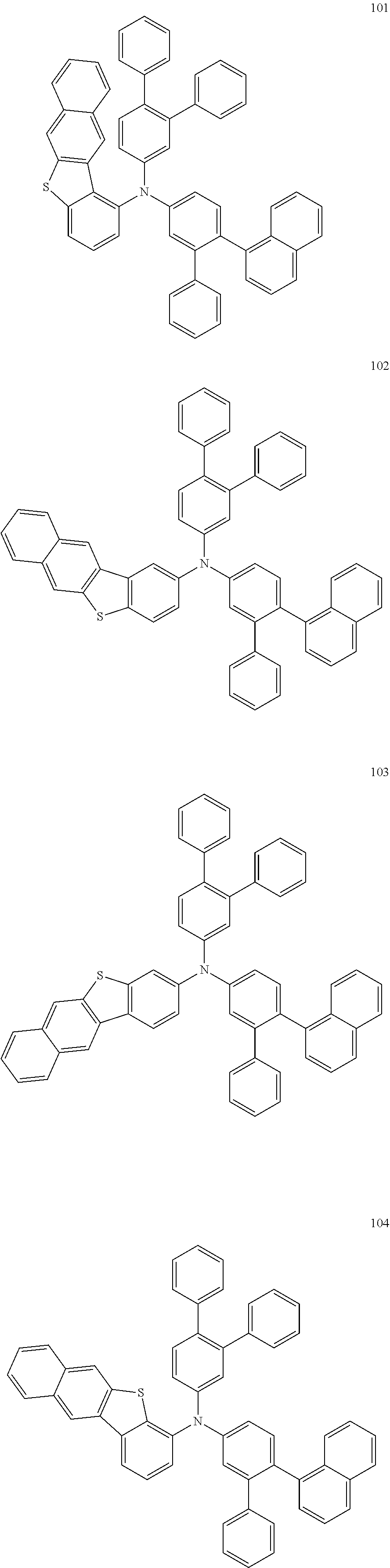 [SMILES: c1ccc(-c2ccc(N(c3ccc(-c4cccc5ccccc45)c(-c4ccccc4)c3)c3cccc4sc5cc6ccccc6cc5c34)cc2-c2ccccc2)cc1 ; c1ccc(-c2ccc(N(c3ccc(-c4cccc5ccccc45)c(-c4ccccc4)c3)c3ccc4sc5cc6ccccc6cc5c4c3)cc2-c2ccccc2)cc1 ; c1ccc(-c2ccc(N(c3ccc(-c4cccc5ccccc45)c(-c4ccccc4)c3)c3ccc4c(c3)sc3cc5ccccc5cc34)cc2-c2ccccc2)cc1 ; c1ccc(-c2ccc(N(c3ccc(-c4cccc5ccccc45)c(-c4ccccc4)c3)c3cccc4c3sc3cc5ccccc5cc34)cc2-c2ccccc2)cc1] 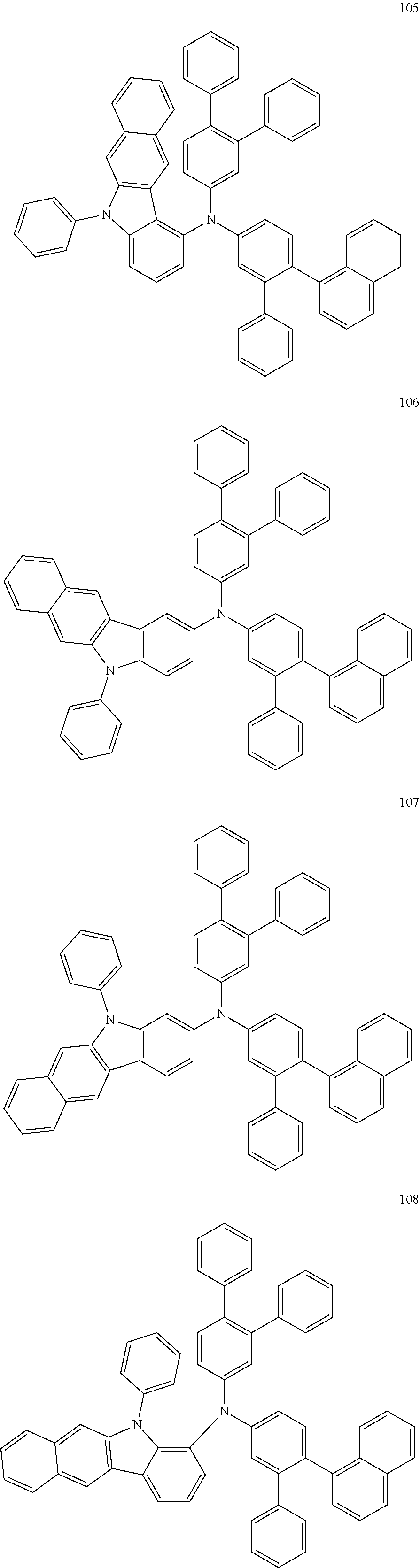 [SMILES: c1ccc(-c2ccc(N(c3ccc(-c4cccc5ccccc45)c(-c4ccccc4)c3)c3cccc4c3c3cc5ccccc5cc3n4-c3ccccc3)cc2-c2ccccc2)cc1 ; c1ccc(-c2ccc(N(c3ccc(-c4cccc5ccccc45)c(-c4ccccc4)c3)c3ccc4c(c3)c3cc5ccccc5cc3n4-c3ccccc3)cc2-c2ccccc2)cc1 ; c1ccc(-c2ccc(N(c3ccc(-c4cccc5ccccc45)c(-c4ccccc4)c3)c3ccc4c5cc6ccccc6cc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5cc6ccccc6cc5n(-c5ccccc5)c34)cc2-c2ccccc2)cc1] 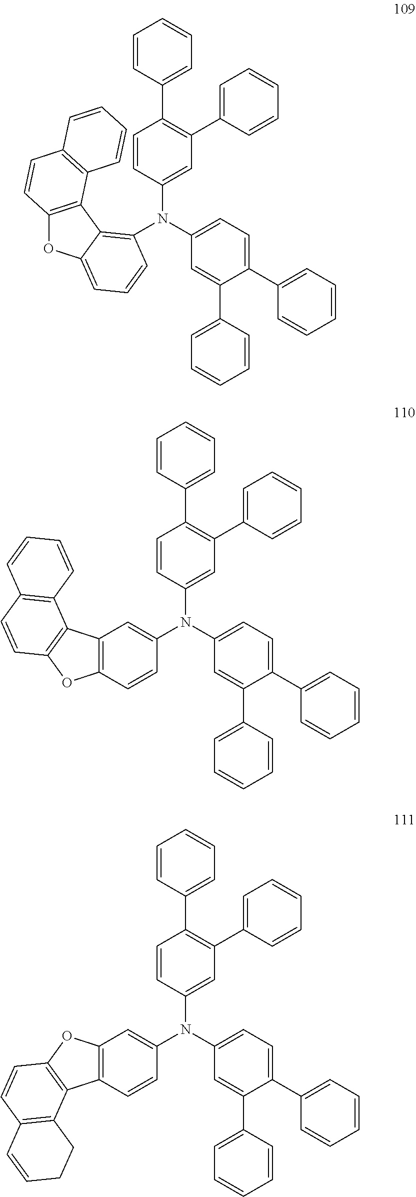 [SMILES: c1ccc(-c2ccc(N(c3ccc(-c4ccccc4)c(-c4ccccc4)c3)c3cccc4oc5ccc6ccccc6c5c34)cc2-c2ccccc2)cc1 ; c1ccc(-c2ccc(N(c3ccc(-c4ccccc4)c(-c4ccccc4)c3)c3ccc4oc5ccc6ccccc6c5c4c3)cc2-c2ccccc2)cc1 ; C1=Cc2ccc3oc4cc(N(c5ccc(-c6ccccc6)c(-c6ccccc6)c5)c5ccc(-c6ccccc6)c(-c6ccccc6)c5)ccc4c3c2CC1] 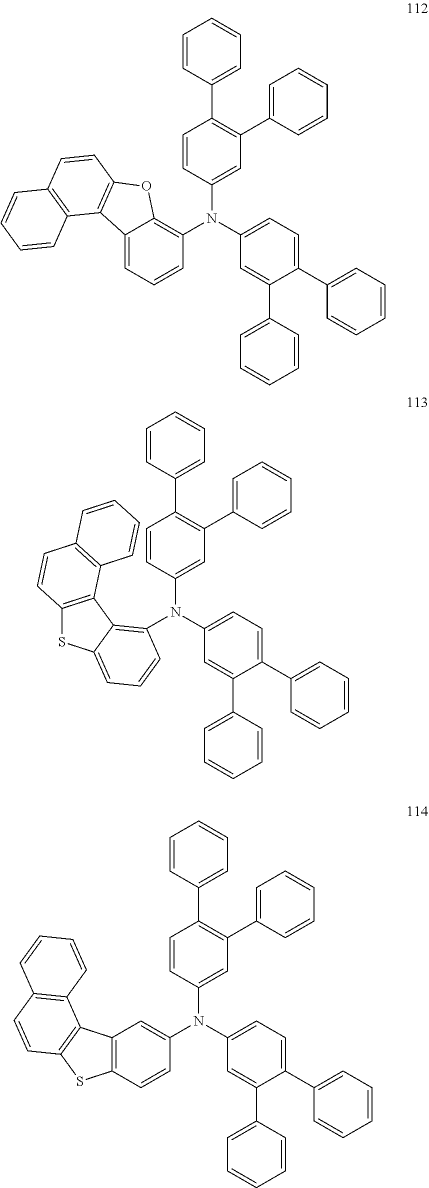 [SMILES: c1ccc(-c2ccc(N(c3ccc(-c4ccccc4)c(-c4ccccc4)c3)c3cccc4c3oc3ccc5ccccc5c34)cc2-c2ccccc2)cc1 ; c1ccc(-c2ccc(N(c3ccc(-c4ccccc4)c(-c4ccccc4)c3)c3cccc4sc5ccc6ccccc6c5c34)cc2-c2ccccc2)cc1 ; c1ccc(-c2ccc(N(c3ccc(-c4ccccc4)c(-c4ccccc4)c3)c3ccc4sc5ccc6ccccc6c5c4c3)cc2-c2ccccc2)cc1] 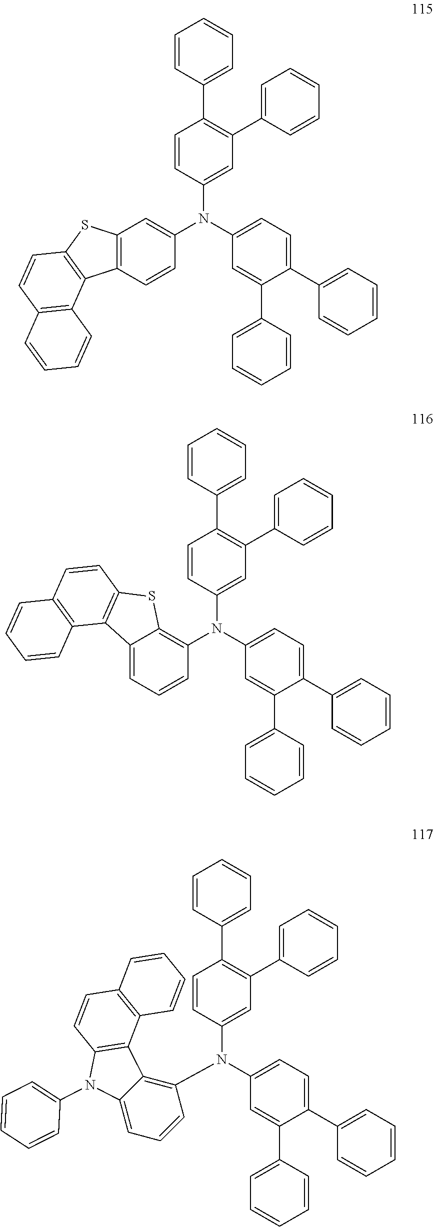 [SMILES: c1ccc(-c2ccc(N(c3ccc(-c4ccccc4)c(-c4ccccc4)c3)c3ccc4c(c3)sc3ccc5ccccc5c34)cc2-c2ccccc2)cc1 ; c1ccc(-c2ccc(N(c3ccc(-c4ccccc4)c(-c4ccccc4)c3)c3cccc4c3sc3ccc5ccccc5c34)cc2-c2ccccc2)cc1 ; c1ccc(-c2ccc(N(c3ccc(-c4ccccc4)c(-c4ccccc4)c3)c3cccc4c3c3c5ccccc5ccc3n4-c3ccccc3)cc2-c2ccccc2)cc1] 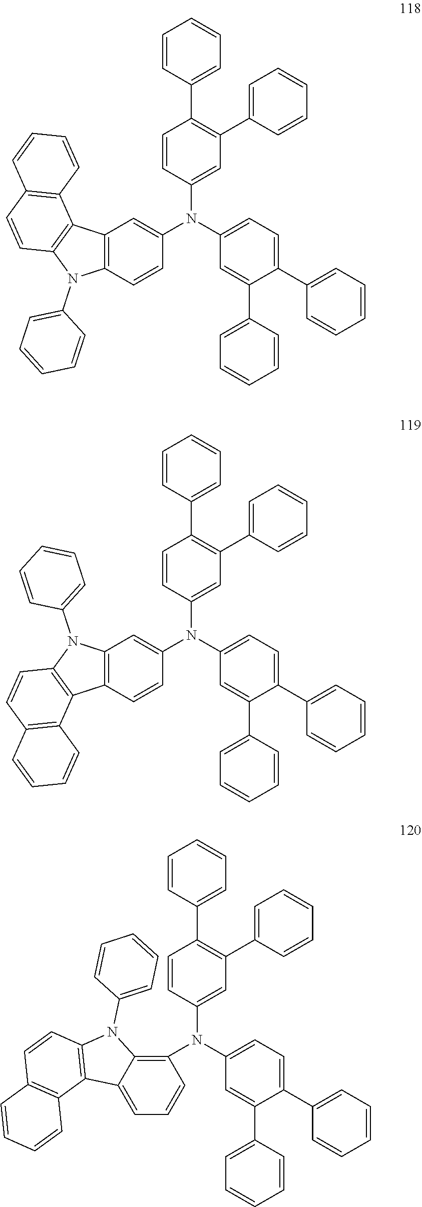 [SMILES: c1ccc(-c2ccc(N(c3ccc(-c4ccccc4)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4ccccc4)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4ccccc4)c(-c4ccccc4)c3)c3cccc4c5c6ccccc6ccc5n(-c5ccccc5)c34)cc2-c2ccccc2)cc1] 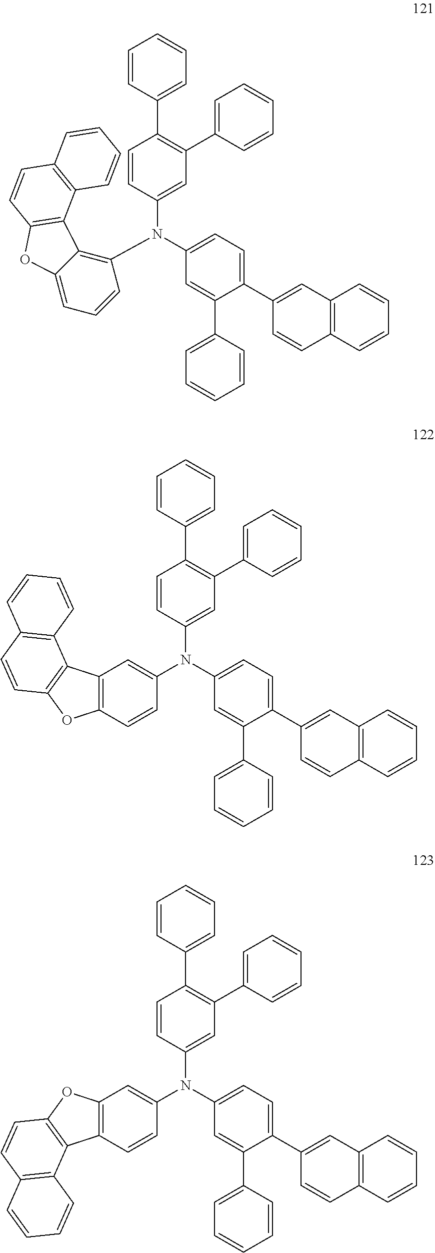 [SMILES: c1ccc(-c2ccc(N(c3ccc(-c4ccc5ccccc5c4)c(-c4ccccc4)c3)c3cccc4oc5ccc6ccccc6c5c34)cc2-c2ccccc2)cc1 ; c1ccc(-c2ccc(N(c3ccc(-c4ccc5ccccc5c4)c(-c4ccccc4)c3)c3ccc4oc5ccc6ccccc6c5c4c3)cc2-c2ccccc2)cc1 ; c1ccc(-c2ccc(N(c3ccc(-c4ccc5ccccc5c4)c(-c4ccccc4)c3)c3ccc4c(c3)oc3ccc5ccccc5c34)cc2-c2ccccc2)cc1] 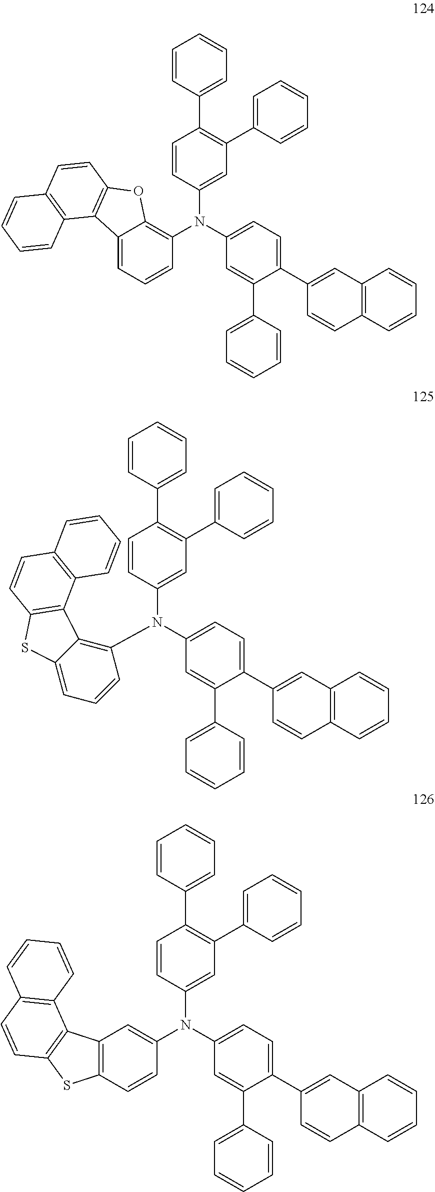 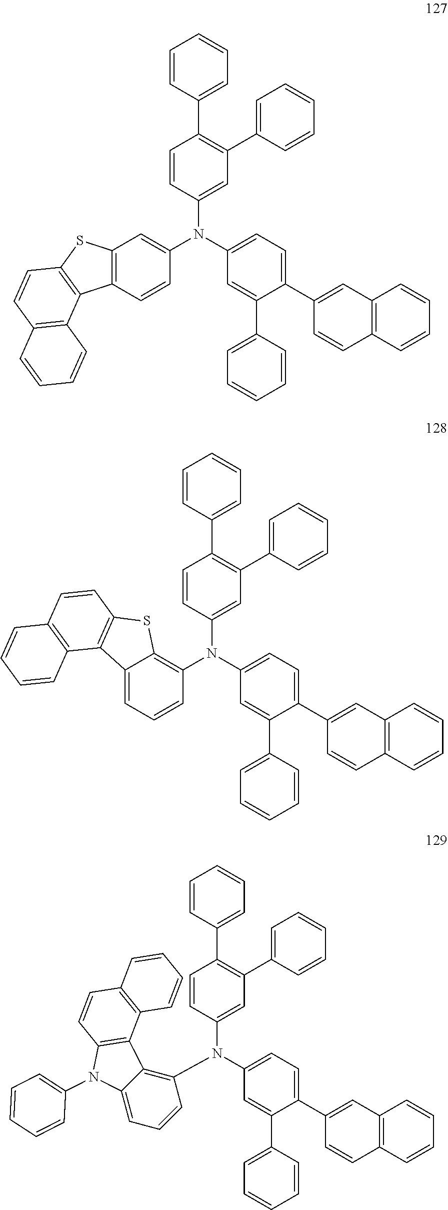 [SMILES: c1ccc(-c2ccc(N(c3ccc(-c4ccc5ccccc5c4)c(-c4ccccc4)c3)c3ccc4c(c3)sc3ccc5ccccc5c34)cc2-c2ccccc2)cc1 ; c1ccc(-c2ccc(N(c3ccc(-c4ccc5ccccc5c4)c(-c4ccccc4)c3)c3cccc4c3sc3ccc5ccccc5c34)cc2-c2ccccc2)cc1 ; c1ccc(-c2ccc(N(c3ccc(-c4ccc5ccccc5c4)c(-c4ccccc4)c3)c3cccc4c3c3c5ccccc5ccc3n4-c3ccccc3)cc2-c2ccccc2)cc1] 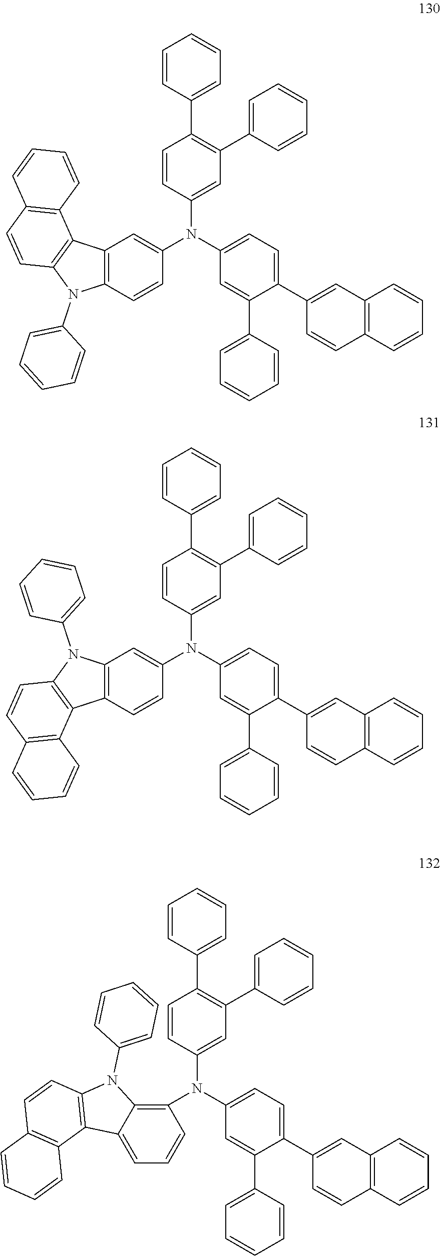 [SMILES: c1ccc(-c2ccc(N(c3ccc(-c4ccc5ccccc5c4)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4ccc5ccccc5c4)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4ccc5ccccc5c4)c(-c4ccccc4)c3)c3cccc4c5c6ccccc6ccc5n(-c5ccccc5)c34)cc2-c2ccccc2)cc1] 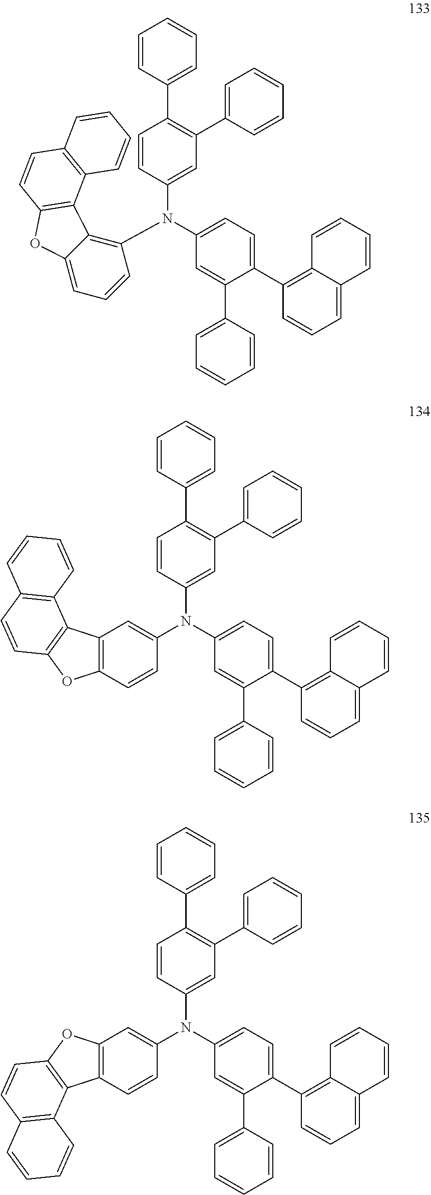 [SMILES: c1ccc(-c2ccc(N(c3ccc(-c4cccc5ccccc45)c(-c4ccccc4)c3)c3cccc4oc5ccc6ccccc6c5c34)cc2-c2ccccc2)cc1 ; c1ccc(-c2ccc(N(c3ccc(-c4cccc5ccccc45)c(-c4ccccc4)c3)c3ccc4oc5ccc6ccccc6c5c4c3)cc2-c2ccccc2)cc1 ; c1ccc(-c2ccc(N(c3ccc(-c4cccc5ccccc45)c(-c4ccccc4)c3)c3ccc4c(c3)oc3ccc5ccccc5c34)cc2-c2ccccc2)cc1] 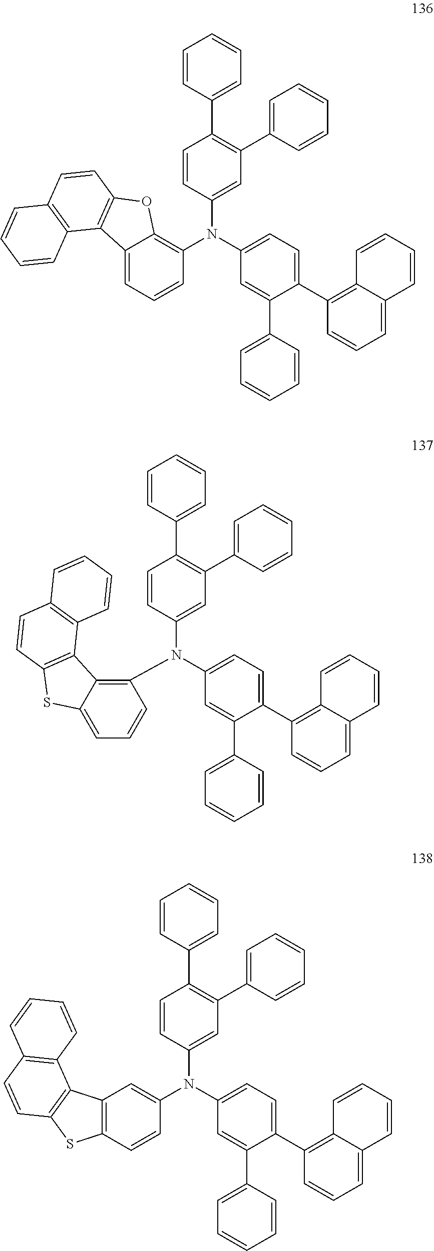 [SMILES: c1ccc(-c2ccc(N(c3ccc(-c4cccc5ccccc45)c(-c4ccccc4)c3)c3cccc4c3oc3ccc5ccccc5c34)cc2-c2ccccc2)cc1 ; c1ccc(-c2ccc(N(c3ccc(-c4cccc5ccccc45)c(-c4ccccc4)c3)c3cccc4sc5ccc6ccccc6c5c34)cc2-c2ccccc2)cc1 ; c1ccc(-c2ccc(N(c3ccc(-c4cccc5ccccc45)c(-c4ccccc4)c3)c3ccc4sc5ccc6ccccc6c5c4c3)cc2-c2ccccc2)cc1] 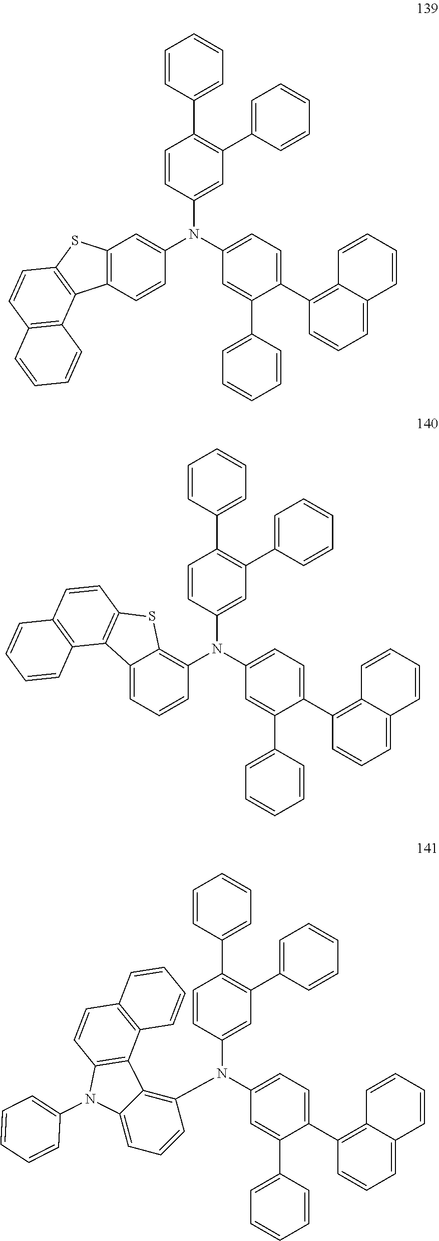 [SMILES: c1ccc(-c2ccc(N(c3ccc(-c4cccc5ccccc45)c(-c4ccccc4)c3)c3ccc4c(c3)sc3ccc5ccccc5c34)cc2-c2ccccc2)cc1 ; c1ccc(-c2ccc(N(c3ccc(-c4cccc5ccccc45)c(-c4ccccc4)c3)c3cccc4c3sc3ccc5ccccc5c34)cc2-c2ccccc2)cc1 ; c1ccc(-c2ccc(N(c3ccc(-c4cccc5ccccc45)c(-c4ccccc4)c3)c3cccc4c3c3c5ccccc5ccc3n4-c3ccccc3)cc2-c2ccccc2)cc1] 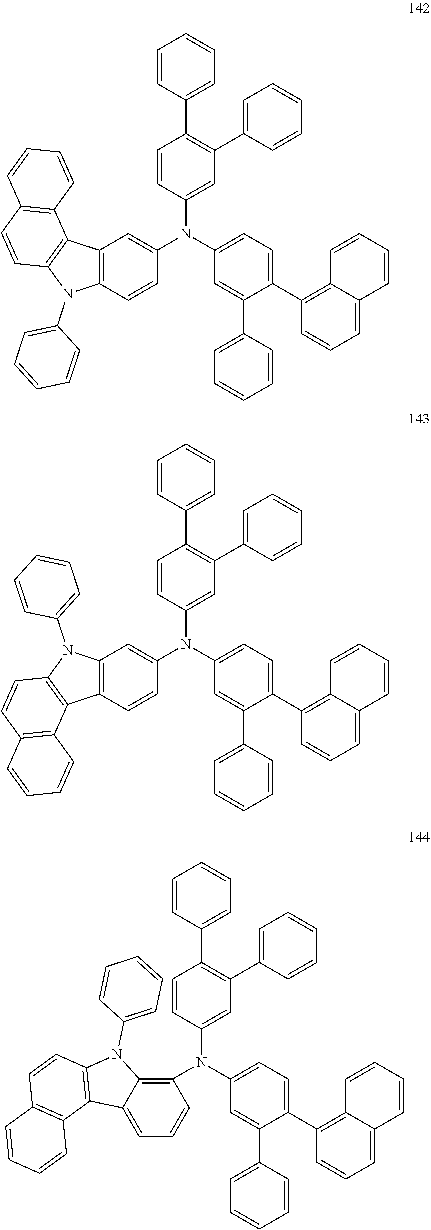 [SMILES: c1ccc(-c2ccc(N(c3ccc(-c4cccc5ccccc45)c(-c4ccccc4)c3)c3ccc4c(c3)c3c5ccccc5ccc3n4-c3ccccc3)cc2-c2ccccc2)cc1 ; c1ccc(-c2ccc(N(c3ccc(-c4cccc5ccccc45)c(-c4ccccc4)c3)c3ccc4c5c6ccccc6ccc5n(-c5ccccc5)c4c3)cc2-c2ccccc2)cc1 ; c1ccc(-c2ccc(N(c3ccc(-c4cccc5ccccc45)c(-c4ccccc4)c3)c3cccc4c5c6ccccc6ccc5n(-c5ccccc5)c34)cc2-c2ccccc2)cc1] 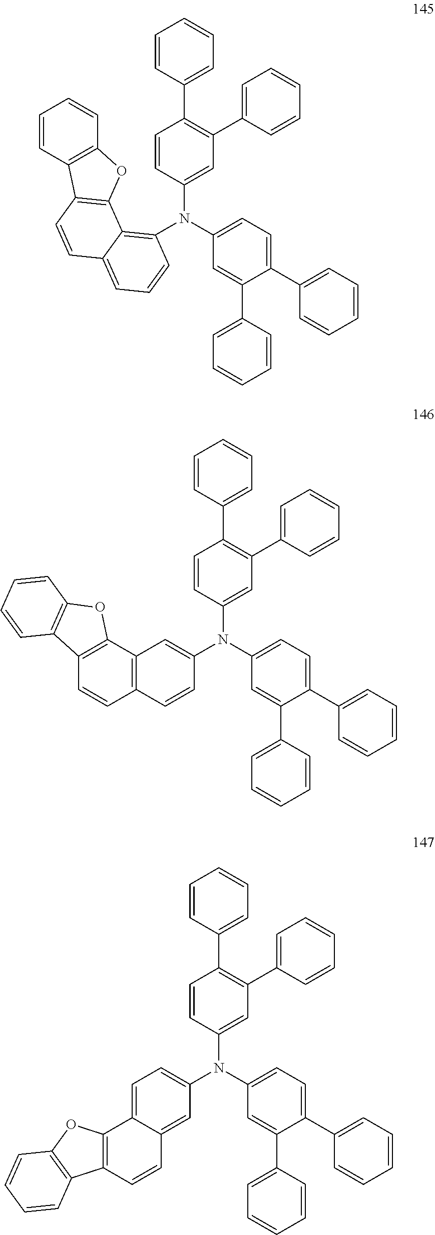 [SMILES: c1ccc(-c2ccc(N(c3ccc(-c4ccccc4)c(-c4ccccc4)c3)c3cccc4ccc5c6ccccc6oc5c34)cc2-c2ccccc2)cc1 ; c1ccc(-c2ccc(N(c3ccc(-c4ccccc4)c(-c4ccccc4)c3)c3ccc4ccc5c6ccccc6oc5c4c3)cc2-c2ccccc2)cc1 ; c1ccc(-c2ccc(N(c3ccc(-c4ccccc4)c(-c4ccccc4)c3)c3ccc4c(ccc5c6ccccc6oc45)c3)cc2-c2ccccc2)cc1] 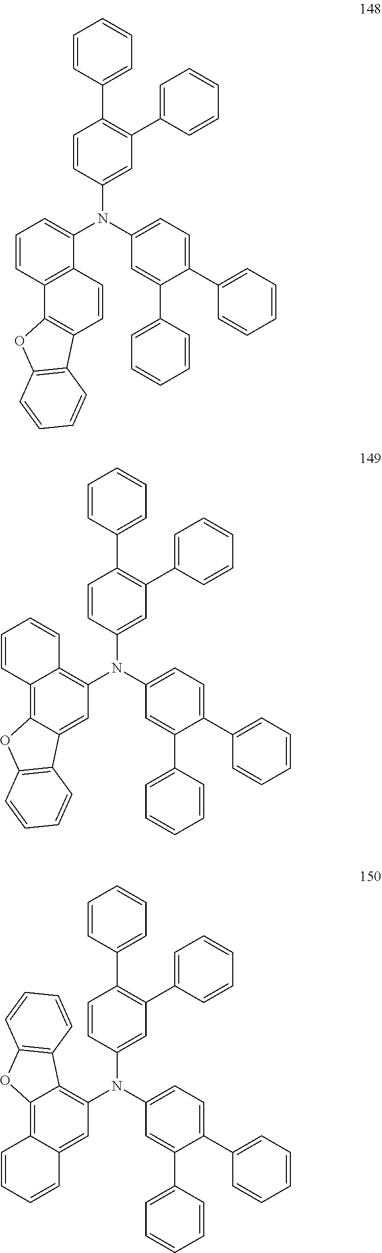 [SMILES: c1ccc(-c2ccc(N(c3ccc(-c4ccccc4)c(-c4ccccc4)c3)c3cccc4c3ccc3c5ccccc5oc43)cc2-c2ccccc2)cc1 ; c1ccc(-c2ccc(N(c3ccc(-c4ccccc4)c(-c4ccccc4)c3)c3cc4c5ccccc5oc4c4ccccc34)cc2-c2ccccc2)cc1 ; c1ccc(-c2ccc(N(c3ccc(-c4ccccc4)c(-c4ccccc4)c3)c3cc4ccccc4c4oc5ccccc5c34)cc2-c2ccccc2)cc1] 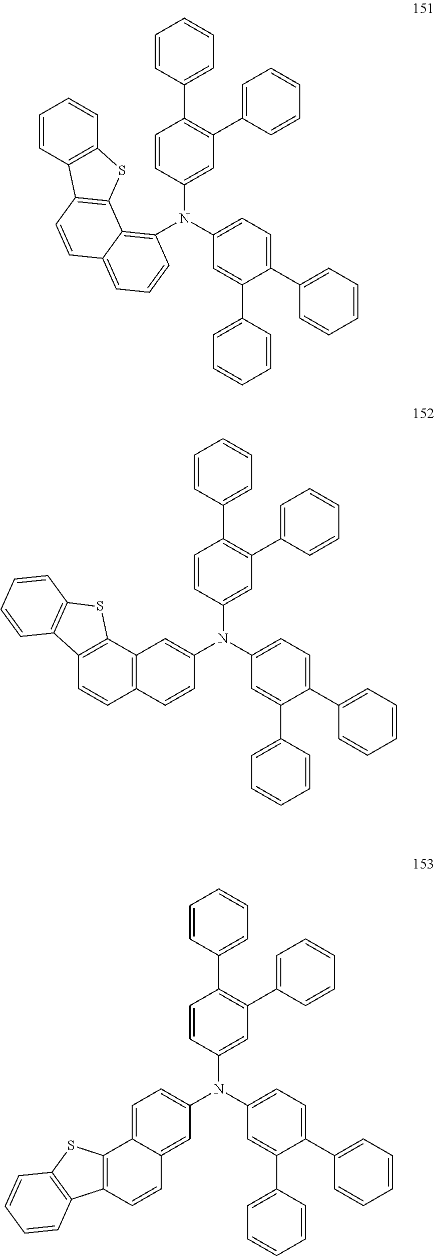 [SMILES: c1ccc(-c2ccc(N(c3ccc(-c4ccccc4)c(-c4ccccc4)c3)c3cccc4ccc5c6ccccc6sc5c34)cc2-c2ccccc2)cc1 ; c1ccc(-c2ccc(N(c3ccc(-c4ccccc4)c(-c4ccccc4)c3)c3ccc4ccc5c6ccccc6sc5c4c3)cc2-c2ccccc2)cc1 ; c1ccc(-c2ccc(N(c3ccc(-c4ccccc4)c(-c4ccccc4)c3)c3ccc4c(ccc5c6ccccc6sc45)c3)cc2-c2ccccc2)cc1] 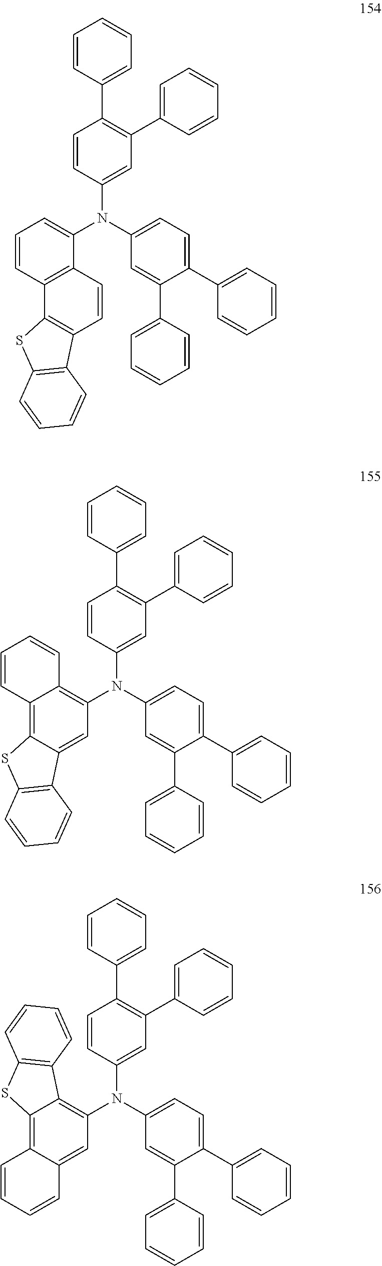 [SMILES: c1ccc(-c2ccc(N(c3ccc(-c4ccccc4)c(-c4ccccc4)c3)c3cccc4c3ccc3c5ccccc5sc43)cc2-c2ccccc2)cc1 ; c1ccc(-c2ccc(N(c3ccc(-c4ccccc4)c(-c4ccccc4)c3)c3cc4c5ccccc5sc4c4ccccc34)cc2-c2ccccc2)cc1 ; c1ccc(-c2ccc(N(c3ccc(-c4ccccc4)c(-c4ccccc4)c3)c3cc4ccccc4c4sc5ccccc5c34)cc2-c2ccccc2)cc1] 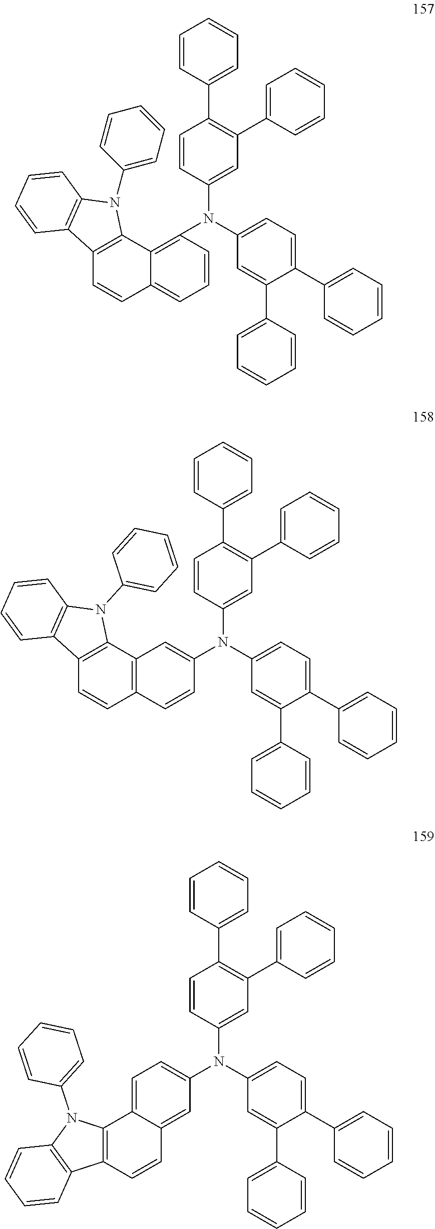 [SMILES: c1ccc(-c2ccc(N(c3ccc(-c4ccccc4)c(-c4ccccc4)c3)c3cccc4ccc5c6ccccc6n(-c6ccccc6)c5c34)cc2-c2ccccc2)cc1 ; c1ccc(-c2ccc(N(c3ccc(-c4ccccc4)c(-c4ccccc4)c3)c3ccc4ccc5c6ccccc6n(-c6ccccc6)c5c4c3)cc2-c2ccccc2)cc1 ; c1ccc(-c2ccc(N(c3ccc(-c4ccccc4)c(-c4ccccc4)c3)c3ccc4c(ccc5c6ccccc6n(-c6ccccc6)c45)c3)cc2-c2ccccc2)cc1] 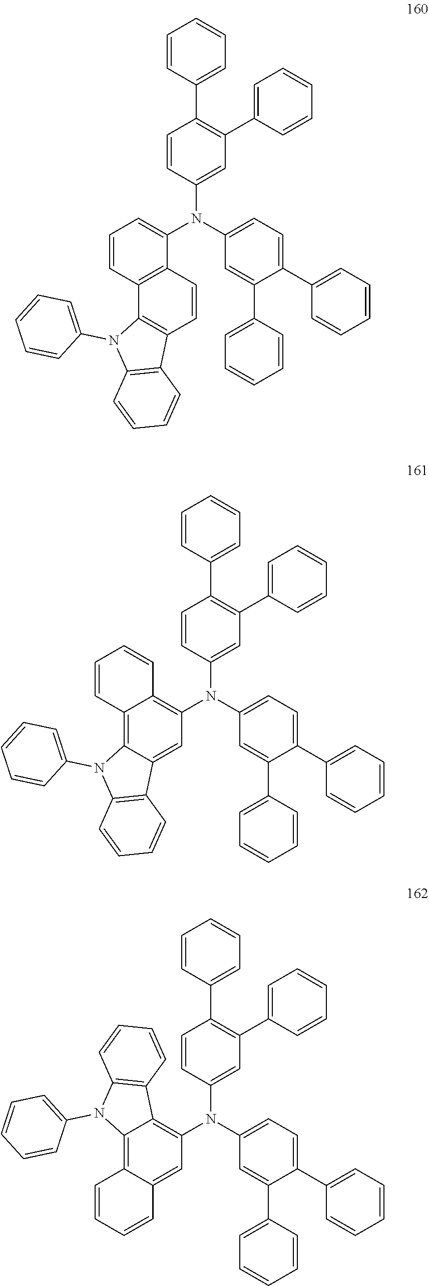 [SMILES: c1ccc(-c2ccc(N(c3ccc(-c4ccccc4)c(-c4ccccc4)c3)c3cccc4c3ccc3c5ccccc5n(-c5ccccc5)c43)cc2-c2ccccc2)cc1 ; c1ccc(-c2ccc(N(c3ccc(-c4ccccc4)c(-c4ccccc4)c3)c3cc4c5ccccc5n(-c5ccccc5)c4c4ccccc34)cc2-c2ccccc2)cc1 ; c1ccc(-c2ccc(N(c3ccc(-c4ccccc4)c(-c4ccccc4)c3)c3cc4ccccc4c4c3c3ccccc3n4-c3ccccc3)cc2-c2ccccc2)cc1] 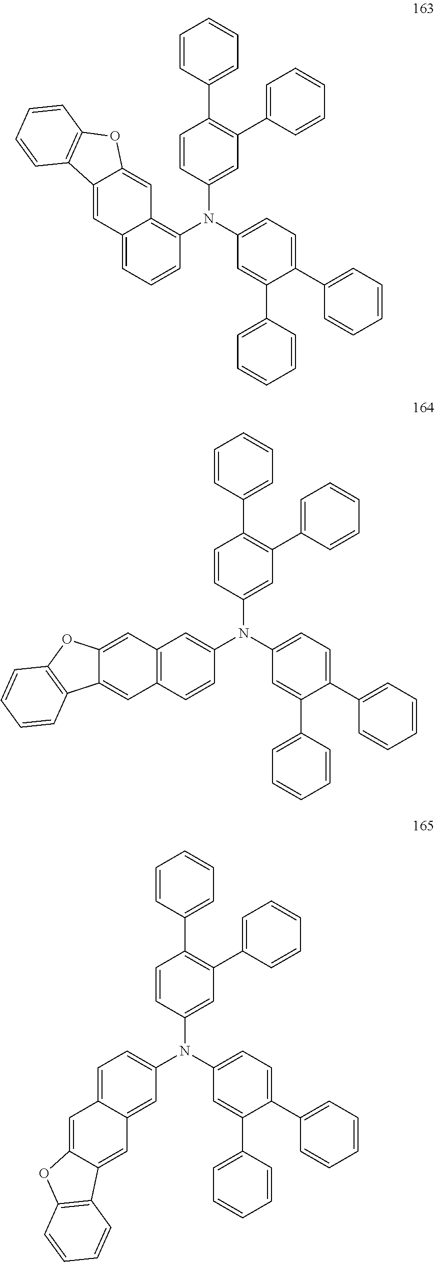 [SMILES: c1ccc(-c2ccc(N(c3ccc(-c4ccccc4)c(-c4ccccc4)c3)c3cccc4cc5c(cc34)oc3ccccc35)cc2-c2ccccc2)cc1 ; c1ccc(-c2ccc(N(c3ccc(-c4ccccc4)c(-c4ccccc4)c3)c3ccc4cc5c(cc4c3)oc3ccccc35)cc2-c2ccccc2)cc1 ; c1ccc(-c2ccc(N(c3ccc(-c4ccccc4)c(-c4ccccc4)c3)c3ccc4cc5oc6ccccc6c5cc4c3)cc2-c2ccccc2)cc1] 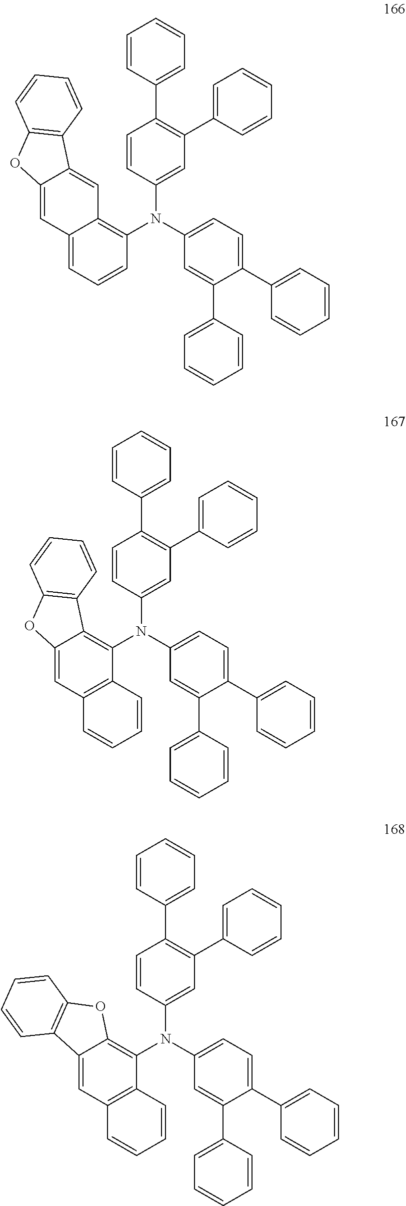 [SMILES: c1ccc(-c2ccc(N(c3ccc(-c4ccccc4)c(-c4ccccc4)c3)c3cccc4cc5oc6ccccc6c5cc34)cc2-c2ccccc2)cc1 ; c1ccc(-c2ccc(N(c3ccc(-c4ccccc4)c(-c4ccccc4)c3)c3c4ccccc4cc4oc5ccccc5c34)cc2-c2ccccc2)cc1 ; c1ccc(-c2ccc(N(c3ccc(-c4ccccc4)c(-c4ccccc4)c3)c3c4ccccc4cc4c3oc3ccccc34)cc2-c2ccccc2)cc1] 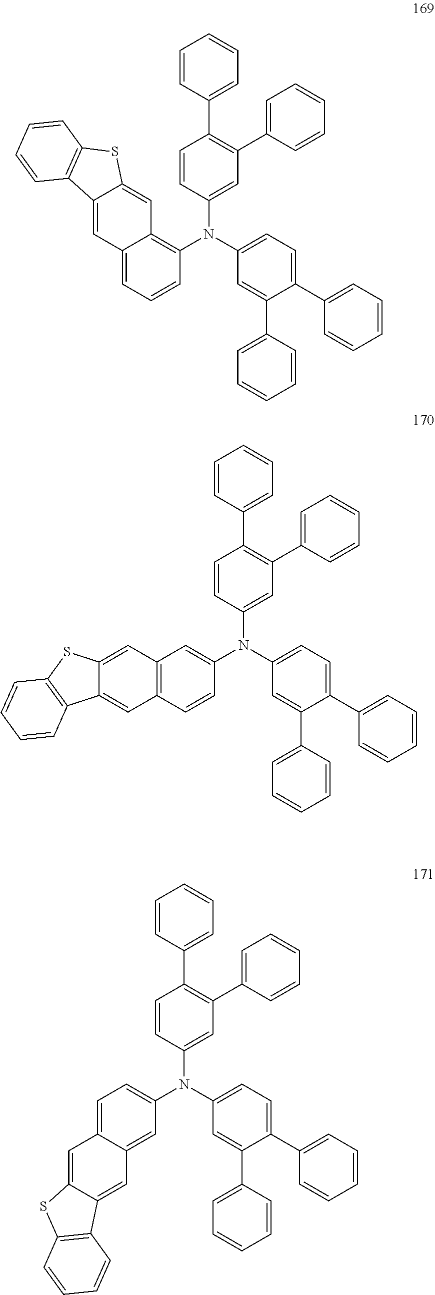 [SMILES: c1ccc(-c2ccc(N(c3ccc(-c4ccccc4)c(-c4ccccc4)c3)c3cccc4cc5c(cc34)sc3ccccc35)cc2-c2ccccc2)cc1 ; c1ccc(-c2ccc(N(c3ccc(-c4ccccc4)c(-c4ccccc4)c3)c3ccc4cc5c(cc4c3)sc3ccccc35)cc2-c2ccccc2)cc1 ; c1ccc(-c2ccc(N(c3ccc(-c4ccccc4)c(-c4ccccc4)c3)c3ccc4cc5sc6ccccc6c5cc4c3)cc2-c2ccccc2)cc1] 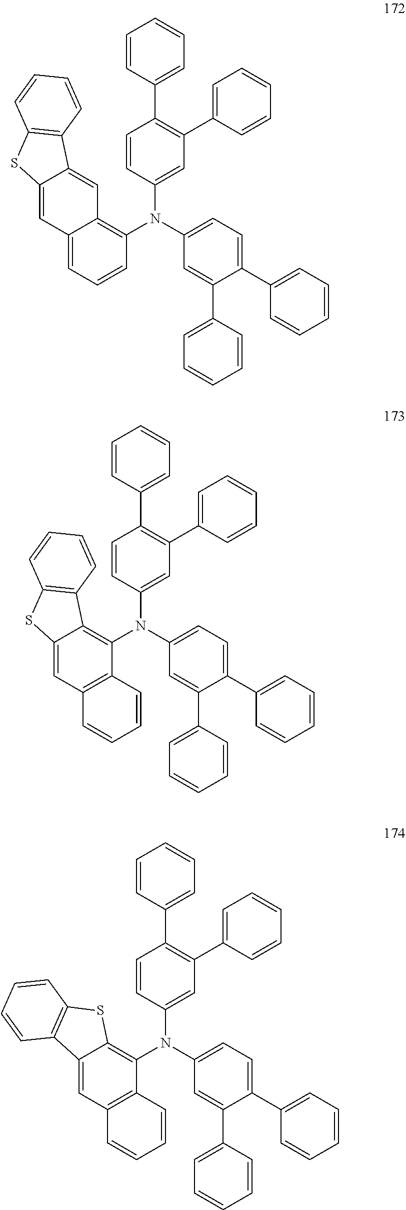 [SMILES: c1ccc(-c2ccc(N(c3ccc(-c4ccccc4)c(-c4ccccc4)c3)c3cccc4cc5sc6ccccc6c5cc34)cc2-c2ccccc2)cc1 ; c1ccc(-c2ccc(N(c3ccc(-c4ccccc4)c(-c4ccccc4)c3)c3c4ccccc4cc4sc5ccccc5c34)cc2-c2ccccc2)cc1 ; c1ccc(-c2ccc(N(c3ccc(-c4ccccc4)c(-c4ccccc4)c3)c3c4ccccc4cc4c3sc3ccccc34)cc2-c2ccccc2)cc1] 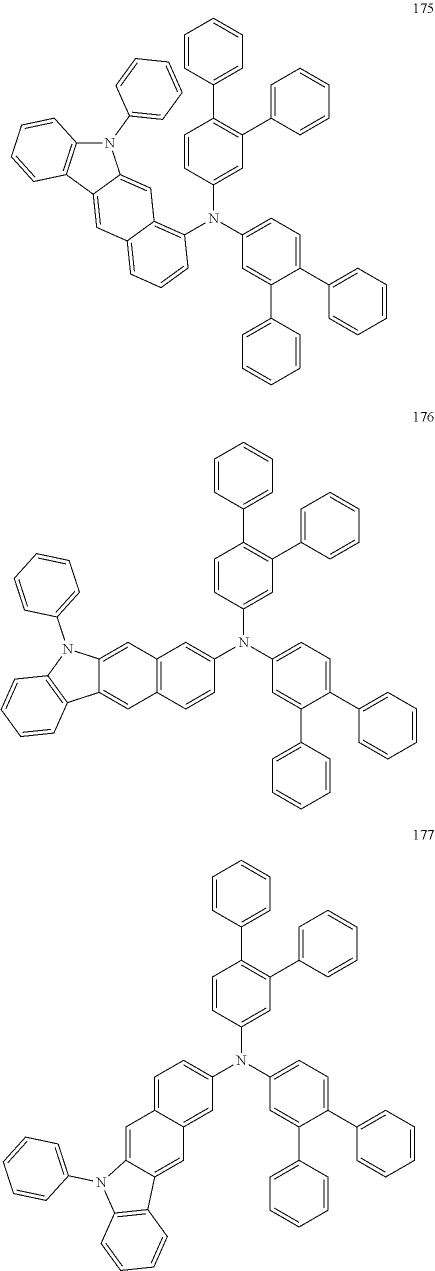 [SMILES: c1ccc(-c2ccc(N(c3ccc(-c4ccccc4)c(-c4ccccc4)c3)c3cccc4cc5c6ccccc6n(-c6ccccc6)c5cc34)cc2-c2ccccc2)cc1 ; c1ccc(-c2ccc(N(c3ccc(-c4ccccc4)c(-c4ccccc4)c3)c3ccc4cc5c6ccccc6n(-c6ccccc6)c5cc4c3)cc2-c2ccccc2)cc1 ; c1ccc(-c2ccc(N(c3ccc(-c4ccccc4)c(-c4ccccc4)c3)c3ccc4cc5c(cc4c3)c3ccccc3n5-c3ccccc3)cc2-c2ccccc2)cc1] 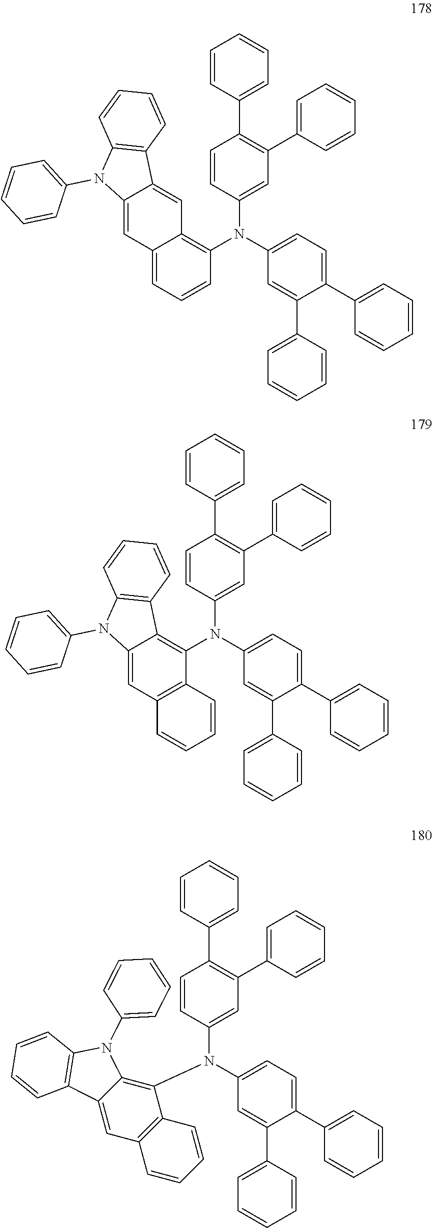 [SMILES: c1ccc(-c2ccc(N(c3ccc(-c4ccccc4)c(-c4ccccc4)c3)c3cccc4cc5c(cc34)c3ccccc3n5-c3ccccc3)cc2-c2ccccc2)cc1 ; c1ccc(-c2ccc(N(c3ccc(-c4ccccc4)c(-c4ccccc4)c3)c3c4ccccc4cc4c3c3ccccc3n4-c3ccccc3)cc2-c2ccccc2)cc1 ; c1ccc(-c2ccc(N(c3ccc(-c4ccccc4)c(-c4ccccc4)c3)c3c4ccccc4cc4c5ccccc5n(-c5ccccc5)c34)cc2-c2ccccc2)cc1] 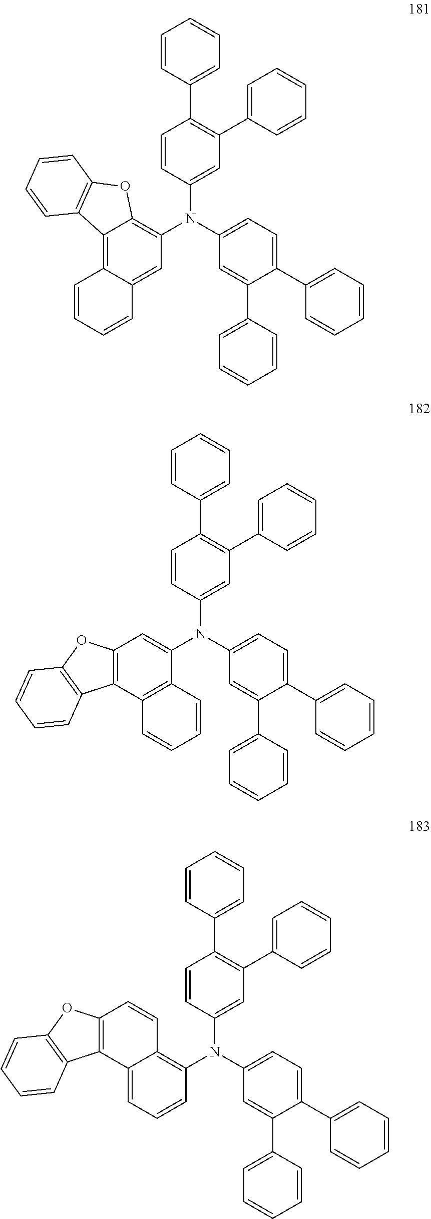 [SMILES: c1ccc(-c2ccc(N(c3ccc(-c4ccccc4)c(-c4ccccc4)c3)c3cc4ccccc4c4c3oc3ccccc34)cc2-c2ccccc2)cc1 ; c1ccc(-c2ccc(N(c3ccc(-c4ccccc4)c(-c4ccccc4)c3)c3cc4oc5ccccc5c4c4ccccc34)cc2-c2ccccc2)cc1 ; c1ccc(-c2ccc(N(c3ccc(-c4ccccc4)c(-c4ccccc4)c3)c3cccc4c3ccc3oc5ccccc5c34)cc2-c2ccccc2)cc1] 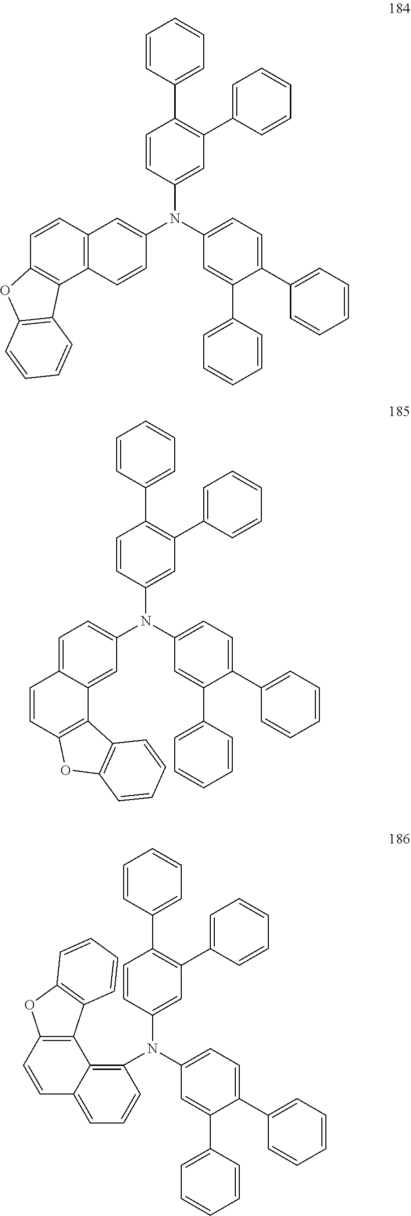 [SMILES: c1ccc(-c2ccc(N(c3ccc(-c4ccccc4)c(-c4ccccc4)c3)c3ccc4c(ccc5oc6ccccc6c54)c3)cc2-c2ccccc2)cc1 ; c1ccc(-c2ccc(N(c3ccc(-c4ccccc4)c(-c4ccccc4)c3)c3ccc4ccc5oc6ccccc6c5c4c3)cc2-c2ccccc2)cc1 ; c1ccc(-c2ccc(N(c3ccc(-c4ccccc4)c(-c4ccccc4)c3)c3cccc4ccc5oc6ccccc6c5c34)cc2-c2ccccc2)cc1] 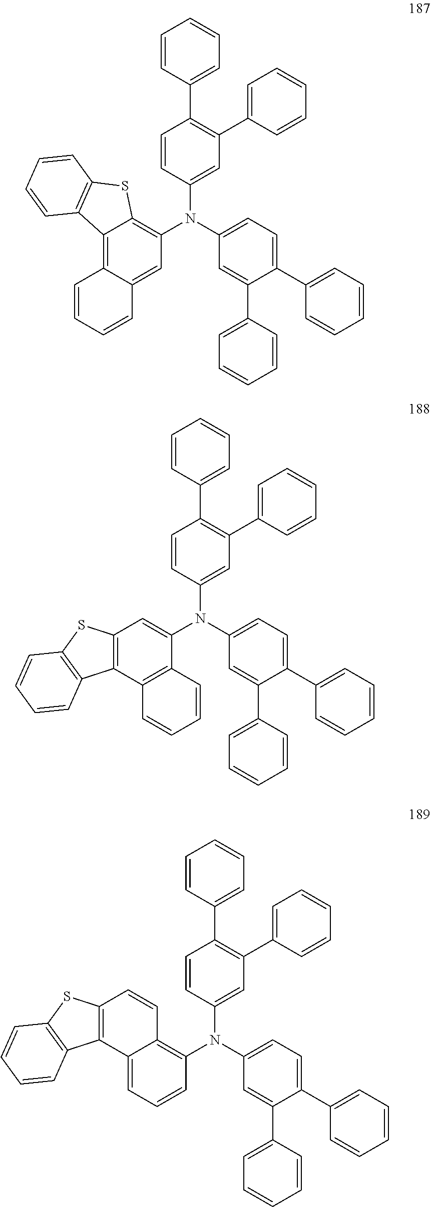 [SMILES: c1ccc(-c2ccc(N(c3ccc(-c4ccccc4)c(-c4ccccc4)c3)c3cc4ccccc4c4c3sc3ccccc34)cc2-c2ccccc2)cc1 ; c1ccc(-c2ccc(N(c3ccc(-c4ccccc4)c(-c4ccccc4)c3)c3cc4sc5ccccc5c4c4ccccc34)cc2-c2ccccc2)cc1 ; c1ccc(-c2ccc(N(c3ccc(-c4ccccc4)c(-c4ccccc4)c3)c3cccc4c3ccc3sc5ccccc5c34)cc2-c2ccccc2)cc1] 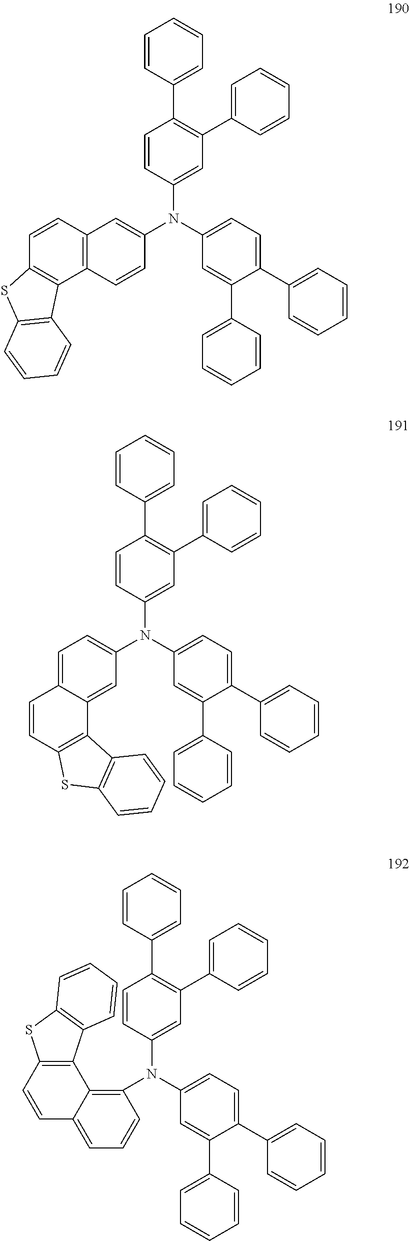 [SMILES: c1ccc(-c2ccc(N(c3ccc(-c4ccccc4)c(-c4ccccc4)c3)c3ccc4c(ccc5sc6ccccc6c54)c3)cc2-c2ccccc2)cc1 ; c1ccc(-c2ccc(N(c3ccc(-c4ccccc4)c(-c4ccccc4)c3)c3ccc4ccc5sc6ccccc6c5c4c3)cc2-c2ccccc2)cc1 ; c1ccc(-c2ccc(N(c3ccc(-c4ccccc4)c(-c4ccccc4)c3)c3cccc4ccc5sc6ccccc6c5c34)cc2-c2ccccc2)cc1] 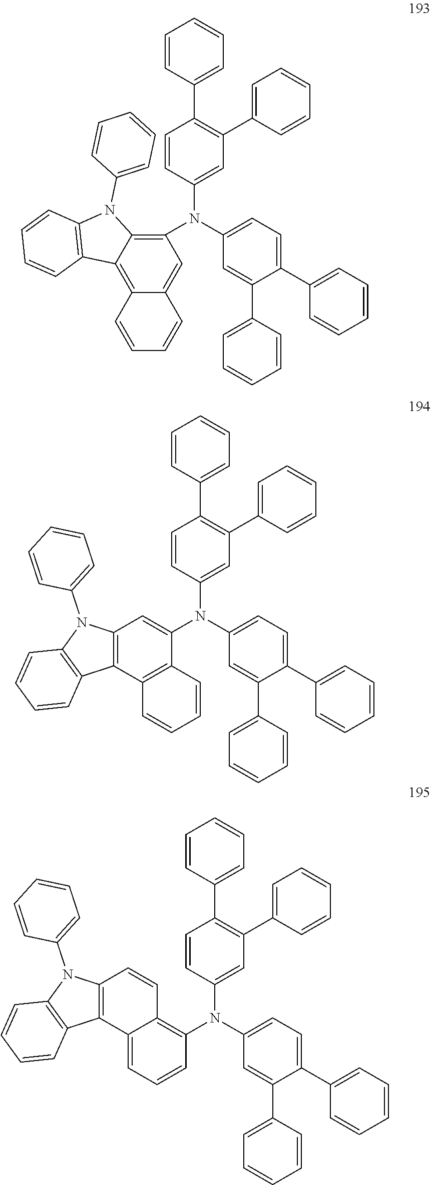 [SMILES: c1ccc(-c2ccc(N(c3ccc(-c4ccccc4)c(-c4ccccc4)c3)c3cc4ccccc4c4c5ccccc5n(-c5ccccc5)c34)cc2-c2ccccc2)cc1 ; c1ccc(-c2ccc(N(c3ccc(-c4ccccc4)c(-c4ccccc4)c3)c3cc4c(c5ccccc35)c3ccccc3n4-c3ccccc3)cc2-c2ccccc2)cc1 ; c1ccc(-c2ccc(N(c3ccc(-c4ccccc4)c(-c4ccccc4)c3)c3cccc4c3ccc3c4c4ccccc4n3-c3ccccc3)cc2-c2ccccc2)cc1] 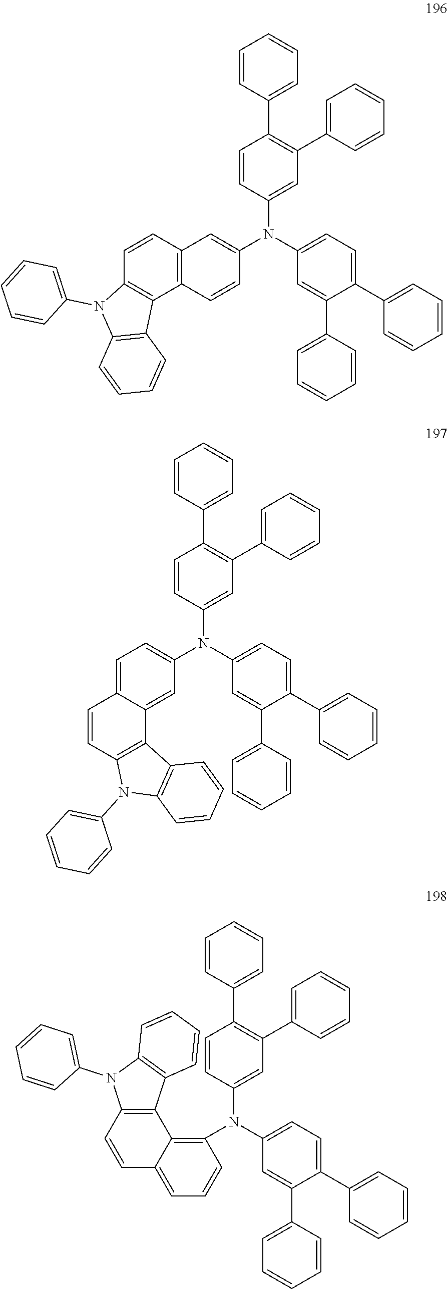 [SMILES: c1ccc(-c2ccc(N(c3ccc(-c4ccccc4)c(-c4ccccc4)c3)c3ccc4c(ccc5c4c4ccccc4n5-c4ccccc4)c3)cc2-c2ccccc2)cc1 ; c1ccc(-c2ccc(N(c3ccc(-c4ccccc4)c(-c4ccccc4)c3)c3ccc4ccc5c(c4c3)c3ccccc3n5-c3ccccc3)cc2-c2ccccc2)cc1 ; c1ccc(-c2ccc(N(c3ccc(-c4ccccc4)c(-c4ccccc4)c3)c3cccc4ccc5c(c6ccccc6n5-c5ccccc5)c34)cc2-c2ccccc2)cc1] 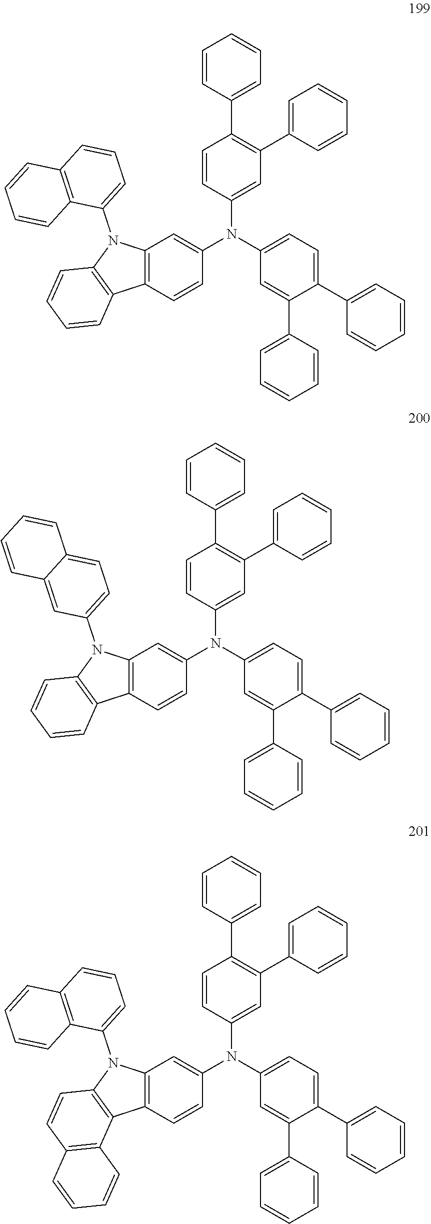 [SMILES: c1ccc(-c2ccc(N(c3ccc(-c4ccccc4)c(-c4ccccc4)c3)c3ccc4c5ccccc5n(-c5cccc6ccccc56)c4c3)cc2-c2ccccc2)cc1 ; c1ccc(-c2ccc(N(c3ccc(-c4ccccc4)c(-c4ccccc4)c3)c3ccc4c5ccccc5n(-c5ccc6ccccc6c5)c4c3)cc2-c2ccccc2)cc1 ; c1ccc(-c2ccc(N(c3ccc(-c4ccccc4)c(-c4ccccc4)c3)c3ccc4c5c6ccccc6ccc5n(-c5cccc6ccccc56)c4c3)cc2-c2ccccc2)cc1] 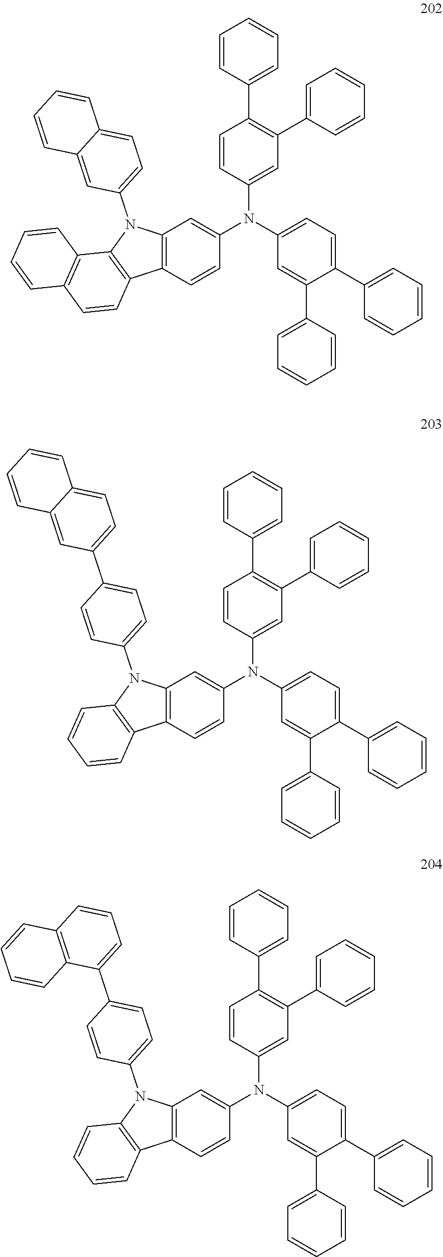 [SMILES: c1ccc(-c2ccc(N(c3ccc(-c4ccccc4)c(-c4ccccc4)c3)c3ccc4c5ccc6ccccc6c5n(-c5ccc6ccccc6c5)c4c3)cc2-c2ccccc2)cc1 ; c1ccc(-c2ccc(N(c3ccc(-c4ccccc4)c(-c4ccccc4)c3)c3ccc4c5ccccc5n(-c5ccc(-c6ccc7ccccc7c6)cc5)c4c3)cc2-c2ccccc2)cc1 ; c1ccc(-c2ccc(N(c3ccc(-c4ccccc4)c(-c4ccccc4)c3)c3ccc4c5ccccc5n(-c5ccc(-c6cccc7ccccc67)cc5)c4c3)cc2-c2ccccc2)cc1] 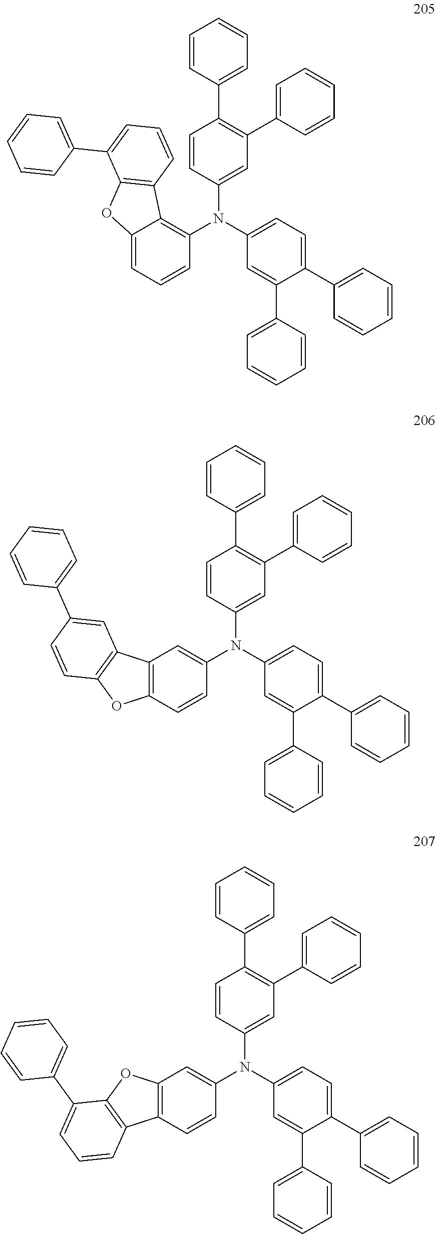 [SMILES: c1ccc(-c2ccc(N(c3ccc(-c4ccccc4)c(-c4ccccc4)c3)c3cccc4oc5c(-c6ccccc6)cccc5c34)cc2-c2ccccc2)cc1 ; c1ccc(-c2ccc3oc4ccc(N(c5ccc(-c6ccccc6)c(-c6ccccc6)c5)c5ccc(-c6ccccc6)c(-c6ccccc6)c5)cc4c3c2)cc1 ; c1ccc(-c2ccc(N(c3ccc(-c4ccccc4)c(-c4ccccc4)c3)c3ccc4c(c3)oc3c(-c5ccccc5)cccc34)cc2-c2ccccc2)cc1] 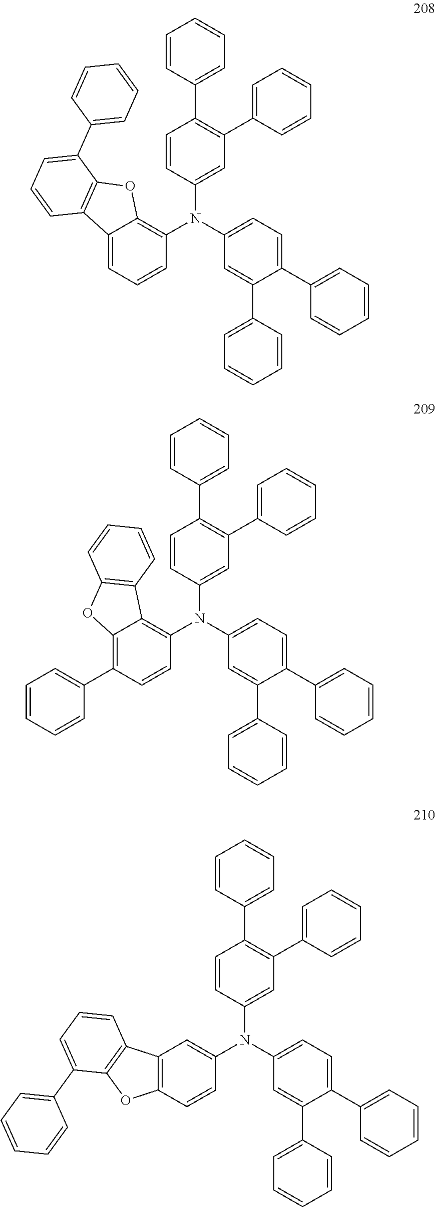 [SMILES: c1ccc(-c2ccc(N(c3ccc(-c4ccccc4)c(-c4ccccc4)c3)c3cccc4c3oc3c(-c5ccccc5)cccc34)cc2-c2ccccc2)cc1 ; c1ccc(-c2ccc(N(c3ccc(-c4ccccc4)c(-c4ccccc4)c3)c3ccc(-c4ccccc4)c4oc5ccccc5c34)cc2-c2ccccc2)cc1 ; c1ccc(-c2ccc(N(c3ccc(-c4ccccc4)c(-c4ccccc4)c3)c3ccc4oc5c(-c6ccccc6)cccc5c4c3)cc2-c2ccccc2)cc1] 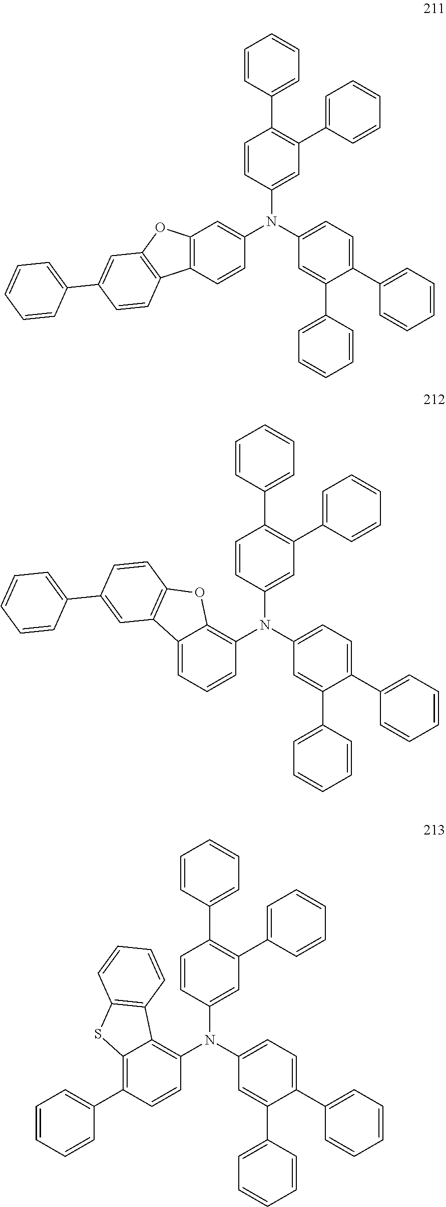 [SMILES: c1ccc(-c2ccc3c(c2)oc2cc(N(c4ccc(-c5ccccc5)c(-c5ccccc5)c4)c4ccc(-c5ccccc5)c(-c5ccccc5)c4)ccc23)cc1 ; c1ccc(-c2ccc3oc4c(N(c5ccc(-c6ccccc6)c(-c6ccccc6)c5)c5ccc(-c6ccccc6)c(-c6ccccc6)c5)cccc4c3c2)cc1 ; c1ccc(-c2ccc(N(c3ccc(-c4ccccc4)c(-c4ccccc4)c3)c3ccc(-c4ccccc4)c4sc5ccccc5c34)cc2-c2ccccc2)cc1] 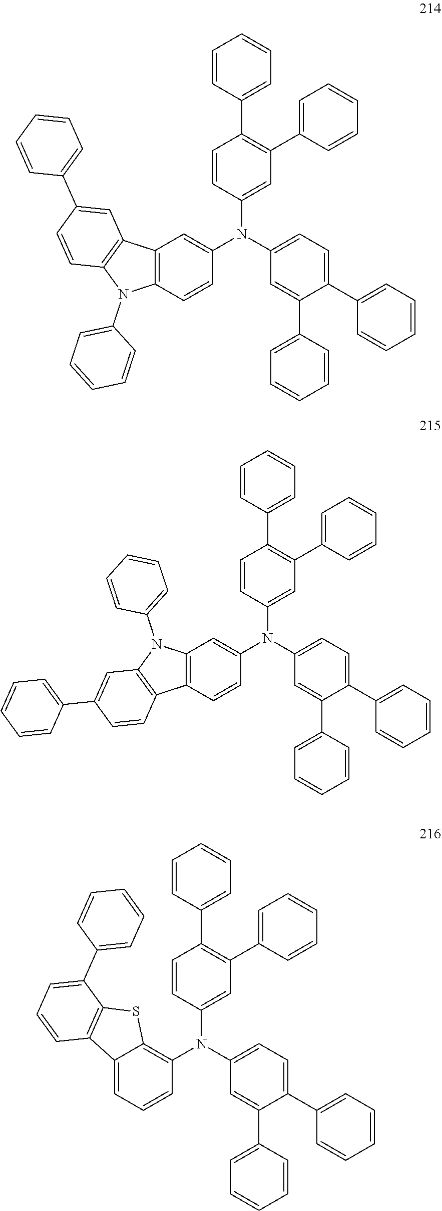 [SMILES: c1ccc(-c2ccc3c(c2)c2cc(N(c4ccc(-c5ccccc5)c(-c5ccccc5)c4)c4ccc(-c5ccccc5)c(-c5ccccc5)c4)ccc2n3-c2ccccc2)cc1 ; c1ccc(-c2ccc3c4ccc(N(c5ccc(-c6ccccc6)c(-c6ccccc6)c5)c5ccc(-c6ccccc6)c(-c6ccccc6)c5)cc4n(-c4ccccc4)c3c2)cc1 ; c1ccc(-c2ccc(N(c3ccc(-c4ccccc4)c(-c4ccccc4)c3)c3cccc4c3sc3c(-c5ccccc5)cccc34)cc2-c2ccccc2)cc1] 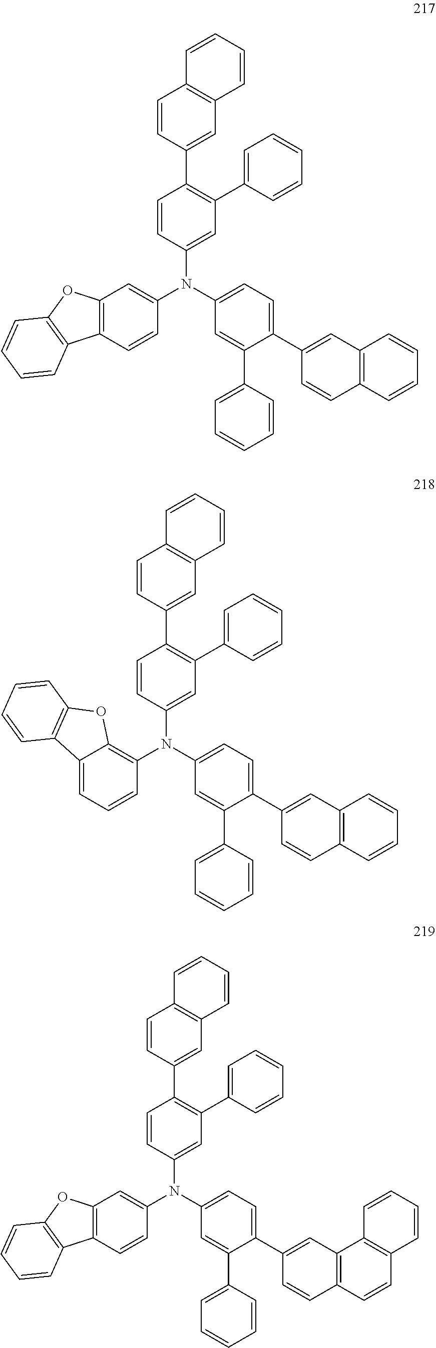 [SMILES: c1ccc(-c2cc(N(c3ccc(-c4ccc5ccccc5c4)c(-c4ccccc4)c3)c3ccc4c(c3)oc3ccccc34)ccc2-c2ccc3ccccc3c2)cc1 ; c1ccc(-c2cc(N(c3ccc(-c4ccc5ccccc5c4)c(-c4ccccc4)c3)c3cccc4c3oc3ccccc34)ccc2-c2ccc3ccccc3c2)cc1 ; c1ccc(-c2cc(N(c3ccc(-c4ccc5ccc6ccccc6c5c4)c(-c4ccccc4)c3)c3ccc4c(c3)oc3ccccc34)ccc2-c2ccc3ccccc3c2)cc1] 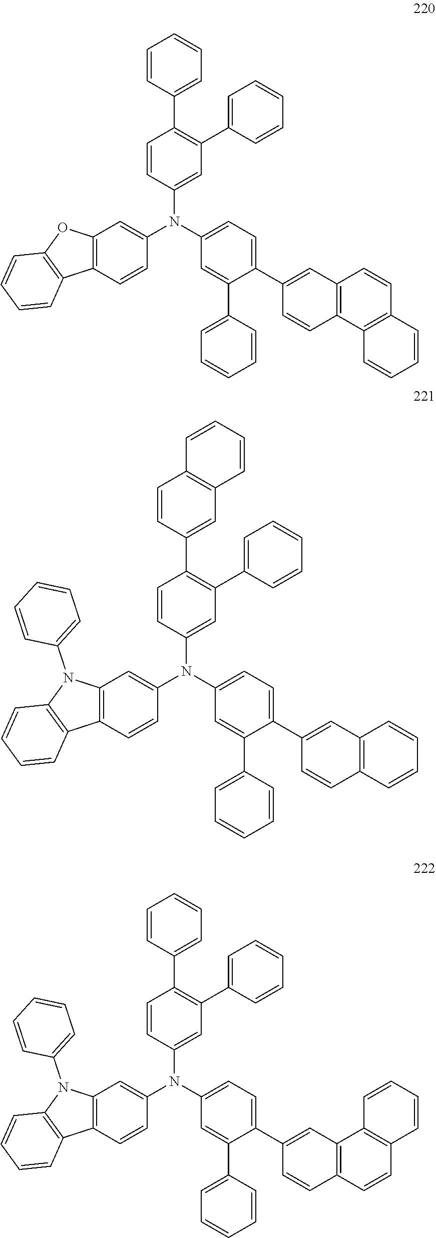 [SMILES: c1ccc(-c2ccc(N(c3ccc(-c4ccc5c(ccc6ccccc65)c4)c(-c4ccccc4)c3)c3ccc4c(c3)oc3ccccc34)cc2-c2ccccc2)cc1 ; c1ccc(-c2cc(N(c3ccc(-c4ccc5ccccc5c4)c(-c4ccccc4)c3)c3ccc4c5ccccc5n(-c5ccccc5)c4c3)ccc2-c2ccc3ccccc3c2)cc1 ; c1ccc(-c2ccc(N(c3ccc(-c4ccc5ccc6ccccc6c5c4)c(-c4ccccc4)c3)c3ccc4c5ccccc5n(-c5ccccc5)c4c3)cc2-c2ccccc2)cc1] 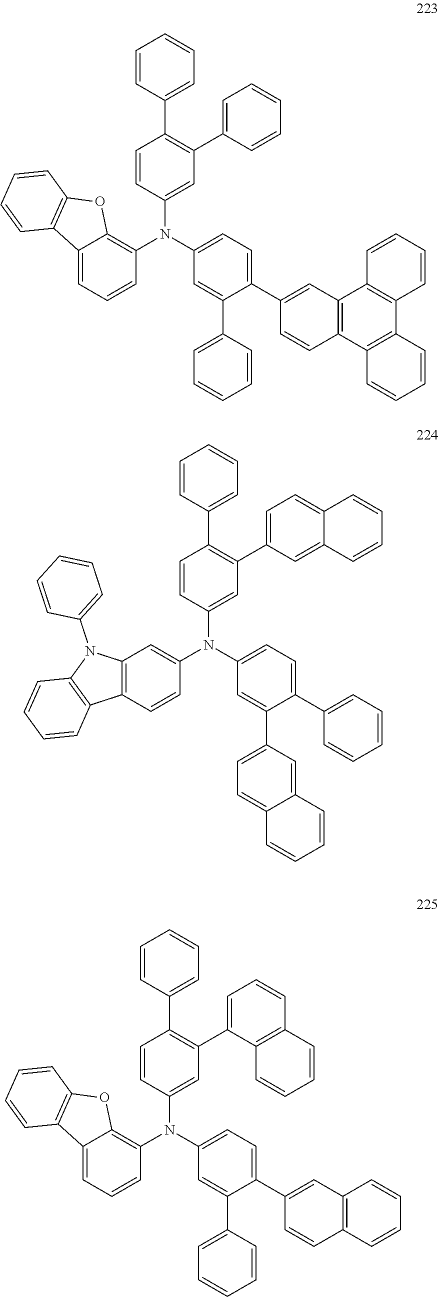 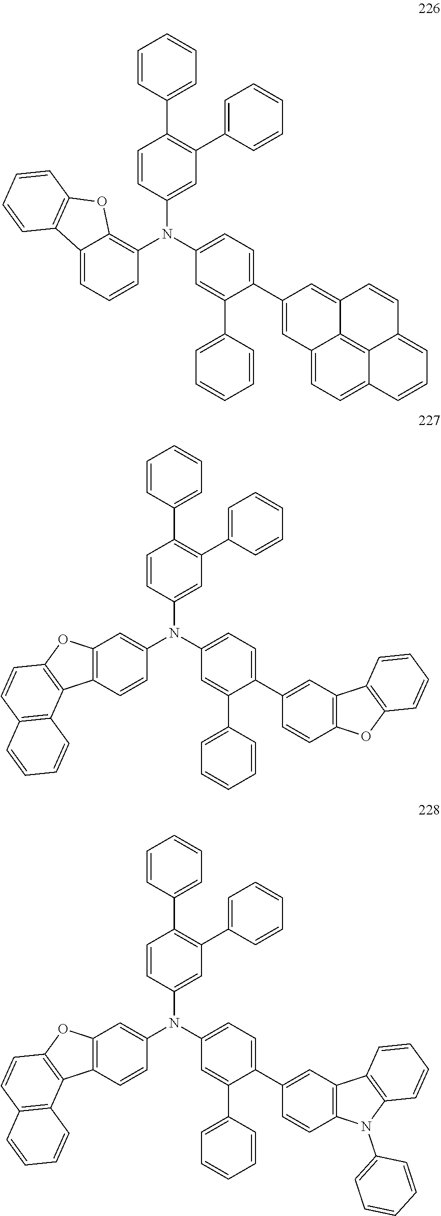 [SMILES: c1ccc(-c2ccc(N(c3ccc(-c4cc5ccc6cccc7ccc(c4)c5c67)c(-c4ccccc4)c3)c3cccc4c3oc3ccccc34)cc2-c2ccccc2)cc1 ; c1ccc(-c2ccc(N(c3ccc(-c4ccc5oc6ccccc6c5c4)c(-c4ccccc4)c3)c3ccc4c(c3)oc3ccc5ccccc5c34)cc2-c2ccccc2)cc1 ; c1ccc(-c2ccc(N(c3ccc(-c4ccc5c(c4)c4ccccc4n5-c4ccccc4)c(-c4ccccc4)c3)c3ccc4c(c3)oc3ccc5ccccc5c34)cc2-c2ccccc2)cc1] 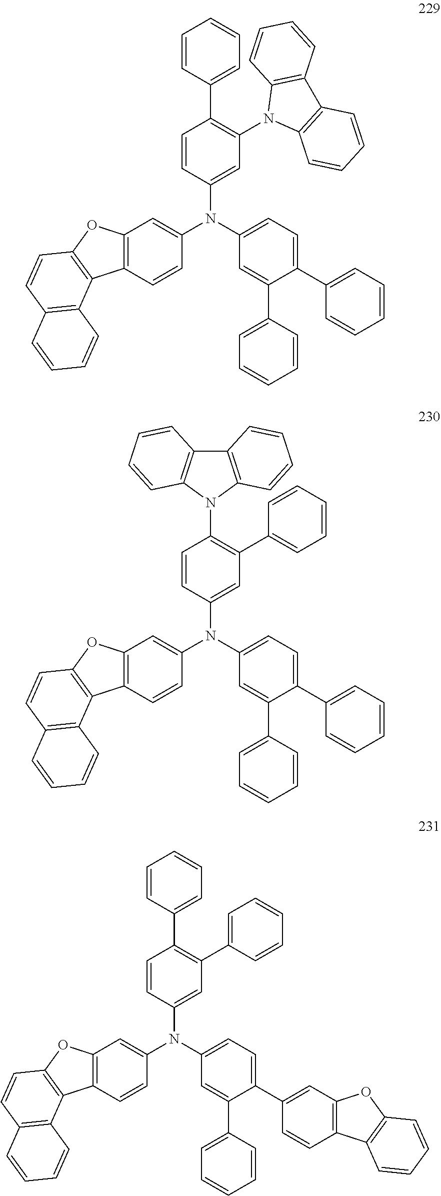 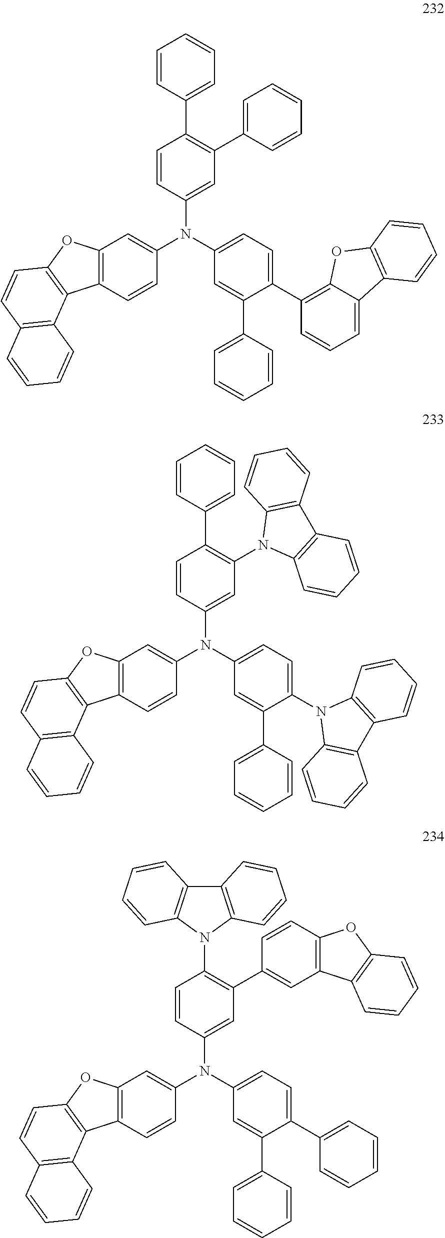 [SMILES: c1ccc(-c2ccc(N(c3ccc(-c4cccc5c4oc4ccccc45)c(-c4ccccc4)c3)c3ccc4c(c3)oc3ccc5ccccc5c34)cc2-c2ccccc2)cc1 ; c1ccc(-c2cc(N(c3ccc(-c4ccccc4)c(-n4c5ccccc5c5ccccc54)c3)c3ccc4c(c3)oc3ccc5ccccc5c34)ccc2-n2c3ccccc3c3ccccc32)cc1 ; c1ccc(-c2ccc(N(c3ccc(-n4c5ccccc5c5ccccc54)c(-c4ccc5oc6ccccc6c5c4)c3)c3ccc4c(c3)oc3ccc5ccccc5c34)cc2-c2ccccc2)cc1] 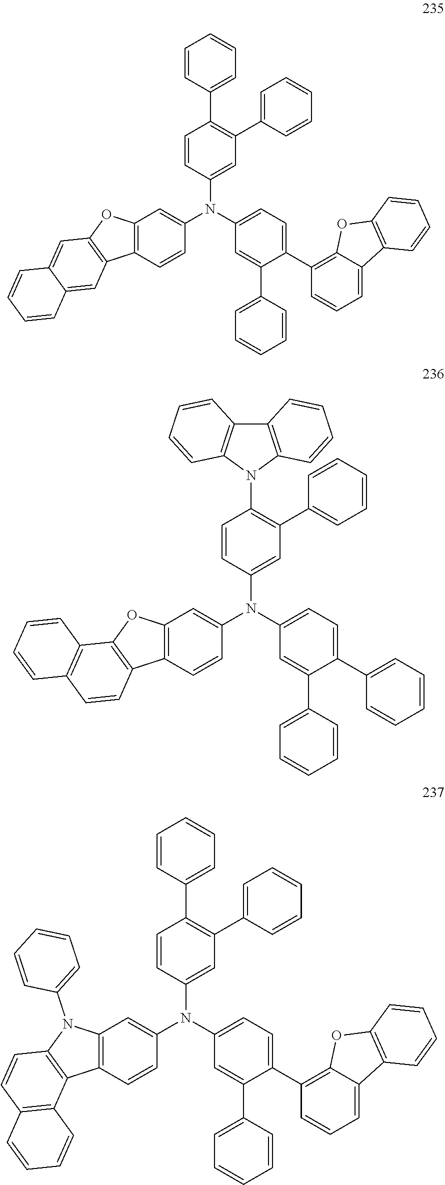 [SMILES: c1ccc(-c2ccc(N(c3ccc(-c4cccc5c4oc4ccccc45)c(-c4ccccc4)c3)c3ccc4c(c3)oc3cc5ccccc5cc34)cc2-c2ccccc2)cc1 ; c1ccc(-c2ccc(N(c3ccc(-n4c5ccccc5c5ccccc54)c(-c4ccccc4)c3)c3ccc4c(c3)oc3c5ccccc5ccc43)cc2-c2ccccc2)cc1 ; c1ccc(-c2ccc(N(c3ccc(-c4cccc5c4oc4ccccc45)c(-c4ccccc4)c3)c3ccc4c5c6ccccc6ccc5n(-c5ccccc5)c4c3)cc2-c2ccccc2)cc1] 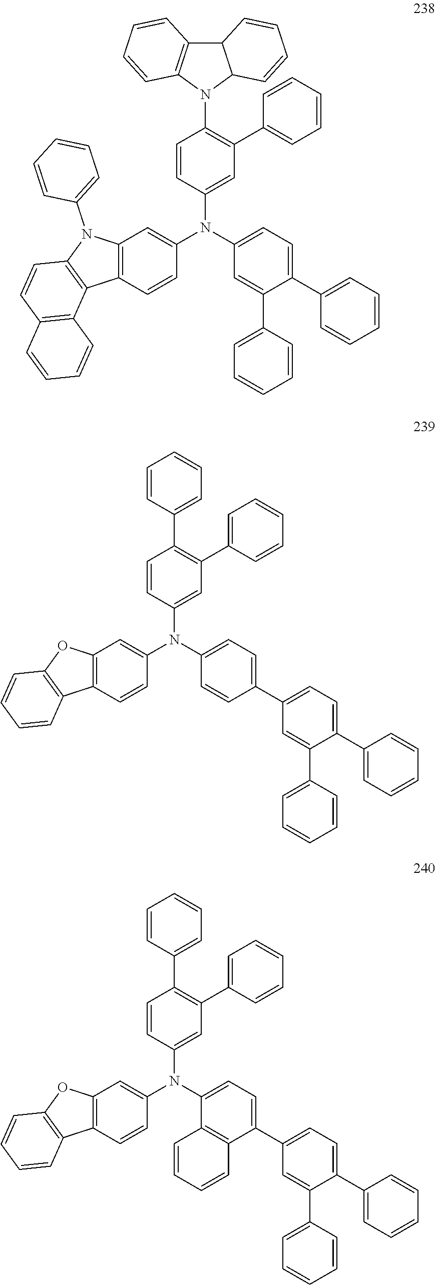 [SMILES: C1=CC2c3ccccc3N(c3ccc(N(c4ccc(-c5ccccc5)c(-c5ccccc5)c4)c4ccc5c6c7ccccc7ccc6n(-c6ccccc6)c5c4)cc3-c3ccccc3)C2C=C1 ; c1ccc(-c2ccc(-c3ccc(N(c4ccc(-c5ccccc5)c(-c5ccccc5)c4)c4ccc5c(c4)oc4ccccc45)cc3)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4ccccc34)cc2-c2ccccc2)cc1] 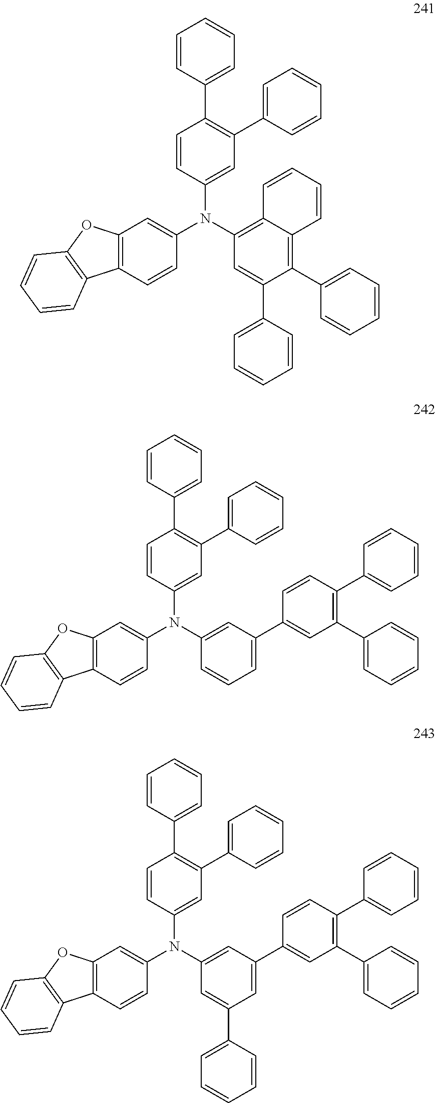 [SMILES: c1ccc(-c2ccc(N(c3ccc4c(c3)oc3ccccc34)c3cc(-c4ccccc4)c(-c4ccccc4)c4ccccc34)cc2-c2ccccc2)cc1 ; c1ccc(-c2ccc(-c3cccc(N(c4ccc(-c5ccccc5)c(-c5ccccc5)c4)c4ccc5c(c4)oc4ccccc45)c3)cc2-c2ccccc2)cc1 ; c1ccc(-c2cc(-c3ccc(-c4ccccc4)c(-c4ccccc4)c3)cc(N(c3ccc(-c4ccccc4)c(-c4ccccc4)c3)c3ccc4c(c3)oc3ccccc34)c2)cc1] 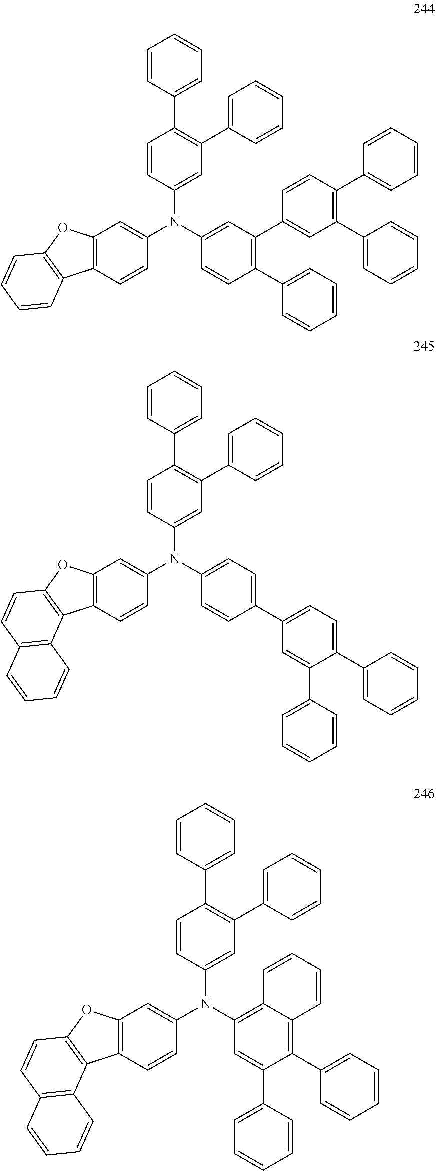 [SMILES: c1ccc(-c2ccc(-c3cc(N(c4ccc(-c5ccccc5)c(-c5ccccc5)c4)c4ccc5c(c4)oc4ccccc45)ccc3-c3ccccc3)cc2-c2ccccc2)cc1 ; c1ccc(-c2ccc(-c3ccc(N(c4ccc(-c5ccccc5)c(-c5ccccc5)c4)c4ccc5c(c4)oc4ccc6ccccc6c45)cc3)cc2-c2ccccc2)cc1 ; c1ccc(-c2ccc(N(c3ccc4c(c3)oc3ccc5ccccc5c34)c3cc(-c4ccccc4)c(-c4ccccc4)c4ccccc34)cc2-c2ccccc2)cc1] 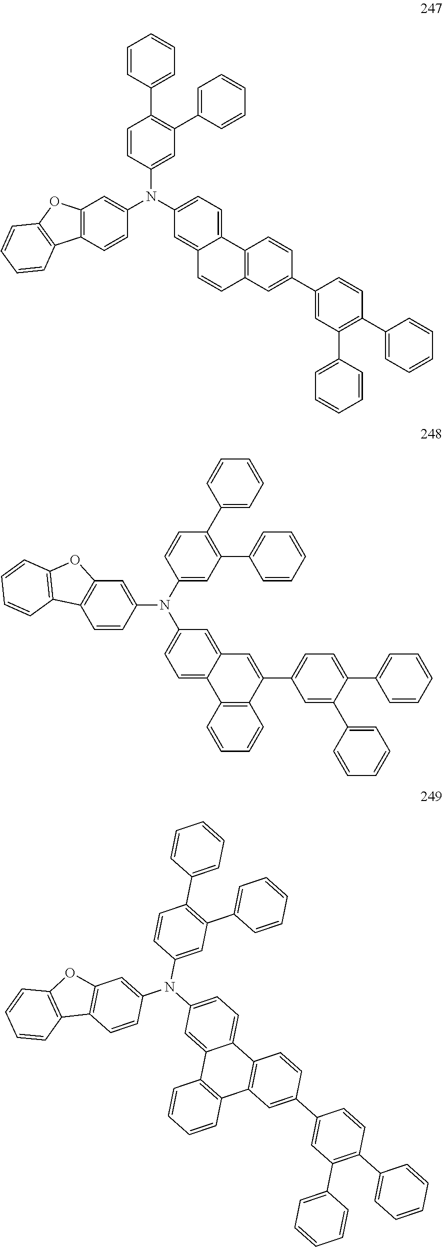 [SMILES: c1ccc(-c2ccc(-c3ccc4c(ccc5cc(N(c6ccc(-c7ccccc7)c(-c7ccccc7)c6)c6ccc7c(c6)oc6ccccc67)ccc54)c3)cc2-c2ccccc2)cc1 ; c1ccc(-c2ccc(-c3cc4cc(N(c5ccc(-c6ccccc6)c(-c6ccccc6)c5)c5ccc6c(c5)oc5ccccc56)ccc4c4ccccc34)cc2-c2ccccc2)cc1 ; c1ccc(-c2ccc(-c3ccc4c5ccc(N(c6ccc(-c7ccccc7)c(-c7ccccc7)c6)c6ccc7c(c6)oc6ccccc67)cc5c5ccccc5c4c3)cc2-c2ccccc2)cc1] 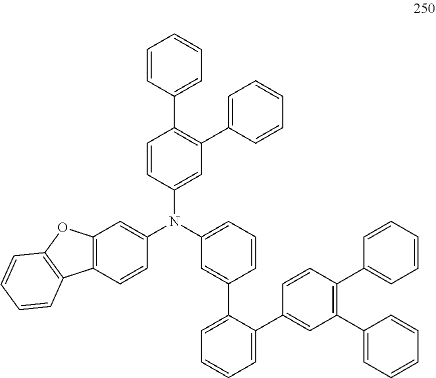 [SMILES: c1ccc(-c2ccc(-c3ccccc3-c3cccc(N(c4ccc(-c5ccccc5)c(-c5ccccc5)c4)c4ccc5c(c4)oc4ccccc45)c3)cc2-c2ccccc2)cc1] 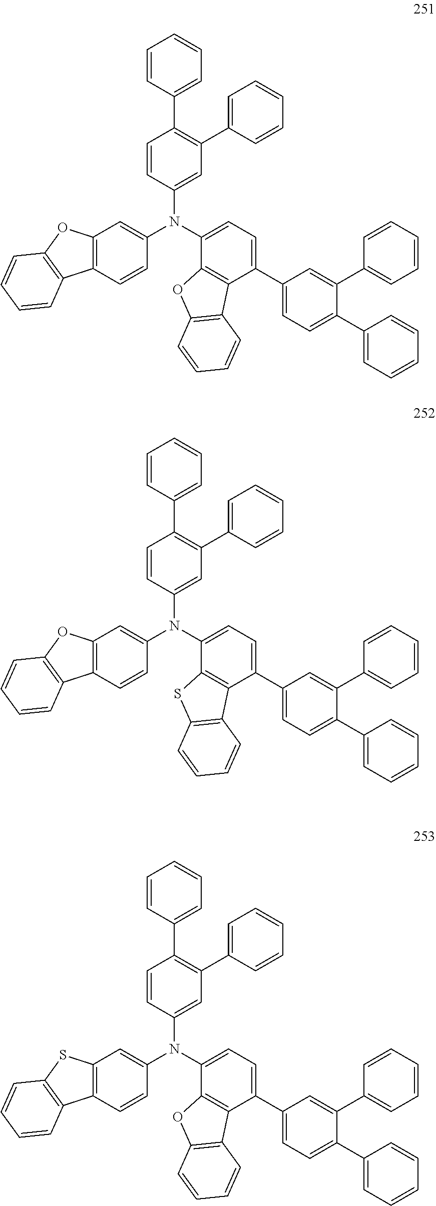 [SMILES: c1ccc(-c2ccc(-c3ccc(N(c4ccc(-c5ccccc5)c(-c5ccccc5)c4)c4ccc5c(c4)oc4ccccc45)c4oc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4sc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)sc4ccccc45)c4oc5ccccc5c34)cc2-c2ccccc2)cc1] 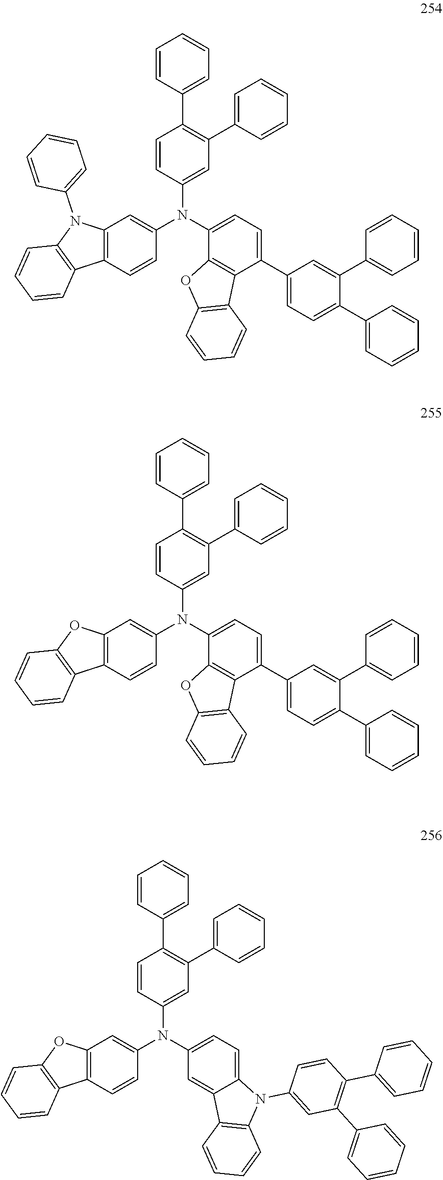 [SMILES: c1ccc(-c2ccc(-c3ccc(N(c4ccc(-c5ccccc5)c(-c5ccccc5)c4)c4ccc5c6ccccc6n(-c6ccccc6)c5c4)c4oc5ccccc5c34)cc2-c2ccccc2)cc1 ; c1ccc(-c2ccc(-c3ccc(N(c4ccc(-c5ccccc5)c(-c5ccccc5)c4)c4ccc5c(c4)oc4ccccc45)c4oc5ccccc5c34)cc2-c2ccccc2)cc1 ; c1ccc(-c2ccc(N(c3ccc4c(c3)oc3ccccc34)c3ccc4c(c3)c3ccccc3n4-c3ccc(-c4ccccc4)c(-c4ccccc4)c3)cc2-c2ccccc2)cc1] 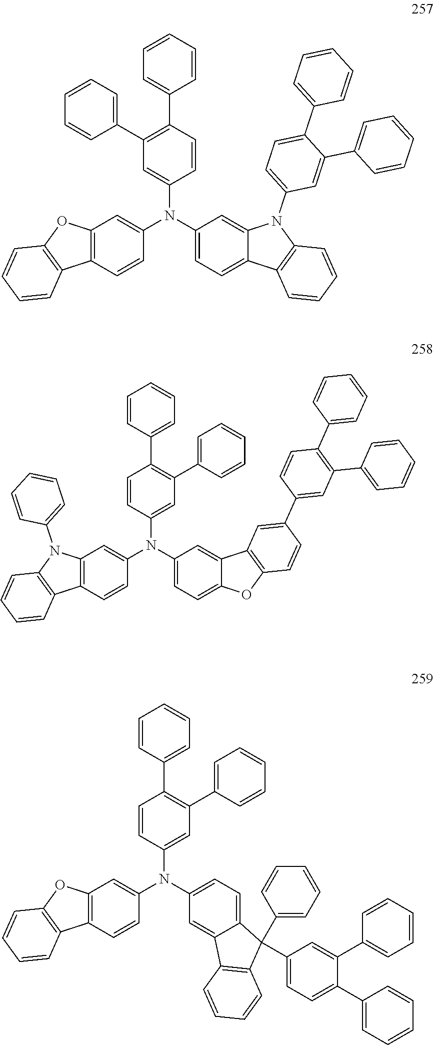 [SMILES: c1ccc(-c2ccc(N(c3ccc4c(c3)oc3ccccc34)c3ccc4c5ccccc5n(-c5ccc(-c6ccccc6)c(-c6ccccc6)c5)c4c3)cc2-c2ccccc2)cc1 ; c1ccc(-c2ccc(-c3ccc4oc5ccc(N(c6ccc(-c7ccccc7)c(-c7ccccc7)c6)c6ccc7c8ccccc8n(-c8ccccc8)c7c6)cc5c4c3)cc2-c2ccccc2)cc1 ; c1ccc(-c2ccc(N(c3ccc4c(c3)-c3ccccc3C4(c3ccccc3)c3ccc(-c4ccccc4)c(-c4ccccc4)c3)c3ccc4c(c3)oc3ccccc34)cc2-c2ccccc2)cc1] 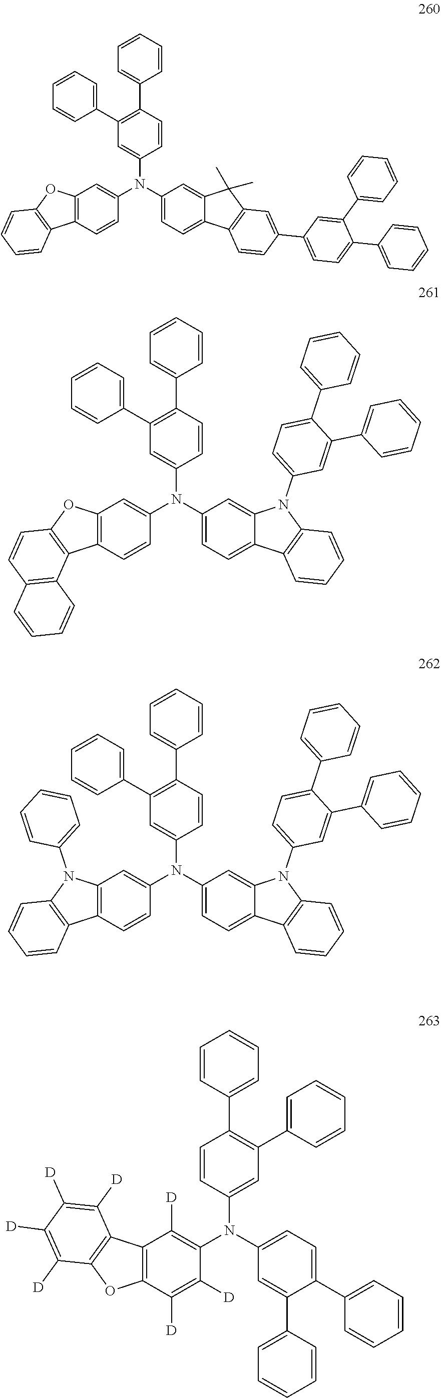 [SMILES: CC1(C)c2cc(-c3ccc(-c4ccccc4)c(-c4ccccc4)c3)ccc2-c2ccc(N(c3ccc(-c4ccccc4)c(-c4ccccc4)c3)c3ccc4c(c3)oc3ccccc34)cc21 ; c1ccc(-c2ccc(N(c3ccc4c(c3)oc3ccc5ccccc5c34)c3ccc4c5ccccc5n(-c5ccc(-c6ccccc6)c(-c6ccccc6)c5)c4c3)cc2-c2ccccc2)cc1 ; c1ccc(-c2ccc(N(c3ccc4c5ccccc5n(-c5ccccc5)c4c3)c3ccc4c5ccccc5n(-c5ccc(-c6ccccc6)c(-c6ccccc6)c5)c4c3)cc2-c2ccccc2)cc1 ; [2H]c1c([2H])c([2H])c2c(oc3c([2H])c([2H])c(N(c4ccc(-c5ccccc5)c(-c5ccccc5)c4)c4ccc(-c5ccccc5)c(-c5ccccc5)c4)c([2H])c32)c1[2H]] 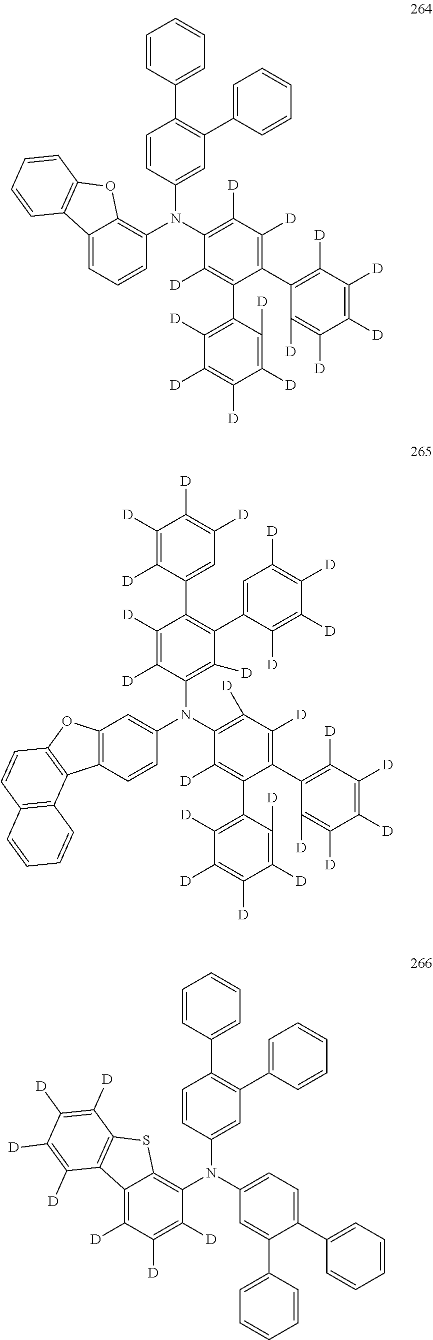 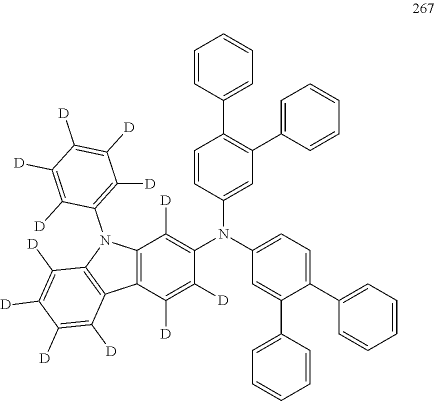 [SMILES: [2H]c1c([2H])c([2H])c(-n2c3c([2H])c([2H])c([2H])c([2H])c3c3c([2H])c([2H])c(N(c4ccc(-c5ccccc5)c(-c5ccccc5)c4)c4ccc(-c5ccccc5)c(-c5ccccc5)c4)c([2H])c32)c([2H])c1[2H]]
wherein in Compound Group 1,
D represents a deuterium atom.

10. An amine compound represented by Formula 1: 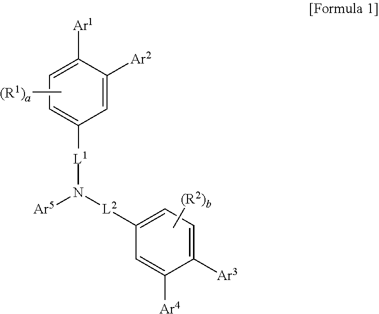
wherein in Formula 1,
L¹ and L² are each independently a direct linkage, a substituted or unsubstituted arylene group having 6 to 30 ring-forming carbon atoms, or a substituted or unsubstituted heteroarylene group having 5 to 30 ring-forming carbon atoms,
a and b are each independently an integer from 0 to 3,
R¹ and R² are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or bonded to an adjacent group to form a ring,
Ar¹ to Ar⁴ are each independently a substituted or unsubstituted aryl group having 6 to 30 ring-forming carbon atoms, or a substituted or unsubstituted heteroaryl group having 5 to 30 ring-forming carbon atoms, and
Ar⁵ is a group represented by one of Formula 2-1 to Formula 2-4: 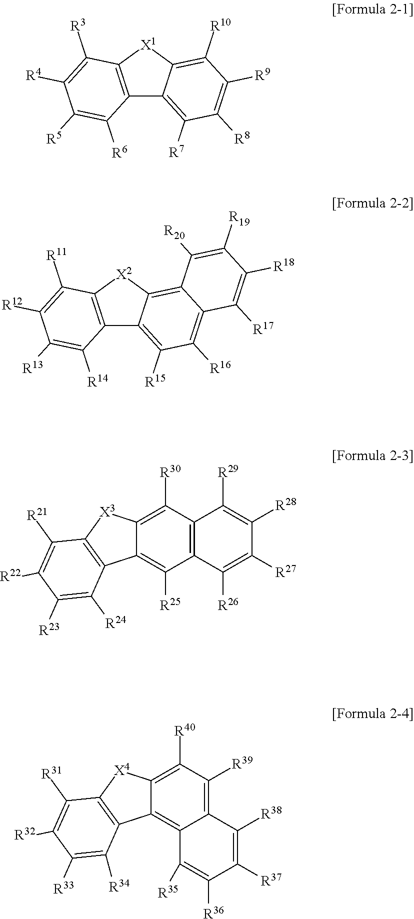
wherein in Formula 2-1 to Formula 2-4,
X¹ to X⁴ are each independently O, S, or N(R⁴¹),
R³ to R¹⁰ are each independently a hydrogen atom, a deuterium atom, or a substituted or unsubstituted aryl group having 6 to 30 ring-forming carbon atoms, and
R¹¹ to R⁴¹ are each independently a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms, except that:
in Formula 2-1, one of R³ to R¹⁰ and R⁴¹ is a position connected to Formula 1,
in Formula 2-2, one of R¹¹ to R²⁰ and R⁴¹ is a position connected to Formula 1,
in Formula 2-3, one of R²¹ to R³⁰ and R⁴¹ is a position connected to Formula 1, and
in Formula 2-4, one of R³¹ to R⁴¹ is a position connected to Formula 1.

11. The amine compound of claim 10, wherein the amine compound is represented by Formula 3-1 or Formula 3-2: 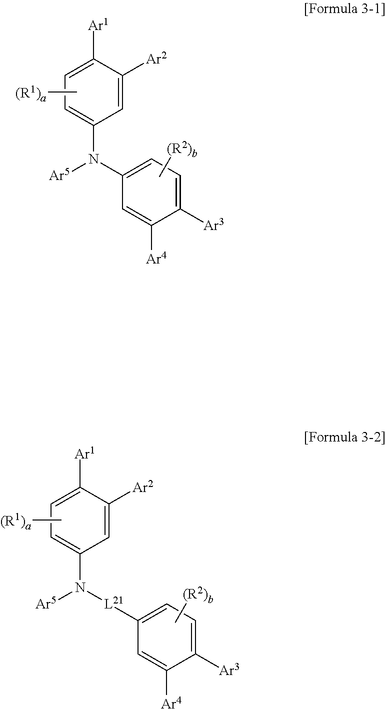
wherein in Formula 3-2,
L²¹ is a substituted or unsubstituted phenylene group, a substituted or unsubstituted biphenylene group, a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group, and
wherein in Formula 3-1 and Formula 3-2,
Ar¹ to Ar⁵, R¹, R², a, and b are the same as defined in Formula 1.

12. The amine compound of claim 10, wherein the amine compound is represented by Formula 4: 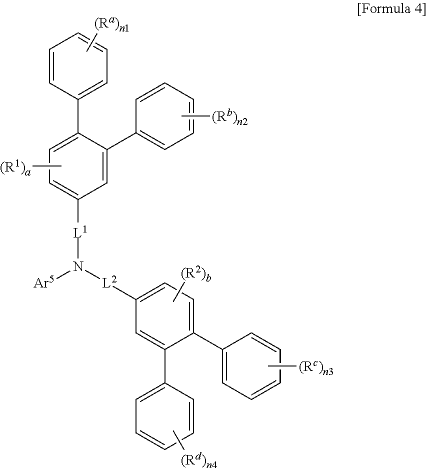
wherein in Formula 4,
n1 to n4 are each independently an integer from 0 to 5,
R^{a} to R^{d} are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring, and
Ar⁵, R¹, R², L¹, L², a, and b are the same as defined in Formula 1.

13. The amine compound of claim 10, wherein Ar¹ to Art are each independently a substituted or unsubstituted phenyl group, a substituted or unsubstituted naphthyl group, a substituted or unsubstituted phenanthryl group, a substituted or unsubstituted pyrenyl group, a substituted or unsubstituted triphenylene group, a substituted or unsubstituted carbazole group, a substituted unsubstituted dibenzothiophene group, or a substituted or unsubstituted dibenzofuran group.

14. The amine compound of claim 10, wherein L¹ and L² are each independently a direct linkage, a substituted or unsubstituted phenylene group, a substituted or unsubstituted biphenylene group a substituted or unsubstituted naphthylene group, a substituted or unsubstituted divalent phenanthrenyl group, a substituted or unsubstituted divalent triphenylene group, a substituted or unsubstituted divalent fluorenyl group, a substituted or unsubstituted divalent carbazole group, a substituted or unsubstituted divalent dibenzothiophene group, or a substituted or unsubstituted divalent dibenzofuran group.

15. The amine compound of claim 10, wherein R¹ and R² are each independently a hydrogen atom or a deuterium atom.

16. The amine compound of claim 10, wherein R³ to R⁴¹ are each independently a hydrogen atom, a deuterium atom, a substituted or unsubstituted phenyl group, or a substituted or unsubstituted naphthyl group.

17. The amine compound of claim 10, wherein when Ar⁵ is a group represented by Formula 2-1, Ar¹ to Art are each independently a substituted or unsubstituted phenyl group, a substituted or unsubstituted naphthyl group, a substituted or unsubstituted phenanthryl group, a substituted or unsubstituted pyrenyl group, or a substituted or unsubstituted triphenylene group.

18. A display device, comprising:
a circuit layer disposed on a base layer; and
a display element layer disposed on the circuit layer and including a light-emitting element, wherein
the light-emitting element includes:
a first electrode;
a second electrode disposed on the first electrode;
an emission layer disposed between the first electrode and second electrode; and
a hole transport region disposed between the first electrode and the emission layer, and
the hole transport region includes an amine compound represented by Formula 1: 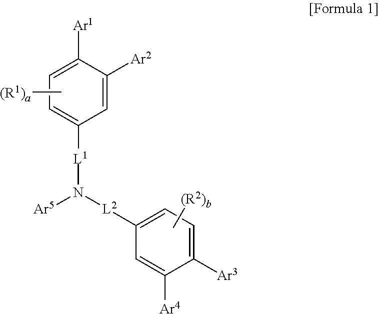
wherein in Formula 1,
L¹ and L² are each independently a direct linkage, a substituted or unsubstituted arylene group having 6 to 30 ring-forming carbon atoms, or a substituted or unsubstituted heteroarylene group having 5 to 30 ring-forming carbon atoms,
a and b are each independently an integer from 0 to 3,
R¹ and R² are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or bonded to an adjacent group to form a ring,
Ar¹ to Ar⁴ are each independently a substituted or unsubstituted aryl group having 6 to 30 ring-forming carbon atoms, or a substituted or unsubstituted heteroaryl group having 5 to 30 ring-forming carbon atoms, and
Ar⁵ is a group represented by one of Formula 2-1 to Formula 2-4: 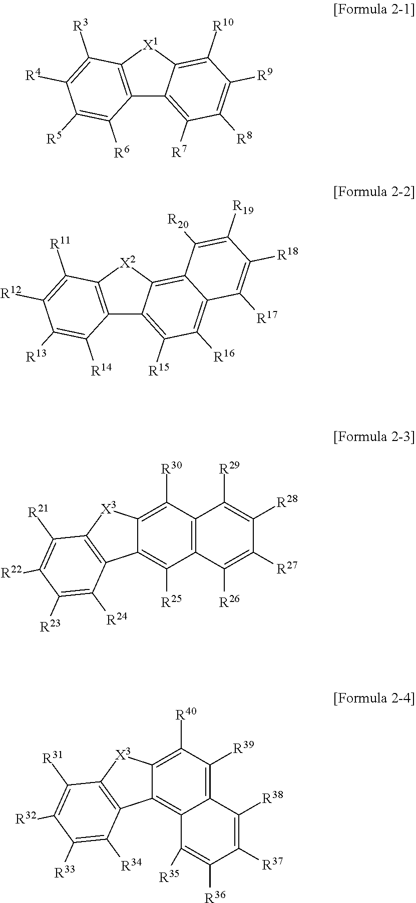
wherein in Formula 2-1 to Formula 2-4,
X¹ to X⁴¹ are each independently O, S, or N(R⁴¹),
R³ to R¹⁰ are each independently a hydrogen atom, a deuterium atom, or a substituted or unsubstituted aryl group having 6 to 30 ring-forming carbon atoms, and
R¹⁰ to R⁴¹ are each independently a hydrogen atom, a deuterium atom, a substituted or unsubstituted aryl group having 6 to 30 ring-forming carbon atoms, or a substituted or unsubstituted heteroaryl group having 5 to 30 ring-forming carbon atoms, except that:
in Formula 2-1, one of R³ to R¹⁰ and R⁴¹ is a position connected to Formula 1,
in Formula 2-2, one of R¹¹ to R²⁰ and R⁴¹ is a position connected to Formula 1,
in Formula 2-3, one of R²¹ to R³⁰ and R⁴¹ is a position connected to Formula 1, and
in Formula 2-4, one of R³¹ to R⁴¹ is a position connected to Formula 1.

19. The display device of claim 18, further comprising:
an optical control layer that includes a quantum dot.

20. The display device of claim 19, further comprising:
a color filter layer disposed on the optical control layer, wherein
the color filter layer includes:
a first filter that transmits red light;
a second filter that transmits green light; and
a third filter that transmits blue light.

21. The display device of claim 18, wherein the display device is a television set, a monitor, a billboard, a personal computer, a laptop computer, a personal digital terminal, a display device for a vehicle, a game console, a portable electronic apparatus, or a camera.
